(19) United States
(12) Patent Application Publication
(10) Pub. No.: US 2007/0111995 A1
(43) Pub. Date: May 17, 2007

(54) Pyrazolo [3,4-B] pyridine Compounds and Their Use as Phosphodiesterase Inhibitors

(76) Inventor: David George Allen, Hertfordshire (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/596,561

(22) PCT Filed: Dec. 17, 2004
(86) PCT No.: PCT/EP04/14490

§ 371 Date: Jun. 16, 2006

(30) Foreign Application Priority Data
Dec. 19, 2003 (EP) PCT/EP03/14867
Mar. 16, 2004 (GB) 0405899.6
Mar. 16, 2004 (GB) 0405936.6
Mar. 25, 2004 (GB) 0406754.2

(51) Int. Cl. A61K 31/541; A61K 31/5377; A61K 31/496; A61K 31/4745; C07D 471/02

(52) U.S. Cl. 514/227.8; 514/303; 514/234.2; 514/253.04; 544/60; 544/126; 544/362; 546/119

(57) ABSTRACT

The invention provides pyrazolo[3,4-b]pyridine compounds of formula (I) having a —C(O)—NH—C(R⁴)(R⁵)-aryl substituent at the 5-position of the pyrazolo[3,4-b]pyridine ring system wherein at least one of R⁴ and R⁵ is not a hydrogen atom (H) compound or a salt thereof: 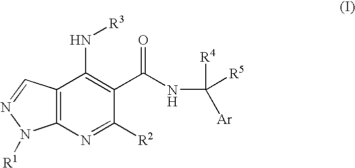
wherein Ar has the sub-formula (x) or (z). 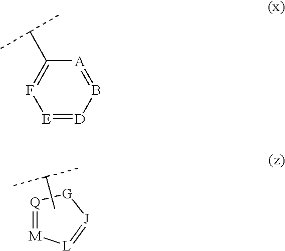 These compounds are useful as inhibitors of PDE4.

The present invention relates to pyrazolo[3,4-b]pyridine compounds, processes for their preparation, intermediates usable in these processes, and pharmaceutical compositions containing the compounds. The invention also relates to the use of the pyrazolo[3,4-b]pyridine compounds in therapy, for example as inhibitors of phosphodiesterase type IV (PDE4) and/or for the treatment and/or prophylaxis of inflammatory and/or allergic diseases such as chronic obstructive pulmonary disease (COPD), asthma, rheumatoid arthritis, allergic rhinitis or atopic dermatitis.

### BACKGROUND OF THE INVENTION

U.S. Pat. No. 3,979,399, U.S. Pat. No. 3,840,546, and U.S. Pat. No. 3,966,746 (E. R. Squibb & Sons) disclose 4-amino derivatives of pyrazolo[3,4-b]pyridine-5-carboxamides wherein the 4-amino group NR₃R₄ can be an acyclic amino group wherein R₃ and R₄ may each be hydrogen, lower alkyl (e.g. butyl), phenyl, etc.; NR₃R₄ can alternatively be a 3-6-membered heterocyclic group such as pyrrolidino, piperidino and piperazino. The compounds are disclosed as central nervous system depressants useful as ataractic, analgesic and hypotensive agents.

U.S. Pat. No. 3,925,388, U.S. Pat. No. 3,856,799, U.S. Pat. No. 3,833,594 and U.S. Pat. No. 3,755,340 (E. R. Squibb & Sons) disclose 4-amino derivatives of pyrazolo[3,4-b]pyridine-5-carboxylic acids and esters. The 4-amino group NR₃R₄ can be an acyclic amino group wherein R₃ and R₄ may each be hydrogen, lower alkyl (e.g. butyl), phenyl, etc.; NR₃R₄ can alternatively be a 5-6-membered heterocyclic group in which an additional nitrogen is present such as pyrrolidino, piperidino, pyrazolyl, pyrimidinyl, pyridazinyl or piperazinyl. The compounds are mentioned as being central nervous system depressants useful as ataractic agents or tranquilisers, as having antiinflammatory and analgesic properties. The compounds are mentioned as increasing the intracellular concentration of adenosine-3′,5′-cyclic monophosphate and for alleviating the symptoms of asthma.

H. Hoehn et al., J. Heterocycl. Chem., 1972, 9(2), 235-253 discloses a series of 1H-pyrazolo[3,4-b]pyridine-5-carboxylic acid derivatives with 4-hydroxy, 4-chloro, 4-alkoxy, 4-hydrazino, and 4-amino substituents.

CA 1003419, CH 553 799 and T.Denzel, Archiv der Pharmazie, 1974, 307(3), 177-186 disclose 4,5-disubstituted 1H-pyrazolo[3,4-b]pyridines unsubstituted at the 1-position.

Japanese laid-open patent application JP-2002-20386-A (Ono Yakuhin Kogyo KK) published on 23 Jan. 2002 discloses pyrazolopyridine compounds of the following formula:

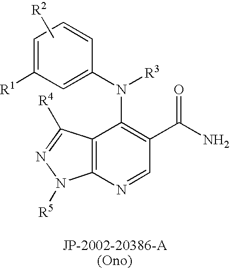

wherein R¹ denotes 1) a group —OR⁶, 2) a group —SR⁷, 3) a C2-8 alkynyl group, 4) a nitro group, 5) a cyano group, 6) a C1-8 alkyl group substituted by a hydroxy group or a C1-8 alkoxy group, 7) a phenyl group, 8) a group —C(O)R⁸, 9) a group —SO₂NR⁹R¹⁰, 10) a group —NR¹¹SO₂R¹², 11) a group —NR¹³C(O)R¹⁴ or 12) a group —CH═NR¹⁵. R⁶ and R⁷ denote i) a hydrogen atom, ii) a C1-8 alkyl group, iii) a C1-8 alkyl group substituted by a C1-8 alkoxy group, iv) a trihalomethyl group, v) a C3-7 cycloalkyl group, vi) a C1-8 alkyl group substituted by a phenyl group or vii) a 3-15 membered mono-, di- or tricyclic hetero ring containing 1-4 nitrogen atoms, 1-3 oxygen atoms and/or 1-3 sulphur atoms. R² denotes 1) a hydrogen atom or 2) a C1-8 alkoxy group. R³ denotes 1) a hydrogen atom or 2) a C1-8 alkyl group. R⁴ denotes 1) a hydrogen atom, 2) a C1-8 alkyl group, 3) a C3-7 cycloalkyl group, 4) a C1-8 alkyl group substituted by a C3-7 cycloalkyl group, 5) a phenyl group which may be substituted by 1-3 halogen atoms or 6) a 3-15 membered mono-, di- or tricyclic hetero ring containing 1-4 nitrogen atoms, 1-3 oxygen atoms and/or 1-3 sulphur atoms. Rs denotes 1) a hydrogen atom, 2) a C1-8alkyl group, 3) a C3-7 cycloalkyl group, 4) a C1-8 alkyl group substituted by a C3-7 cycloalkyl group or 5) a phenyl group which may be substituted by 1-3 substituents. In group R³, a hydrogen atom is preferred. In group R⁴, methyl, ethyl, cyclopropyl, cyclobutyl or cyclopentyl are preferred. The compounds of JP-2002-20386-A are stated as having PDE4 inhibitory activity and as being useful in the prevention and/or treatment of inflammatory diseases and many other diseases.

1,3-Dimethyl-4-(arylamino)-pyrazolo[3,4-b]pyridines with a 5-C(O)NH₂ substituent similar or identical to those in JP-2002-20386-A were disclosed as orally active PDE4 inhibitors by authors from Ono Pharmaceutical Co. in: H. Ochiai et al., Bioorg. Med. Chem. Lett., 5th Jan. 2004 issue, vol. 14(1), pp. 29-32 (available on or before 4th Dec. 2003 from the Web version of the journal: “articles in press”). Full papers on these and similar compounds as orally active PDB4 inhibitors are: H. Ochiai et al., Bioorg. Med. Chem., 2004, 12, 4089-4100 (available online 20 Jun. 2004), and H. Ochiai et al., Chem. Pharm. Bull., 2004, 52(9), 1098-1104 (available online 15 Jun. 2004).

EP 0 076 035 A1 (ICI Americas) discloses pyrazolo[3,4-b]pyridine derivatives as central nervous system depressants useful as tranquilisers or ataractic agents for the relief of anxiety and tension states.

The compound cartazolate, ethyl 4-(n-butylamino)-1-ethyl-1H-pyrazolo[3,4-b]-pyridine-5-carboxylate, is known. J. W. Daly et al., Med. Chem. Res., 1994, 4, 293-306 and D. Shi et al., Drug Development Research, 1997, 42, 41-56 disclose a series of 4-(amino)substituted 1H-pyrazolo[3,4-b]pyridine-5-carboxylic acid derivatives, including ethyl 4-cyclopentylamino-1-methyl-1H-pyrazolo[3,4-b]pyridine-5-carboxylate, and their affinities and antagonist activities at A₁- and A_{2A}-adenosine receptors, and the latter paper discloses their affinities at various binding sites of the GABA_{A}-receptor channel. S. Schenone et al., Bioorg. Med. Chem. Lett., 2001, 11, 2529-2531, and F. Bondavalli et al., J. Med. Chem., 2002, vol. 45 (Issue 22, 24 Oct. 2002, allegedly published on Web Sep. 24, 2002), pp. 4875-4887 disclose a series of 4-amino-1-(2-chloro-2-phenylethyl)-1H-pyrazolo[3,4-b]pyridine-5-carboxylic acid ethyl esters as A₁-adenosine receptor ligands.

WO 02/060900 A2 appears to disclose, as MCP-1 antagonists for treatment of allergic, inflammatory or autoimmune disorders or diseases, a series of bicyclic heterocyclic compounds with a —C(O)—NR⁴—C(O)—NR⁵R⁶ substituent, including isoxazolo[5,4-b]pyridines and 1H-pyrazolo[3,4-b]pyridines (named as pyrazolo[5,4-b]pyridines) with the —C(O)—NR⁴—C(O)—NR⁵R⁶ group as the 5-substituent and optionally substituted at the 1-, 3-, 4-, and/or 6-positions. Bicyclic heterocyclic compounds with a —C(O)NH₂ substituent instead of the —C(O)—NR⁴—C(O)—NR⁵R⁶ substituent are alleged to be disclosed in WO 02/060900 as intermediates in the synthesis of the —C(O)—NR⁴—C(O)—NR⁵R⁶ substituted compounds.

WO 00/15222 (Bristol-Myers Squibb) discloses inter alia pyrazolo[3,4-b]pyridines having inter alia a C(O)—X₁ group at the 5-position and a group E₁ at the 4-position of the ring system. Amongst other things, X₁ can for example be —OR₉, —N(R₉)(R₁₀) or —N(R₅)(-A₂-R₂), and E₁ can for example be —NH-A₁-cycloalkyl, —NH-A₁-substituted cycloalkyl, or —NH-A₁-heterocyclo; wherein A₁ is an alkylene or substituted alkylene bridge of 1 to 10 carbons and A₂ can for example be a direct bond or an alkylene or substituted alkylene bridge of 1 to 10 carbons. The compounds are disclosed as being useful as inhibitors of cGMP phosphodiesterase, especially PDE type V, and in the treatment of various cGMP-associated conditions such as erectile dysfunction. Compounds with a cycloalkyl or heterocyclo group directly attached to —NH— at the 4-position of the pyrazolo[3,4-b]pyridine ring system and/or having PDE4 inhibitory activity do not appear to be disclosed in WO 00/15222.

H. de Mello, A. Echevarria, et al., J. Med. Chein., 2004, believed to be published online on or just before 21 Sep. 2004, discloses 3-methyl or 3-phenyl 4-amino-1H-pyrazolo[3,4-b]pyridine 5-carboxylic esters as potential anti-Leishmania drugs.

Copending patent application PCT/EP2003/014867, filed on 19 Dec. 2003 in the name of Glaxo Group Limited, published on 8 Jul. 2004 as WO 2004/056823 A1, and incorporated herein by reference, discloses and claims pyrazolo[3,4-b]pyridine compounds or salts thereof with a 4-NR³R^{3a} group (R^{3a} is preferably H) and with a group Het at the 5-position of the pyrazolo[3,4-b]pyridine, wherein Het is usually a 5-membered optionally substituted heteroaryl group. PCT/EP2003/014867 also discloses the use of these compounds as PDE4 inhibitors and for the treatment and/or prophylaxis of inter alia COPD, asthma or allergic rhinitis. In “Process F”, on page 58 line 14 to page 59 line 18 of PCT/EP2003/014867 (this passage, plus all definitions elsewhere therein of all compounds, groups and/or substituents mentioned in this passage, being specifically incorporated herein by reference), a compound of general Formula XXVIII:

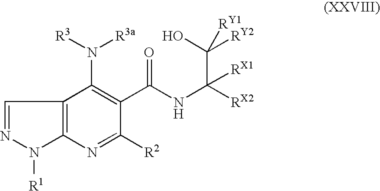

is disclosed for use as an intermediate in the synthesis of a subset of the 5-Het pyrazolo[3,4-b]pyridine compounds claimed in PCT/EP2003/014867 wherein Het is optionally substituted 1,3-oxazol-2-yl. Intermediates 42, 43 and 46 within PCT/EP2003/014867 (WO 2004/056823 A1) also disclose embodiments of the compound of Formula XXVIII as intermediate compounds intended for use in the synthesis of the Examples within PCT/EP2003/014867.

Priority is claimed in the present patent application from PCT/EP2003/014867 filed on 19 Dec. 2003, in particular relying on the above-mentioned passages disclosing a compound of Formula XXVIII wherein R^{3a} is preferably H.

Copending patent application PCTIEP03/11814, filed on 12 Sep. 2003 in the name of Glaxo Group Limited, published on 25 Mar. 2004 as WO 2004/024728 A2, and incorporated herein by reference, discloses pyrazolo[3,4-b]pyridine compounds or salts thereof with a 4-NHR³ group and a 5-C(O)—X group, according to this formula (I):

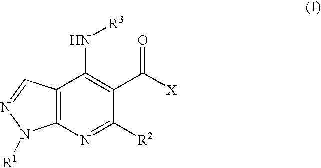

wherein:

R¹ is C₁₋₄alkyl, C₁₋₃fluoroalkyl, —CH₂CH₂OH or —CH₂CH₂CO₂C₁₋₂alkyl;

R² is a hydrogen atom (H), methyl or C₁fluoroalkyl;

R³ is optionally substituted C₃₋₈cycloalkyl or optionally substituted mono-unsaturated-C₅₋₇cycloalkenyl or an optionally substituted heterocyclic group of sub-formula (aa), (bb) or (cc);

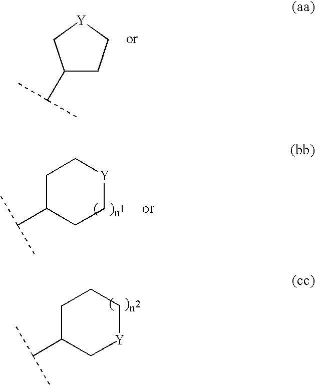

in which n¹ and n² independently are 1 or 2; and in which Y is O, S, SO₂, or NR¹⁰;

or R³ is a bicyclic group (dd) or (ee):

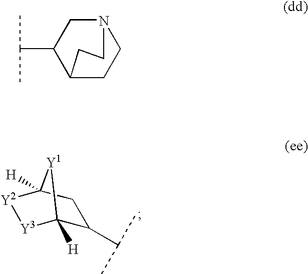

and wherein X is NR⁴R⁵ or OR^{5a}.

In PCT/EP03/11814 (WO 2004/024728 A2), R⁴ is a hydrogen atom (H); C₁₋₆alkyl; C₁₋₃fluoroalkyl; or C₂₋₆alkyl substituted by one substituent R¹¹.

In PCT/EP03/11814 (WO 2004/024728 A2), R⁵ can be: a hydrogen atom (H); C₁₋₈alkyl; C₁₋₈ fluoroalkyl; C₃₋₈cycloalkyl optionally substituted by a C₁₋₂alkyl group; —(CH₂)ₙ⁴-C₃₋₈-cycloalkyl optionally substituted, in the —(CH₂)ₙ⁴-moiety or in the C₃₋₈-cycloalkyl moiety, by a C₁₋₂alkyl group, wherein n⁴ is 1, 2 or 3; C₂₋₆alkyl substituted by one or two independent substituents R¹¹; —(CH₂)ₙ¹¹—C(O)R¹⁶; —(CH₂)ₙ¹²—C(O)NR¹²R¹³; —CHR¹⁹—C(O)NR¹²R¹³; —(CH₂)ₙ¹²—C(O)OR¹⁶; —(CH₂)ₙ¹²—C(O)OH; —CHR¹⁹—C(O)OR¹⁶; —CHR¹⁹—C(O)OH; —(CH₂)ₙ¹²—SO₂—NR¹²R¹³; —(CH₂)ₙ¹²—SO₂R¹⁶; or —(CH₂)ₙ¹²—CN; —(CH₂)ₙ¹³-Het; or optionally substituted phenyl.

Alternatively, in PCT/EP03/11814 (WO 2004/024728 A2), R⁵ can have the sub-formula (x), (y), (y1) or (z):

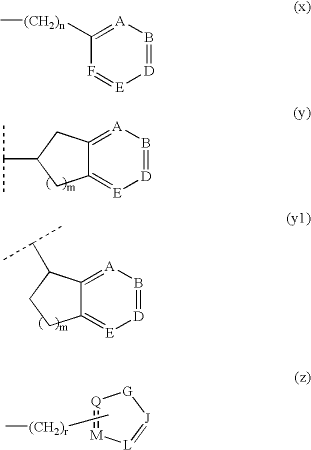

wherein in sub-formula (x), n=0, 1 or 2; in sub-formula (y) and (y1), m=1 or 2; and in sub-formula (z), r=0, 1 or 2; and wherein in sub-formula (x) and (y) and (y1), none, one or two of A, B, D, E and F are independently nitrogen or nitrogen-oxide (N⁺—O⁻) provided that no more than one of A, B, D, E and F is nitrogen-oxide, and the remaining of A, B, D, E and F are independently CH or CR⁶; and provided that when n is 0 in sub-formula (x) then one or two of A, B, D, E and F are independently nitrogen or nitrogen-oxide (N⁺—O⁻) and no more than one of A, B, D, E and F is nitrogen-oxide;

In PCT/EP03/11814 (WO 2004/024728 A2), each R⁶, independently of any other R⁶ present, is: a halogen atom; C₁₋₆alkyl; C₁₋₄fluoroalkyl; C₁₋₄alkoxy; C₁₋₂fluoroalkoxy; C₃₋₆cycloalkyloxy; —C(O)R^{16a}; —C(O)OR³⁰; —S(O)₂—R^{16a}; R^{16a}—S(O)₂—NR^{15a}—; R⁷R⁸N—S(O)₂—; C₁₋₂alkyl-C(O)—R^{15a}N—S(O)₂-; C₁₋₄alkyl-S(O)—; Ph-S(O)—; R⁷R⁸N—CO—; —NR¹⁵—C(O)R¹⁶; R⁷R⁸N; OH; C₁₋₄alkoxymethyl; C₁₋₄alkoxyethyl; C₁₋₂alkyl-S(O)₂—CH₂—; R⁷R⁸N—S(O)₂—CH₂—; C₁₋₂alkyl-S(O)₂—NR^{15a}—CH₂—; —CH₂—OH; —CH₂CH₂—OH; —CH₂—NR⁷R⁸; —CH₂—CH₂—NR⁷R⁸; —CH₂—C(O)OR⁺; —CH₂—C(O)—NR⁷R⁸; —CH₂—NR^{15a}—(CO)—C₁₋₃alkyl; —(CH₂)ₙ¹⁴-Het¹ where n¹⁴ is 0 or 1; cyano (CN); Ar^{5b}; or phenyl, pyridinyl or pyrimidinyl wherein the phenyl, pyridinyl or pyrimidinyl independently are optionally substituted by one or two of fluoro, chloro, C₁₋₂alkyl, C₁fluoroalkyl, C₁₋₂alkoxy or C₁fluoroalkoxy;
or two adjacent R⁶ taken together can be —O—(CMe₂)-O— or —O—(CH₂)ₙ¹⁴—O— where n¹⁴ is 1 or 2.

In PCT/EP03/11814 (WO 2004/024728 A2), in sub-formula (z), G is O or S or NR⁹ wherein R⁹ is a hydrogen atom (H), C₁₋₄alkyl or C₁₋₄fluoroalkyl; none, one, two or three of J, L, M and Q are nitrogen; and the remaining of J, L, M and Q are independently CH or CR⁶ where R⁶, independently of any other R⁶ present, is as defined therein.

The pyrazolo[3,4-b]pyridine compounds of formula (I) and salts thereof disclosed in PCT/EP03/11814 (WO 2004/024728 A2) are disclosed as being inhibitors of phosphodiesterase type IV (PDE4), and as being useful for the treatment and/or prophylaxis of an inflammatory and/or allergic diseases such as chronic obstructive pulmonary disease (COPD), asthma, rheumatoid arthritis, or allergic rhinitis.

### THE INVENTION

We have now found new pyrazolo[3,4-b]pyridine compounds, having a —C(O)—NH—C(R⁴)(R⁵)-aryl substituent at the 5-position of the pyrazolo[3,4-b]pyridine ring system wherein at least one of R⁴ and R⁵ is not a hydrogen atom (H), which compounds inhibit phosphodiesterase type IV (PDE4).

The present invention therefore provides a compound of formula (I) or a salt thereof (in particular, a pharmaceutically acceptable salt thereof):

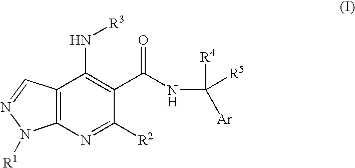

wherein Ar has the sub-formula (x) or (z):

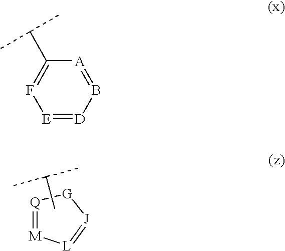

and wherein:

R¹ is C₁₋₃alkyl, C₁₋₃fluoroalkyl, or —CH₂CH₂OH;

R² is a hydrogen atom (H), methyl or C₁fluoroalkyl;

R³ is optionally substituted C₃₋₈cycloalkyl or optionally substituted mono-unsaturated-C₅₋₇cycloalkenyl or an optionally substituted heterocyclic group of sub-formula (aa), (bb) or (cc);

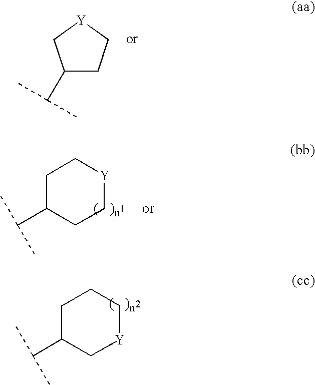

in which n¹ and n² independently are 1 or 2; and in which Y is O, S, SO₂, or NR¹⁰; where R¹⁰ is a hydrogen atom (H), C₁₋₂alkyl, C₁₋₂fluoroalkyl, C(O)NH₂, C(O)—C₁₋₂alkyl, C(O)—C₁fluoroalkyl or —C(O)—CH₂O—C₁alkyl;

and wherein in R³ the C₃₋₈cycloalkyl or the heterocyclic group of sub-formula (aa), (bb) or (cc) is optionally substituted on a ring carbon with one or two substituents independently being oxo (═O); OH; C₁₋₂alkoxy; C₁₋₂fluoroalkoxy; NHR²¹ wherein R²¹ is a hydrogen atom (H) or C₁₋₄ straight-chain alkyl; C₁₋₂alkyl; C₁₋₂fluoroalkyl; —CH₂OH; —CH₂CH₂OH; —CH₂NHR²² wherein R²² is H or C₁alkyl; —C(O)OR²³ wherein R²³ is H; —C(O)NHR²⁴ wherein R²⁴ is H or C₁alkyl; —C(O)R²⁵ wherein R²⁵ is C₁₋₂alkyl; fluoro; hydroxyimino (═N—OH); or (C₁₋₄alkoxy)imino (═N—OR²⁶ where R²⁶ is C₁₋₄alkyl); and wherein any OH, alkoxy, fluoroalkoxy or NHR²¹ substituent is not substituted at the R³ ring carbon attached (bonded) to the —NH— group of formula (I) and is not substituted at either R³ ring carbon bonded to the Y group of the heterocyclic group (aa), (bb) or (cc);

and wherein, when R³ is optionally substituted mono-unsaturated-C₅₋₇cycloalkenyl, then the cycloalkenyl is optionally substituted with one substituent being fluoro or C₁₋₂alkyl or two substituents independently being fluoro or methyl, and the R³ ring carbon bonded to the —NH— group of formula (I) does not partake in the cycloalkenyl double bond;

or R³ is a bicyclic group of sub-formula (ee):

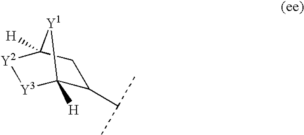

(ee) wherein Y¹, Y² and Y³ independently are CH₂ or oxygen (O) provided that no more than one of Y¹, Y² and Y³ is oxygen (O);

and wherein:

R⁴ is a hydrogen atom (H), methyl, ethyl, n-propyl, isopropyl, C₁₋₂fluoroalkyl, cyclopropyl, —CH₂OR^{4a}, —CH(Me)OR^{4a}, or —CH₂CH₂OR^{4a}; wherein R^{4a} is a hydrogen atom (H), methyl (Me), or C₁fluoroalkyl such as CF₃ or CHF₂; and

R⁵ is a hydrogen atom (H); C₁₋₈alkyl (e.g. C₁₋₆alkyl or C₁₋₄alkyl); C₁₋₃fluoroalkyl; C₃₋₈cycloalkyl optionally substituted by a C₁₋₂alkyl group; or —(CH₂)ₙ⁴—C₃₋₈cycloalkyl optionally substituted, in the —(CH₂)ₙ⁴— moiety or in the C₃₋₈cycloalkyl moiety, by a C₁₋₂alkyl group, wherein n⁴ is 1 or 2;

or R⁵ is C₁₋₄alkyl substituted by one substituent R¹¹; wherein R¹¹ is: hydroxy (OH); C₁₋₆alkoxy; C₁₋₂fluoroalkoxy; phenyloxy; (monofluoro- or difluoro-phenyl)oxy; (monomethyl- or dimethyl-phenyl)oxy; benzyloxy; —NR¹²R¹³; —NR¹⁵—C(O)R¹⁶; —NR¹⁵—C(O)—NH—R¹⁵; or —NR¹⁵—S(O)₂R¹⁶;

or R⁵ is C₂₋₄alkyl substituted on different carbon atoms by two hydroxy (OH) substituents;

or R⁵ is —(CH₂)ₙ¹¹—C(O)R¹⁶; —(CH₂)ₙ¹¹—C(O)NR¹²R¹³; —CHR¹⁹—C(O)NR¹²R¹³; —(CH₂)ₙ¹¹—C(O)OR¹⁶; —(CH₂)ₙ¹¹—C(O)OH; —CHR¹⁹—C(O)OR¹⁶; —CHR¹⁹—C(O)OH; —(CH₂)ₙ¹¹—S(O)₂—NR¹²R¹³; —(CH₂)ₙ¹¹—S(O)₂R¹⁶; or —(CH₂)ₙ¹¹—CN; wherein n¹¹ is 0 1, 2 or 3 (wherein for each R⁵ group n¹¹ is independent of the value of n¹¹ in other R⁵ groups); and wherein R¹⁹ is C₁₋₂alkyl;

or R⁵ is —(CH₂)ₙ¹³-Het, wherein n¹³ is 0, 1 or 2 and Het is a 4-, 5-, 6- or 7-membered saturated or unsaturated heterocyclic ring, other than —NR¹²R¹³, containing one or two ring-hetero-atoms independently selected from O, S, and N; wherein any ring-hetero-atoms present are not bound to the —(CH₂)ₙ¹³— moiety when n¹³ is 0; wherein any ring-nitrogens which are present and which are not unsaturated (i.e. which do not partake in a double bond) and which are not connecting nitrogens (i.e. which are not nitrogens bound to the —(CH₂)ₙ¹³— moiety or to the carbon atom to which R⁵ is attached) are present as NR¹⁷; and wherein one or two of the carbon ring-atoms are independently optionally substituted by C₁₋₂alkyl;

or R⁵ is phenyl (Ph), —CH₂—Ph, -CHMe-Ph, -CHEt-Ph, CMe₂Ph, or —CH₂CH₂-Ph, wherein the phenyl ring Ph is optionally substituted with one or two substituents independently being: a halogen atom; C₁₋₄alkyl (e.g. C₁₋₂alkyl); C₁₋₂fluoroalkyl (e.g. trifluoromethyl); C₁₋₄alkoxy (e.g. C₁₋₂alkoxy); C₁₋₂fluoroalkoxy (e.g. trifluoromethoxy or difluoromethoxy); cyclopropyl; cyclopropyloxy; —C(O)—C₁₋₄alkyl; —C(O)OH; —C(O)—OC₁₋₄alkyl; C₁₋₄alkyl-S(O)₂—; C₁₋₄alkyl-S(O)₂—NR^{8a}—; R^{7a}R^{8a}N—S(O)₂—; R^{7a}R^{8a}N—C(O)—; —NR^{8a}—C(O)—C₁₋₄alkyl; R^{7a}R^{8a}N; OH; nitro (—NO₂); or cyano (—CN);

or R⁴ and R⁵ taken together are —(CH₂)ₚ¹— or —(CH₂)ₚ³—X⁵—(CH₂)ₚ⁴—, in which: X⁵ is O or NR^{17a}; p¹=2, 3, 4, 5 or 6, and p³ and p⁴ independently are 1, 2 or 3 provided that if p³ is 3 then p⁴ is 1 or 2 and if p⁴ is 3 then p³ is 1 or 2;

provided that at least one of R⁴ and R⁵ is not a hydrogen atom (H);

and wherein, in sub-formula (x):

A is C—R^{6A}, nitrogen (N) or nitrogen-oxide (N⁺—O⁻),

B is C—R^{6B}, nitrogen (N) or nitrogen-oxide (N⁺—O⁻),

D is C—R^{6D}, nitrogen (N) or nitrogen-oxide (N⁺—O⁻),

E is C—R^{6E}, nitrogen (N) or nitrogen-oxide (N⁺—O⁻),

F is C—R^{6F}, nitrogen (N) or nitrogen-oxide (N⁺—O⁻),

wherein, R^{6A}, R^{6B}, R^{6D}, R^{6E} and R^{6F} independently are: a hydrogen atom (H), a halogen atom; C₁₋₆alkyl (e.g. C₁₋₄alkyl or C₁₋₂alkyl); C₁₋₄fluoroalkyl (e.g. C₁₋₂fluoroalkyl); C₃₋₆cycloalkyl; C₁₋₄alkoxy (e.g. C₁₋₂alkoxy); C₁₋₂fluoroalkoxy; C₃₋₆cycloalkyloxy; —C(O)R^{16a}; —C(O)OR³⁰; —S(O)₂—R^{16a} (e.g. C₁₋₂alkyl-S(O)₂—); R^{16a}—S(O)₂—NR^{15a}— (e.g. C₁₋₂alkyl-S(O)₂—NH—); R⁷R⁸N—S(O)₂—; C₁₋₂alkyl-C(O)—R^{15a}N—S(O)₂—; C₁₋₄alkyl-S(O)—, Ph—S(O)—, R⁷R⁸N—CO—; —NR^{15a}—C(O)R^{16a}; R⁷R⁸N; nitro (—NO₂); OH (including any tautomer thereof); C₁₋₄alkoxymethyl; C₁₋₄alkoxyethyl; C₁₋₂alkyl-S(O)₂—CH₂—; R⁷R⁸N—S(O)₂—CH₂—; C₁₋₂alkyl-S(O)₂—NR^{15a}—CH₂—; —CH₂—OH; —CH₂CH₂—OH; —CH₂—NR⁷R⁸; —CH₂—CH₂—NR⁷R⁸; —CH₂'C(O)OR³⁰; —CH₂—C(O)—NR⁷R⁸; —CH₂—NR^{15a}—C(O)—C₁₋₃alkyl; —(CH₂)ₙ¹⁴-Het¹ where n¹⁴ is 0 or 1; cyano (—CN); Ar^{5b}; or phenyl, pyridinyl or pyrimidinyl wherein the phenyl, pyridinyl or pyrimidinyl independently are optionally substituted by one or two of fluoro, chloro, C₁₋₂alkyl, C₁fluoroalkyl, C₁₋₂alkoxy or C₁fluoroalkoxy;

and/or two adjacent groups selected from R^{6A}, R^{6B}, R^{6D}, R^{6E} and R^{6F} are taken together and are: —CH═CH—CH═CH—, —CH₂)ₙ^{14a}- where n^{14a} is 3, 4 or 5 (e.g. 3 or 4), —O—(CMe₂)—O—, —O(CH₂)ₙ^{14b}—O— where n^{14b} is 1 or 2; —CH═CH—NR^{15b}—; —N═CH—NR^{15b}—; —CH═N—NR^{15b}—; —N═N—NR^{15b}—; —CH═CH—O—; —N═CH—O—; —CH═CH—S—; or —N═CH—S—; wherein R^{15b} is H or C₁₋₂alkyl;

provided that:

two or more of A, B, D, E and F are independently C—H (carbon-hydrogen), C—F (carbon-fluorine), nitrogen (N), or nitrogen-oxide (N⁺—O⁻);

and no more than two of A, B, D, E and F are independently nitrogen or nitrogen-oxide (N⁺—O⁻),

and no more than one of A, B, D, E and F is nitrogen-oxide (N⁺—O⁻);

and wherein, in sub-formula (z):

G is O or S or NR⁹ wherein R⁹ is a hydrogen atom (H), C₁₋₄alkyl, or C₁₋₂fluoroalkyl;

J is C—R^{6J}, C-[connection point to formula (I)], or nitrogen (N),

L is C—R^{6L}, C-[connection point to formula (I)], or nitrogen (N),

M is C—R⁶M, C-[connection point to formula (I)], or nitrogen (N),

Q is C—R^{6Q}, C-[connection point to formula (I)], or nitrogen (N),

wherein, R^{6J}, R^{6L}, R^{6M} and R^{6Q} independently are: a hydrogen atom (H), a halogen atom; C₁₋₄alkyl (e.g. C₁₋₂alkyl); C₁₋₃fluoroalkyl (e.g. C₁₋₂fluoroalkyl); C₃₋₆cycloalkyl; C₁₋₄alkoxy (e.g. C₁₋₂alkoxy); C₁₋₂fluoroalkoxy; C₃₋₆cycloalkyloxy; OH (including any tautomer thereof); or phenyl optionally substituted by one or two substituents independently being fluoro, chloro, C₁₋₂alkyl, C₁fluoroalkyl, C₁₋₂alkoxy or C₁fluoroalkoxy;

provided that:

two or more of J, L, M and Q are independently C—H, C—F, C—C₁₋₂alkyl (e.g. C-Me), C-[connection point to formula (I)], or nitrogen (N);

and no more than three of J, L, M and Q are nitrogen (N);

and wherein:

R⁷ and R⁸ are independently a hydrogen atom (H); C₁₋₄alkyl (e.g. C₁₋₂alkyl such as methyl); C₃₋₆cycloalkyl; or phenyl optionally substituted by one or two substituents independently being: fluoro, chloro, C₁₋₂alkyl, C₁fluoroalkyl, C₁₋₂alkoxy or C₁fluoroalkoxy;

or R⁷ and R⁸ together are —(CH₂)ₙ⁶— or —C(O)—(CH₂)ₙ⁷— or —C(O)—(CH₂)ₙ¹⁰—C(O)— or —(CH₂)ₙ⁸—X⁷—(CH₂)ₙ⁹— or —C(O)—X⁷—(CH₂)ₙ¹⁰— in which: n⁶ is 3, 4, 5 or 6, n⁷ is 2, 3, 4, or 5, n⁸ and n⁹ and n¹⁰ independently are 2 or 3, and X⁷ is O or NR¹⁴;

R^{7a} is a hydrogen atom (H) or C₁₋₄alkyl;

R^{8a} is a hydrogen atom (H) or methyl;

R¹² and R¹³ independently are H; C₁₋₄alkyl (e.g. C₁₋₂alkyl); C₃₋₆cycloalkyl; or phenyl optionally substituted by one or two substituents independently being: fluoro, chloro, C₁₋₂alkyl, C₁fluoroalkyl, C₁₋₂alkoxy or C₁fluoroalkoxy;

or R¹² and R¹³ together are —(CH₂)ₙ^{6a}— or —C(O)—(CH₂)ₙ^{7a}— or —C(O)—(CH₂)ₙ^{10a}—C(O)— or —(CH₂)ₙ^{8a}—X¹²—(CH₂)ₙ^{9a}— or —C(O)—X¹²—(CH₂)ₙ^{10a}— in which: n^{6a} is 3, 4, 5 or 6, n^{7a} is 2, 3, 4, or 5, n^{8a} and n^{9a} and n^{10a} independently are 2 or 3 and X¹² is O or NR^{14a};

R¹⁴, R^{14a}, R¹⁷ and R^{17a} independently are: a hydrogen atom (H); C₁₋₄alkyl (e.g. C₁₋₂alkyl); C₁₋₂fluoroalkyl (e.g. CF₃); cyclopropyl; —C(O)—C₁₋₄alkyl (e.g. —C(O)Me); —C(O)NR^{7a}R^{8a} (e.g. —C(O)NH₂); or —S(O)₂—C₁₋₄alkyl (e.g. —S(O)₂Me);

R¹⁵, independent of other R¹⁵, is a hydrogen atom (H); C₁₋₄alkyl (e.g. ^{t}Bu or C₁₋₂alkyl e.g. methyl); C₃₋₆cycloalkyl; or phenyl optionally substituted by one or two of: a halogen atom, C₁₋₂alkyl, C₁fluoroalkyl, C₁₋₂alkoxy or C₁fluoroalkoxy;

R^{15a}, independent of other R^{15a}, is a hydrogen atom (H) or C₁₋₄alkyl;

R¹⁶ is: C₁₋₄alkyl (e.g. C₁₋₂alkyl); C₃₋₆cycloalkyl (e.g. C₅₋₆cycloalkyl); C₃₋₆cycloalkyl-CH₂- (e.g. C₅₋₆cycloalkyl-CH₂—); or phenyl or benzyl, wherein the phenyl and benzyl are independently optionally substituted on their ring by one or two substituents independently being fluoro, chloro, methyl, C fluoroalkyl, methoxy or C₁ fluoroalkoxy;

R^{16a} is:

C₁₋₆alkyl (e.g. C₁₋₄alkyl or C₁₋₂alkyl);

C₃₋₆cycloalkyl (e.g. C₅₋₆cycloalkyl) optionally substituted by one oxo (═O), OH or C₁₋₂alkyl substituent (e.g. optionally substituted at the 3- or 4-position of a

C₅₋₆cycloalkyl ring; and/or preferably unsubstituted C₃₋₆cycloalkyl);

C₃₋₆cycloalkyl-CH₂— (e.g. C₅₋₆cycloalkyl-CH₂—);

pyridinyl (e.g. pyridin-2-yl) optionally substituted on a ring carbon atom by one of: a halogen atom, C₁₋₂alkyl, C₁fluoroalkyl, C₁₋₂alkoxy or C₁fluoroalkoxy;

Ar^{5c};

phenyl optionally substituted by one or two substituents independently being: a halogen atom, C₁₋₂alkyl, C₁fluoroalkyl, C₁₋₂alkoxy or C₁fluoroalkoxy;

benzyl optionally substituted on its ring by one or two substituents independently being: a halogen atom, C₁₋₂alkyl, C₁fluoroalkyl, C₁₋₂alkoxy or C₁fluoroalkoxy; or

a 4-, 5-, 6- or 7-membered saturated heterocyclic ring connected at a ring-carbon and containing one or two ring-hetero-atoms independently selected from O, S, and N;

wherein any ring-nitrogens which are present are present as NR²⁷ where R²⁷ is H, C₁₋₂alkyl or —C(O)Me; and wherein the ring is optionally substituted at carbon by one C₁₋₂alkyl or oxo (═O) substituent, provided that any oxo (═O) substituent is substituted at a ring-carbon atom bonded to a ring-nitrogen;

R³⁰, independent of other R³⁰, is a hydrogen atom (H), C₁₋₄alkyl or C₃₋₆cycloalkyl;

Ar^{5b} and Ar^{5c} independently is/are a 5-membered aromatic heterocyclic ring containing one O, S or NR^{15a} in the 5-membered ring, wherein the 5-membered ring can optionally additionally contain one or two N atoms, and wherein the heterocyclic ring is optionally substituted on a ring carbon atom by one of: a halogen atom, C₁₋₂alkyl, C₁fluoroalkyl, —CH₂OH, —CH₂—OC₁₋₂alkyl, OH (including the keto tautomer thereof) or —CH₂—NR²⁸R²⁹ wherein R²⁸ and R²⁹ independently are H or methyl; and

Het¹, is a 4-, 5-, 6- or 7-membered saturated heterocyclic ring connected at a ring-carbon and containing one or two ring-hetero-atoms independently selected from O, S, and N; wherein any ring-nitrogens which are present are present as NR³¹ where R³¹ is H, C₁₋₂alkyl or —C(O)Me; and wherein the ring is optionally substituted at carbon by one C₁₋₂alkyl or oxo (═O) substituent, provided that any oxo (═O) substituent is substituted at a ring-carbon atom bonded to a ring-nitrogen;

provided that:

when R³ is the heterocyclic group of sub-formula (bb), n¹ is 1, and Y is NR¹⁰, then R¹⁰ is not C₁₋₂alkyl or C₁₋₂fluoroalkyl; and

when R³ is the heterocyclic group of sub-formula (aa) and Y is NR¹⁰, then R¹⁰ is not C(O)—C₁₋₂alkyl, C(O)—C₁fluoroalkyl or —C(O)—CH₂O—C₁alkyl; and

when R³ is the heterocyclic group of sub-formula (cc), then Y is O, S, SO₂ or NR¹⁰ wherein R¹⁰ is H;

and provided that:

when R³ is optionally substituted C₃₋₈cycloalkyl or optionally substituted C₅₋₇cycloalkenyl, then any —C(O)OR²³, —C(O)NHR²⁴, —C(O)R²⁵, —CH₂OH or fluoro substituent is: at the 3-position of a R³ cyclobutyl ring; or at the 3- or 4- position of a R³ C₅cycloalkyl (cyclopentyl) or cyclopentenyl ring; or at the 4-position of a R³ C₆cycloalkyl (cyclohexyl) or cyclohexenyl ring; or at the 3-, 4-, 5- or 6- position of a R³ cycloheptyl or cycloheptenyl ring, or at the 3-, 4-, 5-, 6- or 7- position of a R³ cyclooctyl ring (wherein, in this connection, the 1-position of the R³ cycloalkyl or cycloalkenyl ring is deemed to be the connection point to the —NH— in formula (I), that is the ring atom connecting to the —NH— in formula (I));

and provided that:

when R³ is optionally substituted C₃₋₈cycloalkyl, then any OH, alkoxy, fluoroalkoxy, —CH₂CH₂OH or —CH₂NHR²² substituent is: at the 3-position of a R³ cyclobutyl ring; or at the 3- or 4- position of a R³ C₅cycloalkyl (cyclopentyl) ring; or at the 3-, 4- or 5-position of a R³ C₆cycloalkyl (cyclohexyl) ring; or at the 3-, 4-, 5- or 6- position of a R³ cycloheptyl ring, or at the 3-, 4-, 5-, 6- or 7- position of a R³ cyclooctyl ring; and

when R³ is the heterocyclic group of sub-formula (aa), (bb) or (cc), then any OH substituent is: at the 5-position of a six-membered R³ heterocyclic group of sub-formula (cc) wherein n² is 1; or at the 5- or 6- position of a seven-membered R³ heterocyclic group of sub-formula (cc) wherein n² is 2; or at the 6- position of a seven-membered R³ heterocyclic group of sub-formula (bb) wherein n¹ is 2 (wherein, in this connection, the 1-position of the R³ heterocyclic ring is deemed to be the connection point to the —NH— in formula (I), that is the ring atom connecting to the —NH— in formula (I), and the remaining positions of the ring are then numbered so that the ring heteroatom takes the lowest possible number).

In compounds, for example in the compounds of formula (I) (or formula (IA) or formula (IB), see later), an “alkyl” group or moiety may be straight-chain or branched. Alkyl groups, for example C₁₋₈alkyl or C₁₋₆alkyl or C₁₋₄alkyl or C₁₋₃alkyl or C₁₋₂alkyl, which may be employed include C₁₋₆alkyl or C₁₋₄alkyl or C₁₋₃alkyl or C₁₋₂alkyl such as methyl, ethyl, n-propyl, n-butyl, n-pentyl, or n-hexyl or any branched isomers thereof such as isopropyl, t-butyl, sec-butyl, isobutyl, 3-methylbutan-2-yl, 2-ethylbutan-1-yl, or the like.

A corresponding meaning is intended for “alkoxy”, “alkylene”, and like terms derived from alkyl. For example, “alkoxy” such as C₁₋₆alkoxy or C₁₋₄alkoxy or C₁₋₂alkoxy includes methoxy, ethoxy, propyloxy, and oxy derivatives of the alkyls listed above. “Alkylsulfonyl” such as C₁₋₄alkylsulfonyl includes methylsulfonyl (methanesulfonyl), ethylsulfonyl, and others derived from the alkyls listed above. “Alkylsulfonyloxy” such as C₁₋₄alkylsulfonyloxy includes methanesulfonyloxy (methylsulfonyloxy), ethanesulfonyloxy, et al.

“Cycloalkyl”, for example C₃₋₈cycloalkyl, includes cyclopropyl, cyclobutyl, cyclopentyl, cyclohexyl, cycloheptyl, cyclooctyl, and the like. Suitably, a C₃₋₈cycloalkyl group can be C₃₋₆cycloalkyl or C₅₋₆cycloalkyl or C₄₋₇cycloalkyl or C₆₋₇cycloalkyl, that is contains a 3-6 membered or 5-6 membered or 4-7 membered or 6-7 membered carbocyclic ring.

“Fluoroalkyl” includes alkyl groups with one, two, three, four, five or more fluorine substituents, for example C₁₋₄fluoroalkyl or C₁₋₃fluoroalkyl or C₁₋₂fluoroalkyl such as monofluoromethyl, difluoromethyl, trifluoromethyl, pentafluoroethyl, 2,2,2-trifluoroethyl (CF₃ CH₂—), 2,2-difluoroethyl (CHF₂CH₂—), 2-fluoroethyl (CH₂FCH₂—), etc. “Fluoroalkoxy” includes C₁₋₄fluoroalkoxy or C₁₋₂fluoroalkoxy such as trifluoromethoxy, pentafluoroethoxy, monofluoromethoxy, difluoromethoxy, etc. “Fluoroalkylsulfonyl” such as C₁₋₄fluoroalkylsulfonyl includes trifluoromethanesulfonyl, pentafluoroethylsulfonyl, etc.

A halogen atom (“halo”) present in compounds, for example in the compounds of formula (I), means a fluorine, chlorine, bromine or iodine atom (“fluoro”, “chloro”, “bromo” or “iodo”), for example fluoro, chloro or bromo.

When the specification states that atom or moiety A is “bonded” or “attached” to atom or moiety B, it means that atom/moiety A is directly bonded to atom/moiety B usually by means of a covalent bond or a double covalent bond, and excludes A being indirectly attached to B via one or more intermediate atoms/moieties (e.g. excludes A-C-B); unless it is clear from the context that another meaning is intended.

When R¹ is C₁₋₃alkyl or C₁₋₃fluoroalkyl, it can be straight-chained or branched. Where R¹ is C₁₋₃alkyl then it can be methyl, ethyl, n-propyl, or isopropyl. When R¹ is C₁₋₃fluoroalkyl, then R¹ can for example be C₁fluoroalkyl such as monofluoromethyl, difluoromethyl, trifluoromethyl; or R¹ can be C₂fluoroalkyl such as pentafluoroethyl or more preferably C₁fluoroalkyl-CH₂- such as 2,2,2-trifluoroethyl (CF₃CH₂—), 2,2-difluoroethyl (CHF₂CH₂—), or 2-fluoroethyl (CH₂FCH₂—).

R¹ is C₁₋₃alkyl (e.g. methyl, ethyl or n-propyl), C₁₋₃fluoroalkyl or —CH₂CH₂OH. R¹ is suitably C₁₋₃alkyl, C₁₋₂fluoroalkyl, or —CH₂CH₂OH. Preferably, R¹ is C₂₋₃alkyl (e.g. ethyl or n-propyl), C₂fluoroalkyl (e.g. C₁fluoroalkyl-CH₂— such as CF₃—CH₂—) or —CH₂CH₂OH; in particular ethyl, n-propyl or —CH₂CH₂OH. More preferably, R¹ is C₂alkyl (ethyl) or C₂fluoroalkyl. R¹ is most preferably ethyl.

Preferably, R² is a hydrogen atom (H) or methyl, for example a hydrogen atom (H).

Preferably, in R³ there is one substituent or no substituent.

In one suitable embodiment, R³ is the optionally substituted C₃₋₈cycloalkyl or the optionally substituted heterocyclic group of sub-formula (aa), (bb) or (cc).

In one optional embodiment, when R³ is optionally substituted C₃₋₈cycloalkyl, it is not unsubstituted C₅cycloalkyl, i.e. not unsubstituted cyclopentyl. In this case, suitably, R³ is optionally substituted C₆₋₈cycloalkyl or optionally substituted cyclobutyl.

When R³ is optionally substituted C₃₋₈cycloalkyl, it is more suitably optionally substituted C₆₋₇cycloalkyl or optionally substituted cyclobutyl, preferably optionally substituted C₆cycloalkyl (i.e. optionally substituted cyclohexyl).

Suitably, when R³ is optionally substituted C₃₋₈cycloalkyl, then R³ is C₃₋₈cycloalkyl (e.g. C₆₋₇cycloalkyl or cyclobutyl) optionally substituted with one or two substituents independently being oxo (═O); OH; C alkoxy; C fluoroalkoxy (e.g. trifluoromethoxy or difluoromethoxy); NHR²¹ wherein R²¹ is a hydrogen atom (H) or C₁₋₂alkyl (more preferably R²¹ is H); C₁₋₂alkyl such as methyl; C₁fluoroalkyl such as —CH₂F or —CHF₂;

—CH₂OH; —CH₂NHR²² wherein R²² is H; —C(O)OR²³ wherein R²³ is H; —C(O)NHR²⁴ wherein R²⁴ is H or methyl; —C(O)R²⁵ wherein R²⁵ is methyl; fluoro; hydroxyimino (═N—OH); or (C₁₋₄alkoxy)imino such as (C₁₋₂alkoxy)imino (═N—OR²⁶ where R²⁶ is C₁₋₄alkyl such as C₁₋₂alkyl); and wherein any OH, alkoxy, fluoroalkoxy or NHR²¹ substituent is not substituted at the R³ ring carbon attached (bonded) to the —NH— group of formula (I) and is not substituted at either R³ ring carbon bonded to the Y group of the heterocyclic group (aa), (bb) or (cc).

Preferably, when R³ is optionally substituted C₃₋₈cycloalkyl, then R³ is C₃₋₈cycloalkyl (e.g. C₆₋₇cycloalkyl or cyclobutyl) optionally substituted with one or two substituents independently being oxo (═O); OH; NHR²¹ wherein R²¹ is a hydrogen atom (H); C₁₋₂alkyl such as methyl; C₁fluoroalkyl such as —CH₂F or —CHF₂; —C(O)OR²³ wherein R²³ is H; —C(O)NHR²⁴ wherein R²⁴ is H or methyl (preferably H); —C(O)R²⁵ wherein R²⁵ is methyl; fluoro; hydroxyimino (═N—OH); or (C₁₋₂alkoxy)imino (═N—OR²⁶ where R²⁶ is C₁₋₂alkyl).

More preferably, when R³ is optionally substituted C₃₋₈cycloalkyl, then R³ is C₃₋₈cycloalkyl (e.g. C₆₋₇cycloalkyl or cyclobutyl) optionally substituted with one or two substituents independently being (e.g. one substituent being) oxo (═O); OH; NHR²¹ wherein R²¹ is a hydrogen atom (H); methyl; —CH₂F; —CHF₂; —C(O)OR²³ wherein R²³ is H; —C(O)NHR²⁴ wherein R²⁴ is H or methyl (preferably H); fluoro; hydroxyimino (═N—OH); or methoxyimino (═N—OR²⁶ where R²⁶ is methyl).

Still more preferably, when R³ is optionally substituted C₃₋₈cycloalkyl, then R³ is C₃₋₈cycloalkyl (e.g. C₆₋₇cycloalkyl or cyclobutyl) optionally substituted with one or two substituents independently being (e.g. one substituent being) oxo (═O); OH; methyl; —C(O)NHR²⁴ wherein R²⁴ is H; fluoro; hydroxyimino (═N—OH); or methoxyimino (═N—OR²⁶ where R²⁶ is methyl).

Yet more preferably, when R³ is optionally substituted C₃₋₈cycloalkyl, then R³ is C₃₋₈cycloalkyl (e.g. C₆₋₇cycloalkyl or cyclobutyl) optionally substituted with one or two substituents independently being (e.g. one substituent being) OH; —C(O)NHR²⁴ wherein R²⁴ is H; oxo (═O) or hydroxyimino (═N—OH).

In one optional embodiment, in R³, the C₃₋₈cycloalkyl can be unsubstituted.

When R³ is optionally substituted C₃₋₈cycloalkyl or optionally substituted C₅₋₇cycloalkenyl, e.g. optionally substituted C₅₋₈cycloalkyl or C₅₋₇cycloalkyl, such as optionally substituted C₆cycloalkyl (optionally substituted cyclohexyl) or optionally substituted cyclohexenyl, the one or two optional substituents if present suitably can comprise a substituent (for example is or are substituent(s)) at the 3-, 4- and/or 5-position(s), e.g. at the 3- and/or 4- position(s), of the R³ cycloalkyl or cycloalkenyl ring.

(In this connection and generally herein, the 1-position of the R³ ring, e.g. of the R³ cycloalkyl or cycloalkenyl ring, is deemed to be the connection point to the —NH— in formula (I)=the ring atom connecting to the —NH— in formula (I)).

Suitably, for R³, and in particular when R³ is optionally substituted C₃₋₈cycloalkyl or optionally substituted C₅₋₇cycloalkenyl, R³ is not substituted (other than optionally by alkyl or fluoroalkyl) at the ring atom connecting to the —NH— in formula (I), and R³ is not substituted (other than optionally by alkyl, fluoroalkyl or NHR²¹) at the two ring atoms either side of (bonded to) the connecting atom. For example, suitably, for R³, and in particular when R³ is optionally substituted C₃₋₈cycloalkyl or optionally substituted C₅₋₇cycloalkenyl, R³ is not substituted at the ring atom connecting to the —NH— in formula (I), and R³ is not substituted at the two ring atoms either side of (bonded to) the connecting atom.

Suitably, for R³, and in particular when R³ is optionally substituted C₃₋₈cycloalkyl or optionally substituted C₅₋₇cycloalkenyl, the one or two optional R³ substituents if present can comprise a substituent (for example is or are substituent(s)):

(a) at the 3-position of a R³ cyclobutyl ring, or

(b) at the 3- and/or 4- position(s) of a R³ cyclopentyl or cyclopentenyl ring, or

(c) at the 3-, 4- and/or 5- position(s) of a R³ cyclohexyl or cyclohexenyl ring, or

(d) at the 3-, 4-, 5- and/or 6- position(s) of a R³ cycloheptyl or cycloheptenyl ring, or

(e) at the 3-, 4-, 5-, 6- and/or 7- position(s) of a R³ cyclooctyl ring, and/or

(f) at the 1-, 2- and/or highest-numbered- position(s) of a R³ cycloalkyl or cycloalkenyl ring, for alkyl or fluoroalkyl substituent(s), and/or

(g) at the 2- and/or highest-numbered- position(s) of a R³ cycloalkyl or cycloalkenyl ring, for NHR²¹ substituent(s).

When R³ is optionally substituted C₃₋₈cycloalkyl, any OH, alkoxy, fluoroalkoxy, —CH₂CH₂OH or —CH₂NHR²² substituent (particularly any OH substituent) is suitably at the 3-, 4- or 5- position, e.g. 3- or 5-position, of the R³ cycloalkyl (e.g. C₆₋₈cycloalkyl) ring. Optionally, any OH, alkoxy, fluoroalkoxy, —CH₂CH₂OH or —CH₂NHR²² substituent particularly any OH substituent) can be: at the 3-position of a R³ cyclobutyl ring; or at the 3- or 4- position of a R³ C₅cycloalkyl (cyclopentyl) ring; or at the 3-, 4- or 5- position of a R³ C₆cycloalkyl (cyclohexyl) ring (e.g. at the 3- or 5-position of a R³ cyclohexyl ring especially for any OH substituent); or at the 3-, 4-, 5- or 6- position of a R³ cycloheptyl ring, or at the 3-, 4-, 5-, 6- or 7- position of a R³ cyclooctyl ring. Suitably, any OH, alkoxy, fluoroalkoxy, —CH₂CH₂OH or —CH₂NHR²² substituent (particularly any OH substituent) is at the 3- or 4- position of a R³ C₆cycloalkyl (cyclopentyl) ring; or more suitably at the 3-, 4- or 5- position, still more suitably at the 3- or 5-position, of a R³ C₆cycloalkyl (cyclohexyl) ring.

When R³ is optionally substituted C₃₋₈cycloalkyl or optionally substituted C₅₋₇cycloalkenyl, then any —C(O)OR²³, —C(O)NHR²⁴, —C(O)R²⁵, —CH₂OH or fluoro substituent is: at the 3-position of a R³ cyclobutyl ring; or at the 3- or 4- position of a R³ C₅cycloalkyl (cyclopentyl) or cyclopentenyl ring; or at the 4-position of a R³ C₆cycloalkyl (cyclohexyl) or cyclohexenyl ring; or at the 3-, 4-, 5- or 6- position of a R³ cycloheptyl or cycloheptenyl ring, or at the 3-, 4-, 5-, 6- or 7- position of a R³ cyclooctyl ring. Any —C(O)OR²³, —C(O)NHR²⁴, —C(O)R²⁵, —CH₂OH or fluoro substituent, e.g. any —C(O)NHR²⁴ or fluoro substituent, is suitably at the 4-position of a R³ C₆cycloalkyl (cyclohexyl) or cyclohexenyl ring. It is particularly preferable for any —C(O)NHR²⁴ substituent to be at the 4-position of a R³ cyclohexyl ring.

When R³ is optionally substituted C₃₋₈cycloalkyl, any NHR²¹ substituent is at any position other than the 1-position (the ring atom connecting to the —NH— in formula (I)), e.g. at the 2-, 3-, 4-, 5-, 6-, 7- or 8- position. Suitably, any NHR²¹ substituent is at the 2-, 3-, 4-, 5- or 6- position, for example at the 3- or 5- position, of a R³ cyclohexyl ring.

When R³ is optionally substituted C₃₋₈cycloalkyl or optionally substituted C₅₋₇cycloalkenyl, any alkyl or fluoroalkyl substituent can for example be at the 1-, 2-, 3-, 4-, 5-, 6-, 7- or 8- position, for example at the 1-, 2-, 3-, 5- or 6- position, e.g. the 1-position, of the R³ ring. Preferably, any alkyl or fluoroalkyl substituent is at the 1-, 2-, 3-, 5- or 6- position, or more preferably at the 1-, 3- or 5- position, of a R³ cyclohexyl or cyclohexenyl ring.

When R³ is optionally substituted C₃₋₈cycloalkyl, any oxo (═O), hydroxyimino (═N—OH); or (C₁₋₄alkoxy)imino (═N—OR²⁶) substituent is suitably at the 3-, 4- or 5-position, e.g. at the 4-position, of the R³ cycloalkyl (e.g. C₆₋₈cycloalkyl e.g. cyclohexyl) ring. Preferably any such substituent is at the 4-position of a R³ cyclohexyl ring.

When R³ is optionally substituted C₃₋₈cycloalkyl (e.g. C₆₋₇cycloalkyl), R³ is preferably cyclohexyl (i.e. unsubstituted); or cycloheptyl (i.e. unsubstituted); or cyclohexyl substituted by one substituent being oxo (═O), OH, NHR²¹, C₁₋₂alkyl, C₁₋₂fluoroalkyl, —CH₂OH, —C(O)OR²³, —C(O)NHR²⁴, —C(O)R²⁵, fluoro, hydroxyimino (═N—OH), or (C₁₋₄alkoxy)imino (═N—OR²⁶); or cyclohexyl substituted by two fluoro substituents. More preferably, R³ is cyclohexyl (i.e. unsubstituted); or cycloheptyl (i.e. unsubstituted); or cyclohexyl substituted by one substituent being oxo (═O), OH, NHR²¹, C₁₋₂alkyl, C₁₋₂fluoroalkyl, —C(O)OR²³, —C(O)NHR²⁴, fluoro, hydroxyimino (═N—OH), or (C₁₋₂alkoxy)imino (═N—OR²⁶ wherein R²⁶ is C₁₋₂alkyl); or cyclohexyl substituted by two fluoro substituents. Still more preferably R³ is cyclohexyl (i.e. unsubstituted) or cyclohexyl substituted by one oxo (═O), hydroxyimino (═N—OH), —C(O)NH₂, methyl or OH substituent. The optional substituent can for example be at the 3- or 4- position of the R³ cyclohexyl ring. Preferably, any OH substituent is preferably at the 3-position of a R³ cyclohexyl ring, and/or any oxo (═O), hydroxyimino (═N—OH), (C₁₋₄alkoxy)imino (═N—OR²⁶) or —C(O)NH₂ substituent is preferably at the 4-position of a R³ cyclohexyl ring, and/or any alkyl or fluoroalkyl substituent is preferably at the 1-, 3- or 5- position of a R³ cyclohexyl ring.

Alternatively, when R³ is optionally substituted C₃₋₈cycloalkyl, R³ can suitably be cyclobutyl optionally substituted with one substituent being oxo (═O); OH; NHR²¹ wherein R²¹ is a hydrogen atom (H); methyl; —CH₂F; —CHF₂; —C(O)OR²³; —C(O)NHR²⁴ wherein R²⁴ is H or methyl (preferably H); fluoro; hydroxyimino (═N—OH); or methoxyimino (═N—OR²⁶ where R²⁶ is methyl). In this case, preferably R³ is cyclobutyl optionally substituted by one —C(O)NHR²⁴ substituent wherein R²⁴ is H or methyl (preferably H). R³ can for example be cyclobutyl (i.e. unsubstituted) or 3-(aminocarbonyl)cyclobutyl (i.e. 3-(aminocarbonyl)cyclobutan-1-yl) (e.g. in a cis or trans configuration, preferably cis).

When R³ is optionally substituted C₆₋₇cycloalkyl, R³ can for example be 4-hydroxy-cyclohexyl (i.e. 4-hydroxycyclohexan-1-yl), 4-methylcyclohexyl, 2-aminocyclohexyl, or 3-oxocyclohexyl, but R³ is more preferably cyclohexyl (i.e. unsubstituted), cycloheptyl (i.e. unsubstituted), 3-hydroxy-cyclohexyl (i.e. 3-hydroxycyclohexan-1-yl) (e.g. in a cis or trans configuration, preferably cis), 4-oxo-cyclohexyl (i.e. 4-oxocyclohexan-1-yl), 4-(hydroxyimino)cyclohexyl (i.e. 4-(hydroxyimino)cyclohexan-1-yl), 4-(C₁₋₂alkoxyimino)cyclohexyl, 4-(aminocarbonyl)cyclohexyl (i.e. 4-(aminocarbonyl)cyclohexan-1-yl) (e.g. in a cis or trans configuration, preferably cis), 1-methylcyclohexyl, 3-methylcyclohexyl, 4,4-(difluoro)cyclohexyl, or 3-aminocyclohexyl. Alternatively, R³ can preferably be 4-acetylcyclohexyl (e.g. in a cis or trans configuration, preferably cis).

When R³ is optionally substituted C₆₋₇cycloalkyl, R³ is most preferably cyclohexyl (i.e. unsubstituted), 3-hydroxy-cyclohexyl (i.e. 3-hydroxycyclohexan-1-yl) (preferably in a cis configuration), 4-oxo-cyclohexyl (i.e. 4-oxocyclohexan-1-yl), 4-(hydroxyimino)cyclohexyl (i.e. 4-(hydroxyimino)cyclohexan-1-yl), or 4-(aminocarbonyl)cyclohexyl (i.e. 4-(aminocarbonyl)cyclohexan-1-yl) (preferably in a cis configuration).

When R³ is optionally substituted C₅cycloalkyl (optionally substituted cyclopentyl), R³ can for example be cyclopentyl (i.e. unsubstituted) or more suitably 3-hydroxy-cyclopentyl.

When R³ is optionally substituted mono-unsaturated-C₅₋₇cycloalkenyl, preferably it is optionally substituted mono-unsaturated-C₅₋₆cycloalkenyl, more preferably optionally substituted mono-unsaturated-C₆cycloalkenyl (i.e. optionally substituted mono-unsaturated-cyclohexenyl=optionally substituted cyclohexenyl). For example, the R³ cyclohexenyl can be optionally substituted cyclohex-3-en-1-yl.

When R³ is optionally substituted mono-unsaturated-C₅₋₇cycloalkenyl, in one optional embodiment the R³ cycloalkenyl is optionally substituted with one or two substituents independently being fluoro or methyl. Preferably, in this embodiment, if there are two substituents then they are not both methyl.

In another optional embodiment, the R³ cycloalkenyl (e.g. cyclohexenyl) is optionally substituted with one substituent being fluoro or C₁₋₂alkyl (preferably fluoro or methyl); suitably the R³ cycloalkenyl (e.g. cyclohexenyl) can be substituted with one fluoro substituent or is unsubstituted. For example, the R³ optionally substituted cycloalkenyl can be cyclohex-3-en-1-yl (i.e. unsubstituted) or 4-fluoro-cyclohex-3-en-1-yl.

For R³ cycloalkenyl, the optional substituent(s) can for example be at the 1-, 2-, 3-, 4-, 5- or 6- position(s) of the cycloalkenyl ring.

When R³ is the heterocyclic group of sub-formula (aa), (bb) or (cc), then Y is suitably O or NR¹⁰. When R³ is the heterocyclic group of sub-formula (aa) or (bb), then Y is preferably O or N—C(O)—NH₂.

Suitably, R¹⁰ is a hydrogen atom (H), methyl, ethyl, C(O)NH₂, C(O)—C₁₋₂alkyl or C(O)—C₁fluoroalkyl. Preferably, R¹⁰ is not C₁₋₂alkyl or C₁₋₂fluoroalkyl.

More preferably, R¹⁰ is a hydrogen atom (H), C(O)NH₂, C(O)—C₁₋₂alkyl (e.g. C(O)methyl) or C(O)—C₁fluoroalkyl (e.g. C(O)—CF₃). Still more preferably R¹⁰ is H, C(O)NH₂ or C(O)methyl; for example C(O)NH₂.

When R³ is the heterocyclic group of sub-formula (aa), (bb) or (cc), then it is preferable that R³ is the heterocyclic group of sub-formula (aa) or (bb), more preferably of sub-formula (bb).

In sub-formula (bb), n¹ is preferably 1. In sub-formula (cc), n² is preferably 1. That is, six-membered rings are preferred in the R³ heterocyclic group.

Suitably, in R³, the heterocyclic group of sub-formula (aa), (bb) or (cc) can be unsubstituted on a ring carbon. (In this connection, where Y is NR¹⁰, R¹⁰ is not a substituent on a ring carbon).

In the R³ heterocyclic group of sub-formula (aa), (bb) or (cc), the one or two optional substituents (i.e. the one or two optional ring-carbon substituents) preferably comprise (e.g. is or independently are) OH; oxo (═O); C₁₋₂alkyl (e.g. methyl) or C₁₋₂fluoroalkyl (e.g. C₁fluoroalkyl such as —CH₂F or —CHF₂). More preferably, in the R³ heterocyclic group of sub-formula (aa), (bb) or (cc), the one or two optional substituents comprise (e.g. is or independently are) C₁₋₂alkyl (e.g. methyl) or oxo; most preferably the one or two optional substituents comprise (e.g. is or are) oxo (═O).

In the R³ heterocyclic group of sub-formula (aa), (bb) or (cc), any oxo (═O) substituent is preferably on a carbon atom bonded (adjacent) to Y, e.g. is on a carbon atom bonded (adjacent) to Y only when Y is O or NR¹⁰.

In the R³ heterocyclic group of sub-formula (aa), (bb) or (cc), any oxo (═O) substituent can suitably be at the 2-, 3-, 4-, 5- or 6- position of the R³ heterocyclic ring. For example any oxo (═O) substituent(s) can be: at the 2-, 4- or 5- position(s) (e.g. 2-position or 4-position, or two oxo substituents at 2- and 4- positions) of a R³ heterocyclic group of sub-formula (aa), at the 2-, 4-, 5- or 6- position(s) (e.g. 4-position) of a six-membered R³ heterocyclic group of sub-formula (cc) wherein n² is 1, at the 2-, 3-, 5-, 6- or 7-position(s) (e.g. 5-position) of a seven-membered R³ heterocyclic group of sub-formula (bb) wherein n¹ is 2, or at the 2-, 4-, 5-, 6- or 7- position(s) (e.g. 2-position) of a seven-membered R³ heterocyclic group of sub-formula (cc) wherein n² is 2.

(In this connection and generally herein, the 1-position of the R³ heterocyclic ring is deemed to be the connection point to the —NH— in formula (I)=the ring atom connecting to the —NH— in formula (I), and the remaining positions of the ring are then numbered so that the ring heteroatom takes the lowest possible number).

In the R³ heterocyclic group of sub-formula (aa), (bb) or (cc), any alkyl or fluoroalkyl substituent (ring-carbon substituent) can for example be at the 1-, 2-, 3-, 4-, 5- or 6-position, e.g. the 1-position, of the R³ heterocyclic ring, for example at the 1-, 3- or 5- position of a six-membered R³ heterocyclic ring.

In the R³ heterocyclic group of sub-formula (aa), (bb) or (cc), then any OH substituent is: at the 5-position of a six-membered R³ heterocyclic group of sub-formula (cc) wherein n² is 1; at the 5- or 6- position of a seven-membered R³ heterocyclic group of sub-formula (cc) wherein n² is 2; or at the 6- position of a seven-membered R³ heterocyclic group of sub-formula (bb) wherein n¹ is 2.

Any other optional ring-carbon substituents of the R³ heterocyclic group can optionally be positioned on the R³ heterocyclic ring at numerical positions as described herein for when R³ is optionally substituted C₅₋₇cycloalkyl, all necessary changes to the wording being made.

In the R³ heterocyclic group of sub-formula (aa), (bb) or (cc), preferably, only C₁₋₂alkyl, C₁₋₂fluoroalkyl, fluoro or oxo (═O) substitution or no substitution is allowed independently at each of the 2- and highest-numbered- positions of the R³ heterocyclic ring (e.g. at each of the 2- and 6- positions of a six-membered R³ heterocyclic ring), and/or only C₁₋₂alkyl, C₁₋₂fluoroalkyl or fluoro substitution or no substitution is allowed at the 1-position of the R³ heterocyclic ring.

When R³ is the heterocyclic group of sub-formula (aa) and Y is NR¹⁰, then R¹⁰ is not C(O)—C₁₋₂alkyl, C(O)—C₁fluoroalkyl or —C(O)—CH₂O—C₁alkyl.

In one preferable embodiment, when R³ is the heterocyclic group of sub-formula (aa) then Y is O, S, SO₂, NH or NC(O)NH₂ (e.g. O, S, SO₂ or NH).

When R³ is the heterocyclic group of sub-formula (bb), n¹ is 1, and Y is NR¹⁰ (e.g. when NHR³ is

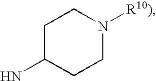

then R¹⁰ is not C₁₋₂alkyl or C₁₋₂fluoroalkyl. When R³ is the heterocyclic group of sub-formula (bb) wherein n¹ is 1 or 2 and Y is NR¹⁰, then preferably R¹⁰ is not C₁₋₂alkyl or C₁₋₂fluoroalkyl.

In one embodiment, when R³ is the heterocyclic group of sub-formula (bb), then preferably Y is O, S, SO₂ or NR¹⁰ wherein R¹⁰ is H, C(O)NH₂, C(O)—C₁₋₂alkyl (e.g. C(O)methyl) or C(O)—C₁fluoroalkyl (e.g. C(O)—CF₃), or more preferably R¹⁰ is H, C(O)NH₂ or C(O)Me, for example C(O)NH₂ or C(O)Me, most preferably C(O)NH₂.

When R³ is the heterocyclic group of sub-formula (cc), then Y is O, S, SO₂ or NR¹⁰ wherein R¹⁰ is H.

Optionally, for sub-formula (bb) and/or for sub-formula (cc), Y is O or NR¹⁰.

When R³ is optionally substituted C₃₋₈cycloalkyl (e.g. C₆₋₇cycloalkyl) or optionally substituted mono-unsaturated-C₅₋₇cycloalkenyl or an optionally substituted heterocyclic group of sub-formula (aa), (bb) or (cc), then a substituent can be in the cis or trans configuration with respect to the —NH— group of formula (I) to which R³ is attached (bonded); this includes mixtures of configurations wherein the stated configuration is the major component. For example, an OH or —C(O)NHR²⁴ substituent on C₆₋₇cycloalkyl can for example be in the cis configuration and/or a NHR²¹ substituent on C₆₋₇cycloalkyl can for example be in the cis or trans configuration, with respect to the —NH— group of formula (I) to which R³ is attached (bonded), including mixtures of configurations wherein the stated configuration is the major component.

When R³ is a bicyclic group of sub-formula (ee), then preferably Y¹, Y² and Y³ are all CH₂.

Preferably, NHR³ is of sub-formula (a), (a1), (b), (c), (c 1), (c 2), (c 3), (c 4), (c 5), (c 6), (c 7), (d), (e), (f), (g), (g1), (g2), (g3), (g4), (h), (i), 0.), (k), (k), (k2), (L), (m), (m1), (m2), (m3), (n), (o), (o1), (o2), (o3), (p), (p1), (p2), (p3), (p4), (p5), (p6), (p9), (p10), (p 11) or (q):

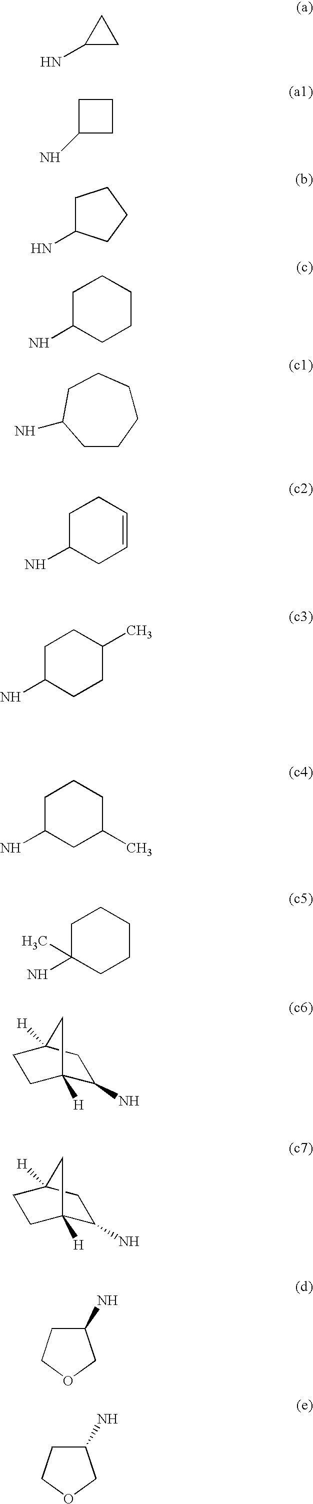

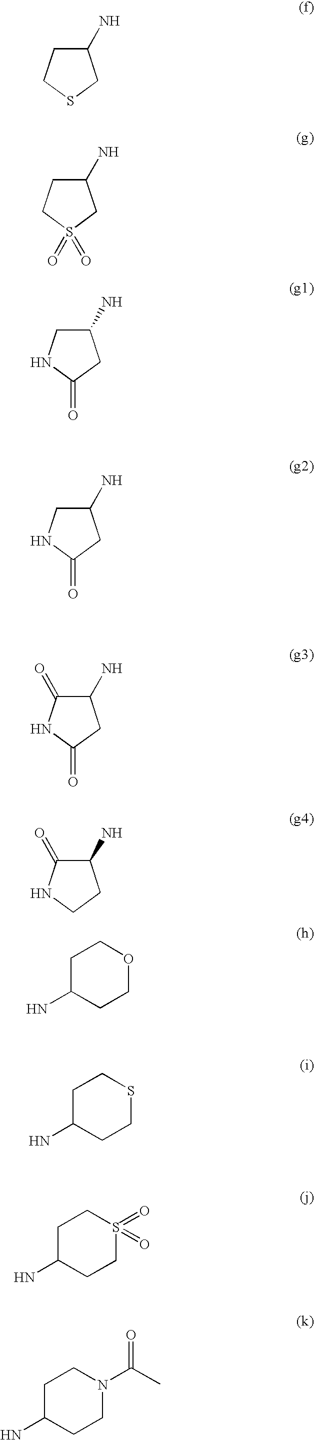

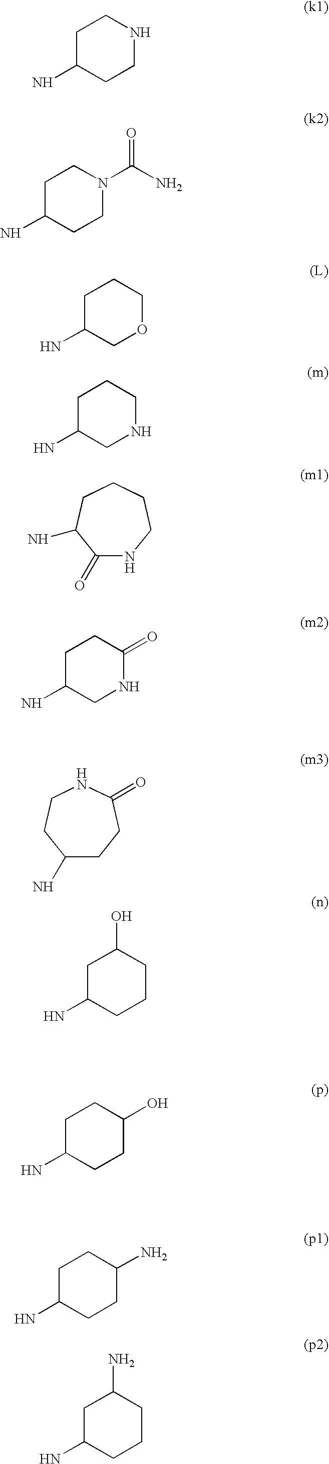

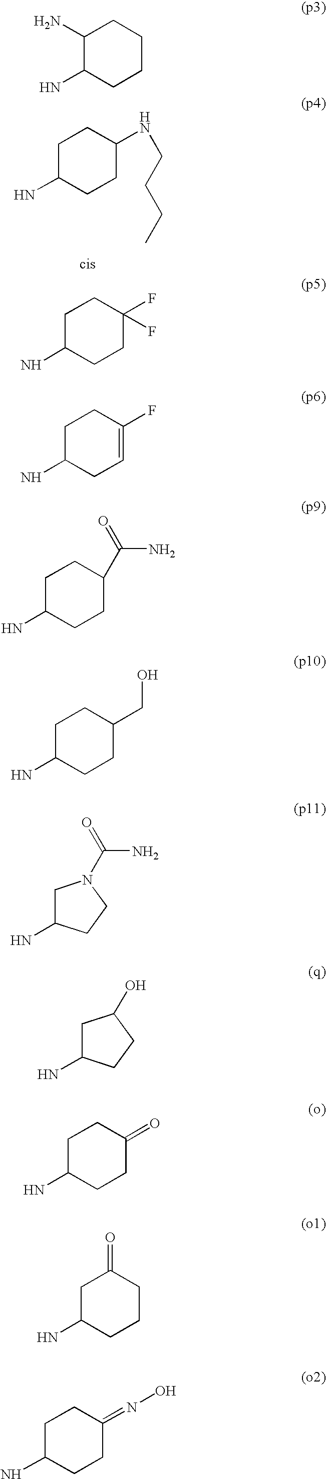

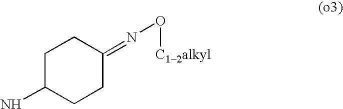

In the sub-formulae (a) to (q) etc above, the —NH— connection point of the NHR³ group to the 4-position of the pyrazolopyridine of formula (I) is underlined.

Preferably, NHR³ is of sub-formula (c), (c1), (c 2), (c 3), (c 4), (c 5), (c 6), (c 7), (d), (e), (f), (g1), (g4), (h), (i), (j), (k), (k), (k2), (L), (m), (ml), (m2), (m3), (n), (o), (o1), (o2), (o3), (p), (p2), (p5), (p6), (p9), (p10), (p 11) or (q); or preferably NHR³ is of sub-formula (a1), (c), (c1), (c 2), (c 3), (c 4), (c 5), (c 6), (c 7), (d), (e), (f), (g1), (g4), (h), (i), (j), (k), (k1), (k2), (L), (m), (m1), (m3), (n), (o), (o1), (o2), (o3), (p), (p1), (p2), (p5), (p6), (p9) (p10), (p11) or (q).

More preferably, NHR³ is of sub-formula (c), (c1), (c 4), (c 5), (h), (i), (j), (k), (k2), (m1), (n), (o), (o2), (o3), (p2), (p5), (p6), (p9), (p11) or (q). NHR³ can for example be of sub-formula (c), (h), (k), (k2), (n), (o), (o2), (p9) or (p 11); or still more preferably (c), (h), (k2), (n), (o), (o2), (p9) or (p11). Most preferably, R³ is tetrahydro-2H-pyran-4-yl or 1-(aminocarbonyl)-4-piperidinyl; that is NHR³ is most preferably of sub-formula (h) or (k2), as shown above.

When NHR³ is of sub-formula (n), then it can be in the trans configuration; but preferably it is in the cis configuration, i.e. preferably it is a cis-(3-hydroxycyclohexan-1-yl)amino group (including mixtures of configurations wherein the cis configuration is the major component), e.g. in any enantiomeric form or mixture of forms such as a racemic mixture.

When NHR³ is of sub-formula (p9), then it can be in the trans configuration; but preferably it is in the cis configuration, i.e. preferably it is a cis-[4-(aminocarbonyl)cyclohexan-1-yl]amino group (including mixtures of configurations wherein the cis configuration is the major component).

In an alternative preferable embodiment, NHR³ is of sub-formula (p12) or (p13):

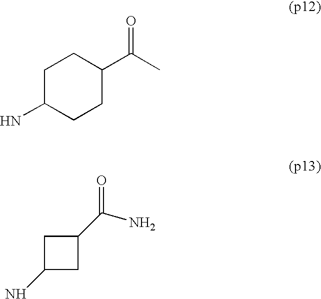

In the sub-formulae (p12) and (p13) above, the —NH— connection point of the NHR³ group to the 4-position of the pyrazolopyridine of formula (I) is underlined.

When NHR³ is of sub-formula (p12) or (p13), then it can be in the trans configuration; but preferably it is in the cis- configuration, i.e. preferably NHR³ is a cis-[4-acetylcyclohexan-1-yl]amino group or a cis-[3-(aminocarbonyl)cyclobutan-1-yl]amino group respectively (each including mixtures of configurations wherein the cis configuration is the major component).

Where R⁴ is C₁₋₂fluoroalkyl, then it can be C₁fluoroalkyl such as monofluoromethyl, difluoromethyl or trifluoromethyl.

R^{4a} can suitably be a hydrogen atom (H) or methyl (Me), more suitably H.

R⁴ can for example be a hydrogen atom (H); methyl, ethyl, C₁fluoroalkyl, —CH₂OH, —CH(Me)OH, —CH₂CH₂OH, or —CH₂OMe; or preferably a hydrogen atom (H), methyl, ethyl, CF₃, —CH₂OH, or —CH₂OMe. More preferably, R⁴ is methyl, ethyl, CF₃, —CH₂OH, or —CH₂OMe; for example methyl, ethyl, CF₃ or —CH₂OH. Still more preferably, R⁴ is methyl or ethyl. Most preferably, R⁴ is ethyl.

Suitably, R⁴ is not a hydrogen atom (H), and more suitably R⁵ is a hydrogen atom (H).

When R⁵ is C₁₋₄alkyl substituted by one substituent R¹¹ or R⁵ is C₂₋₄alkyl (e.g. ethyl or n-propyl) substituted on different carbon atoms by two OH substituents, then suitably R⁵ is C₁₋₄alkyl substituted by one substituent R¹¹.

When R⁵ is C₁₋₄alkyl substituted by one substituent R¹¹, it is suitable that R⁵ is C₁₋₃alkyl (e.g. C₁₋₂alkyl) substituted by one substituent R¹¹. Suitably, R⁵is —(CH₂)ₙ⁵—R¹¹ wherein n⁵ is 1, 2, 3 or 4 or R⁵ is —CH(Me)-R¹¹. Preferably n⁵ is 1, 2 or 3, more preferably 1 or 2, still more preferably 1.

Suitably, R¹¹ is: thydroxy (OH); C₁₋₄alkoxy or C₁₋₂alkoxy (such as t-butyloxy, ethoxy or preferably methoxy); C₁fluoroalkoxy; —NR¹²R¹³; —NR¹⁵—C(O)R¹⁶; or —NR¹⁵—S(O)₂R¹⁶. More suitably, R¹¹ is hydroxy (OH), C₁₋₄alkoxy (e.g. C₁₋₂alkoxy), or —NR¹²R¹³; still more suitably OH, ethoxy, methoxy, NH_{2,} NHMe, NHEt, NMe₂, pyrrolidin-1-yl or piperidin-1-yl; preferably OH, methoxy, NH_{2,} NHMe or NMe₂.

Where R⁵ is C₁₋₈alkyl, then suitably it is C₁₋₆alkyl or C₁₋₅alkyl or C₁₋₄alkyl or C₁₋₃alkyl. Where R⁵ is C₁₋₃fluoroalkyl then suitably it is C₁₋₂fluoroalkyl or C₁fluoroalkyl such as monofluoromethyl, difluoromethyl or trifluoromethyl. Where R⁵ is C₃₋₈cycloalkyl optionally substituted by a C₁₋₂alkyl group, then optionally the C₃₋₈cycloalkyl is not substituted at the connecting ring-carbon. Where R⁵ is optionally substituted C₃₋₈cycloalkyl, then suitably it is C₃₋₈cycloalkyl (i.e. unsubstituted) and/or optionally substituted C₃₋₆cycloalkyl such as optionally substituted cyclopropyl or optionally substituted cyclohexyl.

When R⁵ is optionally substituted —(CH₂)ₙ⁴—C₃₋₈cycloalkyl, then n⁴ is preferably 1, and/or suitably R⁵ is optionally substituted —(CH₂)ₙ⁴—C₃₋₆cycloalkyl such as optionally substituted —(CH₂)ₙ⁴-cyclopropyl or optionally substituted —(CH₂)ₙ⁴—C₆cycloalkyl. When R⁵ is optionally substituted —(CH₂)ₙ⁴—C₃₋₈cycloalkyl, preferably it is not substituted. For example, R⁵ can be (cyclohexyl)methyl-, that is —CH₂-cyclohexyl, or —CH₂-cyclopropyl.

When R¹⁹ is C₁₋₂alkyl, then optionally it can be methyl.

When R⁵ is —(CH₂)ₙ¹¹—C(O)R¹⁶; —(CH₂)ₙ¹¹—C(O)NR¹²R¹³; —CHR¹⁹—C(O)NR¹²R¹³; —(CH₂)ₙ¹¹—C(O)OR¹⁶; —(CH₂)ₙ¹¹—C(O)OH; —CHR¹⁹—C(O)OR¹⁶; —CHR¹⁹—C(O)OH; —(CH₂)ₙ¹¹—S(O)₂—NR¹²R¹³; —(CH₂)ₙ¹¹—S(O)₂R¹⁶; or —(CH₂)ₙ¹¹—CN; then R⁵ can suitably be —(CH₂)ₙ¹¹—C(O)NR¹²R¹³; —(CH₂)ₙ¹¹—C(O)OR¹⁶; —(CH₂)ₙ¹¹—C(O)OH; or —(CH₂)ₙ¹¹—CN; or R⁵ can more suitably be —(CH₂)ₙ¹¹—C(O)NR¹²R¹³; —(CH₂)ₙ¹¹—C(O)OR¹⁶ or —(CH₂)ₙ¹¹—CN; or preferably —(CH₂)ₙ¹¹—C(O)NR¹²R¹³ or —(CH₂)ₙ¹¹—C(O)OR¹⁶.

Preferably, n¹¹ is 0, 1 or 2. In one optional embodiment n¹¹ is 0 or 1, for example 0. In a suitable embodiment, n¹¹ is 2.

When R⁵ is —(CH₂)ₙ¹³-Het, n¹³ can for example be 0 or 1.

Suitably, Het is a 5- or 6-membered saturated or unsaturated heterocyclic ring, and/or preferably Het is a 4-, 5-, 6- or 7-membered saturated heterocyclic ring. Suitably, the heterocyclic ring Het contains one ring-hetero-atom selected from O, S and N. Suitably, the carbon ring-atoms in Het are not substituted. Het can for example be:

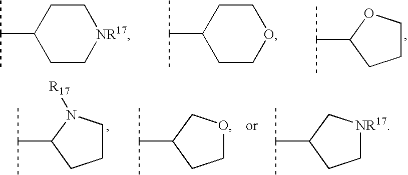

When R⁵ is phenyl (Ph), —CH₂-Ph, -CHMe-Ph, -CHEt-Ph, CMe₂Ph, or —CH₂CH₂-Ph, wherein the phenyl ring Ph is optionally substituted, then suitably Ph is optionally substituted with one of the substituents defined herein. Preferably, R⁵ is phenyl (Ph) or —CH₂-Ph wherein the phenyl ring Ph is optionally substituted with one or two substituents as defined herein.

When R⁵ is phenyl (Ph), —CH₂-Ph, -CHMe-Ph, -CHEt-Ph, CMe₂Ph, or —CH₂CH₂-Ph, wherein the phenyl ring Ph is optionally substituted with one or two substituents, then preferably the phenyl ring Ph is optionally substituted with one or two (e.g. one) substituents independently being: fluoro; chloro; C₁₋₂alkyl (e.g. methyl); C₁fluoroalkyl (e.g. trifluoromethyl); C₁₋₂alkoxy (e.g. methoxy); or C₁fluoroalkoxy (e.g. trifluoromethoxy or difluoromethoxy). Ph can be unsubstituted.

When R⁴ and R⁵ taken together are —(CH₂)ₚ¹— or —(CH₂)ₚ³—X⁵—(CH₂)ₚ⁴—, in which X⁵ is O or NR^{17a}; then preferably R⁴ and R⁵ taken together are —(CH₂)ₚ¹—. In one embodiment of the invention, R⁴ and R⁵ are not taken together to be either —(CH₂)ₚ¹— or —(CH₂)ₚ³—X⁵—(CH₂)ₚ⁴—.

When R⁴ and R⁵ taken together are —(CH₂)ₚ¹—, then p¹ can for example be 2, 4, 5 or 6. p¹ is preferably 2, 4 or 5, more preferably 2 or 4.

When R⁴ and R⁵ taken together are —(CH₂)ₚ³—X⁵—(CH₂)ₚ⁴—, in which X⁵ is O or NR^{17a}; then suitably: p³ is 2, and/or p⁴ is 2, and/or one of p³ and p4 is 1 and the other of p³ and p⁴ is 2, and/or p³ and p⁴ are both 1. Suitably, X⁵ is O. —(CH₂)ₚ³—X⁵—(CH₂)ₚ⁴— can for example be —(CH₂)₂—O—(CH₂)₂—.

In one embodiment of the invention, R⁴ and R⁵ are not taken together as —(CH₂)ₚ¹— or —(CH₂)ₚ³—X⁵—(CH₂)ₚ⁴—.

It is preferable that Ar has the sub-formula (x).

Preferably, in sub-formula (x), two or more (more preferably three or more) of A, B, D, E and F are independently C—H (carbon-hydrogen), C—F (carbon-fluorine) or nitrogen (N).

Suitably, in sub-formula (x), three or more of A, B, D, E and F are independently C—H (carbon-hydrogen), C—F (carbon-fluorine), nitrogen (N), or nitrogen-oxide (N⁺—O⁻).

Preferably, in sub-formula (x), two or more (e.g. three or more) of A, B, D, E and F are independently C—H (carbon-hydrogen), C—F (carbon-fluorine), or nitrogen (N); and one or more (e.g. two or more) others of A, B, D, E and F are independently C—H (carbon-hydrogen), C—F (carbon-fluorine), C—Cl (carbon-chlorine), C-Me, C—OMe, or nitrogen (N). More preferably, in sub-formula (x), two or more (e.g. three or more) of A, B, D, E and F are C—H (carbon-hydrogen); and one or more (e.g. two or more) others of A, B, D, E and F are independently C—H (carbon-hydrogen), C—F (carbon-fluorine), C—Cl (carbon-chlorine), C-Me, C—OMe, or nitrogen (N).

Preferably, in sub-formula (x), two or more (e.g. three or more, e.g. four or more) of A, B, D, E and F are C—H.

Preferably, in sub-formula (x), no more than one (more preferably none) of A, B, D, E and F are independently nitrogen or nitrogen-oxide (N⁺—O⁻).

Preferably, in sub-formula (x), none of A, B, D, E and F are nitrogen-oxide (N⁺—O⁻).

Preferably, Ar has the sub-formula (x) which is sub-formula (x1), (x2), (x3), (x4), (x5), (x6), (x7), (x8), (x9), (x10), (x11), (x12), (x12a), (x13), (x14), (x15) or (x16):

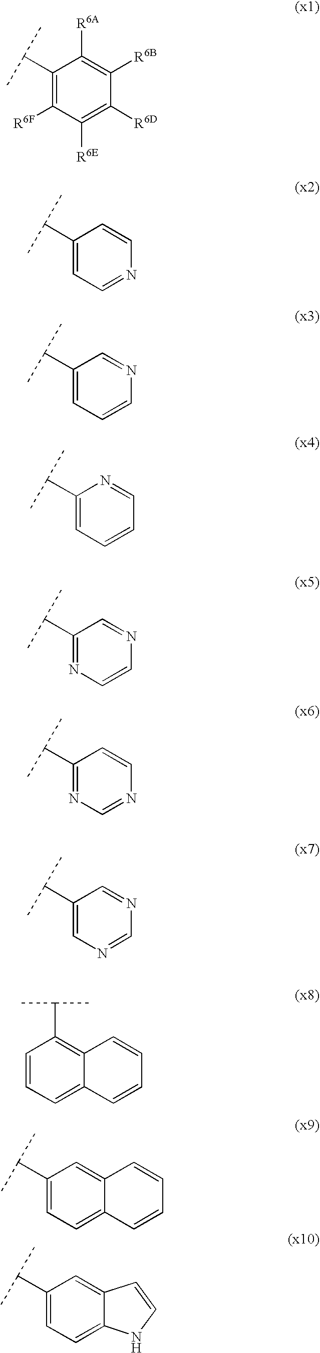

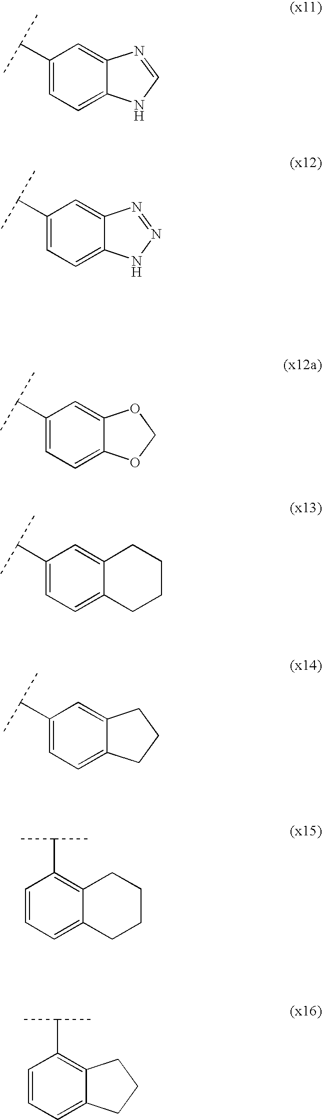

In one preferable embodiment, Ar has the sub-formula (x) which is sub-formula (x1), (x2), (x3), (x4), (x5), (x6), (x7), (x8), (x9), (x10), (x11), (x12), (x13), (x14), (x15), or (x16).

More preferably, Ar has the sub-formula (x) which is sub-formula (x1), (x2), (x3), (x8), (x13), or (x14). Still more preferably, Ar has the sub-formula (x) which is sub-formula (x1), (x8), (x13), or (x14). Most preferably, Ar has the sub-formula (x) which is sub-formula (x1).

In sub-formula (x), preferably, R^{6A}, R^{6B}, R^{6D}, R^{6E} and/or R^{6F}, independently of each other, is or are: a hydrogen atom (H), a fluorine, chlorine, bromine or iodine atom, methyl, ethyl, n-propyl, isopropyl, C₄alkyl, trifluoromethyl, —CH₂OH, methoxy, ethoxy, n-propoxy, isopropoxy, C₁fluoroalkoxy (e.g. trifluoromethoxy or difluoromethoxy), cyclohexyloxy; cyclopentyloxy; nitro (—NO₂), OH, C₁₋₃alkylS(O)₂— (such as MeS(O)₂—), C₁₋₃alkylS(O)₂—NH— such as Me-S(O)₂—NH—, Me₂N—S(O)₂—, H₂N—S(O)₂—, —CONH₂, —CONHMe, —C(O)OH, cyano (—CN), NMe₂, or C₁₋₂alkyl-S(O)₂—CH₂— such as Me-S(O)₂—CH₂—.

More preferably, R^{6A}, R^{6B}, R^{6D}, R^{6E} and/or R^{6F}, independently of each other, is or are: a hydrogen atom (H), a fluorine, chlorine, bromine or iodine atom, methyl, ethyl, n-propyl, isopropyl, isobutyl, trifluoromethyl, —CH₂OH, methoxy, ethoxy, n-propoxy, isopropoxy, C₁fluoroalkoxy (e.g. trifluoromethoxy or difluoromethoxy), nitro (—NO₂), OH, C₁₋₃alkylS(O)₂— such as MeS(O)₂—, C₁₋₂alkylS(O)₂—NH— such as Me-S(O)₂—NH—, —CONH₂, cyano (—CN), or C₁₋₂alkylS(O)₂—CH₂— such as Me-S(O)₂—CH₂.

Still more preferably, R^{6A}, R^{6B}, R^{6D}, R^{6E} and/or R^{6F}, independently of each other, is or are: a hydrogen atom (H), a fluorine, chlorine or bromine atom, methyl, ethyl, n-propyl, isopropyl, trifluoromethyl, —CH₂OH, methoxy, ethoxy, n-propoxy, difluoromethoxy, OH or MeS(O)₂—.

When two adjacent groups selected from R^{6A}, R^{6B}, R^{6D}, R^{6E} and R^{6F} are taken together, then, preferably, when taken together they are: —CH═CH—CH═CH—, —CH₂)ₙ^{14a}— where n¹⁴a is 3, 4 or 5 (e.g. 3 or 4), —O—(CMe₂)—O—, —O—(CH₂)ₙ^{14b}—O— where n^{14b} is 1 or 2; —CH═CH—NR^{15b}—; —N═CH—NR^{15b}—; —N═N—NR^{15b} wherein R^{15b} is H or C₁₋₂alkyl (preferably R^{15b} is H). More preferably, in this embodiment, two adjacent groups selected from R^{6A}, R^{6B}, R^{6D}, R^{6E} and R^{6F} are taken together and are: —CH═CH—CH═CH₂— or —(CH₂)ₙ^{14a}— where n^{14a} is 3, 4 or 5 (e.g. 3 or 4).

In sub-formula (x), e.g. in sub-formula (x1), suitably, one, two or three of R^{6B}, R^{6D} and R^{6E} are other than a hydrogen atom (H).

In sub-formula (x), e.g. in sub-formula (x1), suitably, one or both of R^{6A} and R^{6F} are independently a hydrogen atom (H), a fluorine atom (F), or methyl. For example, one or both of R^{6A} and R^{6F} can be a hydrogen atom (H).

In sub-formula (x), e.g. in sub-formula (x1), suitably the ring or ring system is unsubstituted, monosubstituted, disubstituted or trisubstituted; or preferably the ring or ring system is unsubstituted, monosubstituted or disubstituted; more preferably monosubstituted or disubstituted. In sub-formula (x), e.g. in sub-formula (x1), for monosubstitution of the ring or ring system, then the one substituent selected from R^{6A}, R^{6B}, R^{6D}, R^{6E} and R^{6F} is suitably present at the 3- or 4-position with respect to the —(CR⁴R⁵)— side-chain (i.e., for a 4-position substituent, D is CR^{6D} where R^{6D} is other than H), or is a 2-methyl, 2-ethyl, 2-fluoro or 2-chloro substituent. In sub-formula (x), e.g. in sub-formula (x1), for disubstitution of the ring or ring system, then 3,4-disubstitution, 2,4-disubstitution, 2,3-disubstitution or 3,5-disubstitution is suitable. In sub-formula (x), 2,5-disubstitution is also suitable.

In one preferable embodiment, Ar has the sub-formula (x1) and is: phenyl, monoalkyl-phenyl-, mono(fluoroalkyl)-phenyl-, monohalo-phenyl-, monoalkoxy-phenyl-, mono(fluoroalkoxy)-phenyl-, mono(N,N-dimethylamino)-phenyl-, mono(methyl-SO₂—NH—)-phenyl-, mono(methyl-SO₂—)-phenyl-, dialkyl-phenyl-, monoalkyl-monohalo-phenyl-, mono(fluoroalkyl)-monohalo-phenyl-, dihalo-phenyl-, dihalo-monoalkyl-phenyl-, dihalo-mono(hydroxymethyl)-phenyl- (e.g. 2,3-dichloro-6-(hydroxymethyl)-phenyl-), or dialkoxy-phenyl- such as 3,4-dimethoxy-phenyl-. The substituents can preferably be further defined, as defined in preferable embodiments herein.

In one preferable embodiment, Ar is of sub-formula (x1) and is: monoalkyl-phenyl-, mono(fluoroalkyl)-phenyl-, monohalo-phenyl-, monoalkoxy-phenyl-, mono(fluoroalkoxy)-phenyl-, dialkyl-phenyl-, monoalkyl-monohalo-phenyl-, dihalo-phenyl- or dihalo-monoalkyl-phenyl-.

More preferably, in this embodiment, Ar is:

monoC₁₋₄alkyl-phenyl- or monoC₁₋₃alkyl-phenyl- such as 4-C₁₋₄alkyl-phenyl- (e.g. 4-C₁₋₃alkyl-phenyl-) or 2-C₁₋₂alkyl-phenyl-;

monoC₁fluoroalkyl-phenyl- such as 4-C₁fluoroalkyl-phenyl-;

monoC₁₋₃alkoxy-phenyl- such as 4-C₁₋₃alkoxy-phenyl- or 3-C₁₋₃alkoxy-phenyl-;

mono(C₁fluoroalkoxy)-phenyl- such as 4-C₁fluoroalkoxy-phenyl-;

diC₁₋₃alkyl-phenyl- or diC₁₋₂alkyl-phenyl- or dimethyl-phenyl- such as 3,4-dimethyl-phenyl-, 2,4-dimethyl-phenyl-, 3,5-dimethyl-phenyl-, 2,3-dimethyl-phenyl- or 2,5-dimethyl-phenyl-; for example 3,4-dimethyl-phenyl-, 2,4-dimethyl-phenyl-, 2,3-dimethyl-phenyl- or 3,5-dimethyl-phenyl-;

monoC₁₋₃alkyl-monohalo-phenyl-, such as monoC₁₋₂alkyl-monohalo-phenyl- and/or monoC₁₋₃alkyl-monochloro-phenyl- or monoC₁₋₃alkyl-monofluoro-phenyl-, for example 4-methyl-3-chloro-phenyl-, 3-methyl-4-chloro-phenyl-, or 2-methyl-4-chloro-phenyl-;

dihalo-phenyl- such as 2-chloro-4-fluorophenyl- or 2,4-difluoro-phenyl- or 4-bromo-2-fluorophenyl- or preferably 4-chloro-2-fluorophenyl-; for example dichloro-phenyl-such as 3,4-dichloro-phenyl- or 2,4-dichloro-phenyl- or 2,6-dichloro-phenyl- or preferably 2,3-dichloro-phenyl-; or

dihalo-monoC₁₋₂alkyl-phenyl- e.g. 2,4-dichloro-6-methyl-phenyl-.

In an alternative preferable embodiment, Ar has the sub-formula (x1) and is triC₁₋₂allyl-phenyl- such as trimethylphenyl-, e.g. 2,4,6-trimethylphenyl-.

In an alternative embodiment, Ar has the sub-formula (z).

Preferably, in sub-formula (z), three or more (for example all) of J, L, M and Q are independently C—H, C—F, C—C₁₋₂alkyl (e.g. C-Me), C-[connection point to formula (I)], or nitrogen (N).

Preferably, in sub-formula (z), no more than two (for example no more than one) of J, L, M and Q are nitrogen (N).

Suitably, Q is C-[connection point to formula (I)].

Suitably, R⁹ is a hydrogen atom (H) or methyl.

Suitably, R^{6J}, R^{6L}, R^{6M} and/or R^{6Q} independently is or are: a hydrogen atom (H); fluoro; chloro; C₁₋₂alkyl (e.g. methyl); C₁fluoroalkyl (e.g. CF₃); C₁₋₂alkoxy (methoxy); C₁fluoroalkoxy (e.g. CF₂HO—); OH (including any tautomer thereof); or phenyl optionally substituted by one substituent being fluoro, methyl, C₁fluoroalkyl, methoxy or C₁fluoroalkoxy. More suitably, R^{6J}, R^{6L}, R^{6M} and/or R^{6Q} independently is or are H, OH (including any keto tautomer thereof), or more preferably C₁₋₂alkyl (e.g. methyl) or C₁fluoroalkyl.

When Ar has the sub-formula (z), then sub-formula (z) can suitably be one of the following:

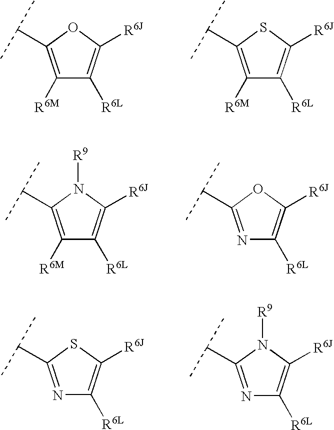

Suitably, R^{7a} is H or C₁₋₂alkyl, more suitably H or methyl. Suitably, R^{8a} is H.

Preferably, R⁷ and/or R⁸ are independently a hydrogen atom (I); C₁₋₂alkyl such as methyl; C₃₋₆cycloalkyl; or phenyl optionally substituted by one or two (e.g. one) substituents independently being: fluoro, chloro, C₁₋₂alkyl, C₁fluoroalkyl, C₁₋₂alkoxy or

C₁fluoroalkoxy, or R⁷ and R⁸ together are —(CH₂)ₙ⁶- or —(CH₂)ₙ⁸—X⁷—(CH₂)ₙ⁹— wherein X⁷ is NR¹⁴ or preferably O.

When R⁷ is cycloalkyl or optionally substituted phenyl, then preferably R⁸ is neither cycloalkyl nor optionally substituted phenyl. In this case, R⁸ can for example be H.

More preferably, R⁷ and/or R⁸ independently are a hydrogen atom (H) or C₁₋₂alkyl. It is preferable that R⁸ is a hydrogen atom (H).

Preferably n⁶ is 4 or 5. Preferably n⁷ is 3 or 4. Preferably, n⁸, n⁹ and/or n¹⁰ independently is/are 2.

Preferably, R¹² and/or R¹³ independently are H; C₁₋₂alkyl such as methyl; C₃₋₆cycloalkyl; or phenyl optionally substituted by one or two (e.g. one) substituents independently being: fluoro, chloro, C₁₋₂alkyl, C₁fluoroalkyl, C₁₋₂alkoxy or C₁fluoroalkoxy; or R¹² and R¹³ together are —(CH₂)ₙ^{6a} or —(CH₂)ₙ^{8a}—X¹²—(CH₂)ₙ^{9a}— in which X¹² is NR^{14a} or preferably 0.

When R¹² is cycloalkyl or optionally substituted phenyl, then preferably R¹³ is neither cycloalkyl nor optionally substituted phenyl. In this case, R¹³ can for example be H.

More preferably, R¹² and/or R¹³ independently are a hydrogen atom (H) or C₁₋₂alkyl. It is preferable that R¹³ is a hydrogen atom (H).

Preferably n^{6a} is 4 or 5. Preferably n^{7a} is 3 or 4. Preferably, n^{8a}, n^{9a} and/or n^{10a} independently is/are 2.

In one embodiment of the invention, NR⁷R⁸ and/or NR¹²R¹³ can for example independently be

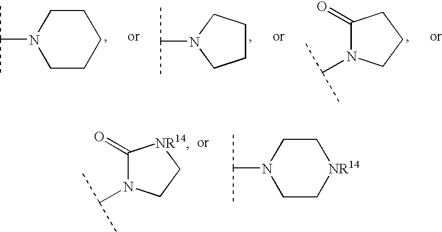

(i.e. R¹² and R¹³ together are —(CH₂)₂—N(R¹⁴)—(CH₂)₂—, or R⁷ and R⁸ together are

—(CH₂)₂—N(R^{14a})—(CH₂)₂— respectively), or

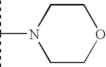

(i.e. R¹² and R¹³ together or R⁷ and R⁸ together are —(CH₂)₂—O—(CH₂)₂—), or NMe₂.

Suitably, R¹⁴, R^{14a}, R¹⁷ and/or R^{17a} independently are: a hydrogen atom (H); C₁₋₂alkyl; C₁fluoroalkyl (e.g. CF₃); —C(O)Me; —C(O)NH₂; or —S(O)₂Me. More suitably, R¹⁴, R^{14a}, R¹⁷ and/or R^{17a} independently is/are: H, C₁₋₂alkyl, or —C(O)Me; or for example H or C₁₋₂alkyl.

Suitably, R¹⁵ is a hydrogen atom (H) or C₁₋₄alkyl (e.g. ^{t}Bu or C₁₋₂alkyl e.g. methyl); more suitably, R¹⁵ is a hydrogen atom (R).

Where R^{15a}, independent of other R^{15a}, is a hydrogen atom (H) or C₁₋₄alkyl, it can for example be H, tBu or C₁₋₂alkyl such as methyl. Suitably, R^{15a}, independent of other R^{15a}, is H or C₁₋₂alkyl, more preferably H.

Preferably, R^{15b} is H.

Suitably, R¹⁶ is C₁₋₄allyl (e.g. C₁₋₂alkyl) or C₃₋₆cycloalkyl (e.g. C₅₋₆cycloalkyl); more suitably R¹⁶ is C₁₋₄alkyl (e.g. C₁₋₂alkyl).

Suitably, R^{16a} is:

C₁₋₄alkyl (e.g. C₁₋₂alkyl);

C₃₋₆cycloalkyl (e.g. C₅₋₆cycloalkyl) optionally substituted by one oxo (═O), OH or methyl substituent (e.g. optionally substituted at the 3- or 4-position of a C₅₋₆cycloalkyl ring; and/or preferably unsubstituted C₃₋₆cycloalkyl);

C₃₋₆cycloalkyl-CH₂- (e.g. C₅₋₆cycloalkyl-CH2-); pyridinyl (e.g. pyridin-2-yl) optionally substituted on a ring carbon atom by one of: a halogen atom, C₁₋₂alkyl, C₁fluoroalkyl, C₁₋₂alkoxy or C₁fluoroalkoxy;

Ar5c;

phenyl optionally substituted by one or two substituents independently being: a halogen atom,C₁₋₂alkyl, C₁fluoroalkyl, C₁₋₂alkoxy or C₁fluoroalkoxy;

benzyl optionally substituted on its ring by one or two substituents independently being: a halogen atom, C₁₋₂alkyl, C₁fluoroalkyl, C₁₋₂alkoxy or C₁fluoroalkoxy; or

a 5- or 6-membered saturated heterocyclic ring connected at a ring-carbon and containing one or two ring-hetero-atoms independently selected from O, S, and N; wherein any ring-nitrogens which are present are present as NR²⁷ where R²⁷ is H, C₁₋₂alkyl or —C(O)Me (preferably H or C₁₋₂alkyl); and wherein the ring is not substituted at carbon.

Preferably, R^{16a} is: C₁₋₄alkyl (e.g. C₁₋₂alkyl); unsubstituted C₃₋₆cycloalkyl (e.g. unsubstituted C₅₋₆cycloalkyl); phenyl optionally substituted by one or two substituents independently being: a halogen atom, C₁₋₂alkyl, C₁fluoroalkyl, C₁₋₂alkoxy or C₁fluoroalkoxy; or benzyl optionally substituted on its ring by one or two substituents independently being: a halogen atom, C₁₋₂alkyl, C₁fluoroalkyl, C₁₋₂alkoxy or C₁fluoroalkoxy. Preferably, R^{16a} is C₁₋₄alkyl (e.g. C₁₋₂alkyl).

Suitably, R³⁰, independent of other R³⁰, is a hydrogen atom (H) or C₁₋₄alkyl, for example H, t-butyl or C₁₋₂alkyl.

Preferably, the compound of formula (I) or the salt thereof is racemic at the carbon atom bearing the R⁴ and R⁵ groups, or (more preferably) the compound of formula (I) or the salt thereof is a compound of formula (IA) or a salt thereof:

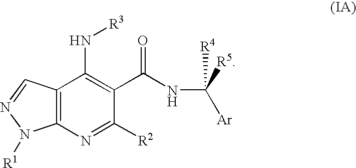

Formula (IA) means that more than 50% of the compound or salt present has the stereochemistry shown at the carbon atom bearing the R⁴ and R⁵ groups.

In Formula (IA), on a molarity basis, preferably 70% or more, more preferably 75% or more, still more preferably 85% or more, yet more preferably 90% or more, for example 95% or more such as 98% or more, of the compound or salt present has the stereochemistry shown at the carbon atom bearing the R⁴ and R⁵ groups.

Preferably, in Formula (IA), the stereochemistry at the carbon atom bearing the R⁴ and R⁵ groups is such that there is an enantiomeric excess (e.e.) of 50% or more at the carbon atom bearing the R⁴ and R⁵ groups (ignoring the stereochemistry at any other carbon atoms). More preferably, the enantiomeric excess (e.e.) is 70% or more or 80% or more, still more preferably 90% or more, yet more preferably 95% or more, at the carbon atom bearing the R⁴ and R⁵ groups (ignoring the stereochemistry at any other carbon atoms).

“Enantiomeric excess” (e.e.) is defined as the percentage of the major isomer present minus the percentage of the minor isomer present. For example, if 95% of major isomer is present and 5% of the minor isomer is present, then the e.e. would be 90%.

In formula (IA), it is preferable that R⁴ is not a hydrogen atom (H). In formula (IA), more preferably R⁴ is methyl, ethyl, C₁fluoroalkyl (such as CF₃), —CH₂OH, or —CH₂OMe; still more preferably R⁴ is methyl, ethyl, CF₃ or —CH₂OH; yet more preferably R⁴ is methyl or ethyl; and most preferably R⁴ is ethyl.

In formula (IA), it is particularly preferable that R⁵ is a hydrogen atom (H) and R⁴ is not a hydrogen atom (H). In formula (IA), it is more preferable that R⁵ is a hydrogen atom (H); and R⁴ is methyl, ethyl, C₁fluoroalkyl (such as CF₃), —CH₂OH, or —CH₂OMe (e.g. methyl, ethyl, CF₃ or —CH₂OH). In formula (IA), it is most preferable that R⁵ is a hydrogen atom (H); and R⁴ is methyl or ethyl (preferably ethyl).

In formula (A), when R⁴ is not a hydrogen atom (H), and optionally when R⁵ is a hydrogen atom (H), it is particularly preferable that Ar, such as having sub-formula (x1), is a monocycle. That is, in formula (IA) and when R⁴ is not a hydrogen atom (H), it is particularly preferable that two adjacent groups selected from R^{6A}, R^{6B}, R^{6D}, R^{6E} and R^{6F} are not taken together to form part of a second ring.

The Examples 1, 8, 24, 28, 63, 127, 129, 174, and 178 disclosed herein, having and/or believed to have the formula (IA) wherein R⁵ is H, and wherein R⁴ is methyl, ethyl, —CH₂OH, or —CH₂OMe, and wherein Ar is a monocycle, generally have greater PDE4B inhibitory activity than the comparable Examples 6, 7,29, 26, 64, 126, 124, 170, and 177 which have and/or are believed to have the opposite stereochemistry (including a majority of the opposite stereochemistry) at the CR⁴R⁵ (benzylic) carbon atom.

In an especially preferable embodiment, HN—CR⁴R⁵—Ar is the HN—CR⁴R⁵—Ar group as defined in any one of Examples 1 to 314 and/or as defined in any one of Examples 315 to 382.

It is particularly preferred that the compound of formula (I) or the salt thereof is:

1-ethyl-N-[(1R)-1-phenylpropyl]-4-(tetrahydro-2H-pyran-4-ylamino)-1H-pyrazolo[3,4-b]pyridine-5-carboxamide

1-ethyl-N-(1-methyl-1-phenylethyl)-4-(tetrahydro-2H-pyran-4-ylamino)-1H-pyrazolo[3,4-b]pyridine-5-carboxamide

1-ethyl-N-{1-[4-(methylsulfonyl)phenyl]ethyl}-4-(tetrahydro-2H-pyran-4-ylamino)-1H-pyrazolo[3,4-b]pyridine-5-carboxamide

N-(diphenylmethyl)-1-ethyl-4-(tetrahydro-2H-pyran-4-ylamino)-1H-pyrazolo[3,4-b]pyridine-5-carboxamide

1-ethyl-N-[1-(3-pyridinyl)ethyl]-4-(tetrahydro-2H-pyran-4-ylamino)-1H-pyrazolo[3,4-b]pyridine-5-carboxamide

1-ethyl-N-[(1S)-1-phenylpropyl]-4-(tetrahydro-2H-pyran-4-ylamino)-1H-pyrazolo[3,4-b]pyridine-5-carboxamide

1-ethyl-N-[(1S)-1-phenylethyl]-4-(tetrahydro-2H-pyran-4-ylamino)-1H-pyrazolo[3,4-b]pyridine-5-carboxamide

1-ethyl-N-[(1R)-1-phenylethyl]-4-(tetrahydro-2H-pyran-4-ylamino)-1H-pyrazolo[3,4-b]pyridine-5-carboxamide

1-ethyl-N-[1-methyl-1-(4-pyridinyl)ethyl]-4-(tetrahydro-2H-pyran-4-ylamino)-1H-pyrazolo[3,4-b]pyridine-5-carboxamide

1-ethyl-N-[(1R)-1-phenylethyl]-4-(tetrahydro-2H-pyran-4-ylamino)-1H-pyrazolo[3,4-b]pyridine-5-carboxamide

N-[1-(4-chlorophenyl)propyl]-1-ethyl-4-(tetrahydro-2H-pyran-4-ylamino)-1H-pyrazolo[3,4-b]pyridine-5-carboxamide

1-ethyl-N-{1-[4-(ethyloxy)phenyl]ethyl}-4-(tetrahydro-2H-pyran-4-ylamino)-1H-pyrazolo[3,4-b]pyridine-5-carboxamide

1-ethyl-N-(3-hydroxy-1-phenylpropyl)-4-(tetrahydro-2H-pyran-4-ylamino)-1H-pyrazolo[3,4-b]pyridine-5-carboxamide

1-ethyl-N-[1-(3-hydroxyphenyl)ethyl]-4-(tetrahydro-2H-pyran-4-ylamino)-1H-pyrazolo[3,4-b]pyridine-5-carboxamide

N-[2-(dimethylamino)-1-phenylethyl]-1-ethyl-4-(tetrahydro-2H-pyran-4-ylamino)-1H-pyrazolo[3,4-b]pyridine-5-carboxamide

1-ethyl-N-[1-phenyl-2-(1-pyrrolidinyl)ethyl]-4-(tetrahydro-2H-pyran-4-ylamino)-1H-pyrazolo[3,4-b]pyridine-5-carboxamide

1-ethyl-N-[1-(hydroxymethyl)-1-phenylpropyl]-4-(tetrahydro-2H-pyranylanino)-1H-pyrazolo[3,4-b]pyridine-5-carboxamide

1-ethyl-N-{1-[4-(propyloxy)phenyl]ethyl}-4-(tetrahydro-2H-pyran-4-ylamino)-1H-pyrazolo[3,4-b]pyridine-5-carboxamide

methyl 3-({[1-ethyl4-(tetrahydro-2H-pyran-4-ylamino)-1H-pyrazolo[3,4-b]pyridin-5-yl]carbonyl}amino)-3-phenylpropanoate

1-ethyl-N-[1-(4-fluorophenyl)ethyl]-4-(tetrahydro-2H-pyran-4-ylamino)-1H-pyrazolo[3,4-b]pyridine-5-carboxamide

N-[1-(4-chlorophenyl)ethyl]-1-ethyl-4-(tetrahydro-2H-pyran-4-ylamino)-1H-pyrazolo[3,4-b]pyridine-5-carboxamide

ethyl ({[1 -ethyl-4-(tetrahydro-2H-pyran-4-ylamino)-1H-pyrazolo[3,4-b]pyridin-5-yl]carbonyl}amino)(phenyl)acetate

1-ethyl-N-{(1R)-1-[3-(methyloxy)phenyl]ethyl}-4-(tetrahydro-2H-pyran-4-ylamino)- 1H pyrazolo[3,4-b]pyridine-5-carboxamide

1-ethyl-N-[(1S)-2-(methyloxy)-1-phenylethyl]-4-(tetrahydro-2H-pyran-4-ylamino)-1H-pyrazolo[3,4-b]pyridine-5-carboxamide

N-[(1R)-2-amino-2-oxo-1-phenylethyl]-1-ethyl-4-(tetrahydro-2H-pyran-4-ylamino)-1H-pyrazolo[3,4-b]pyridine-5-carboxamide

1-ethyl-N-[(1R)-2-hydroxy-1-phenylethyl]-4-(tetrahydro-2H-pyran-4-ylamino)-1H-pyrazolo[3,4-b]pyridine-5-carboxamide

1-ethyl-N-[(1R)-1-(4-nitrophenyl)ethyl]-4-(tetrahydro-2H-pyran-4-ylamino)-1H-pyrazolo[3,4-b]pyridine-5-carboxamide

1-ethyl-N-[(1S)-2-hydroxy-1-phenylethyl]-4-(tetrahydro-2H-pyran-4-ylamino)-1H-pyrazolo[3,4-b]pyridine-5-carboxamide

1-ethyl-N-[(1R)-2-(methyloxy)-1-phenylethyl]-4-(tetrahydro-2H-pyran-4-ylamino)-1H-pyrazolo[3,4-b]pyridine-5-carboxamide

1-ethyl-N-(2-hydroxy-1,1-diphenylethyl)-4-(tetrahydro-2H-pyran-4-ylamino)-1H-pyrazolo[3,4-b]pyridine-5-carboxamide

N-[1-(3-cyanophenyl)ethyl]-1-ethyl-4-(tetrahydro-2H-pyran-4-ylamino)-1H-pyrazolo[3,4-b]pyridine-5-carboxamide

N-[cyano(phenyl)methyl]-1-ethyl-4-(tetrahydro-2H-pyran-4-ylamino)-1H-pyrazolo[3,4-b]pyridine-5-carboxamide

N-{cyclopropyl[4(methyloxy)phenyl]methyl}-1-ethyl-4-(tetrahydro-2-H-pyran-4-ylamino)-1H-pyrazolo[3,4-b]pyridine-5-carboxamide

1-ethyl-N-[1-(1-naphthalenyl)ethyl]-4-(tetrahydro-2H-pyran-4-ylamino)-1H-pyrazolo[3,4-b]pyridine-5-carboxamide

N-(1,2-diphenylethyl)-1-ethyl-4-(tetrahydro-2H-pyran-4-ylamino)-1H-pyrazolo[3,4-b]pyridine-5-carboxamide

1-ethyl-N-{1-[4-(methyloxy)phenyl]butyl}-4-(tetrahydro-2H-pyran-4-ylamino)-1H-pyrazolo[3,4-b]pyridine-5-carboxamide

1-ethyl-N-[(1R)-1-(1-naphthalenyl)ethyl]-4-(tetrahydro-2H-pyran-4-ylamino)-1H-pyrazolo[3,4-b]pyridine-5-carboxamide

1-ethyl-N-[(1S)-1-(1-naphthalenyl)ethyl]-4-(tetrahydro-2H-pyran-4-ylamino)-1H-pyrazolo[3,4-b]pyridine-5-carboxamide

N-[1-(aminocarbonyl)-1-phenylpropyl]-1-ethyl-4-(tetrahydro-2H-pyran-4-ylamino)-1H-pyrazolo[3,4-b]pyridine-5-carboxamide

1-ethyl-N-(1-phenylcyclopentyl)-4-(tetrahydro-2H-pyran-4-ylamino)-1H-pyrazolo[3,4-b]pyridine-5-carboxamide

1-ethyl-N-(4-phenyltetrahydro-2H-pyran-4-yl)-4-(tetrahydro-2H-pyran-4-ylamino)-1H-pyrazolo[3,4-b]pyridine-5-carboxamiide

1-ethyl-N-(1-phenylcyclopropyl)-4-(tetrahydro-2H-pyran-4-ylamino)-1H-pyrazolo[3,4-b]pyridine-5-carboxamide

N-{1-[4-(cyclohexyloxy)-3-methylphenyl]ethyl}-1-ethyl-4-(tetrahydro-2H-pyran-4-ylamino)-1H-pyrazolo[3,4-b]pyridine-5-carboxamide

N-{1-[3-(cyclohexyloxy)-4-(methyloxy)phenyl]ethyl}-1-ethyl-4-(tetrahydro-2H-pyran-4-ylamino)-1H-pyrazolo[3,4-b]pyridine-5-carboxamide

N-[1-(2,3-dichlorophenyl)ethyl]-1-ethyl-4-(tetrahydro-2H-pyran-4-ylamino)-1H-pyrazolo[3,4-b]pyridine-5-carboxamide

N-{1-[4-(cyclohexyloxy)-3-hydroxyphenyl]ethyl}-1-ethyl-4-(tetrahydro-2H-pyran-4-ylamino)-1H-pyrazolo[3,4-b]pyridine-5-carboxamide

N-{1-[4-(cyclopentyloxy)phenyl]ethyl}-1-ethyl-4-(tetrahydro-2H-pyran-4-ylamino)-pyrazolo[3,4-b]pyridine-5-carboxamide

1-ethyl-N-[1 -(4-methylphenyl)ethyl]-4-(tetrahydro-2H-pyran-4-ylamino)-1H-pyrazolo[3,4-b]pyridine-5-carboxamide

N-{1-[4-(1 ,1-dimethylethyl)phenyl]cycloheptyl}-1-ethyl-4-(tetrahydro-2H-pyran-4-ylamino)-1H-pyrazolo[3,4-b]pyridine-5-carboxamide

N-[1-(4-bromophenyl)ethyl]-1-ethyl-4-(tetrahydro-2H-pyran-4-ylamino)-1H-pyrazolo[3,4-b]pyridine-5-carboxamide

1-ethyl-N-[(1S)-1-(4-iodophenyl)ethyl]-4-(tetrahydro-2H-pyran-4-ylamino)-1H-pyrazolo[3,4-b]pyridine-5-carboxamide

N-{1-[4-(aminosulfonyl)phenyl]ethyl}-1-ethyl-4-(tetrahydro-2H-pyran-4-ylamino)-1H-pyrazolo[3,4-b]pyridine-5-carboxamide

1-ethyl-N-(1-methyl-1-phenylpropyl)-4-(tetrahydro-2H-pyran-4-ylamino)-1H-pyrazolo[3,4-b]pyridine-5-carboxamide

N-[1-(1 ,3-benzodioxol-5-yl)cyclohexyl]-1-ethyl-4-(tetrahydro-2H-pyran-4-ylamino)-1H-pyrazolo[3 ,4-b]pyridine-5-carboxamide

1-ethyl-N-{1-[4-(methyloxy)phenyl]cyclohexyl}-4-(tetrahydro-2H-pyran-4-ylamino)-1H-pyrazolo[3,4-b]pyridine-5-carboxamide

1-ethyl-N-[1-(4-fluorophenyl)cyclohexyl]-4-(tetrahydro-2H-pyran-4-ylamino)-1H-pyrazolo[3 ,4-b]pyridine-5-carboxamide

N-[1-(3-chlorophenyl)cyclopentyl]-1-ethyl-4-(tetrahydro-2H-pyran-4-ylamino)-1H-pyrazolo[3,4-b]pyridine-5-carboxamide

N-[1-(2-chlorophenyl)cyclopentyl]-1-ethyl-4-(tetrahydro-2H-pyran-4-ylamino)-1H-pyrazolo[3,4-b]pyridine-5-carboxamide

N-{1-[4-(1,1-dimnethylethyl)phenyl]cyclohexyl}-1-ethyl-4-(tetrahydro-2H-pyran-4-ylamino)-1H-pyrazolo[3,4-b]pyridine-5-carboxamide 1-ethyl-N-{1-[4-(1-methylethyl)phenyl]ethyl}-4-(tetrahydro-2H-pyran-4-ylamino)-1H-pyrazolo[3,4-b]pyridine-5-carboxamide

N-[1-(3,5-dimethylphenyl)ethyl]-1-ethyl-4-(tetrahydro-2H-pyran-4-ylamino)-1H-pyrazolo[3 ,4-b]pyridine-5-carboxamide

1-ethyl-N-[(1S,2R)-2-hydroxy-1-phenylpropyl]-4-(tetrahydro-2H-pyran-4-ylamino)-1H-pyrazolo[3,4-b]pyridine-5-carboxamide

1-ethyl-N-{(1R)-1-[4-(methyloxy)phenyl]ethyl}-4-(tetrahydro-2H-pyran-4-ylamino)-1H-pyrazolo[3,4-b]pyridine-5-carboxamide

1-ethyl-N-{(1S)-1-[4-(methyloxy)phenyl]ethyl}-4-(tetrahydro-2H-pyran-4-ylamino)-1H-pyrazolo[3,4-b]pyridine-5-carboxamide

1-ethyl-N-(1-phenylhexyl)-4-(tetrahydro-2H-pyran-4-ylamino)-1H-pyrazolo[3,4-b]pyridine-5-carboxamide

1-ethyl-N-(1-phenylpentyl)-4-(tetrahydro-2H-pyran-4-ylamino)-1H-pyrazolo[3,4-b]pyridine-5-carboxamide

1-ethyl-N-(2-methyl-1-phenylpropyl)-4-(tetrahydro-2H-pyran-4-ylamino)-1H-pyrazolo[3,4-b]pyridine-5-carboxamide

1-ethyl-N-(1-phenylbutyl)-4-(tetrahydro-2H-pyran-4-ylamino)-1H-pyrazolo[3,4-b]pyridine-5-carboxamide

1-ethyl-4-(tetrahydro-2H-pyran-4-ylamino)-N-(2,2,2-trifluoro-1-phenylethyl)-1H-pyrazolo[3,4-b]pyridine-5-carboxamide

N-[cyclopropylphenyl)methyl]-1-ethyl-4-(tetrahydro-2H-pyran-4-ylamino)-1H-pyrazolo[3,4-b]pyridine-5-carboxamide

1-ethyl-N-[1-(4-fluorophenyl)propyl]-4-(tetrahydro-2H-pyran-4-ylamino)-1H-pyrazolo[3,4-b]pyridine-5-carboxamide

N-[1-(2,3-dichlorophenyl)propyl]-1-ethyl-4-(tetrahydro-2H-pyran-4-ylamino)-1H-pyrazolo[3,4-b]pyridine-5-carboxamide

1-ethyl-N-[(1R)-1-(4-methylphenyl)ethyl]-4-(tetrahydro-2H-pyran-4-ylamino)-1H-pyrazolo[3,4-b]pyridine-5-carboxamide

1-ethyl-N-(1-phenylethyl)-4-(tetrahydro-2H-pyran-4-ylamino)-1H-pyrazolo[3,4-b]pyridine-5-carboxamide

N-[(1R)-1-(4-bromophenyl)ethyl]-1-ethyl-4-(tetrahydro-2H-pyran-4-ylamino)-1H-pyrazolo[3,4-b]pyridine-5-carboxamide

N-[1-(4-chlorophenyl)-2-hydroxyethyl]-1-ethyl-4-(tetrahydro-2H-pyran-4-ylamino)-1H-pyrazolo[3,4-b]pyridine-5-carboxamide

N-[1-(3,4-dichlorophenyl)-2-hydroxyethyl]-1-ethyl-4-(tetrahydro-2H-pyran-4-ylamino)-1H-pyrazolo[3,4-b]pyridine-5-carboxamide 1-ethyl-N-{1-[3-(methyloxy)phenyl]propyl}-4-(tetrahydro-2H-pyran-4-ylamino)-1H-pyrazolo[3,4-b]pyridine-5-carboxamide

1-ethyl-N-{1 -[4-(methyloxy)phenyl]propyl}-4-(tetrahydro-2H-pyran-4-ylamino)-1H-pyrazolo[3,4-b]pyridine-5-carboxamide

N-[1 -(4-bromophenyl)propyl]-1-ethyl-(tetrahydro-2H-pyran-4-ylamino)-1H-pyrazolo[3,4-b]pyridine-5-carboxamide

1-ethyl-N- {1-[4-(propyloxy)phenyl]propyl}-4-(tetrahydro-2H-pyran-4-ylamino)-1H-pyrazolo[3,4-b]pyridine-5-carboxamide

N-[1-(3,5-dimethylphenyl)propyl]-1-ethyl-4-(tetrahydro-2H-pyran-4-ylamino)-1H-pyrazolo[3,4-b]pyridine-5-carboxamide

1-ethyl-N-[1-(4-methylphenyl)propyl]-4-(tetrahydro-2H-pyran-4-ylamino)- 1H-pyrazolo[3,4-b]pyridine-5-carboxamide

1-ethyl-N-{1-[4-(1-methylethyl)phenyl]propyl}-4-(tetrahydro-2H-pyran-4-ylamino)- pyrazolo[3,4-b]pyridine-5-carboxamide

1-ethyl-N-[1-(2-methylphenyl)ethyl]-4-(tetrahydro-2H-pyran-4-ylamino)-1H-pyrazolo[3,4-b]pyridine-5-carboxamide

N-(1-{4-[(difluoromethyl)oxy]phenyl}ethyl)-1-ethyl-4-(tetrahydro-2H-pyran-4-ylamino)-1H-pyrazolo[3,4-b]pyridine-5-carboxamide

1-ethyl-4-(tetrahydro-2H-pyran-4-ylamino)-N-{1-[4-(trifluoromethyl)phenyl]ethyl}- 1H pyrazolo[3,4-b]pyridine-5-carboxamide

1-ethyl-N-[1-(2-methylphenyl)propyl]-4-(tetrahydro-2H-pyran-4-ylamino)-1H-pyrazolo[3,4-b]pyridine-5-carboxamide

1-ethyl-N- {1-[4-(ethyloxy)phenyl]propyl}-4-(tetrahydro-2H-pyran-4-ylamino)-1H-pyrazolo[3,4-b]pyridine-5-carboxamide

N-(1-{4-[(difluoromethyl)oxy]phenyl}propyl)-1-ethyl-4-(tetrahydro-2H-pyran-4-ylamino)-1H-pyrazolo[3,4-b]pyridine-5-carboxamide

1-ethyl-4-(tetrahydro-2H-pyran-4-ylamino)-N-{1-[4-(trifluoromethyl)phenyl]propyl}-1H-pyrazolo[3,4b]pyridine-5-carboxamide

N-[1-(3,4-dimethylphenyl)propyl]-1-ethyl-4-(tetrahydro-2H-pyran-4-ylamino)-1H-pyrazolo[3,4-b]pyridine-5-carboxamide

N-[1-(2,3;.dimethylphenyl)ethyl]-1-ethyl-4-(tetrahydro-2H-pyran-4-ylamino)-1H-pyrazolo[3,4-b]pyridine-5-carboxamide

N-[1-(2,4-dimethylphenyl)ethyl]-1-ethyl-4-(tetrahydro-2H-pyran-4-ylamino)-1H-pyrazolo[3,4-b]pyridine-5-carboxamide

N-[1-(4-chloro-2-fluorophenyl)ethyl]-1-ethyl-4-(tetrahydro-2H-pyran-4-ylamino)-1H-pyrazolo[3,4-b]pyridine-5-carboxamide

N-[1-(3-chloro-4-methylphenyl)ethyl]-1-ethyl-4-(tetrahydro-2H-pyran-4-ylamino)-1H-pyrazolo[3,4-b]pyridine-5-carboxamide

N-[1-(2,3-dimethylphenyl)propyl]-1-ethyl-4-(tetrahydro-2H-pyran-4-ylamino)-1H-pyrazolo[3,4-b]pyridine-5-carboxamide

N-[1-(2,4-dimethylphenyl)propyl]-1-ethyl-4-(tetrahydro-2H-pyran-4-ylamino)-1H-pyrazolo[3,4-b]pyridine-5-carboxamide

N-[1-(4-chloro-2-fluorophenyl)propyl]-1-ethyl-4-(tetrahydro-2H-pyran-4-ylamino)-1H-pyrazolo[3,4-b]pyridine-5-carboxamide

N-[1-(3-chloro-4-methylphenyl)propyl]-1-ethyl-4-(tetrahydro-2H-pyran-4-ylamino)-1H-pyrazolo[3,4-b]pyridine-5-carboxamide

1-ethyl-N-[1-(3-hydroxyphenyl)propyl]-4-(tetrahydro-2H-pyran-4-ylamino)-1H-pyrazolo[3,4-b]pyridine-5-carboxamide

N-[1-(2,3-dihydro-1H-inden-5-yl)ethyl]-1-ethyl-4-(tetrahydro-2H-pyran-4-ylamino)-1H-pyrazolo[3,4-b]pyridine-5-carboxamide

1-ethyl-N-[1-(5,6,7,8-tetrahydro-2-naphthalenyl)ethyl]-4-(tetrahydro-2H-pyran-4-ylamino)-1H-pyrazolo[3,4-b]pyridine-5-carboxamide

N-[1-(4-bromophenyl)-2,2,2-trifluoroethyl]-1-ethyl-4-(tetrahydro-2H-pyran-4-ylamino)-1H-pyrazolo[3,4-b]pyridine-5-carboxamide

1-ethyl-4-(tetrahydro-2H-pyran-4-ylamino)-N-{2,2,2-trifluoro-1-[3-(methyloxy)phenyl]ethyl}-1H-pyrazolo[3,4-b]pyridine-5-carboxamide

4-(cyclohexylamino)-1-ethyl-N-{1-[4-(methylsulfonyl)phenyl]ethyl}-1H-pyrazolo[3,4-b]pyridine-5-carboxamide

4-(cyclohexylamino)-1-ethyl-N-[(1R)-1-phenylpropyl]-1H-pyrazolo[3,4-b]pyridine-5-carboxamide

4-(cyclohexylamino)-N-(diphenylmethyl)-1-ethyl-1H-pyrazolo[3,4-b]pyridine-5-carboxamide

4-(cyclohexylamino)-1-ethyl-N-[(1R)-1-phenylethyl]-1H-pyrazolo[3,4-b]pyridine-5-carboxamide

ethyl ({[4-(cyclohexylamino)-1-ethyl-1H-pyrazolo[3,4-b]pyridin-5-yl]carbonyl}amino)(phenyl)acetate

N-[1-(4-chlorophenyl)ethyl]-4-(cyclohexylamino)-1-ethyl-1H-pyrazolo[3,4-b]pyridine-5-carboxamide

4-(cyclohexylamino)-1-ethyl-N-(1-methyl-1-phenylethyl)-1H-pyrazolo[3,4-b]pyridine-5-carboxamide

4-(cyclohexylamino)-1-ethyl-N-[1-(4-fluorophenyl)ethyl]-1H-pyrazolo[3,4-b]pyridine-5-carboxamide

N-[1-(4-chlorophenyl)propyl]-4-(cyclohexylamino)-1-ethyl-1H-pyrazolo[3,4-b]pyridine-5-carboxamide

4-(cyclohexylamino)-N-(1,2-diphenylethyl)-1-ethyl-1H-pyrazolo[3,4-b]pyridine-5-carboxamide

4-(cyclohexylamino)-1-ethyl-N-{1-[4-(propyloxy)phenyl]ethyl}-1H-pyrazolo[3,4-b]pyridine-5-carboxamide

methyl 3-( {[4-(cyclohexylamino)-1-ethyl-1H-pyrazolo[3,4-b]pyridin-5-yl]carbonyl}amino)-3-phenylpropanoate

4-(cyclohexylamino)-1-ethyl-N-[1-(hydroxymethyl)-1-phenylpropyl]-1H-pyrazolo[3 ,4-b]pyridine-5-carboxamide

4-(cyclohexylamino)-1-ethyl-N-(3-hydroxy-1-phenylpropyl)-1H-pyrazolo[3,4-b]pyridine-5-carboxamide

4-(cyclohexylamino)-1-ethyl-N-{1-[4-(ethyloxy)phenyl]ethyl}-1H-pyrazolo[3,4-b]pyridine-5-carboxamide

4-(cyclohexylamino)-1-ethyl-N-[1-(3-hydroxyphenyl)ethyl]-1H-pyrazolo[3,4-b]pyridine-5-carboxamide

4-(cyclohexylamino)-1-ethyl-N-[1-phenyl-2-(1-pyrrolidinyl)ethyl]-1H-pyrazolo[3,4-b]pyridine-5-carboxamide

4-(cyclohexylamino)-N-[2-(dimethylamino)-1-phenylethyl]-1-ethyl-1H-pyrazolo[3,4-b]pyridine-5-carboxamide

4-(cyclohexylamino)-1-ethyl-N-[(1R)-2-(methyloxy)-1-phenylethyl]-1H-pyrazolo[3,4-b]pyridine-5-carboxamide

N-[(1R)-2-amino-2-oxo-1-phenylethyl]-4-(cyclohexylamino)-1-ethyl-1H-pyrazolo[3,4-b]pyridine-5-carboxamide

4-(cyclohexylamino)-1-ethyl-N-[(1R)-2-hydroxy-1-phenylethyl]-1H-pyrazolo[3,4-b]pyridine-5-carboxamide

4-(cyclohexylamino)-1-ethyl-N-[(1S)-2-hydroxy-1-phenylethyl]-1H-pyrazolo[3,4-b]pyridine-5-carboxamide

4-(cyclohexylamino)-1-ethyl-N-{(1R)-1-[3-(methyloxy)phenyl]ethyl}-1H-pyrazolo[3,4-b]pyridine-5-carboxamide

4-(cycrohexylamino)-1-ethyl-N-[(1S)-2-(methyloxy)-1-phenylethyl]-1H-pyrazolo[3,4-b]pyridine-5-carboxamide

4-(cyclohexylamino)-1-ethyl-N-[(1R)-1-(4-nitrophenyl)ethyl]-1H-pyrazolo[3,4-b]pyridine-5-carboxamide

4-(cyclohexylamino)-1-ethyl-N-[(1S)-1-(1-naphthalenyl)ethyl]-1H-pyrazolo[3,4-b]pyridine-5-carboxamide

4-(cyclohexylamino)-1-ethyl-N-[phenyl(4-phenyl-1 ,3-thiazol-2-yl)methyl]-1H-pyrazolo[3 ,4-b]pyridine-5-carboxamide

N-[cyano(phenyl)methyl]-4-(cyclohexylamino)-1-ethyl-1H-pyrazolo[3,4-b]pyridine-5-carboxamide

4-(cyclohexylamino)-1-ethyl-N-[1-(1-naphthalenyl)ethyl]-1H-pyrazolo[3,4-b]pyridine-5-carboxamide

4-(cyclohexylamino)-1-ethyl-N-(2-hydroxy-1,1-diphenylethyl)-1H-pyrazolo[3,4-b]pyridine-5-carboxamide

4-(cyclohexylamino)-1-ethyl-N-{(1R)-1-[4-(methyloxy)phenyl]ethyl}-1H-pyrazolo[3,4-b]pyridine-5-carboxamide

4-(cyclohexylamino)-1-ethyl-N-[1-(4-fluorophenyl)propyl]-1H-pyrazolo[3,4-b]pyridine-5-carboxamide

4-(cyclohexylamino)-N-[1-(2,3-dichlorophenyl)propyl]-1-ethyl-1H-pyrazolo[3,4-b]pyridine-5-carboxamide

4-(cyclohexylamino)-1-ethyl-N-[(1R)-1-(4-methylphenyl)ethyl]-1H-pyrazolo[3,4-b]pyridine-5-carboxamide

4-(cyclohexylamino)-1-ethyl-N-(1-phenylethyl)-1H-pyrazolo[3,4-b]pyridine-5-carboxamide

N-[(1R)-1-(4-bromophenyl)ethyl]-4-(cyclohexylamino)-1-ethyl-1H-pyrazolo[3,4-b]pyridine-5-carboxamide

4-(cyclohexylamino)-N-[1-(2,3-dichlorophenyl)ethyl]-1-ethyl-1H-pyrazolo[3,4-b]pyridine-5-carboxamide

4-(cyclohexylamino)-1-ethyl-N-{1-[3-(methyloxy)phenyl]propyl}-1H-pyrazolo[3,4-b]pyridine-5-carboxamide

4-(cyclohexylamino)-1-ethyl-N-{1-[4-(methyloxy)phenyl]propyl}-1H-pyrazolo[3,4-b]pyridine-5-carboxamide

N-[1-(4-bromophenyl)propyl]-4-(cyclohexylamino)-1-ethyl-1H-pyrazolo[3,4-b]pyridine-5-carboxamide

4-(cyclohexylamino)-1-ethyl-N-{1-[4-(propyloxy)phenyl]propyl}-1H-pyrazolo[3,4-b]pyridine-5-carboxamide

4-(cyclohexylamino)-N-[1-(3,5-dimethylphenyl)propyl]-1-ethyl-1H-pyrazolo[3,4-b]pyridine-5-carboxamide

4-(cyclohexylamino)-1-ethyl-N-[l -(4-methylphenyl)propyl]-1H-pyrazolo[3,4-b]pyridine-5-carboxamide 4-(cyclohexylamino)-1-ethyl-N-{1-[4-(1 -methylethyl)phenyl]propyl}-1H-pyrazolo[3,4-b]pyridine-5-carboxamide

4-(cyclohexylamino)-1-ethyl-N-[1-(2-methylphenyl)ethyl]-1H-pyrazolo[3,4-b]pyridine-5-carboxamide

4-(cyclohexylamino)-N-(1-{4-[(difluoromethyl)oxy]phenyl}ethyl)-1-ethyl-1H-pyrazolo[3,4-b]pyridine-5-carboxamide

4-(cyclohexylamino)-1-ethyl-N-{1-[4-(trifluoromethyl)phenyl]ethyl}-1H-pyrazolo[3,4-b]pyridine-5-carboxamide

4-(cyclohexylamino)-1-ethyl-N-[1-(2-methylphenyl)propyl]-1H-pyrazolo[3,4-b]pyridine-5-carboxamide

4-(cyclohexylamino)-1-ethyl-N-{1-[4-(ethyloxy)phenyl]propyl}-1H-pyrazolo[3,4-b]pyridine-5-carboxamide

4-(cyclohexylamino)-N-(1-{4-[(difluoromethyl)oxy]phenyl}propyl)-1-ethyl-1H-pyrazolo[3,4-b]pyridine-5-carboxamide

4-(cyclohexylamino)-1-ethyl-N-{1-[⁴-(trifluoromethyl)phenyl]propyl}-1H-pyrazolo[3,4-b]pyridine-5-carboxamide

4-(cyclohexylamino)-N-[1-(3,4-dimethylphenyl)propyl]-1-ethyl-1H-pyrazolo[3,4-b]pyridine-5-carboxamide

4-(cyclohexylamino)-N-[1-(2,3-dimethylphenyl)ethyl]-1-ethyl-1H-pyrazolo[3,4-b]pyridine-5-carboxamide

4-(cyclohexylamino)-N-[1-(2,4-dimethylphenyl)ethyl]-1-ethyl-1H-pyrazolo[3,4-b]pyridine-5-carboxamide

N-[1-(4-chloro-2-fluorophenyl)ethyl]-4-(cyclohexylamino)-1-ethyl-1H-pyrazolo[3,4-b]pyridine-5-carboxamide

N-[1-(3-chloro-4-methylphenyl)ethyl]-4-(cyclohexylamino)-1-ethyl-1H-pyrazolo[3 ,4-b]pyridine-5-carboxamide

4-(cyclohexylamino)-N-[1-(2,3-dimethylphenyl)propyl]-1-ethyl-1H-pyrazolo[3,4-b]pyridine-5-carboxamide

4-(cyclohexylamino)-N-[1-(2,4-dimethylphenyl)propyl]-1-ethyl-1H-pyrazolo[3,4-b]pyridine-5-carboxamide

N-[1-(4-chloro-2-fluorophenyl)propyl]-4-(cyclohexylamino)-1-ethyl-1H-pyrazolo[3,4-b]pyridine-5-carboxamide

N-[1-(³-chloro-4-methylphenyl)propyl]-4-(cyclohexylamino)-1-ethyl-1H-pyrazolo[3,4-b]pyridine-5-carboxamide

4-(cyclohexylamino)-1-ethyl-N-[1-(3-hydroxyphenyl)propyl]-1H-pyrazolo[3,4-b]pyridine-5-carboxamnide

N-[1-(4-chlorophenyl)-2-hydroxyethyl]-4-(cyclohexylamino)-1-ethyl-1H-pyrazolo[3,4-b]pyridine-5-carboxamide

4-(cyclohexylamino)-N-[1-(2,3-dihydro-1H-inden-5-yl)ethyl]-1-ethyl-1H-pyrazolo[3,4-b]pyridine-5-carboxamide

4-(cyclohexylamino)-1-ethyl-N-[1-(5,6,7,8-tetrahydro-2-naphthalenyl)ethyl]-1H-pyrazolo[3,4-b]pyridine-5-carboxamide

4-[(1-acetyl-4-piperidinyl)amino]-1 -ethyl-N-[(1S)-1-phenylpropyl]-1H-pyrazolo[3,4-b]pyridine-5-carboxamide

4-[(1-acetyl-4-piperidinyl)amino]-1-ethyl-N-[(1R)-1-phenylethyl]-1H-pyrazolo[3,4-b]pyridine-5-carboxamide

4-[(1-acetyl-4-piperidinyl)amino]-N-(diphenylmethyl)-1-ethyl-1H-pyrazolo[3,4-b]pyridine-5-carboxamide

4-[(1-acetyl-4-piperidinyl)amino]-1-ethyl-N-{1-[4-(methylsulfonyl)phenyl]ethyl}pyrazolo[3,4-b]pyridine-5-carboxamide

4-[(1 -acetyl-4-piperidinyl)amino]-1-ethyl-N-[(1R)-1-phenylpropyl]-1H-pyrazolo[3,4-b]pyridine-5-carboxamide

N-[1-(4-chlorophenyl)ethyl]-1-ethyl-4-[(4-oxocyclohexyl)amino]-1H-pyrazolo[3,4-b]pyridine-5-carboxamide

N-[1-(4-chlorophenyl)propyl]-1-ethyl-4-[(4-oxocyclohexyl)amino]-1H-pyrazolo[3,4-b]pyridine-5-carboxamide

1-ethyl-N-[(1s)-1-(4-nitrophenyl)ethyl]-4-[(4-oxocyclohexyl)amino]-1H-pyrazolo[3,4-b]pyridine-5-carboxamide

1-ethyl-N-[(1R)-1-(4-nitrophenyl)ethyl]-4-[(4-oxocyclohexyl)amino]-1H-pyrazolo[3,4-b]pyridine-5-carboxamide

1-ethyl-N- {1-[4-(ethyloxy)phenyl]ethyl}-4-[(4-oxocyclohexyl)amino]-1H-pyrazolo[3,4-b]pyridine-5-carboxamide

1-ethyl-4-[(4-oxocyclohexyl)amino]-N-{1-[4-(propyloxy)phenyl]ethyl}-1H-pyrazolo[3,4-b]pyridine-5-carboxamide

1-ethyl-N-[1-(4-fluorophenyl)ethyl]-4-[(4-oxocyclohexyl)amino]-1H-pyrazolo[3,4-b]pyridine-5-carboxamiide

1-ethyl-N-[(1R)-2-hydroxy-1-phenylethyl]-4-[(4-oxocyclohexyl)amino]-1H-pyrazolo[3,4-b]pyridine-5-carboxamiide

1-ethyl-4-[(4-oxocyclohexyl)amino]-N-(1-phenylpropyl)-1H-pyrazolo[3,4-b]pyridine-5-carboxamide

(2R)-[({1-ethyl-4-[(4-oxocyclohexyl)amino]-1H-pyrazolo[3,4-b]pyridin-5-yl}carbonyl)amino][3-(methyloxy)phenyl]ethanoic acid

1-ethyl-N-{1-[4-(1-methylethyl)phenyl]ethyl}-4-[(4-oxocyclohexyl)amino]-1H-pyrazolo[3,4-b]pyridine-5-carboxamide

1-ethyl-N-[1-(2-methylphenyl)ethyl]-4-[(4-oxocyclohexyl)amino]-1H-pyrazolo[3,4-b]pyridine-5-carboxamide

N-[1-(3,5-dimethylphenyl)ethyl]-1-ethyl-4-[(4-oxocyclohexyl)amino]-1H-pyrazolo[3,4-b]pyridine-5-carboxamide

1-ethyl-N-{(1R)-1-[4-(methyloxy)phenyl]ethyl}-4-[(4-oxocyclohexyl)amino]-1H-pyrazolo[3,4-b]pyridine-5-carboxamide

1-ethyl-N-[l -(4-fluorophenyl)propyl]-4-[(4-oxocyclohexyl)amino]-1H-pyrazolo[3,4-b]pyridine-5-carboxamide

N-[1-(2,3-dichlorophenyl)propyl]-1-ethyl-4-[(4-oxocyclohexyl)amino]-1H-pyrazolo[3,4-b]pyridine-5-carboxamide

1-ethyl-N-[(1R)-1-(4-methylphenyl)ethyl]-4-[(4-oxocyclohexyl)amino]-1H-pyrazolo[3,4-b]pyridine-5-carboxamide

1-ethyl-4-[(4-oxocyclohexyl)amino]-N-(1-phenylethyl)-1H-pyrazolo[3,4-b]pyridine-5-carboxamide

N-[(1R)-1-(4-bromophenyl)ethyl]-1-ethyl-4-[(4-oxocyclohexyl)amino]-1H-pyrazolo[3,4-b]pyridine-5-carboxamide

1-ethyl-N-[(1S)-2-hydroxy-1-phenylethyl]-4-[(4-oxocyclohexyl)amino]-1H-pyrazolo[3,4-b]pyridine-5-carboxamide

N-[1-(4-chlorophenyl)-2-hydroxyethyl]-1-ethyl-4-[(4-oxocyclohexyl)amino]-1H-pyrazolo[3,4-b]pyridine-5-carboxamide

N-{4-[(difluoromethyl)oxy]phenyl}ethyl)-1-ethyl-4-[(4-oxocyclohexyl)amino]-1H-pyrazolo[3,4-b]pyridine-5-carboxamide

1-ethyl-4-[(4-oxocyclohexyl)amino]-N-{1-[4-(trifluoromethyl)phenyl]ethyl}-1H-pyrazolo[3 ,4-b]pyridine-5-carboxamide

1-ethyl-N-[1-(2-methylphenyl)propyl]-4-[(4-oxocyclohexyl)amino]-1H-pyrazolo[3,4-b]pyridine-5-carboxamide

1-ethyl-N-{1-[4-(ethyloxy)phenyl]propyl}-4-[(4-oxocyclohexyl)amino]-1H-pyrazolo[3,4-b]pyridine-5-carboxamide

N-(1-{4-[(difluoromethyl)oxy]phenyl}propyl)-1-ethyl-4-[(4-oxocyclohexyl)amino]-1H-pyrazolo[3,4-b]pyridine-5-carboxamide

1-ethyl-4-[(4-oxocyclohexyl)amino]-N-{1-[4-(trifluoromethyl)phenyl]propyl}-1H-pyrazolo[3,4-b]pyridine-5-carboxamide

N-[1-(3,4-dimethylphenyl)propyl]-1-ethyl-4-[(4-oxocyclohexyl)amino]-1H-pyrazolo[3,4-b]pyridine-5-carboxamide

1-ethyl-4-[(4-oxocyclohexyl)amino]-N-[(1R)-1-phenylpropyl]-1H-pyrazolo[3,4-b]pyridine-5-carboxamide

1-ethyl-N-{(1R)-1-[3-(methyloxy)phenyl]ethyl}-4-[(4-oxocyclohexyl)amino]-1H-pyrazolo[3,4-b]pyridine-5-carboxamide

N-[1-(2,3-dimethylphenyl)ethyl]-1-ethyl-4-[(4-oxocyclohexyl)amino]-1H-pyrazolo[3,4-b]pyridine-5-carboxamide

N-[1-(2,4-dimethylphenyl)ethyl]-1-ethyl-4-[(4-oxocyclohexyl)amino]-1H-pyrazolo[3 ,4-b]pyridine-5-carboxamide

N-[1-(4-chloro-2-fluorophenyl)ethyl]-1-ethyl-4-[(4-oxocyclohexyl)amino]-1H-pyrazolo[3,4-b]pyridine-5-carboxamide

N-[1-(3-chloro-4-methylphenyl)ethyl]-1-ethyl-4-[(4-oxocyclohexyl)amino]-1H-pyrazolo[3,4-b]pyridine-5-carboxamide

N-[1-(2,3-dimethylphenyl)propyl]-1-ethyl-4-[(4-oxocyclohexyl)amino]-1H-pyrazolo[3,4-b]pyridine-5-carboxamide

N-[1-(2,4-dimethylphenyl)propyl]-1-ethyl-4-[(4-oxocyclohexyl)amino]-1H-pyrazolo[3,4-b]pyridine-5-carboxamide

N-[1-(4-chloro-2-fluorophenyl)propyl]-1-ethyl-4-[(4-oxocyclohexyl)amino]-1H-pyrazolo[3,4-b]pyridine-5-carboxamide

N-[1-(3-chloro-methylphenyl)propyl]-1-ethyl-4-[(4-oxocyclohexyl)amino]-1H-pyrazolo[3 ,4-b]pyridine-5-carboxamide

1-ethyl-N-[1-(3-hydroxyphenyl)ethyl]-4-[(4-oxocyclohexyl)amino]-1H-pyrazolo[3,4-b]pyridine-5-carboxamnide

1-ethyl-N-[1-(3-hydroxyphenyl)propyl]-4-[(4-oxocyclohexyl)amino]-1H-pyrazolo[3,4-b]pyridine-5-carboxamide

N-[1-(2,3-dichlorophenyl)ethyl]-1-ethyl-4-[(4-oxocyclohexyl)amino]-1H-pyrazolo[3,4-b]pyridine-5-carboxamide

1-ethyl-N-{1-[3-(methyloxy)phenyl]propyl}-4-[(4-oxocyclohexyl)amino]-1H-pyrazolo[3 ,4-b]pyridine-5-carboxamide

1-ethyl-N-{1-[4-(methyloxy)phenyl]propyl}-4-[(4-oxocyclohexyl)amino]-1H-pyrazolo[3,4-b]pyridine-5-carboxamide

N-[1-(4-bromophenyl)propyl]-1-ethyl-4-[(4-oxocyclohexyl)amino]-1H-pyrazolo[3,4-b]pyridine-5-carboxamide

1-ethyl-4-[(4-oxocyclohexyl)amino]-N-{1-[4-(propyloxy)phenyl]propyl}-1H-pyrazolo[3,4-b]pyridine-5-carboxamide

N-[1-(3,5-dimethylphenyl)propyl]-1-ethyl-4-[(4-oxocyclohexyl)amino]-1H-pyrazolo[3,4-b]pyridine-5-carboxamide

1-ethyl-N-[1-(4-methylphenyl)propyl]-4-[(4-oxocyclohexyl)amino]-1H-pyrazolo[3,4-b]pyridine-5-carboxamide

1-ethyl-N-{1-[4-(1-methylethyl)phenyl]propyl}-4-[(4-oxocyclohexyl)amino]-1H-pyrazolo[3,4-b]pyridine-5-carboxamide

1-ethyl-N-(1-{4-[(1-methylethyl)oxy]phenyl}ethyl)-4-[(4-oxocyclohexyl)amino]-1H-pyrazolo[3,4-b]pyridine-5-carboxamide

1-ethyl-4-[(4-oxocyclohexyl)amino]-N-[1-(5,6,7,8-tetrahydro-2-naphthalenyl)ethyl]-1H-pyrazolo[3,4-b]pyridine-5-carboxamide

N-[1-(4-bromophenyl)-2,2,2-trifluoroethyl]-1-ethyl-4-[(4-oxocyclohexyl)amino]-1H-pyrazolo[3,4-b]pyridine-5-carboxamide

1-ethyl-4-[(4-oxocyclohexyl)amino]-N-{2,2,2-trifluoro-1-[3-(methyloxy)phenyl]ethyl}-1H-pyrazolo[3,4-b]pyridine-5-carboxamide

1-ethyl-4-{[4-(hydroxyimino)cyclohexyl]amino}-N-[1-(5,6,7,8-tetrahydro-2-naphthalenyl)ethyl]-1H-pyrazolo[3,4-b]pyridine-5-carboxamide

1-ethyl-4-{[4-(hydroxyimino)cyclohexyl]amino}-N-[(1S)-2-hydroxy-1-phenylethyl]-1H-pyrazolo[3 ,4-b]pyridine-5-carboxamide

N-[1 -(2,3-dihydro-1H-inden-5-yl)ethyl]-1-ethyl-4-{[4-(hydroxyimino)cyclohexyl]amino}-1H-pyrazolo[3,4-b]pyridine-5-carboxamide

N-[1-(4-chlorophenyl)-2-hydroxyethyl]-1-ethyl-4-{[4-(hydroxyimino)cyclohexyl]amino}-1H-pyrazolo[3,4-b]pyridine-5-carboxamide

1-ethyl-N-{1-[4-(ethyloxy)phenyl]ethyl}-4-{[4-(hydroxyimino)cyclohexyl]amino}-1H-pyrazolo[3,4-b]pyridine-5-carboxamide 1-ethyl-4-{[4-(hydroxyimino)cyclohexyl]amino}-N-{1-[4-(propyloxy)phenyl]ethyl}-1H-pyrazolo[3,4-b]pyridine-5-carboxamide

1-ethyl-N-[1 -(4-fluorophenyl)ethyl]-4-{[4-(hydroxyimino)cyclohexyl]amino}-1H-pyrazolo[3,4-b]pyridine-5-carboxamide

1-ethyl-4- {[4-(hydroxyimino)cyclohexyl]amino}-N-[(1R)-2-hydroxy-1-phenylethyl]-1H-pyrazolo[3,4-b]pyridine-5-carboxamide

1-ethyl-4-{[4-(hydroxyimino)cyclohexyl]amino}-N-(1-phenylpropyl)-1H-pyrazolo[3,4-b]pyridine-5-carboxamide

1-ethyl-4-{[4-(hydroxyimino)cyclohexyl]amino}-N-{1-[4-(1-methylethyl)phenyl]ethyl}-1H-pyrazolo[3,4-b]pyridine-5-carboxamide

N-[1-(3,5-dimethylphenyl)ethyl]-1-ethyl-4-{[4-(hydroxyimino)cyclohexyl]amino}-1H-pyrazolo[3,4-b]pyridine-5-carboxamnide

1-ethyl-4-{[4-(hydroxyimino)cyclohexyl]amino}-N-{(1R)-1-[4-(methyloxy)phenyl]ethyl}-1H-pyrazolo[3,4-b]pyridine-5-carboxamide

1-ethyl-N-[1-(4-fluorophenyl)propyl]-4-{[4-(hydroxyimino)cyclohexyl]amino}-1H-pyrazolo[3,4-b]pyridine-5-carboxamide

N-[1-(2,3-dichlorophenyl)propyl]-1-ethyl-4-{[4-(hydroxyimino)cyclohexyl]amino}-1H-pyrazolo[3,4-b]pyridine-5-carboxamide

1-ethyl-4-{[4-(hydroxyimino)cyclohexyl]amino}-N-[(1R)-1-(4-methylphenyl)ethyl]-1H-pyrazolo[3,4-b]pyridine-5-carboxamide

1-ethyl-4-{[4-(hydroxyimino)cyclohexyl]amino}-N-(1-phenylethyl)-1H-pyrazolo[3,4-b]pyridine-5-carboxamide

N-[(1R)-1-(4-bromophenyl)ethyl]-1-ethyl-4-{[4-(hydroxyimino)cyclohexyl]amino}-1H-pyrazolo[3,4-b]pyridine-5-carboxamide

N-[1-(2,3-dichlorophenyl)ethyl]-1-ethyl-4-{[4-(hydroxyimino)cyclohexyl]amino}-1H-pyrazolo[3,4-b]pyridine-5-carboxamnide

N-[1-(4-chlorophenyl)propyl]-1-ethyl-4-{[4-(hydroxyimino)cyclohexyl]amino}-1H-pyrazolo[3,4-b]pyridine-5-carboxamide

N-[1-(4-chlorophenyl)ethyl]-1-ethyl-4-{[4-(hydroxyimino)cyclohexyl]amino}-1H-pyrazolo[3,4-b]pyridine-5-carboxamide

1-ethyl-4-{[4-(hydroxyimino)cyclohexyl]amino}-N-{1-[3-(methyloxy)phenyl]propyl}-1H-pyrazolo[3,4-b]pyridine-5-carboxamide

1-ethyl-4-{[4-(hydroxyimino)cyclohexyl]amino}-N-{1-[4-(methyloxy)phenyl]propyl}-1H-pyrazolo[3,4-b]pyridine-5-carboxamide

N-[1-(4-bromophenyl)propyl]-1-ethyl-4-{[4-(hydroxyimino)cyclohexyl]amino}-1H-pyrazolo[3,4-b]pyridine-5-carboxamide

1-ethyl-4-{[4-(hydroxyimino)cyclohexyl]amino}-N-{1-[4-(propyloxy)phenyl]propyl}-1H-pyrazolo[3,4-b]pyridine-5-carboxamide

N-[1-(3,5-dimethylphenyl)propyl]-1-ethyl-4-{[4-(hydroxyimino)cyclohexyl]amino}-1H-pyrazolo[3,4-b]pyridine-5-carboxamide

1-ethyl-4-{[4-(hydroxyimino)cyclohexyl]amino}-N-[1-(4-methylphenyl)propyl]-1H-pyrazolo[3,4-b]pyridine-5-carboxamide

1-ethyl-4-{[4-(hydroxyimino)cyclohexyl]amino}-N-{1-[4-(1-methylethyl)phenyl]propyl}-1H-pyrazolo[3,4-b]pyridine-5-carboxamide

1-ethyl-4-{[4-(hydroxyimino)cyclohexyl]amino}-N-[1-(2-methylphenyl)ethyl]-1H-pyrazolo[3,4-b]pyridine-5-carboxamide

N-(1-{4-[(difluoromethyl)oxy]phenyl}ethyl)-1-ethyl-4-{[4-(hydroxyimino)cyclohexyl]amino}-1H-pyrazolo[3,4-b]pyridine-5-carboxamide

1-ethyl-4-{[4-hydroxyimino)cyclohexyl]amino}-N-{1-[4-(trifluoromethyl)phenyl]ethyl}-1H-pyrazolo[3,4-b]pyridine-5-carboxamide

1-ethyl-4- {[4-(hydroxyimino)cyclohexyl]amino}-N-[1-(2-methylphenyl)propyl]-1H-pyrazolo[3,4-b]pyridine-5-carboxamide

1-ethyl-N-{1-[4-(ethyloxy)phenyl]propyl}-4-{[4-(hydroxyimino)cyclohexyl]amino}-1H-pyrazolo[3,4-b]pyridine-5-carboxamide

N-(1-{4-[(difluoromethyl)oxy]phenyl}propyl)-1-ethyl-4-{[4-(hydroxyimino)cyclohexyl]amino}-1H-pyrazolo[3,4-b]pyridine-5-carboxamide

1-ethyl-4-{[4-(hydroxyimino)cyclohexyl]amino}-N-{1-[4-(trifluoromethyl)phenyl]propyl}-1H-pyrazolo[3,4-b]pyridine-5-carboxamide

N-[1-(3,4-dimethylphenyl)propyl]-1-ethyl-4-{[4-(hydroxyimino)cyclohexyl]amino}-1H-pyrazolo[3,⁴-b]pyridine-5-carboxamide

1-ethyl-4-{[⁴-(hydroxyimino)cyclohexyl]amino}-N-[(1R)-1-phenylpropyl]-1H-pyrazolo[3,4-b]pyridine-5-carboxamide

1-ethyl-4-{[4-(hydroxyimino)cyclohexyl]amino}-N- {(1R)-1-[3-(methyloxy)phenylethyl}-1H-pyrazolo[3,4-b]pyridine-5-carboxamide

N-[1-(2,3-diethylphenyl)ethyl]-1-ethyl-4-{[4-hydroxyimino)cyclohexyl]amino}-1H-pyrazolo[3,4-b]pyridine-5-carboxamide

N-[1-(2,4-dimethylphenyl)ethyl]-1-ethyl-4-{[⁴-(hydroxyimino)cyclohexyl]amino}-1H-pyrazolo[3,4-b]pyridine-5-carboxamide

N-[1-(4-chloro-2-fluorophenyl)ethyl]-1-ethyl-4-{[4-(hydroxyimino)cyclohexyl]amino}-1H-pyrazolo[3,4-b]pyridine-5-carboxamide

N-[1-(³-chloro-4-methylphenyl)ethyl]-1-ethyl-4-{[4-(hydroxyimino)cyclohexyl]amino}-1H-pyrazolo[3,4-b]pyridine-5-carboxamide

N-[1-(2,3-dimethylphenyl)propyl]-1-ethyl-4-{[4-(hydroxyimino)cyclohexyl]amino}-1H-pyrazolo[3,4-b]pyridine-5-carboxamide

N-[1-(2,4-dimethylphenyl)propyl]-1-ethyl-4-{[4-(hydroxyimino)cyclohexyl]amino}-1H-pyrazolo[3,4-b]pyridine-5-carboxamide

N-[1-(4-chloro-2-fluorophenyl)propyl]-1-ethyl-4-{[4-(hydroxyimino)cyclohexyl amino}-1H-pyrazolo[3,4-b]pyridine-5-carboxamide

N-[1-(3-chloro-4-methylphenyl)propyl]-1-ethyl-4-{[4-(hydroxyimino)cyclohexyl]amino}-1H-pyrazolo[3,4-b]pyridine-5-carboxamide

1-ethyl-4-{[4-(hydroxyimino)cyclohexyl]amino}-N-[1-(3-hydroxyphenyl)ethyl]-1H-pyrazolo[3,⁴-bipyridine-5-carboxamide

1-ethyl-4-{[4-(hydroxyimino)cyclohexyl]amino}-N-[1-(3-hydroxyphenyl)propyl]-1H-pyrazolo[3,4-b]pyridine-5-carboxamide

N-[1-(2,4-dimethylphenyl)ethyl]-1-ethyl-4-{[4-(hydroxyimino)cyclohexyl]amino}-1H-pyrazolo[3,4-b]pyridine-5-carboxamide

N-[1-(2,4-dimethylphenyl)ethyl]-1-ethyl-4-{[4-(hydroxyimino)cyclohexyl]amino}-1H-pyrazolo[3,4-bipyridine-5-carboxamide

N-[1-(3,5-dimethylphenyl)ethyl]-1 -ethyl-4-{[4-(hydroxyimino)cyclohexyl]amino}-1H-pyrazolo[3,4-b]pyridine-5-carboxamide

N-[1-(3,5-dimethylphenyl)ethyl]-1-ethyl-4-{[4-(hydroxyimino)cyclohexyl]amino}-1H-pyrazolo[3,4-b]pyridine-5-carboxamide

1-ethyl-4-{[4-(hydroxyimino)cyclohexyl]amino}-N-(1-{4-[(1-methylethyl)oxy]phenyl}ethyl)-1H-pyrazolo[3,4-b]pyridine-5-carboxamide

1-ethyl-4-{[4-(hydroxyimino)cyclohexyl]amino}-N-(1-{4-[(1-methylethyl)oxy]phenyl}ethyl)-1H-pyrazolo[3,4-b]pyridine-5-carboxamide

1-ethyl-N-[1-(4-fluorophenyl)ethyl]-4-{[4-(hydroxyimino)cyclohexyl]amino}-1H-pyrazolo[3,4-b]pyridine-5-carboxamide

1-ethyl-N-[1-(4-fluorophenyl)ethyl]-4-{[4-(hydroxyimino)cyclohexyl]amino}-1H-pyrazolo[3,4-b]pyridine-5-carboxamide

N-[1-(4-chlorophenyl)propyl]-1-ethyl-4-f{[(1S,3R)- and/or (1R,3S)-3-hydroxycyclohexyl]amino}-1H-pyrazolo[3,4-b]pyridine-5-carboxamide

1-ethyl-4-{[(1S,3R)- and/or (1R,3S)-3-hydroxycyclohexyl]amino}-N-[(1R)-1-(4-methylphenyl)ethyl]-1H-pyrazolo[3,4-b]pyridine-5-carboxamide

N-[1-(2,4-dimethylphenyl)ethyl]-1-ethyl-4-{[(1S,3R)- and/or (1R,3S)-3-hydroxycyclohexyl]amino}-1H-pyrazolo[3,4-b]pyridine-5-carboxamide (Isomer 1)

N-[1-(2,4-dimethylphenyl)ethyl]-1-ethyl-4-{[(1S,3R)- and/or (1R,3S)-3-hydroxycyclohexyl]amino}-1H-pyrazolo[3,4-b]pyridine-5-carboxamide (Isomer 2)

N-[1-(3,4-dimethylphenyl)propyl]-1-ethyl-4-{[(1S,3R)- and/or (1R,3S)-3-hydroxycyclohexyl]amino}-1H-pyrazolo[3,4-b]pyridine-5-carboxamide

N-[1-(4-chlorophenyl)propyl]-1-ethyl-6-methyl-4-(tetrahydro-2H-pyran-4-ylamino)-1H-pyrazolo[3,4-b]pyridine-5-carboxamide

N-[1-(4-chlorophenyl)ethyl]-1-ethyl-6-methyl-4-(tetrahydro-2H-pyran-4-ylamino)-1H-pyrazolo[3,4-b]pyridine-5-carboxamide

N-[1-(4-chlorophenyl)ethyl]-1-ethyl-4-(tetrahydro-2H-pyran-4-ylamino)-1H-pyrazolo[3,4-b]pyridine-5-carboxamide (Enantiomer 1)

N-[1-(4-chlorophenyl)ethyl]-1-ethyl-4-(tetrahydro-2H-pyran-4-ylamino)-1H-pyrazolo[3,4-b]pyridine-5-carboxamide (Enantiomer 2)

N-[1-(4-chlorophenyl)propyl]-1-ethyl-4-(tetrahydro-2H-pyran-4-ylamino)-1H-pyrazolo[3,4-b]pyridine-5-carboxamide (Enantiomer 1)

N-[1-(4-chlorophenyl)propyl]-1-ethyl-4-(tetrahydro-2H-pyran4-ylamino)-1H-pyrazolo[3,4-b]pyridine-5-carboxamide (Enantiomer 2)

1-ethyl-N-{1-[4-(ethyloxy)phenyl]ethyl}-4-(tetrahydro-2H-pyran-4-ylamino)-1H-pyrazolo[3,4-b]pyridine-5-carboxamide (Enantiomer 1)

1-ethyl-N-{1-[4-(ethyloxy)phenyl]ethyl}-4-(tetrahydro-2H-pyran-4-ylamino)-1H-pyrazolo[3,4-b]pyridine-5-carboxamide (Enantiomer 2)

N-[1-(2,4-dimethylphenyl)ethyl]-1-ethyl-4-[(4-oxocyclohexyl)amino]-1H-pyrazolo[3,4-b]pyridine-5-carboxamide (Enantiomer 1)

N-[1-(2,4-dimethylphenyl)ethyl]-1-ethyl-4-[(4-oxocyclohexyl)amino]-1H-pyrazolo[3,4-b]pyridine-5-carboxamide (Enantiomer 2)

N-[1-(3,5-dimethylphenyl)ethyl]-1-ethyl-4-[(4-oxocyclohexyl)amino]-1H-pyrazolo[3,4-b]pyridine-5-carboxamide (Enantiomer 1)

N-[1-(3,5-dimethylphenyl)ethyl]-1-ethyl-4-[(4-oxocyclohexyl)amino]-1H-pyrazolo[3,4-b]pyridine-5-carboxamide (Enantiomer 2)

1-ethyl-N-(1-{4-[(1-methylethyl)oxy]phenyl}ethyl)-4-[(4-oxocyclohexyl)amino]-1H-pyrazolo[3,4-b]pyridine-5-carboxamide (Enantiomer 1)

1-ethyl-N-(1-{4-[(1-methylethyl)oxy]phenyl}ethyl)-4-[(4-oxocyclohexyl)amino]-1H-pyrazolo[3,4-b]pyridine-5-carboxamide (Enantiomer 2)

1-ethyl-N-[1-(4-fluorophenyl)ethyl]-4-[(4-oxocyclohexyl)amino]-1H-pyrazolo[3,4-b]pyridine-5-carboxamide (Enantiomer 1)

1-ethyl-N-[1-(4-fluorophenyl)ethyl]-4-[(4-oxocyclohexyl)amino]-1H-pyrazolo[3,4-b]pyridine-5-carboxamide (Enantiomer 2)

N-[1-(2,4-dimethylphenyl)propyl]-1-ethyl-4-(tetrahydro-2H-pyran-4-ylamino)-1H-pyrazolo[3,4-b]pyridine-5-carboxamide (Enantiomer 1)

N-[1-(2,4-dimethylphenyl)propyl]-1-ethyl-4-(tetrahydro-2H-pyran-4-ylamino)-1H-pyrazolo[3,4-b]pyridine-5-carboxamide (Enantiomer 2)

1-ethyl-4-{[(1S,3R)- and/or (IR,3S)-3-hydroxycyclohexyl]amino}-N-[(1R)-1-(4-methylphenyl)ethyl]-1H-pyrazolo[3,4-b]pyridine-5-carboxamide (Diastereoisomer 1)

1-ethyl-4-{[(1S,3R)- and/or (1R,3S)-3-hydroxycyclohexyl]amino}-N-[(1R)-1-(4-methylphenyl)ethyl]-1H-pyrazolo[3,4-b]pyridine-5-carboxamide (Diastereoisomer 2)

N-[1-(2,4-dimethylphenyl)propyl]-1-ethyl-4-(tetrahydro-2H-pyran-4-ylamino)-1H-pyrazolo[3,4-b]pyridine-5-carboxamide (Enantiomer 2) hydrochloride

4-{[1-(aminocarbonyl)-4-piperidinyl]amino}-1-ethyl-N-[(1R)-1-(4-methylphenyl)ethyl]-1H-pyrazolo[3,4-b]pyridine-5-carboxamide

4-{[1-(aminocarbonyl)-4-piperidinyl]amino}-1-ethyl-N-[(1R)-1-phenylethyl]-1H-pyrazolo[3,4-b]pyridine-5-carboxamide

4-{[1-(aminocarbonyl)-4-piperidinyl]amino}-N-[(1R)-1-(4-bromophenyl)ethyl]-1-ethyl-1H-pyrazolo[3,4-b]pyridine-5-carboxamide

4-{[1-(aminocarbonyl)-4-piperidinyl]amino}-N-[1-(2,4-dimethylphenyl)propyl]-1-ethyl-1H-pyrazolo[3,4-b]pyridine-5-carboxamide

4-{[1-(aminocarbonyl)-4-piperidinyl]amino}-N-[1-(3-chloro-4-methylphenyl)propyl]-1-ethyl-1H-pyrazolo[3,4-b]pyridine-5-carboxamide

4-{1-(aminocarbonyl)-4-piperidinyl]amino}-N-[1-(4-chloro-2-fluorophenyl)propyl]-1-ethyl-1H-pyrazolo[3,4-b]pyridine-5-carboxamide, or

4-{[4-(aminocarbonyl)cyclohexyl]amino}-1-ethyl-N-[(1R)-1-phenylethyl]-1H-pyrazolo[3,4-b]pyridine-5-carboxamide (for example, 4-{cis-[4-(aminocarbonyl)cyclohexyl]amino}-l-ethyl-N-[(1R)-1-phenylethyl]-1H-pyrazolo[3,4-b]pyridine-5-carboxamide);

as a compound or a salt thereof, e.g. a pharmaceutically acceptable salt thereof.

The structures of the above-listed specific compounds, or embodiments thereof, are given in Examples 1 to 314A hereinafter.

It is particularly preferred that the compound of formula (I) or the salt thereof is one of Examples 1 to 314 or Example 314A, as a compound or a salt thereof, e.g. a pharmaceutically acceptable salt thereof. The structures of these specific compounds, or embodiments thereof, are given in Examples 1 to 314 hereinafter, and their names are given in the Examples section.

In one embodiment, is still further preferred that the compound of formula (I) or the salt thereof is a compound of Example 73,98, 283, 304, 306, 307, 310 or 311 (or is a compound of Example 75), as defined by the structures and/or names described herein, or a salt thereof, e.g. a pharmaceutically acceptable salt thereof. The structures and names of these Examples are described in the Examples section. These Examples can for example be for inhaled administration e.g. to a mammal such as a human, and/or can be contained in a pharmaceutical composition suitable and/or adapted for inhaled administration, and/or can be in a particle-size-reduced form (e.g. in a size-reduced form obtained or obtainable by micronisation, e.g. see “Particle size reduction” section below).

In an alternative preferable embodiment, the compound of formula (I) or the salt thereof is:

N-[(1S)-1-(2,4-dimethylphenyl)propyl]-1-ethyl-4-(tetrahydro-2H-pyran-4-ylamino)-1H-pyrazolo[3,4-b]pyridine-5-carboxamide

N-[(1R)-1-(2,4-dimethylphenyl)propyl]-1-ethyl-4-(tetrahydro-2H-pyran-4-ylamino)-1H-pyrazolo[3,4-b]pyridine-5-carboxamide

N-[(1R)-1-(2,5-dimethylphenyl)ethyl]-1-ethyl-4-(tetrahydro-2H-pyran-4-ylamino)-1H-pyrazolo[3,4-b]pyridine-5-carboxamide

1-ethyl-4-(tetrahydro-2H-pyran-4-ylamino)-N-[(1R)-1-(2,4,6-trimethylphenyl)ethyl]-1H-pyrazolo[3,4-b]pyridine-5-carboxamide

1-ethyl-N-[(1R)-1-(2-ethylphenyl)ethyl]-4-(tetrahydro-2H-pyran-4-ylamino)-1H-pyrazolo[3,4-b]pyridine-5-carboxamide

1-ethyl-N-[(1R)-1-(4-ethylphenyl)ethyl]-4-(tetrahydro-2H-pyran-4-ylamino)-1H-pyrazolo[3,4-b]pyridine-5-carboxamide

1-ethyl-N-[(1R)-1-(4-methylphenyl)propyl]-4-(tetrahydro-2H-pyran-4-ylamino)-1H-pyrazolo[3,4-b]pyridine-5-carboxamide

1-ethyl-N-[(1R)-1-(4-ethylphenyl)propyl]-4-(tetrahydro-2H-pyran-4-ylamino)-1H-pyrazolo[3,4-b]pyridine-5-carboxamide

1-ethyl-N-{(1R)-1-[4-(1-methylethyl)phenyl]propyl}-4-(tetrahydro-2H-pyran-4-ylamino)-1H-pyrazolo[3,4-b]pyridine-5-carboxamide

N-[(1R)-1-(4-chloro-2-fluorophenyl)propyl]-1-ethyl-4-(tetrahydro-2H-pyran-4-ylamino)-1H-pyrazolo[3,4-b]pyridine-5-carboxamide

N-[(1R)-1-(2,6-dimethylphenyl)propyl]-1-ethyl-4-(tetrahydro-2H-pyran-4-ylamino)-1H-pyrazolo[3,4-b]pyridine-5-carboxamide

N-[(1R)-1-(2,5-dimethylphenyl)propyl)-1-ethyl-4-(tetrahydro-2H-pyran-4-ylamino)-1H-pyrazolo[3,4-b]pyridine-5-carboxamide

1-ethyl-N-[(1R)-1-(2-ethylphenyl)propyl]-4-(tetrahydro-2H-pyran-4-ylamino)-1H-pyrazolo[3,4-b]pyridine-5-carboxamide

1-ethyl-4-(tetrahydro-2H-pyran-4-ylamino)-N-[(1R)-1-(2,4,6-triiethylphenyl)propyl]-1H-pyrazolo[3,4-b]pyridine-5-carboxamide

4-{[1-(aminocarbonyl)-4-piperidinyl]amino}-N-[(1R)-1-(2,5-dimethylphenyl)ethyl]-1-ethyl-1H-pyrazolo[3,4-b]pyridine-5-carboxamide

4-{[1-(aminocarbonyl)-4-piperidinyl]amino}-1-ethyl-N-[(1R)-1-(4-ethylphenyl)ethyl]-1H-pyrazolo[3,4-b]pyridine-5-carboxamide

4-{[1-(aminocarbonyl)-4-piperidinyl]amino}-1-ethyl-N-[(1R)-1-(2-ethylphenyl)ethyl]-1H-pyrazolo[3,4-b]pyridine-5-carboxamide

4-{[1-(aminocarbonyl)-4-piperidinyl]amino}-1-ethyl-N-[(1R)-1-(2,4,6-trimethylphenyl)ethyl]-1H-pyrazolo[3,4-b]pyridine-5-carboxamide

4-{[1-(aminocarbonyl)-4-piperidinyl]amino}-N-[(1R)-1-(2,4-dimethylphenyl)propyl]-1-ethyl-1 H-pyrazolo[3,4-b]pyridine-5-carboxamide

4-{1[1-(aminocarbonyl)-4-piperidinyl]amino}-N-[1-(4-chlorophenyl)ethyl]-1-ethyl-1-pyrazolo[3,4-b]pyridine-5-carboxamide

4-{[1-(aminocarbonyl)-4-piperidinyl]amino}-1-ethyl-N-[(1R)-1-phenylpropyl]-1H-pyrazolo[3,4-b]pyridine-5-carboxamnide

4-{[1-(aminocarbonyl)-4-piperidinyl]amino}-N-[1-(4-chlorophenyl)propyl]-1 -ethyl-1H-pyrazolo[3,4-b]pyridine-5-carboxamide

4-{[1 -(aminocarbonyl)-4-piperidinyl]amino}-1-ethyl-N-[1-(4-fluorophenyl)propyl]-pyrazolo[3,4-b]pyridine-5-carboxamide

4-{[l-(aminocarbonyl)-4-piperidinyl]amino}-1-ethyl-N-[(1R)-1-(4-methylphenyl)propyl]-1H-pyrazolo[3,4-b]pyridine-5-carboxamide

4-{1[1-(aminocarbonyl)-4-piperidinyl]amino}-1-ethyl-N-[(1 R)-1-(4-ethylphenyl)propyl]-1H-pyrazolo[3,4-b]pyridine-5-carboxamide

4-{[1-(aminocarbonyl)-4-piperidinyl]amino}-1-ethyl-N-{(1R)-1-[4-(1-methylethyl)phenyl]propyl}-1H-pyrazolo[3,4-b]pyridine-5-carboxamide

4-{[1-(aminocarbonyl)-4-piperidinyl]amino}-N-[(1R)-1-(4-chloro-2-fluorophenyl)propyl]-1-ethyl-1H-pyrazolo[3,4-b]pyridine-5-carboxamide

4-{[1-(aminocarbonyl)-4-piperidinyl]amino}-N-[(1R)-1-(2,6-dimethylphenyl)propyl]-1-ethyl-1H-pyrazolo[3,4-b]pyridine-5-carboxamide

4-{[1-(aminocarbonyl)-4-piperidinyl]amino}-N-[(1R)-1-(2,5-dimethylphenyl)propyl]-1-ethyl-1H-pyrazolo[3,4-b]pyridine-5-carboxamide

4-{[1-(aminocarbonyl)-4-piperidinyl]amino}-1-ethyl-N-[(1R)-1-(2-ethylphenyl)propyl]-1H-pyrazolo[3,4-b]pyridine-5-carboxamide

4-{[1-(aminocarbonyl)-4-piperidinyl]amino}-1-ethyl-N-[(1R)-1-(2,4,6-trimethylphenyl)propyl]-1H-pyrazolo[3,4-b]pyridine-5-carboxamide

4-{[4-(aminocarbonyl)cyclohexyl]amino}-N-[1-(4-chlorophenyl)propyl]-1-ethyl-1H-pyrazolo[3,4-b]pyridine-5-carboxamide

4-{[4-(aminocarbonyl)cyclohexyl]amino}-1-ethyl-N-[(1R)-1-phenylpropyl]-1H-pyrazolo[3,4-b]pyridine-5-carboxamide

4-{[4-(aminocarbonyl)cyclohexyl]amino}-N-(1-{4-[(difluoromethyl)oxy]phenyl}ethyl)-1-ethyl-1H-pyrazolo[3,4-b]pyridine-5-carboxamide

4-{[4-(aminocarbonyl)cyclohexyl]amino}-N-[1-(4-chlorophenyl)ethyl]-1-ethyl-1H-pyrazolo[3,4-b]pyridine-5-carboxamide

4-{[4-(aminocarbonyl)cyclohexyl]amino}-1-ethyl-N-[ 1 -(4-fluorophenyl)propyl]-1H-pyrazolo[3,4-b]pyridine-5-carboxamide

4-{[4-(aminocarbonyl)cyclohexyl]amino}-N-[(1R)-1-(4-bromophenyl)ethyl]-1-ethyl-1H-pyrazolo[3,4-b]pyridine-5-carboxamide

4-{[cis-4-(aminocarbonyl)cyclohexyl]amino}-N-[(1R)-1-(2,4-dimethylphenyl)propyl]-1-ethyl-1H-pyrazolo[3,4-b]pyridine-5-carboxamide

4-{[cis-4-(aminocarbonyl)cyclohexyl]amino}-1-ethyl-N-[(1R)-1-(4-methylphenyl)ethyl]-1H-pyrazolo[3,4-b]pyridine-5-carboxamide

4-{[cis-4-(aminocarbonyl)cyclohexyl]amino}-1-ethyl-N-[(1R)-1-phenylethyl]-1H-pyrazolo[3,4-b]pyridine-5-carboxamide

4-{[cis-4-(aminocarbonyl)cyclohexyl]amino}-N-[(1R)-1-(4-bromophenyl)ethyl]-1-ethyl-1H-pyrazolo[3,4-b]pyridine-5-carboxamide

4-{[trans-4-(aminocarbonyl)cyclohexyl]amino}-N-[(1R)-1-(2,4-dimethylphenyl)propyl]-1-ethyl-1H-pyrazolo[3,4-b]pyridine-5-carboxamide

4-{[trans4-(aminocarbonyl)cyclohexyl]amino}-1-ethyl-N-[(1R)-1-(4-methylphenyl)ethyl]-1H-pyrazolo[3,4-b]pyridine-5-carboxamide

4-{[trans4(aminocarbonyl)cyclohexyl]amino}-1-ethyl-N-[(1R)-1-phenylethyl]-1H-pyrazolo[3,4-b]pyridine-5-carboxamide

4-{[trans-4-(aminocarbonyl)cyclohexyl]amino}-N-[(1R)-1-(4-bromophenyl)ethyl]-1-ethyl-1H-pyrazolo[3,4-b]pyridine-5-carboxamide

4-{[(3S)-1-(aminocarbonyl)pyrrolidin-3-yl]amino}-N-[1-(2,4-dimethylphenyl)propyl]-1-ethyl-1H-pyrazolo[3,4-b]pyridine-5-carboxamide

4-{[(3S)-1-(aminocarbonyl)pyrrolidin-3-yl]amino}-1-ethyl-N-[(1R)-1-(4-methylphenyl)ethyl]-1H-pyrazolo[3,4-b]pyridine-5-carboxamide

4-{[(3S)-1-(aminocarbonyl)pyrrolidin-3-yl]amino}-N-[1-(3,4-dimethylphenyl)propyl]ethyl-1H-pyrazolo[3,4-b]pyridine-5-carboxamide

4-{[(3S)-1-(aminocarbonyl)pyrrolidin-3-yl]amino}-N-[(1R)-1-(4-bromophenyl)ethyl]-1-ethyl-1H-pyrazolo[3,4-b]pyridine-5-carboxamide

4-{[(3R)-1-(aminocarbonyl)pyrrolidin-3-yl]amino}-N-[1-(2,4-dimethylphenyl)propyl]-1-ethyl-1H-pyrazolo[3,4-b]pyridine-5-carboxamide

4-{[(3R)-1-(aminocarbonyl)pyrrolidin-3-yl]amino}-1-ethyl-N-[(1R)-1-(4-methylphenyl)ethyl]-1H-pyrazolo[3,4-b]pyridine-5-carboxamide

4-{[(3R)-1-(aminocarbonyl)pyrrolidin-3-yl]amino}-N-[1-(3,4-dimethylphenyl)propyl]-1-ethyl-1H-pyrazolo[3,4-b]pyridine-5-carboxamide

4-{[(3R)-1-(aminocarbonyl)pyrrolidin-3-yl]amino}-N-[(1R)-1-(4-bromophenyl)ethyl]-1-ethyl-1H-pyrazolo[3,4-b]pyridine-5-carboxamide

4-{[cis-3-(aminocarbonyl)cyclobutyl]amino}-1-ethyl-N-[(1R)-1-(4-methylphenyl)ethyl]-1H-pyrazolo[3,4-b]pyridine-5-carboxamide

4-{[cis-3-(aminocarbonyl)cyclobutyl]amino}-N-[1-(2,4-dimethylphenyl)propyl]-1-ethyl-1H-pyrazolo[3,4-b]pyridine-5-carboxamide

4-[(trans-4-acetylcyclohexyl)amino]-1-ethyl-N-[(1R)-1-(4-methylphenyl)ethyl]-1H-pyrazolo[3,4-b]pyridine-5-carboxamide

4-[(4-acetylcyclohexyl)amino]-N-[(1R)-1-(2,4-dimethylphenyl)propyl]-1-ethyl-1H-pyrazolo[3,4-b]pyridine-5-carboxamide

4-[(cis-4-acetylcyclohexyl)amino]-1-ethyl-N-[(1R)-1-(4-methylphenyl)ethyl]-1H-pyrazolo[3,4-b]pyridine-5-carboxamide

1-ethyl-4-{[trans-3-hydroxycyclohexyl]amino}-N-[(1R)-1-(4-methylphenyl)ethyl]-1H-pyrazolo[3,4-b]pyridine-5-carboxamide

N-[(1S)-1-(2,4-dimethylphenyl)ethyl]-1-ethyl-4-{[trans-3-hydroxycyclohexyl]amino}-1H-pyrazolo[3,4-b]pyridine-5-carboxamide

N-[(1R)-1-(2,4-dimethylphenyl)ethyl]-1-ethyl-4-{[trans-3-hydroxycyclohexyl]amino}-1H-pyrazolo[3,4-b]pyridine-5-carboxamide

N-[(1R)-1-(4-bromophenyl)ethyl]-1-ethyl-4-{[trans-3-hydroxycyclohexyl]amino}-1H-pyrazolo[3,4-b]pyridine-5-carboxamide

N-[1-(3,4-dimethylphenyl)propyl]-1-ethyl-4-{[trans-3-hydroxycyclohexyl]amino}-1H-pyrazolo[3,4-b]pyridine-5-carboxamide

N-[4-(dimethylamino)-1-(3-methylphenyl)-4-oxobutyl]-1-ethyl-4-(tetrahydro-2H-pyran-4-ylamino)-1H-pyrazolo[3,4-b]pyridine-5-carboxamide

4-{[1-(aminocarbonyl)-4-piperidinyl]amino}-N-[4-(dimethylamino)-1-(3-methylphenyl)-4-oxobutyl]-1-ethyl-1H-pyrazolo[3,4-b]pyridine-5-carboxamide

1-ethyl-N-[(1R)-1-(4-methylphenyl)ethyl]-4-(4-piperidinylamino)-1H-pyrazolo[3,4-b]pyridine-5:-carboxamide hydrochloride, or

N-[1-(2,4-dimethylphenyl)propyl]-1-ethyl-4-(4-piperidinylamino)-1H-pyrazolo[3,4-b]pyridine-5-carboxamide hydrochloride;

as a compound or a salt thereof, e.g. a pharmaceutically acceptable salt thereof.

The structures of the above specific compounds, or embodiments thereof, are given in Examples 315 to 372 and Examples 374 to 382 hereinafter, and their names are given in the Examples section.

In a preferred embodiment of the above list of compounds (Examples 315 to 372 and Examples 374 to 382), it is further preferred that the compound of formula (I) or the salt thereof is a compound of Example 316, 317, 318, 319, 320, 321, 322, 323, 324, 325, 326, 327, 328, 329, 330, 331, 332, 333, 334, 335, 336, 337, 338, 339, 341, 342, 343, 344, 345, 351, 352, or 353, as defined by the structures and/or names described herein, or a salt thereof, e.g. a pharmaceutically acceptable salt thereof. Of these, Examples 316-333, 335, 338-345, and 351-353, are believed to consist essentially of an enantiomer which is believed to have the (R)-stereochemistry at the benzylic carbon atom. It is still further preferred that the compound of formula (I) or the salt thereof is a compound of Example 316, 321, 324, 326, 327, 328, 330, 331, 332, 333, 334, 335, 336, 337, 338, 339, 343, 344 or 345, as defined by the structures and/or names described herein, or a salt thereof, e.g. a pharmaceutically acceptable salt thereof. The structures and names of these Examples are described in the Examples section.

In a preferred embodiment of the above list of compounds (Examples 315 to 372 and Examples 374 to 382), is yet further preferred that the compound of formula (I) or the salt thereof is:

4-{[1-(aminocarbonyl)-4-piperidinyl]amino}-N-[(1R)-1-(2,4-dimethylphenyl)propyl]-1-ethyl-1H-pyrazolo[3,4-b]pyridine-5-carboxamide (Example 333), or a salt thereof such as a pharmaceutically acceptable salt thereof.

Example 333 is believed to consist essentially of an enantiomer which is believed to have the (R)-stereochemistry at the benzylic carbon atom. See Example 333 below for the believed structure. Example 333 or a salt thereof can for example be for inhaled administration e.g. to a mammal such as human, and/or can be contained in a pharmaceutical composition suitable and/or adapted for inhaled administration, and/or can be in a particle-size-reduced form (e.g. in a size-reduced form obtained or obtainable by micronisation, e.g. see “Particle size reduction” section below).

According to one optional embodiment of the invention, the compound of formula (I) or salt thereof can be a compound of Formula (XXVI) or a salt thereof:

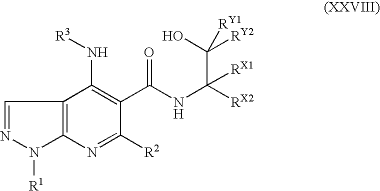

wherein:

R^{X1} is a hydrogen atom (H), C₁₋₂alkyl or C₁fluoroalkyl (preferably H);

R^{Y1} is a hydrogen atom (H) or C₁₋₂alkyl;

R^{Y2} is a hydrogen atom (H); C₁₋₃alkyl (e.g. C₁₋₂alkyl or methyl); or —(CH₂)ₙ^{7aa}—OH;

wherein n^{7aa} is 1, 2 or 3;

and

R^{X2} is Ar^{A}, wherein:
(i) Ar^{A} is phenyl optionally substituted by one or two substituents independently being: fluoro, chloro, bromo, C₁₋₂alkyl, C₁₋₂fluoroalkyl, C₁₋₂alkoxy,

C₁₋₂fluoroalkoxy; OH; —NR^{11aa}R^{11bb} (wherein R^{11aa} is H or C₁₋₂alkyl and R^{11bb} is H, C₁₋₂alkyl, —C(O)—C₁₋₂alkyl or —S(O)₂—C₁₋₂alkyl); cyano; —C(O)—NR^{11cc}R^{11dd} (wherein R^{11cc} and R^{11dd} independently are H or C₁₋₂alkyl); —C(O)—OR^{11ee} wherein R^{11ee} is H or C₁₋₂alkyl; or —S(O)₂—R^{11ff} (wherein R^{11ff} is C₁₋₂alkyl, NH₂, NHMe or NMe₂); or the phenyl Ar^{A} is optionally substituted at two adjacent Ar ring atoms by the two ends of a chain which is: —(CH₂)₄—, —(CH₂)₃—, or —CH═CH—CH═CH—; or
(ii) Ar^{A} is an optionally substituted 5-membered heterocyclic aromatic ring containing 1, 2, 3 or 4 heteroatoms (e.g. 1, 2 or 3 heteroatoms) selected from O, N or S; and wherein when the heterocyclic aromatic ring Ar^{A} contains 2, 3 or 4 heteroatoms (e.g. 2 or 3 heteroatoms), one is selected from O, N and S and the remaining heteroatom(s) are N; and wherein the heterocyclic aromatic ring Ar^{A} is optionally substituted by one or two groups independently being C₁₋₄alkyl (e.g. C₁₋₂alkyl) or OH (including any keto tautomer of an OH-substituted aromatic ring).

A compound of formula (XXVIII can suitably be:

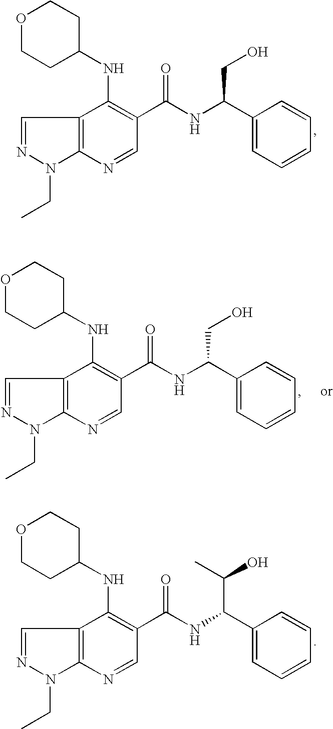

These three compounds are:

1-Ethyl-N-[(1R)-2-hydroxy-1-phenylethyl]-4-(tetrahydro-2H-pyran-4-ylamino)-1H-pyrazolo[3,4-b]pyridine-5-carboxamide,

1-Ethyl-N-[(1S)-2-hydroxy-1-phenylethyl]-4-(tetrahydro-2H-pyran-4-ylamino)-1H-pyrazolo[3,4b]pyridine-5-carboxamide, and

1-Ethyl-N-[(1S,2R)-2-hydroxy-1-phenylpropyl]-4-(tetrahydro-2H-pyran-4-ylamino)-1H-pyrazolo[3,4-b]pyridine-5-carboxamide.

These three compounds are disclosed as Intermediates 42, 43 and 46 respectively in copending international patent application PCT/EP2003/014867 (=PCT/EP03/14867), filed on 19 Dec. 2003 in the name of Glaxo Group Limited and published on 8 Jul. 2004 as WO 2004/056823 A1, the content of which is incorporated herein by reference. The compounds of Formula (XXVIII) are also disclosed in PCT/EP2003/014867 (e.g. see page 59 thereof) and are incorporated herein by reference.

According to an alternative optional embodiment of the invention, the compound of formula (I) or salt thereof is not a compound of Formula (XXVIII) or a salt thereof.

A further aspect of the present invention provides a compound of formula (13) or a salt thereof (in particular, a pharmaceutically acceptable salt thereof):

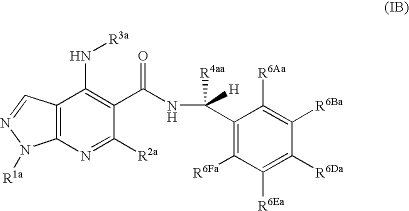

wherein:

R^{1a} is C₂₋₃alkyl, C₂fluoroalkyl or —CH₂CH₂OH;

R^{2a} is a hydrogen atom (H) or methyl;

NBR^{3a} is of sub-formula (p14), in which the —NH— connection point of the NHR^{3a} group to the 4-position of the pyrazolopyridine of formula (IB) is underlined:

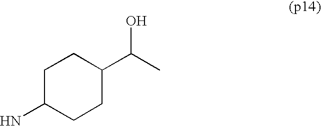

R^{4aa} is methyl, ethyl, C₁fluoroalkyl (such as CF₃), —CH₂OH, or —CH₂OMe;

R^{6Aa}, R^{6Ba}, R^{6Da}, R^{6Ea} and R^{6Fa}, independently of each other, are: a hydrogen atom (H), a fluorine, chlorine, bromine or iodine atom, methyl, ethyl, n-propyl, isopropyl, isobutyl, trifluoromethyl, —CH₂OH, methoxy, ethoxy, n-propoxy, isopropoxy, C₁fluoroalkoxy (e.g. trifluoromethoxy or difluoromethoxy), nitro (—NO₂), OH, C₁₋₃alkylS(O)₂- such as MeS(O)₂—, C₁₋₂alkylS(O)₂—NH— such as Me-S(O)₂—NH—, —CONH₂, cyano (—CN), or C₁₋₂alkylS(O)₂—CH₂— such as Me-S(O)₂—CH₂;

provided that two or more (e.g. three or more) of R^{6Aa}, R^{6Ba}, R^{6Da}, R^{6Ea} and R^{6Fa} are a hydrogen atom (H);

and wherein, in Formula (IB), on a molarity basis, more than 50% of the compound or salt present has the stereochemistry shown at the carbon atom bearing the R^{4aa} group.

In R^{1a}, C₂₋₃alkyl can for example be ethyl or n-propyl. In R^{1a}, C₂fluoroalkyl can for example be C₁fluoroalkyl-CH₂— such as CF₃—CH₂—. Preferably, R^{1a} is ethyl, n-propyl or —CH₂CH₂OH. R^{1a} is most preferably ethyl.

R^{2a} can for example be H.

The NHR^{3a} group of sub-formula (p14) is preferably in the cis configuration, i.e. is a [cis-4-(1-hydroxyethyl)cyclohexyl]amino group (including mixtures of configurations wherein the cis configuration is the major component).

Preferably, R^{4aa} is methyl, ethyl, CF₃ or —CH₂OH; more preferably R^{4aa} is methyl or ethyl; most preferably R^{4aa} is ethyl.

Preferably, R^{6Aa}, R^{6Ba}, R^{6Da}, R^{6Ea} and/or R^{6Fa}, independently of each other, is or are: a hydrogen atom (H), a fluorine, chlorine or bromine atom, methyl, ethyl, n-propyl, isopropyl, trifluoromethyl, —CH₂OH, methoxy, ethoxy, n-propoxy, difluoromethoxy, OH or MeS(O)₂—.

Preferably, three or more of R^{6Aa}, R^{6Ba}, R^{6Da}, R^{6Ea} and R^{6Fa} are a hydrogen atom (H).

In formula (IB), the phenyl ring attached to —(CHR^{4aa})— is suitably unsubstituted, monosubstituted, disubstituted or trisubstituted; or preferably the phenyl ring is unsubstituted, monosubstituted or disubstituted; more preferably monosubstituted or disubstituted.

In formula (IB), for monosubstitution of the phenyl ring, then preferably either R^{6Ba} or R^{6Da} is a fluorine, chlorine or bromine atom, methyl, ethyl, n-propyl, isopropyl, trifluoromethyl, —CH₂OH, methoxy, ethoxy, n-propoxy, difluoromethoxy, OH or MeS(O)₂- (preferably a fluorine, chlorine or bromine atom, methyl, ethyl, n-propyl, isopropyl, trifluoromethyl, methoxy, ethoxy or difluoromethoxy) and the remainder of R^{6Aa}, R^{6Ba}, R^{6Da}, R^{6Ea} and R^{6Fa} are H. Alternatively, for monosubstitution of the phenyl ring in formula (II), then preferably R^{6Aa} can be a fluorine or chlorine atom, methyl, ethyl, trifluoromethyl, methoxy or difluoromethoxy, and R^{6Ba}, R^{6Da}, R^{6Ea} and R^{6Fa} are H.

In formula (IB), for disubstitution of the phenyl ring, then 3,4-disubstitution, 2,4-disubstitution, 2,3-disubstitution, 2,5-disubstitution or 3,5-disubstitution of the phenyl ring is suitable. For example, in formula (IB), the phenyl ring can be 3,4-dimethylphenyl (R^{6Ba} and R^{6Da} are methyl, and R^{6Aa}, R^{6Ea} and R^{6Fa} are H) or 2,4-dimethylphenyl (R^{6Aa} and R^{6Da} are methyl, and R^{6Ba}, R^{6Ea} and R^{6Fa} are H) or 2,5-dimethylphenyl (R^{6Aa} and R^{6Ea} are methyl, and R^{6Ba}, R^{6Da} and R^{6Fa} are H) or 3,5-dimethylphenyl (R^{6Ba} and R^{6Ea} are methyl, and R^{6Aa}, R^{6Da} and R^{6Fa} are H) or 2-fluoro-4-chlorophenyl (R^{6Aa} is a fluorine atom, R^{6Da} is a chlorine atom, and R^{6Ba}, R^{6Ea} and R^{6Fa} are H) or 3-chloro-4-methylphenyl (R^{6Ba} is a chlorine atom and R^{6Da} is methyl, and R^{6Aa}, R^{6Ea} and R^{6Fa} are H).

In Formula (IB), on a molarity basis, preferably 70% or more, more preferably 75% or more, still more preferably 85% or more, yet more preferably 90% or more, for example 95% or more such as 98% or more, of the compound or salt present has the stereochemistry shown at the carbon atom bearing the R^{4aa} group.

Preferably, in Formula (IB), the stereochemistry at the carbon atom bearing the R^{4aa} group is such that there is an enantiomeric excess (e.e.) of 50% or more at the carbon atom bearing the R^{4aa} group (ignoring the stereochemistry at any other carbon atoms). More preferably, the enantiomeric excess (e.e.) is 70% or more or 80% or more, still more preferably 90% or more, yet more preferably 95% or more, at the carbon atom bearing the R^{4aa} group (ignoring the stereochemistry at any other carbon atoms). As stated before, “enantiomeric excess” (e.e.) is defined as the percentage of the major isomer present minus the percentage of the minor isomer present. For example, if 95% of major isomer is present and 5% of the minor isomer is present, then the e.e. would be 90%.

The compound formula (IB) or the salt thereof is preferably 4-{[cis-4-(1-hydroxyethyl)cyclohexyl]amino}-N-[1-(2,4-dimethylphenyl)propyl]-1-ethyl-1H-pyrazolo[3,4-b]pyridine-5-carboxamide or a salt thereof (e.g. a pharmaceutically acceptable salt thereof), having more than 50% by molarity in the (R)-stereochemistry at the benzylic carbon atom. See for example Example 373 hereinafter.

All references hereinafter to salts, solvates, isomers, tautomeric forms, molecular weights, synthetic process routes, medical uses, pharmaceutical compositions and dosing, and combinations, etc. can also relate to/include the compound formula (IB) or the salt thereof as an alternative to the compound formula (I) or the salt thereof.

Salts, Solvates, Isomers, Tautomeric Forms, Molecular Weights, Etc.

Because of their potential use in medicine, the salts of the compounds of formula (I) are preferably pharmaceutically acceptable. Suitable pharmaceutically acceptable salts can include acid or base addition salts.

A pharmaceutically acceptable acid addition salt can be formed by reaction of a compound of formula (I) with a suitable inorganic or organic acid (such as hydrobromic, hydrochloric, sulfuric, nitric, phosphoric, succinic, maleic, formic, acetic, propionic, fumaric, citric, tartaric, lactic, benzoic, salicylic, glutamaic, aspartic, p-toluenesulfonic, benzenesulfonic, methanesulfonic, ethanesulfonic, naphthalenesulfonic such as 2-naphthalenesulfonic, or hexanoic acid), optionally in a suitable solvent such as an organic solvent, to give the salt which is usually isolated for example by crystallisation and filtration. A pharmaceutically acceptable acid addition salt of a compound of formula (I) can comprise or be for example a hydrobromide, hydrochloride, sulfate, nitrate, phosphate, succinate, maleate, formate, acetate, propionate, fumarate, citrate, tartrate, lactate, benzoate, salicylate, glutamate, aspartate, p-toluenesulfonate, benzenesulfonate, methanesulfonate, ethanesulfonate, naphthalenesulfonate (e.g. 2-naphthalenesulfonate) or hexanoate salt.

A pharmaceutically acceptable base addition salt can be formed by reaction of a compound of formula (I) with a suitable inorganic or organic base (e.g. triethylamine, ethanolamine, triethanolamine, choline, arginine, lysine or histidine), optionally in a suitable solvent such as an organic solvent, to give the base addition salt which is usually isolated for example by crystallisation and filtration.

Other suitable pharmaceutically acceptable salts include pharmaceutically acceptable metal salts, for example pharmaceutically acceptable alkali-metal or alkaline-earth-metal salts such as sodium, potassium, calcium or magnesium salts; in particular pharmaceutically acceptable metal salts of one or more carboxylic acid moieties that may be present in the the compound of formula (I).

Other non-pharmaceutically acceptable salts, eg. oxalates, may be used, for example in the isolation of compounds of the invention, and are included within the scope of this invention.

The invention includes within its scope all possible stoichiometric and non-stoichiometric forms of the salts of the compounds of formula (I).

Also included within the scope of the invention are all solvates, hydrates and complexes of compounds and salts of the invention.

Certain groups, substituents, compounds or salts included in the present invention may be present as isomers. The present invention includes within its scope all such isomers, including racemates, enantiomers and mixtures thereof.

In the compounds or salts, pharmaceutical compositions, uses, methods of treatment/prophylaxis, methods of preparing, etc. according to the present invention, where a defined isomeric configuration e.g. stereochemical configuration is described or claimed, the invention includes a mixture comprising (a) a major component of the compound or salt which is in the described or claimed configuration, together with (b) one or more minor components of the compound or salt which is/are not in the described or claimed configuration. Preferably, in such a mixture, the major component of the compound or salt which is in the described or claimed configuration represents 70% or more, or 75% or more, more preferably 85% or more, still more preferably 90% or more, yet more preferably 95% or more, yet more preferably 98% or more, of the total amount of compound or salt present in the mixture on a molarity basis.

The percentage of one isomeric/stereochemical component in a mixture of different isomeric/stereochemical components, and if appropriate enantiomeric and/or diastereomeric excesses, can be measured using techniques known in the art. Such methods include the following:

(1) Measurement using NMR (e.g. ¹H NMR) spectroscopy in the presence of chiral agent. One can measure a nuclear magnetic resonance (NMR) spectrum (preferably a ¹H NMR spectrum, and/or a solution-phase NMR spectrum e.g. in CDCl₃ or D6-DMSO solvent) of the compound/salt mixture in the presence of a suitable chiral agent which “splits” the NMR peaks of a given atom in different isomers into different peak positions. The chiral agent can be: i) an optically pure reagent which reacts with the compound/salt e.g. to form a mixture of diastereomers, ii) a chiral solvent, iii) a chiral molecule which forms a transient species (e.g. diastereomeric species) with the compound/salt, or iv) a chiral shift reagent. See e.g. J. March, ♭Advanced Organic Chemistry?, 4th edn., 1992, pages 125-126 and refs. 138-146 cited therein. A chiral shift reagent can be a chiral lanthanide shift reagent such as tris[3-trifluoroacetyl-d-camphorato]europium-(II) or others as described in Morrill, “Lanthanide Shift Reagents in Stereochemical Analysis”, VCH, New York, 1986. Whatever the chiral agent is that is used, usually, the relative integrals (intensities) for the NMR peaks of a given atom or group in different isomers can provide a measurement of the relative amounts of each isomer present.

(2) Measurement using chiral chromatography, especially on an analytical scale. A suitable chiral column which separates the different isomeric components can be used to effect separation, e.g. using gas or liquid chromatography such as HPLC, and/or e.g. on an analytical scale. The peaks for each isomer can be integrated (area under each peak); and a comparison or ratio of the integrals for the different isomers present can give a measurement of the percentage of each isomeric component present. See for example: “Chiral Chromatography”, Separation Science Series Author: T. E. Beesley and R. P. W. Scott, John Wiley & Sons, Ltd., Chichester, UK, 1998, electronic Book ISBN: 0585352690, Book ISBN: 0471974277.

(3) Separation of pre-existing diastereomeric mixtures which are compounds/salts of the invention can be achieved (usually directly, without derivatisation) using separation techniques such as gas or liquid chromatography. Diastereomeric ratios and/or excesses can thereby be derived e.g. from the relative peak areas or relative separated masses.

(4) Conversion with a chiral/optically-active agent and subsequent separation of the resulting isomers, e.g. diastereomers. Conversion can be via derivatisation of a derivatisable group (e.g. —OH, —NHR) on the compound/salt with an optically-active derivatising group (e.g. optically active acid chloride or acid anhydride); or can be via formation of an acid or base addition salt of the compound by treatment of the compound with an optically-active acid or base, such as + or − di-para-toluoyl tartaric acid. After derivatisation, separation of the resulting isomers e.g. diastereomers, can be using gas or liquid chromatography (usually non-chiral); or (especially with isomeric salts) can be by selective crystallisation of a single isomeric e.g. diastereoisomeric salt. Determination of isomeric ratios and/or excesses can be using chromatography peak areas or measurement of mass of each separated isomer.

See e.g. J. March, “Advanced Organic Chemistry”, 4th edn., 1992, pages 120-121 and 126, and refs. 105-115 and 147-149 cited therein.

(5) Measurement of optical activity [alpha] of mixture and comparison with optical activity of pure isomer [alpha]max if available (e.g. see J. March, “Advanced Organic Chemistry”, 4th edn., 1992, page 125 and refs. 138-139 cited therein). This assumes a substantially linear relationship between [alpha] and concentration.

Certain of the groups, e.g. heteroaromatic ring systems, included in compounds of formula (I) or their salts may exist in one or more tautomeric forms. The present invention includes within its scope all such tautomeric forms, including mixtures.

Especially when intended for oral medicinal use, the compound of formula (I) can optionally have a molecular weight of 1000 or less, for example 800 or less, in particular 650 or less or 600 or less. Molecular weight here refers to that of the unsolvated “free base” compound, that is excluding any molecular weight contributed by any addition salts, solvent (e.g. water) molecules, etc.

Synthetic Process Routes

The following processes can be used to make the compounds of the invention:

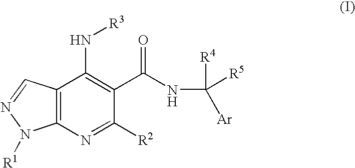

Some of the following synthetic processes may be exemplified for compounds of Formula (I) wherein R² is a hydrogen atom (H). However, some or all of these processes can also be used with appropriate modification, e.g. of starting materials and reagents, for making compounds of Formula (I) wherein R² is methyl.

Process A

To form a compound of formula (I), a carboxylic acid of formula (II) can be converted into an activated compound of formula (E) wherein X¹ is a leaving group substitutable by an amine (as defined below), and subsequently the activated compound can be reacted with an amine of formula ArCR⁴R⁵NH₂:

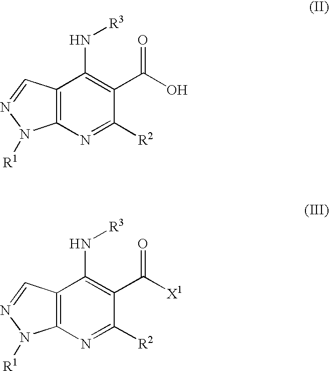

For example, the activated compound (the compound of formula (III)) can be the acid chloride (X¹═Cl). This can be formed from the carboxylic acid of formula (II) e.g. by reaction with thionyl chloride, either in an organic solvent such as chloroform or without solvent. Alternatively, the activated compound (the compound of formula (III)) can be an activated ester wherein the leaving group X¹ is

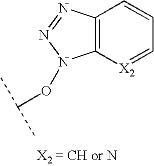

The latter activated compound of formula (III) can be formed from the carboxylic acid of formula (I) either:

(a) by reaction of the carboxylic acid with a carbodiimide such as EDC, which is 1-ethyl-3-(3′-dimethylaminopropyl)carbodiimide and is also 1-(3-dimethylaminopropyl)-3-ethylcarbodiimide, or a salt thereof e.g. hydrochloride salt, preferably followed by reaction of the resulting product with 1-hydroxybenzotriazole (HOBT); reaction (a) usually being carried out in the presence of a solvent (preferably anhydrous) such as dimethyl formamide (DMF) or acetonitrile and/or preferably under anhydrous conditions and/or usually at room temperature (e.g. about 20 to about 25° C.);

or:

(b) by reaction with 2-(1H-benzotriazole-1-yl)-1,1,3,3-tetramethyluronium tetrafluoroborate (TBTU) or O-(7-Azabenzotriazol-1-yl)-N,N,N′,N′-tetramethyluronium hexafluorophosphate (HATU) ,in the presence of a base such as diisopropylethylamine (ⁱPr₂NEt=DIPEA), and usually in the presence of a solvent such as dimethyl formamide (MF) or acetonitrile and/or preferably under anhydrous conditions and/or usually at room temperature (e.g. about 20 to about 25° C.).

Compounds of formula (II) can be prepared by hydrolysis of a compound of formula (IV), an ester:

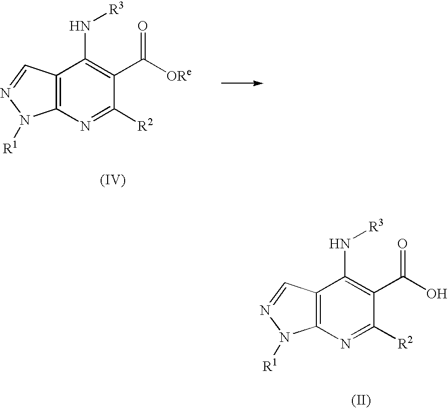

This process preferably involves reaction of compound of formula (IV) with either:
(a) a base, such as sodium hydroxide or potassium hydroxide, in a solvent, e.g. an aqueous solvent such as aqueous ethanol or aqueous dioxane or
(b) an acid, such as hydrochloric acid, in a solvent, e.g. an aqueous solvent such as aqueous dioxane.

Compounds of formula (IV) can be prepared according to a method, for example as described by Yu et. al. in J. Med Chem., 2001, 44, 1025-1027, by reaction of a compound of formula (V) with an amine of formula R³NH₂. The reaction is preferably carried out in the presence of a base such as triethylamine or N,N-diisopropylethylamine, and/or in an organic solvent such as ethanol, dioxane or acetonitrile. The reaction may require heating e.g. to ca. 60-100° C., for example ca. 80-90° C.:

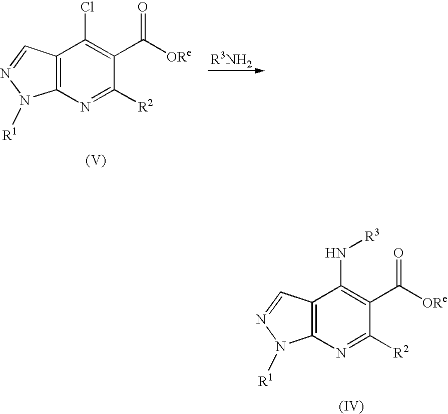

Compounds of formula (V) are also described in the above reference. They can be prepared by reaction of a compound of formula (VI) with (R²)(OEt)C═C(CO₂R^{e})₂, which can for example be diethyl(ethoxymethylene)malonate (wherein R² is H and R^{e} is Et) or diethyl 2-(1-ethoxyethylidene)malonate (wherein R² is Me and R^{e} is Et), with heating, followed by reaction with phosphorous oxychloride, again with heating:

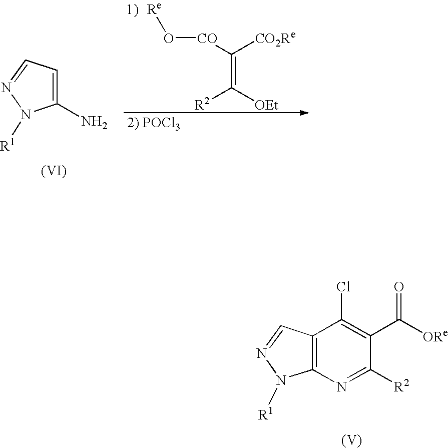

For examples of the compound (VI) to compound (V) process, see for example: (i) the Intermediate 1 synthesis and G. Yu et. al., J. Med Chem., 2001, 44, 1025-1027 hereinafter, where R²═H and R¹=ethyl; and see (ii) the Intermediate 10 synthesis hereinafter where R²=Me and R¹=ethyl; and see (iii) Intermediate 182 synthesis hereinafter wherein R²═H and R¹=methyl (i.e. reaction of 5-amino-1-methyl pyrazole with diethylethoxymethylene malonate).

Where the desired amino pyrazole of formula (VI) is not commercially available, preparation of the amino pyrazole (VI) can be achieved, for example, using methods described by Dorgan et. al. in J. Chem. Soc., Perkin Trans. 1, (4), 938-42; 1980, by reaction of cyanoethyl hydrazine with a suitable aldehyde of formula R⁴⁰CHO in a solvent such as ethanol, with heating, followed by reduction, for example reduction with sodium in a solvent such as t-butanol. R⁴⁰ should be chosen so as to contain one less carbon atom than R¹, for example R⁴⁰=methyl will afford R¹=ethyl.

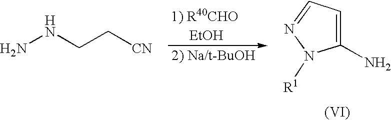

Alternatively, e.g. where the desired amino pyrazole of Formula (VI) is not commercially available, preparation of the 4-amino 5-ester/acid compounds of Formulae (IV) and (II) can be achieved from a (different R¹) 4-chloro 5-ester compound of Formula (V) (e.g. Intermediate 1, wherein R¹=ethyl), using a generalised version of the reaction scheme shown in Intermediate 170 and shown below. In this method:

the 4-chloro 5-ester pyrazolopyridine of Formula (V) (e.g. Intermediate 1) is optionally converted to the 4-alkoxy (e.g. C₁₋₄alkoxy such as ethoxy) pyrazolopyridine;

the R¹ group is removed (e.g. using N-bromosuccinimide (NBS) and preferably base e.g. Na₂CO₃) (e.g. to give Intermediate 1A—an alternative synthesis for which is given under “Intermediate 1A” hereinafter);

the 4-amino NHR³ group is inserted by displacing the 4-chloro or 4-alkoxy group by reaction with R³NH₂;

and the resulting pyrazolopyridine is alkylated at N-1 by reacting it with R¹—X⁴¹, where X⁴¹ is a group displaceable by the N-1 nitrogen of the pyrazolopyridine, in order to re-insert the desired R¹ group [i.e. to prepare the 4-amino 5-ester compound of Formula (IV)]. X⁴¹ can for example be a halogen, e.g. C1, Br or I; or X⁴¹ can be —O—S(O)₂—R⁴¹ where R⁴¹ is C₁₋₄alkyl, C₁₋₂fluoroalkyl, or phenyl optionally substituted by C₁₋₂alkyl. The N-1 alkylation reation with R¹—X⁴¹ is preferably carried out in the presence of base

see the (IX) to (IV) reaction hereinafter for examples of suitable bases.

The scheme below (Intermediate 170 scheme) shows a suitable exemplary route and conditions for this R¹ removal and re-insertion route, for insertion of R¹=n-propyl and R³=tetrahydro-2H-pyran-4-yl:

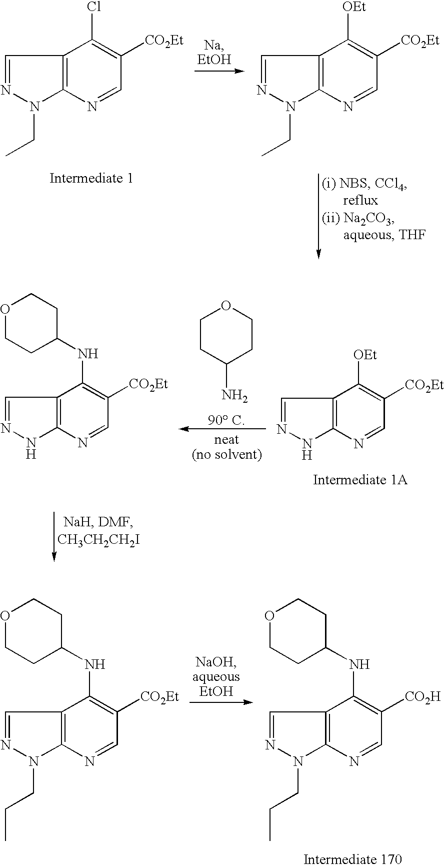

In an alternative embodiment of Process A, the 4-chloro substituent in the compound of formula (V) can be replaced by another halogen atom, such as a bromine atom, or by another suitable leaving group which is displaceable by an amine of formula R³NH₂. The leaving group displaceable by the amine can for example be R^{LA}, in a compound of formula (Va), wherein R^{LA} is an alkoxy group OR³⁵ such as OC₁₋₄alkyl (in particular OEt) or a group —O—S(O)₂—R³⁷. Here, R³⁷ is C₁₋₈alkyl (e.g. C₁₋₄alkyl or C₁₋₂alkyl such as methyl), C₁₋₆fluoroalkyl (e.g. C₁₋₄fluoroalkyl or C₁₋₂fluoroalkyl such as CF₃ or C₄F₉), or phenyl wherein the phenyl is optionally substituted by one or two of independently C₁₋₂alkyl, halogen or C₁₋₂alkoxy (such as phenyl or 4-methyl-phenyl). The reaction of the compound of formula (Va) with the amine of formula R³NH₂ may be carried out with or without solvent and may require heating:

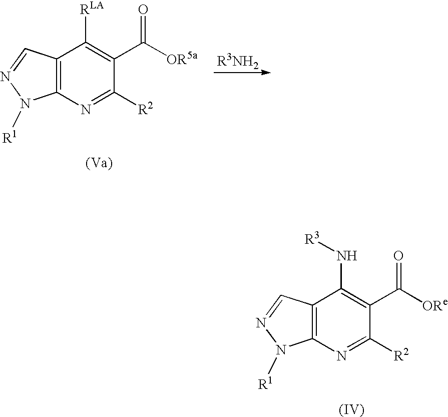

In another alternative embodiment of Process A, the compound of formula (IV), described herein, can be prepared by reaction of a compound of formula (IX) with an alkylating agent of formula R¹—X³, where X³ is a leaving group displaceable by the 1-position pyrazolopyridine nitrogen atom of the compound of formula (IX):

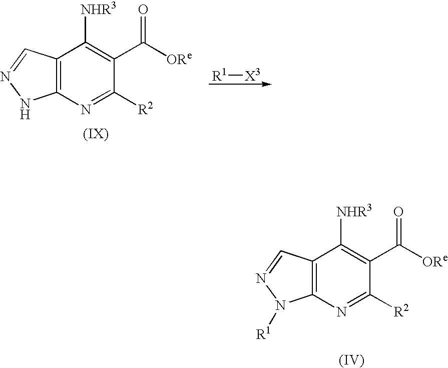

A suitable alkylating agent of formula R¹—X³ can be used. For example, X³ can be a halogen atom such as a chlorine atom or more preferably a bromine or iodine atom, or X³ can be —O—S(O)₂—R³⁶ wherein R³⁶ is C₁₋₈alkyl (e.g. C₁₋₄alkyl or C₁₋₂alkyl such as methyl), C₁₋₆fluoroalkyl (e.g. C₁₋₄fluoroalkyl or C₁₋₂fluoroalkyl such as CF₃ or C₄F₉), or phenyl wherein the phenyl is optionally substituted by one or two of independently C₁₋₂alkyl, halogen or C₁₋₂alkoxy (such as phenyl or 4-methyl-phenyl). The reaction is preferably carried out in the presence of a base; the base can for example comprise or be potassium carbonate, sodium carbonate, sodium hydride, potassium hydride, or a basic resin or polymer such as polymer-bound 2-tert-butylimino-2-diethylamino-1,3-dimethyl-perhydro-1,3,2-diazaphosphorine. The reaction is preferably carried out in the presence of a solvent, e.g. an organic solvent such as DMF; the solvent is preferably anhydrous.

Compounds of formula (IX) can be prepared, using a method analogous to that used for the preparation of compounds of formula (IV) from compounds of formula (V), by reaction of a compound of formula (X) (which is the same as compound of formula (V) but wherein R¹═H) with an amine of formula R³NH₂. The reaction is suitably carried out in the presence of a base such as triethylamine or N,N-diisopropylethylamine, and/or in an organic solvent such as ethanol, dioxane or acetonitrile. The reaction may require heating e.g. to ca. 60-100° C., for example ca. 80-90° C.:

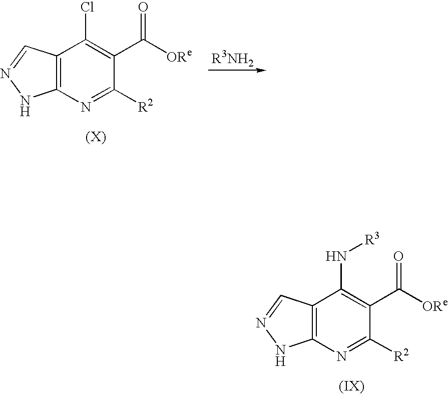

Alternatively, in formula (X), the 4-chloro can be replaced by 4-C₁₋₄alkoxy such as 4-ethoxy; these modified compounds, of formula (Xa), can optionally be made as described above, e.g. see the Intermediate 170 scheme shown and described above or Intermediate 1A below.

Process B

Compounds of formula (I) can be prepared by reaction of a compound of formula (VII) with an amine of formula R³NH₂. In the compound of formula (VII), R^{LB} is a leaving group which is displaceable by the amine of formula R³NH₂. R^{LB} can be a bromine atom (Br) or more particularly a chlorine atom (Cl), or alternatively R^{LB} can be an alkoxy group OR³⁵ such as OC₁₋₄alkyl (in particular OEt) or a group —O—S(O)₂—R³⁷. Here, R³⁷ is C₁₋₈alkyl (e.g. C₁₋₄alkyl or C₁₋₂alkyl such as methyl), C₁₋₆fluoroalkyl (e.g. C₁₋₄fluoroalkyl or C₁₋₂fluoroalkyl such as CF₃ or C₄F₉), or phenyl wherein the phenyl is optionally substituted by one or two of independently C₁₋₂alkyl, halogen or C₁₋₂alkoxy (such as phenyl or 4-methyl-phenyl). The reaction of (VII) to (I) is preferably carried out in the presence of a base, such as triethylamine or N,N-diisopropylethylamine, and/or in an organic solvent such as ethanol, THF, dioxane or acetonitrile. The reaction may require heating, e.g. to ca. 60-100° C. or ca. 80-90 ° C., for example for 8-48 or 12-24 hours:

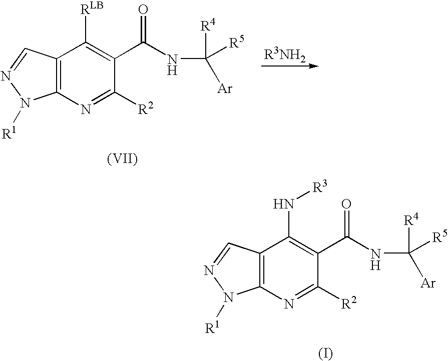

Compounds of formula (VII), wherein R^{LB} is a chlorine atom (compound of formula (VIIa), can be prepared in a two step procedure as described by Bare et. al. in J. Med. Chem. 1989, 32, 2561-2573. This process involves 2 steps. In the first step, a compound of formula (VIII) is reacted with thionyl chloride (or another agent suitable for forming an acid chloride from a carboxylic acid), either in an organic solvent such as chloroform or THF, or as a neat solution. This reaction may require heating and the thus-formed intermediate may or may not be isolated. Step two involves reaction with an amine of formula ArCR⁴R⁵NH₂, in an organic solvent such as THF or chloroform and may also involve the use of a base such as triethylamine or diisopropylethylamine:

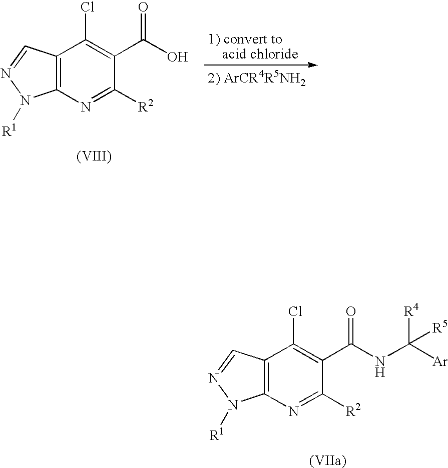

Compounds of formula (VIII) can be prepared by hydrolysis of an ester of formula (V) according to the method described by Yu et. al. in J. Med Chem., 2001, 44, 1025-1027. This procedure preferably involves reaction with a base, such as sodium hydroxide or potassium hydroxide, in a solvent e.g. an aqueous solvent such as aqueous ethanol or aqueous dioxane:

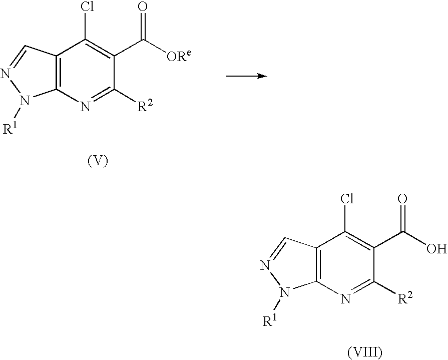

Compounds of formula (V) can be prepared as described in Process A above.

Process C

A compounds of formula (I) can be prepared by reaction of a compound of formula (IXa) with an alkylating agent of formula R¹—X³, where X³ is a leaving group displaceable by the 1-position pyrazolopyridine nitrogen atom of the compound of formula (IXa):

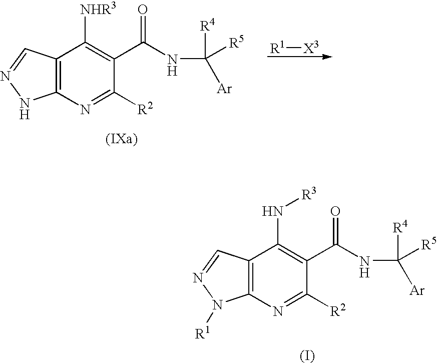

A suitable alkylating agent of formula R¹—X³ can be used. For example, X³ can be a halogen atom such as a chlorine atom or more preferably a bromine or iodine atom, or X³ can be —O—S(O)₂—R³⁶ wherein R³⁶ is C₁₋₈alkyl (e.g. C₁₋₄alkyl or C₁₋₂alkyl such as methyl), C₁₋₆fluoroalkyl (e.g. C₁₋₄fluoroalkyl or C₁₋₂fluoroalkyl such as CF₃ or C₄F₉), or phenyl wherein the phenyl is optionally substituted by one or two of independently C₁₋₂alkyl, halogen or C₁₋₂alkoxy (such as phenyl or 4-methyl-phenyl). The reaction is preferably carried out in the presence of a base; the base can for example comprise or be potassium carbonate, sodium carbonate, sodium hydride, potassium hydride, or a basic resin or polymer such as polymer-bound 2-tert-butylimino-2-diethylamino-1,3-dimethyl-perhydro-1,3,2-diazaphosphorine. The reaction is preferably carried out in the presence of a solvent, e.g. an organic solvent such as DMF; the solvent is preferably anhydrous.

Compounds of formula (Ixa) can be prepared from a compound of formula (IX): 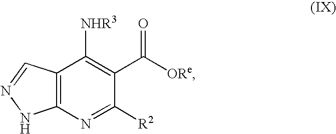
by hydrolysis of the ester and conversion of the resulting carboxylic acid to the amide of formula (IXa) by activation of the acid and reaction with an amine of formula ArCR⁴R⁵NH₂. The ester (IX) to acid to amide (IXa) conversion can suitably use the reagents and reaction conditions mentioned in Process A above for conversion of (IV) to (II) to (III) to (I).

The ester compound of formula (IX) can be prepared using the method described in the alternative embodiment of Process A, above.

Process D: Conversion of One Compound of Formula (I), (II) or (IV) or Salt Thereof Into Another Compound of Formula (I), (II) or (IV) or Salt Thereof

One compound of formula (I), (II) or (IV) or salt thereof (or a protected version thereof, such as an N-protected version e.g. BOC-N-protected) can be converted into a or another compound of formula (I), (II) or (IV) or salt thereof. This conversion preferably comprises or is one or more of the following processes D1 to D7:

D1. Conversion of a ketone into the corresponding oxime (e.g. Examples 231-281).

D2. An oxidation process. For example, the oxidation process can comprise or be oxidation of an alcohol to a ketone (e.g. using Jones reagent) or oxidation of an alcohol or a ketone to a carboxylic acid. The oxidation process can e.g. comprise or be conversion of a nitrogen-containing compound of formula (I) or salt thereof to the corresponding N-oxide (e.g. using meta-chloroperoxybenzoic acid), for example conversion of a pyridine-containing compound to the corresponding pyridine N-oxide (e.g. see Examples 210-212 of PCT/EP03/11814 (WO 2004/024728 A2), filed on 12 Sep. 2003 and incorporated herein by reference, for suitable process details).

D3. A reduction process, for example reduction of a ketone or a carboxylic acid to an alcohol.

D4. Acylation, for example acylation of an amine (e.g. see Examples 329-349 and Example 353 of PCT/EP03/11814 (WO 2004/024728 A2), filed on 12 Sep. 2003 and incorporated herein by reference, for suitable process details), or acylation of a hydroxy group.

D5. Alkylation, for example alkylation of an amine or of a hydroxy group.

D6. Hydrolysis, e.g. hydrolysis of an ester to the corresponding carboxylic acid or salt thereof (e.g. see Examples 351, 488, 489, 650, 651 of PCT/EP03/11814 (WO 2004/024728 A2), filed on 12 Sep. 2003 and incorporated herein by reference, for suitable process details).

D7. Deprotection, e.g. deprotection of (e.g. deacylation of or t-butyloxycarbonyl (BOC) removal from) an amine group. BOC deprotection can be carried out under acidic conditions e.g. using hydrogen chloride in an organic solvent such as dioxan—Examples 381 and 382 herein are examples of such a BOC deprotection process.

D8. Formation of an ester or amide, for example from the corresponding carboxylic acid.

D9. Sulfonylation, e.g. sulfonamide formation by reaction of an amine with a sulfonyl halide e.g. a sulfonyl chloride (e.g. see Examples 322-328 of PCT/EP03/11814 (WO 2004/024728 A2), filed on 12 Sep. 2003 and incorporated herein by reference, for suitable process details).

and/or

D10. Beckmann rearrangement of one compound of formula (I) into another compound of formula (I), for example using cyanuric chloride (2,4,6-trichloro-1,3,5-triazine) together with a formamide such as DMF, e.g. at room temperature (see L. D. Luca, J. Org. Chem., 2002, 67, 6272-6274). The Beckmann rearrangement can for example comprise conversion of a compound of formula (I) wherein NHR³ is of sub-formula (o2) 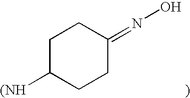
into a compound of formula (I) wherein NHR³ is of sub-formula (m3) 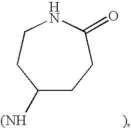
and suitable process details can be as illustrated in Examples 658 and 659 of PCT/EP03/11814 (WO 2004/024728 A2), filed on 12 Sep. 2003 and incorporated herein by reference.

The present invention therefore also provides a method of preparing a compound of formula (I) or a salt thereof: 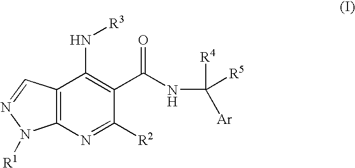
wherein R¹, R², R³, R⁴, R⁵ and Ar are as defined herein, the method comprising:

(a) reaction of an activated compound of formula (III), 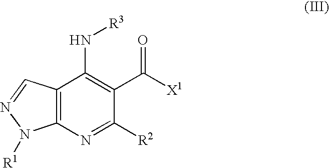
wherein X¹ is a leaving group substitutable by an amine, with an amine of formula ArCR⁴R⁵NH₂;

(b) reaction of a compound of formula (VII): 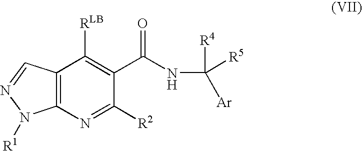
, wherein R^{LB} is a leaving group which is displaceable by an amine of formula R³NH₂, with an amine of formula R³NH₂;

(c) reaction of a compound of formula (IXa) with an alkylating agent of formula R¹—X³, where X³ is a leaving group displaceable by the 1-position pyrazolopyridine nitrogen atom of the compound of formula (IXa): 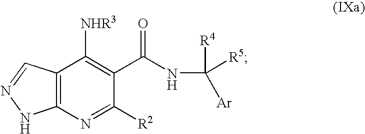
or

(d) conversion of one compound of formula (I) or salt thereof (or a protected version thereof, such as an N-protected version e.g. BOC-N-protected) into a or another compound of formula (I) or salt thereof;

and optionally converting the compound of formula (I) into a salt thereof e.g. a pharmaceutically acceptable salt thereof.

Preferred, suitable or optional features of methods (a), (b), (c) and (d), independently of each other, are as described above for Processes A, B, C, and D, with all necessary changes being made.

The present invention also provides: (e) a method of preparing a pharmaceutically acceptable salt of a compound of formula (I) comprising conversion of the compound of formula (I) or a salt thereof into the desired pharmaceutically acceptable salt thereof. (See for example Example 307 herein).

The present invention also provides a compound of formula (I) or a salt thereof, prepared by a method as defined herein.

Medical Uses

The present invention also provides a compound of formula (I) or a pharmaceutically acceptable salt thereof for use as an active therapeutic substance in a mammal such as a human. The compound or salt can be for use in the treatment and/or prophylaxis of any of the diseases/conditions described herein (e.g. for use in the treatment and/or prophylaxis of an inflammatory and/or allergic disease in a mammal such as a human; or e.g. for use in the treatment and/or prophylaxis of cognitive impairment or depression in a mammal such as a human) and/or for use as a phosphodiesterase inhibitor e.g. for use as a phosphodiesterase 4 (PDE4) inhibitor. “Therapy” may include treatment and/or prophylaxis.

Also provided is the use of a compound of formula (I) or a pharmaceutically acceptable salt thereof in the manufacture of a medicament (e.g. pharmaceutical composition) for the treatment and/or prophylaxis of any of the diseases/conditions described herein in a mammal such as a human, e.g. for the treatment and/or prophylaxis of an inflammatory and/or allergic disease in a mammal such as a human, or e.g. for the treatment and/or prophylaxis of cognitive impairment or depression in a mammal.

Also provided is a method of treatment and/or prophylaxis of any of the diseases/conditions described herein in a mammal (e.g. human) in need thereof, e.g. a method of treatment and/or prophylaxis of an inflammatory and/or allergic disease, cognitive impairment or depression in a mammal (e.g. human) in need thereof, which method comprises administering to the mammal (e.g. human) a therapeutically effective amount of a compound of formula (I) as herein defined or a pharmaceutically acceptable salt thereof.

Phosphodiesterase 4 inhibitors are thought to be useful in the treatment and/or prophylaxis of a variety of diseases/conditions, especially inflammatory and/or allergic diseases, in mammals such as humans, for example: asthma, chronic obstructive pulmonary disease (COPD) (e.g. chronic bronchitis and/or emphysema), atopic dermatitis, urticaria, allergic rhinitis, allergic conjunctivitis, vernal conjunctivitis, eosinophilic granuloma, psoriasis, rheumatoid arthritis, septic shock, ulcerative colitis, Crohn's disease, reperfusion injury of the myocardium and brain, chronic glomerulonephritis, endotoxic shock, adult respiratory distress syndrome, multiple sclerosis, cognitive impairment (e.g. in a neurological disorder such as Alzheimer's disease), depression, or pain (e.g. inflammatory pain). Ulcerative colitis and/or Crohn's disease are collectively often referred to as inflammatory bowel disease.

In the treatment and/or prophylaxis, the inflammatory and/or allergic disease can suitably be chronic obstructive pulmonary disease (COPD), asthma, rheumatoid arthritis, allergic rhinitis or atopic dermatitis in a mammal (e.g. human). In the treatment and/or prophylaxis, the inflammatory and/or allergic disease is suitably chronic obstructive pulmonary disease (COPD), asthma, rheumatoid arthritis or allergic rhinitis in a mammal (e.g. human). More preferably, the treatment and/or prophylaxis is of COPD or asthma in a mammal (e.g. human).

PDE4 inhibitors are thought to be effective in the treatment of asthma (e.g. see M. A. Giembycz, Drugs, Feb. 2000, 59(2), 193-212; Z. Huang et al., Current Opinion in Chemical Biology, 2001, 5: 432-438; H. J. Dyke et al., Expert Opinion on Investigational Drugs, January 2002, 11(1), 1-13; C. Burnoufet al., Current Pharmaceutical Design, 2002, 8(14), 1255-1296; A. M. Doherty, Current Opinion Chem. Biol., 1999, 3(4), 466-473; P. J. Barnes, Naure Reviews—Drug Discovery, October 2004, 831-844; and references cited in the aforementioned publications).

PDE4 inhibitors, for example cilomilast and roflumilast, are thought to be effective in the treatment of COPD. For example, see S. L. Wolda, Emerging Drugs, 2000, 5(3), 309-319; Z. Huang et al., Current Opinion in Chemical Biology, 2001, 5: 432-438; H. J. Dyke et al., Expert Opinion on Investigational Drugs, January 2002, 11(1), 1-13; C. Burnouf et al., Current Pharmaceutical Design, 2002, 8(14), 1255-1296; A. M. Doherty, Current Opinion Chem. Biol., 1999, 3(4), 466-473; A. M. Vignola, Respiratory Medicine, 2004, 98, 495-503; D. Spina, Drugs, 2003, 63(23), 2575-2594; and references cited in the aforementioned publications; and G. Krishna et al., Expert Opinion on Investigational Drugs, 2004, 13(3), 255-267 (see especially pp.259-261 and refs. 102-111 and 201 therein). COPD is often characterised by the presence of airflow obstruction due to chronic bronchitis and/or emphysema (e.g., see S. L. Wolda, Emerging Drugs, 2000, 5(3), 309-319).

PDE4 inhibitors are thought to be effective in the treatment of allergic rhinitis (e.g. see B. M. Schmidt et al., J, Allergy & Clinical Immunology, 108(4), 2001, 530-536).

PDE4 inhibitors are thought to be effective in the treatment of rheumatoid arthritis and multiple sclerosis (e.g. see H. J. Dyke et al., Expert Opinion on Investigdtional Drugs, January 2002, 11(1), 1-13; C. Burnouf et al., Current Pharmaceutical Design, 2002, 8(14), 1255-1296; and A. M. Doherty, Current Opinion Chem. Biol., 1999, 3(4), 466-473; and references cited in these publications).

See e.g. A. M. Doherty, Current Opinion Chem. Biol., 1999, 3(4), 466-473 and references cited therein for atopic dermatitis use.

For treatment and/or prophylaxis of atopic dermatitis, topical administration (e.g. topical administration to the skin e,g. to affected skin) can be used.

PDE4 inhibitors have been suggested as having analgesic properties and thus being effective in the treatment of pain (A. Kumar et al., Indian J. Exp. Biol., 2000, 38(1), 26-30).

In the invention, the treatment and/or prophylaxis can be of cognitive impairment e.g. cognitive impairment in a neurological disorder such as Alzheimer's disease. For example, the treatment and/or prophylaxis can comprise cognitive enhancement e.g. in a neurological disorder. See for example: H. T. Zhang et al. in: Psychopharmacology, June 2000, 150(3), 311-316 and Neuropsychopharmacology, 2000, 23(2), 198-204; and T. Egawa et al., Japanese J. Pharmacol., 1997, 75(3), 275-81.

PDE4 inhibitors such as rolipram have been suggested as having antidepressant properties (e.g. J. Zhu et al., CNS Drug Reviews, 2001, 7(4), 387-398; O'Donnell, Expert Opinion on Investigational Drugs, 2000, 9(3), 621-625; H. T. Zhang et al., Neuropsyclzopharmacology, October 2002, 27(4), 587-595; J. M. O'Donnell and H.-T. Zhang, Trends Pharmacol. Sci., March 2004, 25(3), 158-163; and T. E. Renau, Curr. Opinion Invest. Drugs, 2004, 5(1), 34-39).

PDE4 inhibition has been suggested for the treatment of inflammatory bowel disease (e.g. ulcerative colitis and/or Crohn's disease), see K. H. Banner and M. A. Trevethick, Trends Pharmacol. Sci., August 2004, 25(8), 430-436.

Pharmaceutical Compositions and Dosing

For use in medicine, the compounds of the present invention are usually administered as a pharmaceutical composition.

The present invention therefore provides in a further aspect a pharmaceutical composition comprising a compound of formula (I) or a pharmaceutically acceptable salt thereof and one or more pharmaceutically acceptable carriers and/or excipients.

The pharmaceutical composition can be for use in the treatment and/or prophylaxis of any of the conditions described herein.

The invention also provides a method of preparing a pharmaceutical composition comprising a compound of formula (I), as herein defined, or a pharmaceutically acceptable salt thereof, and one or more pharmaceutically acceptable carriers and/or excipients,

the method comprising mixing the compound or salt with the one or more pharmaceutically acceptable carriers and/or excipients.

The invention also provides a pharmaceutical composition prepared by said method.

The compounds of formula (I) and/or the pharmaceutical composition may be administered, for example, by oral, parenteral (e.g. intravenous, subcutaneous, or intramuscular), inhaled, topical (e.g. skin topical), or nasal administration. Accordingly, the pharmaceutical composition is preferably suitable for oral, parenteral (e.g. intravenous, subcutaneous, or intramuscular), inhaled, topical (e.g. skin topical), or nasal administration.

More preferably, the pharmaceutical composition is suitable for inhaled or oral administration, e.g. to a mammal such as a human. Inhaled administration involves topical administration to the lung e.g. by aerosol or dry powder composition.

A pharmaceutical composition suitable for oral administration can be liquid or solid; for example it can be a syrup, suspension or emulsion, a tablet, a capsule or a lozenge.

A liquid formulation (e.g. oral) will generally consist of a suspension or solution of the compound or pharmaceutically acceptable salt in a suitable pharmaceutically acceptable liquid carrier(s), for example an aqueous solvent such as water, ethanol or glycerine, or a non-aqueous solvent, such as polyethylene glycol or an oil. The formulation may also contain a suspending agent, preservative, flavouring and/or colouring agent.

In one embodiment, the pharmaceutical composition is in unit dose form, such as a tablet or capsule for oral administration, e.g. for oral administration to a human.

A pharmaceutical composition suitable for oral administration being a tablet can comprise one or more pharmaceutically acceptable carriers and/or excipients suitable for preparing tablet formulations. The carrier can for example be or include lactose, cellulose (for example microcrystalline cellulose), or mannitol. The tablet can also or instead contain one or more pharmaceutically acceptable excipients, for example a binding agent such as hydroxypropylmethylcellulose or povidone (polyvinylpyrrolidone), a lubricant e.g. an alkaline earth metal stearate such as magnesium stearate, and/or a tablet disintegrant such as sodium starch glycollate, croscarmellose sodium, or crospovidone (cross-linked polyvinylpyrrolidone). The pharmaceutical composition being a tablet can be prepared by a method comprising the steps of: (i) mixing the compound of formula (I), as herein defined, or a pharmaceutically acceptable salt thereof, with the one or more pharmaceutically acceptable carriers and/or excipients, (ii) compressing the resulting mixture (which is usually in powder form) into tablets, and (iii) optionally coating the tablet with a tablet film-coating material.

A pharmaceutical composition suitable for oral administration being a capsule can be prepared using encapsulation procedures. For example, pellets or powder containing the active ingredient can be prepared using a suitable pharmaceutically acceptable carrier and then filled into a hard gelatin capsule. Alternatively, a dispersion or suspension can be prepared using any suitable pharmaceutically acceptable carrier, for example an aqueous gum or an oil and the dispersion or suspension then filled into a soft gelatin capsule.

A parenteral composition can comprise a solution or suspension of the compound or pharmaceutically acceptable salt in a sterile aqueous carrier or parenterally acceptable oil. Alternatively, the solution can be lyophilised; the lyophilised parenteral pharmaceutical composition can be reconstituted with a suitable solvent just prior to administration.

A topical pharmaceutical composition, e.g. skin topical pharmaceutical composition, can for example be an ointment, a cream (i.e. an oil-in-water pharmaceutical composition), an aqueous gel, or a DMSO-containing solution such as a DMSO/acetone solution (DMSO=dimethyl sulphoxide). A topical pharmaceutical composition, e.g. an oil-in-water composition, can optionally include a skin-penetration enhancer such as propylene glycol, and/or (e.g. for an oil-in-water composition) an emulsifier (e.g. surfactant) such as sodium dodecyl sulphate (SDS). A topical ointment can for example comprise polyethylene glycol and/or propylene glycol. In a topical pharmaceutical composition, such as an ointment or an oil-in-water composition, the compound of formula (I) or the salt thereof can optionally be present at 0.25 to 5%, for example 0.5 to 2.5%, by weight of the total composition. In a topical pharmaceutical composition, the compound of formula (I) or the salt thereof can optionally be Example 73, 75, 98, 283, 304, 306, 307, 310, 311, 316, 321, 324, 326, 327, 328, 330, 331, 332, 333, 334, 335, 336, 337, 338, 339, 343, 344 or 345, as the compound or a pharmaceutically acceptable salt thereof. A topical pharmaceutical composition, e.g. skin topical pharmaceutical composition, can for example be for treatment and/or prophylaxis of atopic dermatitis e.g. in a mammal such as a human.

Compositions for nasal or inhaled administration may conveniently be formulated as aerosols, drops, gels or dry powders.

Aerosol formulations, e.g. for inhaled administration, can comprise a solution or fine suspension of the active substance in a pharmaceutically acceptable aqueous or non-aqueous solvent. Aerosol formulations can be presented in single or multidose quantities in sterile form in a sealed container, which can take the form of a cartridge or refill for use with an atomising device or inhaler. Alternatively the sealed container may be a unitary dispensing device such as a single dose nasal inhaler or an aerosol dispenser fitted with a metering valve (metered dose inhaler) which is intended for disposal once the contents of the container have been exhausted.

Where the dosage form comprises an aerosol dispenser, it preferably contains a suitable propellant under pressure such as compressed air, carbon dioxide, or an organic propellant such as a chlorofluorocarbon (CFC) or hydrofluorocarbon (HFC). Suitable CFC propellants include dichlorodifluoromethane, trichlorofluoromethane and dichlorotetrafluoroethane. Suitable HFC propellants include 1,1,1,2,3,3,3-heptafluoropropane and 1,1,1,2-tetrafluoroethane. The aerosol dosage forms can also take the form of a pump-atomiser.

Particle Size Reduction of Compound of Formula (a) or Salt Thereof

For use in, for example, pharmaceutical compositions suitable and/or adapted for inhaled administration, it is preferred that the compound or salt of formula (I) is in a particle-size-reduced form, and more preferably the size-reduced form is obtained or obtainable by micronisation. Micronisation usually involves subjecting the compound/salt to collisional and/or abrasional forces in a fast-flowing circular or spiral/vortex-shaped airstream often including a cyclone component. The preferable particle size of the size-reduced (e.g. micronised) compound or salt is defined by a D50 value of about 0.5 to about 10 microns, e.g. about 1 to about 7 microns or about 1 to about 5 microns (e.g. as measured using laser diffraction). For example, it is preferable for the compound or salt of formula (I) to have a particle size defined by: a D10 of about 0.3 to about 3 microns (e.g. about 0.5 to about 2 microns, or about 1 micron), and/or a D50 of about 0.5 to about 10 microns or about 1 to about 7 microns or (e.g. about 1 to about 5 microns or about 2 to about 5 microns or about 2 to about 4 microns), and/or a D90 of about 1 to about 30 microns or about 2 to about 20 microns or about 2 to about 15 microns or about 3 to about 15 microns (e.g. about 5 to about 15 microns or about 5 to about 10 microns or about 2 to about 10 microns); for example as measured using laser diffraction.

In particle size measurements, D90, D50 and D10 respectively mean that 90%, 50% and 10% ofthe material is less than the micron size specified. D50 is the median particle size. DV90, DV50 and DV10 respectively mean that 90%, 50% and 10% by volume of the material is less than the micron size specified. DM90, DM50 and DM10 respectively mean that 90%, 50% and 10% by weight of the material is less than the micron size specified.

Laser diffraction measurement of particle size can use a dry method (wherein a suspension of the compound/salt in an airflow crosses the laser beam) or a wet method [wherein a suspension of the compound/salt in a liquid dispersing medium, such as isooctane or (e.g. if compound is soluble in isooctane) 0.1% Tween 80 in water, crosses the laser beam]. With laser diffraction, particle size is preferably calculated using the Fraunhofer calculation; and/or preferably a Malvern Mastersizer or Sympatec apparatus is used for measurement. For example, particle size measurement and/or analysis by laser diffraction can use any or all of (preferably all of) the following: a Malvern Mastersizer longbed version, a dispersing medium of 0.1% Tween 80 in water, a stir rate of ca. 1500 rpm, ca. 3 mins sonification prior to final dispersion and analysis, a 300 RF (Reverse Fourier) lens, and/or the Fraunhofer calculation with Malvern software.

An illustrative non-limiting example of a small-scale micronisation process is now given:

Micronisation Examples: Micronisation of Example 73, 75, 98, 283, 304, 306, 307, 308, 309, 310, 311, 312, 313, 314, 314A or 333

Purpose: To micronise Example 73, 75, 98, 283, 304, 306, 307, 308, 309, 310, 311, 312, 313, 314 or 314A or 333 (described hereinafter), usually in an amount of approximately 600-1000 mg thereof, using a Jetpharma MC1 micronizer.

The parent (unmicronised) and micronised materials are analyzed for particle size by laser diffraction and crystallinity by PXRD.

Equipment and Material

| Equipment/material | Description and specification |
|---|---|
| Jetpharma MC1 Micronizer | Nitrogen supply: Air tank with 275 psi |
| | rate tubing |
| Analytical balance | Sartorius Analytical |
| Top loader balance | Mettler PM400 |
| Digital Caliper | VWR Electronic caliper |
| Materials to be micronised | Example 307 |
| (Procedure 1 - carried out) | |
| Materials to be micronised | Example 73, Example 75, Example 283 or |
| (alternative embodiments of | Example 333 |
| Procedure 1 - carried out) | |
| Materials to be micronised | Example 73, 98, 283, 304, 306, 307, |
| (Procedure 2 - not carried out) | 308, 309, 310, 311, 312, 313, 314 or |
| | 314A |

The Jetpharma MC1 Micronizer comprises a horizontal disc-shaped milling housing having: a tubular compound inlet (e.g. angled at ca. 30 degrees to the horizontal) for entry of a suspension of unmicronised compound of formula (I) or salt in a gasflow, a separate gas inlet for entry of gases, a gas outlet for exit of gases, and a collection vessel (micronizer container) for collecting micronised material. The milling housing has two chambers: (a) an outer annular chamber in gaseous connection with the gas inlet, the chamber being for receiving pressurised gas (e.g. air or nitrogen), and (b) a disc-shaped inner milling chamber within and coaxial with the outer chamber for micronising the input compound/salt, the two chambers being separated by an annular wall. The annular wall (ring R) has a plurality of narrow-bored holes connecting the inner and outer chambers and circumferentially-spaced-apart around the annular wall. The holes opening into the inner chamber are directed at an angle (directed part-way between radially and tangentially), and in use act as nozzles directing pressurised gas at high velocity from the outer chamber into the inner chamber and in an inwardly-spiral path (vortex) around the inner chamber (cyclone). The compound inlet is in gaseous communication with the inner chamber via a nozzle directed tangentially to the inner chamber, within and near to the annular wall/ring R. Upper and lower broad-diameter exit vents in the central axis of the inner milling chamber connect to (a) (lower exit) the collection vessel which has no air outlet, and (b) (upper exit) the gas outlet. Inside and coaxial with the tubular compound inlet and longitudinally-movable within it is positioned a venturi inlet (V) for entry of gases. The compound inlet also has a bifurcation connecting to an upwardly-directed material inlet port for inputting material.

In use, the narrow head of the venturi inlet (V) is preferably positioned below and slightly forward of the material inlet port, so that when the venturi delivers pressurised gas (e.g. air or nitrogen) the feed material is sucked from the material inlet port into the gas stream through the compound inlet and is accelerated into the inner milling chamber tangentially at a subsonic speed. Inside the milling chamber the material is further accelerated to a supersonic speed by the hole/nozzle system around the ring (R ) (annular wall) of the milling chamber. The nozzles are slightly angled so that the acceleration pattern of the material is in the form of an inwardly-directed vortex or cyclone. The material inside the milling chamber circulates rapidly and particle collisions occur during the process, causing larger particles to fracture into smaller ones. “Centrifugal” acceleration in the vortex causes the larger particles to remain at the periphery of the inner chamber while progressively smaller particles move closer to the centre until they exit the milling chamber, generally through the lower exit, at low pressure and low velocity. The particles that exit the milling chamber are heavier than air and settle downward thorugh the lower exit into the collection vessel (micronizer container), while the exhaust gas rises (together with a minority of small particles of micronised material) and escapes into the atmosphere at low pressure and low velocity.

Procedure:

The micronizer is assembled. The narrow head of the venturi inlet is positioned below and slightly forward of the material inlet port and is measured with a micro-caliper to make sure that it is inserted correctly. The ring (R) and venturi (V) pressures are adjusted according to the values specified in the experimental design (refer to experimental section below) by adjusting the valves on the pressure gauges on the micronizer. The setup is checked for leakage by observing if there is any fluctuation in the reading of the pressure gauges.

Note that the venturi (V) pressure is kept at least 2 bars greater than the ring (R) pressure to prevent regurgitation of material, e.g. outwardly from the material inlet port.

Balance performance is checked with calibration weights. Specified amount of the parent material (see e.g. section on experimental run Procedure 1 for Example 307) is fed into the input container of the micronizer using a spatula. The input container plus material is weighed. The equipment pressure is monitored during the micronization process.

Upon completion of the micronising run, the nitrogen supply is shut off and the micronised material is allowed to settle into the micronizer container. The micronised powder in the micronizer container (collection vessel) and the cyclone (above the recovery vessel) are collected together into a pre-weighed and labelled collection vial. The weight of the micronised material is recorded. The input container is re-weighed in order to calculate the amount of input material by difference. The micronizer is disassembled and residual PDE4 compound on the micronizer inner surface is rinsed with 70/30 isopropyl alcohol/water and collected into a flask. The micronizer is then thoroughly cleaned in a Lancer washing machine and dried before subsequent runs are performed.

Optional Experimental Parameters

Procedure 1: Experimental Parameters and Results for Example 307

This experiment, Procedure 1, using Example 307 as the compound to be micronised, has been carried out generally using a procedure and an apparatus generally as described above or similar to those described, using generally the following experimental parameters and giving the following results:

| | Material | Venturi | Particle Size | Particle Size | Recovery |
|---|---|---|---|---|---|
| | input | Pressure (V)/ | Data (microns) | Data (microns) | yield of |
| | amount | ring (R) | (unmicronised | (micronised | micronised |
| Procedure no. | (g) | Pressure (bar) | material) | material) | material* |
| 1 | ca. 0.9 g | V = 5 to 7 bar | D10 = 2.48 | D10 = 0.84 | 58% |
| | | R = 3 to 4 bar | D50 = 8.98 | D50 = 1.56 | |
| | | | D90 = 24.14 | D90 = 2.74 | |

*% yield = [(Material from collection vessel + Material from cyclone)/Material input amount] × 100.
In general, very approximately 50-75% yields are achievable using this method, including material from collection vessel and material from inside walls of cyclone.

The above optional parameters can be varied using the skilled person's knowledge.

In alternative embodiments of Procedure 1, Procedure 1 or variations thereof generally using generally similar conditions, have also been carried out for the following Examples:

### Example 73

### Example 75

### Example 283

### Example 333

Procedure 2: Optional Experimental Parameters

Parent (unmicronised) material (Procedure 2): Example 73, 98, 283, 304, 306, 307, 308, 309, 310, 311, 312, 313, 314 or 314A (note -not carried out) Balance(s): Sartorius analytical

| | | Venturi | | |
|---|---|---|---|---|
| | Material | Pressure (V)/ | | |
| Procedure | input | ring (R) | Intended | |
| no. | amount (g) | Pressure (bar) | feed-rate | Notes |
| 2 | ca. 0.9 g | V = 8 to 10 bar | 180 to 200 | Note that this |
| | | R = 5.5 to 6 | mg/min | Procedure 2 was |
| | | bar | | not carried out |

The above optional parameters can be varied using the skilled person's knowledge.

Procedure 2 includes possible parameters and conditions, and micronisation of possible Examples, and has not been carried out.

Alternative embodiment: Any of the Examples of the compounds or salts of the invention disclosed herein are optionally micronised as described above.

Dry Powder Inhalable Compositions

For pharmaceutical compositions suitable and/or adapted for inhaled administration, it is preferred that the pharmaceutical composition is a dry powder inhalable composition. Such a composition can comprise a powder base such as lactose or starch, the compound of formula (I) or salt thereof (preferably in particle-size-reduced form, e.g. in micronised form), and optionally a performance modifier such as L-leucine, mannitol, trehalose and/or magnesium stearate. Preferably, the dry powder inhalable composition comprises a dry powder blend of lactose and the compound of formula (I) or salt thereof. The lactose is preferably lactose hydrate e.g. lactose monohydrate and/or is preferably inhalation-grade and/or fine-grade lactose. Preferably, the particle size of the lactose is defined by 90% or more (by weight or by volume) of the lactose particles being less than 1000 microns (micrometres) (e.g. 10-1000 microns e.g. 30-1000 microns) in diameter, and/or 50% or more of the lactose particles being less than 500 microns (e.g. 10-500 microns) in diameter. More preferably, the particle size of the lactose is defined by 90% or more of the lactose particles being less than 300 microns (e.g. 10-300 microns e.g. 50-300 microns) in diameter, and/or 50% or more of the lactose particles being less than 100 microns in diameter. Optionally, the particle size of the lactose is defined by 90% or more of the lactose particles being less than 100-200 microns in diameter, and/or 50% or more of the lactose particles being less than 40-70 microns in diameter. Most importantly, it is preferable that about 3 to about 30% (e.g. about 10%) (by weight or by volume) of the particles are less than 50 microns or less than 20 microns in diameter. For example, without limitation, a suitable inhalation-grade lactose is E9334 lactose (10% fines) (Borculo Domo Ingredients, Hanzeplein 25, 8017 J D Zwolle, Netherlands).

In the dry powder inhalable composition, preferably, the compound of formula (I) or salt thereof is present in about 0.1% to about 70% (e.g. about 1% to about 50%, e.g. about 5% to about 40%, e.g. about 20 to about 30%) by weight of the composition.

An illustrative non-limiting example of a dry powder inhalable composition follows:

Dry Powder Formulation Example—Dry powder Lactose Blend Preparation

Using a size-reduced e.g. micronised form of the compound of formula (I) or salt thereof (e.g. as prepared in the Micronisation Example above), the dry powder blend is prepared by mixing the required amount of the compound/salt (e.g. 10 mg, 1% w/w) with inhalation-grade lactose containing 10% fines (e.g. 990 mg, 99% w/w) in a Teflon™ (polytetrafluoroethene) pot in a Mikro-dismembrator ball-mill (but without a ball bearing) at ¾speed (ca. 2000-2500 rpm) for about 4 hours at each blend concentration. The Mikro-dismembrator (available from B. Braun Biotech International, Schwarzenberger Weg 73-79, D-34212 Melsungen, Germany; www.bbraunbiotech.com) comprises a base with an upwardly-projecting and sidewardly-vibratable arm to which is attached the Teflon™ pot. The vibration of the arm achieves blending.

Other blends can include: 10% w/w compound/salt (50 mg)+90% w/w lactose (450 mg, inhalation-grade lactose containing 10% fines).

Serial dilution of the 1% w/w blend can achieve e.g. 0.1% and 0.3% w/w blends.

Dry Powder Inhalation Devices

Optionally, in particular for dry powder inhalable compositions, a pharmaceutical composition for inhaled administration can be incorporated into a plurality of sealed dose containers (e.g. containing the dry powder composition) mounted longitudinally in a strip or ribbon inside a suitable inhalation device. The container is rupturable or peel-openable on demand and the dose, e.g. of the dry powder composition, can be administered by inhalation via a device such as the DISKUS™ device, marketed by GlaxoSmithKline. The DISKUS™ inhalation device is usually substantially as described in GB 2,242,134 A. In such device at least one container for the pharmaceutical composition in powder form (the at least one container preferably being a plurality of sealed dose containers mounted longitudinally in a strip or ribbon) is defined between two members peelably secured to one another; the device comprises: means defining an opening station for the said at least one container; means for peeling the members apart at the opening station to open the container; and an outlet, communicating with the opened container, through which a user can inhale the pharmaceutical composition in powder form from the opened container.

Unit Dose Form and Dosing Regimens

Preferably the composition is in unit dose form such as a tablet or capsule for oral administration, e.g. for oral adiministration to a human.

In the pharmaceutical composition, a or each dosage unit for oral or parenteral administration preferably contains from 0.01 to 3000 mg, more preferably 0.5 to 1000 mg, of a compound of the formula (I) or a pharmaceutically acceptable salt thereof, calculated as the free base. A or each dosage unit for nasal or inhaled administration preferably contains from 0.001 to 50 mg, more preferably 0.01 to 5 mg, of a compound of the formula (I) or a pharmaceutically acceptable salt thereof, calculated as the free base.

A pharmaceutically acceptable compound or salt of the invention is preferably administered to a mammal (e.g. human) in a daily oral or parenteral dose of 0.001 mg to 50 mg per kg body weight per day (mg/kg/day), for example 0.01 to 20 mg/kg/day or 0.03 to 10 mg/kg/day or 0.1 to 2 mg/kg/day, of the compound of the formula (I) or a pharmaceutically acceptable salt thereof, calculated as the free base.

A pharmaceutically acceptable compound or salt of the invention is preferably administered to a mammal (e.g. human) in a daily nasal or inhaled dose of: 0.0001 to 5 mg/kg/day or 0.0001 to 1 mg/kg/day, e.g. 0.001 to 1 mg/kg/day or 0.001 to 0.3 mg/kg/day or 0.001 to 0.1 mg/kg/day or 0.005 to 0.3 mg/kg/day, of the compound of the formula (I) or a pharmaceutically acceptable salt thereof, calculated as the free base.

The pharmaceutically acceptable compounds or salts of the invention is preferably administered in a daily dose (for an adult patient) of, for example, an oral or parenteral dose of 0.01 mg to 3000 mg per day or 0.5 to 1000 mg per day e.g. 2 to 500 mg per day, or a nasal or inhaled dose of 0.001 to 300 mg per day or 0.001 to 50 mg per day or 0.01 to 30 mg per day or 0.01 to 5 mg per day or 0.02 to 2 mg per day, of the compound of the formula (I) or a pharmaceutically acceptable salt thereof, calculated as the free base.

Combinations

The compounds, salts and/or pharmaceutical compositions according to the invention may also be used in combination with another therapeutically active agent, for example, a β₂ adrenoreceptor agonist, an anti-histamine, an anti-allergic or an anti-inflammatory agent.

The invention thus provides, in a further aspect, a combination comprising a compound of formula (I) or a pharmaceutically acceptable salt thereof together with another therapeutically active agent, for example, a β₂-adrenoreceptor agonist, an anti-histamine, an anti-allergic, an anti-inflammatory agent or an antiinfective agent.

Preferably, the β₂-adrenoreceptor agonist is salmeterol (e.g. as racemate or a single enantiomer such as the R-enantiomer), salbutamol, formoterol, salmefamol, fenoterol or terbutaline, or a salt thereof (e.g. pharmaceutically acceptable salt thereof), for example the xinafoate salt of salmeterol, the sulphate salt or free base of salbutamol or the fumarate salt of formoterol. Long-acting β₂-adrenoreceptor agonists are preferred, especially those having a therapeutic effect over a 12-24 hour period such as salmeterol or formoterol. Preferably, the β₂-adrenoreceptor agonist is for inhaled administration, e.g. once per day and/or for simultaneous inhaled administration; and more preferably the β₂-adrenoreceptor agonist is in particle-size-reduced form e.g. as defined herein. Preferably, the β₂-adrenoreceptor agonist combination is for treatment and/or prophylaxis of COPD or asthma. Salmeterol or a pharmaceutically acceptable salt thereof, e.g. salmeterol xinofoate, is preferably adiministered to humans at an inhaled dose of 25 to 50 micrograms twice per day (measured as the free base). The combination with a β₂-adrenoreceptor agonist can be as described in WO 00/12078.

Preferred long acting β₂-adrenoreceptor agonists include those described in WO 02/066422A, WO 03/024439, WO 02/070490 and WO 02/076933.

Especially preferred long-acting β₂-adrenoreceptor agonists include compounds of formula(XX) (described in WO 02/066422): 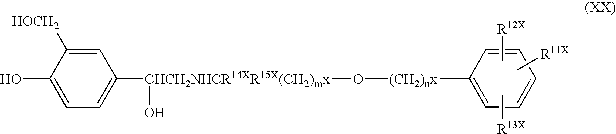
or a salt or solvate thereof, wherein in formula (XX):

m^{x} is an integer of from 2 to 8;

n^{x} is an integer of from 3 to 11,

with the proviso that m^{x}+n^{x} is 5 to 19,

R^{11X} is —XSO₂NR^{16X}R^{17X} wherein X is —(CH₂)ₚx- or C₂₋₆ alkenylene;

R^{16X} and R^{17X} are independently selected from hydrogen, C₁₋₆alkyl, C₃₋₇cycloalkyl, C(O)NR^{18X}R^{19X}, phenyl, and phenyl (C₁₋₄alkyl)-,

or R^{16X} and R^{17X}, together with the nitrogen to which they are bonded, form a 5-, 6-, or 7-membered nitrogen containing ring, and R^{16X} and R^{17X} are each optionally substituted by one or two groups selected from halo, C₁₋₆alkyl, C₁₋₆haloalkyl, C₁₋₆alkoxy, hydroxy-substituted C₁₋₆alkoxy, —CO₂R^{18X}, —SO₂NR^{18X}R^{19X}, —CONR^{18X}R^{19X}, —NR^{18X}C(O)R^{19X}, or a 5-, 6- or 7-membered heterocylic ring;

R^{18X} and R^{19X} are independently selected from hydrogen, C₁₋₆alkyl,

C₃₋₆cycloalkyl, phenyl, and phenyl (C₁₋₄alkyl)-; and

p^{x} is an integer of from 0 to 6, preferably from 0 to 4;

R^{12X} and R^{13X} are independently selected from hydrogen, C₁₋₆alkyl, C₁₋₆alkoxy, halo, phenyl, and C₁₋₆haloalkyl; and

R^{14X} and R^{15X} are independently selected from hydrogen and C₁₋₄alkyl with the proviso that the total number of carbon atoms in R^{14X} and R^{15X} is not more than 4.

Preferred β₂-adrenoreceptor agonists disclosed in WO 02/066422 include: 3-(4-{[6-({(2R)-2-hydroxy-2-[4-hydroxy-3-(hydroxymethyl)-phenyl]ethyl}amino)hexyl]oxy}butyl)benzenesulfonamide and 3-(3-{[7-({(2R)-2-hydroxy-2-[4-hydroxy-3-hydroxymethyl)phenyl]ethyl}-amino)heptyl]oxy}propyl)benzenesulfonamide.

A preferred β₂-adrenoreceptor agonist disclosed in WO 03/024439 is:

4-{(1R)-2-[(6-{2-[(2,6-dichlorobenzyl)oxy]ethoxy}hexyl)amino]-1-hydroxyethyl}-2-(hydroxymethyl)phenol.

A combination of a compound of formula (I) or salt together with an anti-histamine is preferably for oral administration (e.g. as a combined composition such as a combined tablet), and can be for treatment and/or prophylaxis of allergic rhinitis. Examples of anti-histamines include methapyrilene, or H1 antagonists such as cetirizine, loratadine (e.g. Clarityn™), desloratadine (e.g. Clarinex™) or fexofenadine (e.g. Allegra™).

The invention also provides, in a further aspect, a combination comprising a compound of formula (I) or a pharmaceutically acceptable salt thereof together with an anticholinergic compound, e.g. a muscarinic (M) receptor antagonist in particular an M₁, M₂, M₁/M₂, or M₃ receptor antagonist, more preferably a M₃ receptor antagonist, still more preferably a M₃ receptor antagonist which selectively antagonises (e.g. antagonises 10 times or more strongly) the M₃ receptor over the M₁ and/or M₂ receptor. For combinations of anticholinergic compounds/muscarinic (M) receptor antagonist with PDE4 inhibitors, see for example WO 03/011274 A2 and WO 02/069945 A2/US 2002/0193393 A1 and US 2002/052312 A1, and some or all of these publications give examples of anticholinergic compounds/muscarinic (M) receptor antagonists which may be used with the compounds of formula (I) or salts, and/or suitable pharmaceutical compositions. For example, the muscarinic receptor antagonist can comprise or be an ipratropium salt (e.g. ipratropium bromide), an oxitropium salt (e.g. oxitropium bromide), or more preferably a tiotropium salt (e.g. tiotropium bromide); see e.g. EP 418 716 A1 for tiotropium.

The anticholinergic compound or muscarinic (M) receptor antagonist, e.g. M₃ receptor antagonist, is preferably for inhaled administration, more preferably in particle-size-reduced form e.g. as defined herein. More preferably, both the muscarinic (M) receptor antagonist and the compound of formula (I) or the pharmaceutically acceptable salt thereof are for inhaled administration. Preferably, the anticholinergic compound or muscarinic receptor antagonist and the compound of formula (I) or salt are for simultaneous administration. The muscarinic receptor antagonist combination is preferably for treatment and/or prophylaxis of COPD.

Other suitable combinations include, for example, a combination comprising a compound of formula (1) or a pharmaceutically acceptable salt thereof together with another anti-inflammatory agent such as an anti-inflammatory corticosteroid; or a non-steroidal anti-inflammatory drug (NSAID) such as a leukotriene antagonist (e.g. montelukast), an INOS inhibitor, a tryptase inhibitor, a elastase inhibitor, a beta-2 integrin antagonist, a adenosine 2a agonist, a CCR3 antagonist, or a 5-lipoxogenase inhibitor; or an antiinfective agent (e.g. an antibiotic or an antiviral). An INOS inhibitor is preferably for oral administration. Suitable iNOS inhibitors (inducible nitric oxideisynthase inhibitors) include those disclosed in WO 93/13055, WO 98/30537, WO 02/50021, WO 95/34534 and WO 99/62875. Suitable CCR3 inhibitors include those disclosed in WO 02/26722.

In a combination comprising a compound of formula (I) or a pharmaceutically acceptable salt thereof together with an anti-inflammatory corticosteroid (which is preferably for treatment and/or prophylaxis of asthma, COPD or allergic rhinitis), then preferably the anti-inflammatory corticosteroid is fluticasone, fluticasone propionate (e.g. see U.S. Pat. No. 4,335,121), beclomethasone, beclomethasone 17-propionate ester, beclomethasone 17,21-dipropionate ester, dexamethasone or an ester thereof, mometasone or an ester thereof, ciclesonide, budesonide, flunisolide, or a compound as described in WO 02/12266 A1 (e.g. as claimed in any of claims 1 to 22 therein), or a pharmaceutically acceptable salt of any of the above. If the anti-inflammatory corticosteroid is a compound as described in WO 02/12266 A1, then preferably it is Example 1 therein {which is 6α,9α-difluoro-17α-[(2-furanylcarbonyl)oxy]-11β-hydroxy-16α-methyl-3-oxo-androsta-1,4-diene-17β-carbothioic acid S-fluoromethyl ester} or Example 41 therein {which is 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester}, or a pharmaceutically acceptable salt thereof. The anti-inflammatory corticosteroid is preferably for intranasal or inhaled administration. Fluticasone propionate is preferred and is preferably for inhaled administration to a human either (a) at a dose of 250 micrograms once per day or (b) at a dose of 50 to 250 micrograms twice per day.

Also provided is a combination comprising a compound of formula (I) or a pharmaceutically acceptable salt thereof together with β₂-adrenoreceptor agonist and an anti-inflammatory corticosteroid, for example as described in WO 03/030939 A1. Preferably this combination is for treatment and/or prophylaxis of asthma, COPD or allergic rhinitis. The β₂-adrenoreceptor agonist and/or the anti-inflammatory corticosteroid can be as described above and/or as described in WO 03/030939 A1. Most preferably, in this “triple” combination, the β₂-adrenoreceptor agonist is salmeterol or a pharmaceutically acceptable salt thereof (e.g. salmeterol xinafoate) and the anti-inflammatory corticosteroid is fluticasone propionate.

The combinations referred to above may conveniently be presented for use in the form of a pharmaceutical composition and thus a pharmaceutical composition comprising a combination as defined above together with one or more pharmaceutically acceptable carriers and/or excipients represent a further aspect of the invention.

The individual compounds of such combinations may be administered either sequentially or simultaneously in separate or combined pharmaceutical composition.

In one embodiment, the combination as defined herein can be for simultaneous inhaled administration and is disposed in a combination inhalation device. Such a combination inhalation device is another aspect of the invention. Such a combination inhalation device can comprise a combined pharmaceutical composition for simultaneous inhaled administration (e.g. dry powder composition), the composition comprising all the individual compounds of the combination, and the composition being incorporated into a plurality of sealed dose containers mounted longitudinally in a strip or ribbon inside the inhalation device, the containers being rupturable or peel-openable on demand; for example such inhalation device can be substantially as described in GB 2,242,134 A (DISKUS™) and/or as described above. Alternatively, the combination inhalation device can be such that the individual compounds of the combination are administrable simultaneously but are stored separately (or wholly or partly stored separately for triple combinations), e.g. in separate pharmaceutical compositions, for example as described in PCT/EP03/00598 filed on 22 Jan. 2003, published as WO 03/061743 (e.g. as described in the claims thereof e.g. claim 1).

The invention also provides a method of preparing a combination as defined herein,

the method comprising either

(a) preparing a separate pharmaceutical composition for administration of the individual compounds of the combination either sequentially or simultaneously, or

(b) preparing a combined pharmaceutical composition for administration of the individual compounds of the combination simultaneously,

wherein the pharmaceutical composition comprises the combination together with one or more pharmaceutically acceptable carriers and/or excipients.

The invention also provides a combination as defined herein, prepared by a method as defined herein.

Biological Test Methods

PDE 3, PDE 4B, PDE 4D, PDE 5, PDE 6 Primary Assay Methods

The activity of the compounds can be measured in the assay methods shown below. Preferred compounds of the invention are selective PDE4 inhibitors, i.e. they inhibit PDE4 (e.g. PDE4B and/or PDE4D, preferably PDE4B) more strongly than they inhibit PDE3 and/or more strongly than they inhibit PDE5 and/or more strongly than they inhibit PDE6.

Possible PDE Enzyme Sources and Literature References

Human recombinant PDE4B, in particular the 2B splice variant thereof (HSPDE4B2B), is disclosed in WO 94/20079 and also M. M. McLaughlin et al., “A low Km, rolipram-sensitive, cAMP-specific phosphodiesterase from human brain: cloning and expression of cDNA, biochemical characterisation of recombinant protein, and tissue distribution of mRNA”, J. Biol. Chem., 1993, 268, 6470-6476. For example, in Example 1 of WO 94/20079, human recombinant PDE4B is described as being expressed in the PDE-deficient yeast Saccharomyces cerevisiae strain GL62, e.g. after induction by addition of 150 uM CuSO₄, and 100,000×g supernatant fractions of yeast cell lysates are described for use in the harvesting of PDE4B enzyme.

Human recombinant PDE4D (HSPDE4D3A) is disclosed in P. A. Baecker et al., “Isolation of a cDNA encoding a human rolipram-sensitive cyclic AMP phoshodiesterase (PDE IVD)”, Gene, 1994, 138, 253-256.

Human recombinant PDE5 is disclosed in K. Loughney et al., “Isolation and characterisation of cDNAs encoding PDE5A, a human cGMP-binding, cGMP-specific 3′,5′-cyclic nucleotide phosphodiesterase”, Gene, 1998, 216, 139-147.

PDE3 can be purified from bovine aorta as described by H. Coste and P. Grondin, “Characterisation of a novel potent and specific inhibitor of type V phosphodiesterase”, Biochem. Pharmacol., 1995, 50, 1577-1585.

PDE6 can be purified from bovine retina as described by: P. Catty and P. Deterre, “Activation and solubilization of the retinal cGMP-specific phosphodiesterase by limited proteolysis”, Eur. J. Biochem., 1991, 199, 263-269; A. Tar et al. “Purification of bovine retinal cGMP phosphodiesterase”, Methods in Enzymology, 1994, 238, 3-12; and/or D. Srivastava et al. “Effects of magnesium on cyclic GMP hydrolysis by the bovine retinal rod cyclic GMP phosphodiesterase”, Biochem. J., 1995, 308, 653-658.

Inhibition of PDE 3, PDE 4B, PDE 4D, PDE 5 or PDE 6 Activity: Radioactive Scintillation Proximity Assay (SPA)

The ability of compounds to inhibit catalytic activity at PDE4B or 4D (human recombinant), PDE3 (from bovine aorta), PDE5 (human recombinant) or PDE6 (from bovine retina) can optionally be determined by Scintillation Proximity Assay (SPA) in 96-well format.

Test compounds (as a solution in DMSO, preferably about 2 microlitre (ul) volume of DMSO solution) are preincubated at ambient temperature (room temperature, e.g. 19-23° C.) in Wallac Isoplates (code 1450-514) with PDE enzyme in 50 mM Tris-HCl buffer pH 7.5, 8.3 mM MgCl₂, 1.7 mM EGTA, 0.05% (w/v) bovine serum albumin for 10-30 minutes (usually 30 minutes). The enzyme concentration is adjusted so that no more than 20% hydrolysis of the substrate defined below occurs in control wells without compound, during the incubation. For the PDE3, PDE4B and PDE4D assays, [5′,8-³H]Adenosine 3′,5′-cyclic phosphate (Amersham Pharmacia Biotech, code TRK.559; or Amersham Biosciences UK Ltd, Pollards Wood, Chalfont St Giles, Buckinghamshire HP8 4SP, UK) is added to give 0.05 uCi per well and about 10 nM final concentration. For the PDE5 and PDE6 assays, [8-³H]Guanosine 3′,5′-cyclic phosphate (Amersham Pharmacia Biotech, code TRK.392) is added to give 0.05 uCi per well and about 36 nM final concentration. Plates containing assay mixture, preferably approx. 100 ul volume of assay mixture, are mixed on an orbital shaker for 5 minutes and incubated at ambient temperature for 1 hour. Phosphodiesterase SPA beads (Amersham Pharmacia Biotech, code RPNQ 0150) are added (about 1 mg per well) to terminate the assay. Plates are sealed and shaken and allowed to stand at ambient temperature for 35 minutes to 1 hour (preferably 35 minutes) to allow the beads to settle. Bound radioactive product is measured using a WALLAC TRILUX 1450 Microbeta scintillation counter. For inhibition curves, 10 concentrations (1.5 nM-30 uM) of each compound are assayed. Curves are analysed using ActivityBase and XLfit (ID Business Solutions Limited, 2 Ocean Court, Surrey Research Park, Guildford, Surrey GU2 7QB, United Kingdom) Results are expressed as pIC₅₀ values.

In an alternative to the above radioactive SPA assay, PDE4B or PDE4D inhibition can be measured in the following Fluorescence Polarisation (FP) assay:

Inhibition of PDE4B or PDE4D Activity: Fluorescence Polarisation (FP) Assay

The ability of compounds to inhibit catalytic activity at PDE4B (human recombinant) or PDE4D (human recombinant) can optionally be determined by IMAP Fluorescence Polarisation (FP) assay (IMAP Explorer kit, available from Molecular Devices Corporation, Sunnydale, Calif., USA; Molecular Devices code: R8062) in 384-well format.

The MAP FP assay is able to measure PDE activity in an homogenous, non-radioactive assay format. The FP assay uses the ability of immobilised trivalent metal cations, coated onto nanoparticles (tiny beads), to bind the phosphate group of Fl-AMP that is produced on the hydrolysis of fluorescein-labelled (Fl) cyclic adenosine mono-phosphate (Fl-cAMP) to the non-cyclic Fl-AMP form. Fl-cAMP does not bind. Binding of Fl-AMP product to the beads (coated with the immobilised trivalent cations) slows the rotation of the bound Fl-AMP and leads to an increase in the fluorescence polarisation ratio of parallel to perpendicular light. Inhibition of the PDE reduces/inhibits this signal increase.

Test compounds (small volume, e.g. ca. 0.5 to 1 ul, preferably ca. 0.5 ul, of solution in DMSO) are preincubated at ambient temperature (room temperature, e.g. 19-23° C.) in black 384-well microtitre plates (supplier: NUNC, code 262260) with PDE enzyme in 10 mM Tris-HCl buffer pH 7.2, 10 mM MgCl₂, 0.1% (w/v) bovine serum albumin, and 0.05% NaN₃ for 10-30 minutes. The enzyme level is set by experimentation so that reaction is linear throughout the incubation. Fluorescein adenosine 3′,5′-cyclic phosphate (from Molecular Devices Corporation, Molecular Devices code: R7091) is added to give about 40 nM final concentration (final assay volume usually ca. 20-40 ul, preferably ca. 20 ul). Plates are mixed on an orbital shaker for 10 seconds and incubated at ambient temperature for 40 minutes. IMAP binding reagent (as described above, from Molecular Devices Corporation, Molecular Devices code: R7207) is added (60 ul of a 1 in 400 dilution in binding buffer of the kit stock solution) to terminate the assay. Plates are allowed to stand at ambient temperature for 1 hour. The Fluorescence Polarisation (FP) ratio of parallel to perpendicular light is measured using an Analyst™ plate reader (from Molecular Devices Corporation). For inhibition curves, 10 concentrations (1.5 nM-30 uM) of each compound are assayed. Curves are analysed using ActivityBase and XLfit (ID Business Solutions Limited, 2 Ocean Court, Surrey Research Park, Guildford, Surrey GU2 7QB, United Kingdom). Results are expressed as pIC₅₀ values.

In the FP assay, reagents are usually dispensed using Multidrop™ (available from Thermo Labsystems Oy, Ratastie 2, PO Box 100, Vantaa 01620, Finland).

For a given PDE4 inhibitor, the PDE4B (or PDE4D) inhibition values measured using the SPA and FP assays can differ slightly. However, in a regression analysis of 100 test compounds (not necessarily compounds of the invention), the pIC₅₀ inhibition values measured using SPA and FP assays have been found generally to agree within about 0.5 log units, for each of PDE4B and PDE4D (linear regression coefficient 0.966 for PDE4B and 0.971 for PDE4D; David R. Mobbs et al., “Comparison of the IMAP Fluorescence Polarisation Assay with the Scintillation Proximity Assay for Phosphodiesterase Activity”, poster presented at 2003 Molecular Devices UK & Europe User Meeting, 2nd October 2003, Down Hall, Harlow, Essex, United Kingdom).

Biological Data obtained for some of the Examples (PDE4B inhibitory activity, either as one reading or as an average of several (e.g. ca. 2-6) readings) are generally as follows, based on measurements only, generally using SPA and/or FP assays generally as described above or generally similar to those described above. In each of the SPA and FP assays, absolute accuracy of measurement is not possible, and the readings given are thought to be accurate only up to about ±0.5 of a log unit, depending on the number of readings made and averaged:

| | PDE4B pIC₅₀ |
|---|---|
| Example number | (±about 0.5) |
| 1, 8, 24, 28, 63, 75 | 8.3 to 9.1 |
| 6, 7, 26, 29, 64, 25 | 7.15 to 7.5 |
| 13, 50 | 8.3 to 9.1 |
| 2, 37, 38 | 7.6 to 7.9 |
| 48, 73, 98, 139, 191, 210, | 8.7 to 10.0 |
| 218, 221, 252, 261, 282, | |
| 283, 304, 306 | |
| Examples 308 to 314, and | 8.0 to 9.45 |
| Examples 368, 369, 379, | |
| 380, 382 | |
| Examples 316 to 345 | 9.0 to 10.1 |
| Examples 346 to 355 | 8.5 to 9.3 |
| Examples 356 to 359 | 6.8 to 7.4 |
| Examples 360 to 367 | 7.2 to 9.0 |
| Examples 370 to 373 | 6.9 to 7.9 |
| Examples 375 to 378 | 7.0 to 8.3 |

A large majority or substantially all of the Examples have been tested for PDE4B inhibition, normally using the radioactive SPA assay and/or the FP assay generally as described above or generally similar to those described above. A large majority or substantially all of the Examples tested have PDE4B inhibitory activities in the range of pIC₅₀=about 6 (± about 0.5) to about 10.1 (± about 0.5). Where an Example is described in the Examples section below as capable of being made using a possible reagent source which is an Intermediate (e.g. which might have a defined or enriched or no benzylic carbon atom (CR4R5) stereochemistry), then, without any guarantee, the PDE4B inhibition pIC50 values mentioned above are thought to be, in general, those obtained for the Example when made using that Intermediate specified in the Examples section.

Only selected ones of the PDE4B-tested Examples have also been tested, on an optional basis, for one or more of: PDE3, PDE5 or PDE6 inhibition using the above-described or other assays.

Of the Examples tested for PDE4B and PDE5 inhibition, those selected Examples wherein R³=cyclohexyl (NHR³=sub-formula (c)), tetrahydro-2H-pyran-4-yl (NHR³=group (h)), 4-oxocyclohexyl (NHR³=sub-formula (o)), cis-3-hydroxy-cyclohexyl (NHR³=sub-formula (n) in cis configuration), 4-(hydroxyimino)cyclohexyl (NHR³=sub-formula (o2), 4-(aminocarbonyl)cyclohexyl (NHR³=sub-formula (p9), especially with majority of cis isomer or cis/trans mixtures), or 1-(aminocarbonyl)-4-piperidinyl (NHR³ is of sub-formula (k2)), and wherein R¹ is ethyl, R² is H and having preferred —NH—C(R⁴)(R⁵)—Ar groups, sometimes or often exhibit selectivity for PDE4B over PDE5, as measured in the above enzyme inhibition assays and/or in generally-similar assays or other assays.

Emesis: Some known PDE4 inhibitors can cause emesis and/or nausea to greater or lesser extents, especially after systemic exposure e.g. after oral administration (e.g. see Z. Huang et al., Current Opinion in Chemical Biology, 2001, 5: 432-438, see especially pages 433-434 and refs cited therein). Therefore, it would be preferable, but not essential, if a PDE4 inhibitory compound or salt of the invention were to cause only limited or manageable emetic side-effects, e.g. after oral or parenteral administration. Emetic side-effects can for example be measured by the emetogenic potential of the compound or salt when administered to ferrets; for example one can measure the time to onset, extent, frequency and/or duration of vomiting, retching and/or writhing in ferrets after oral or parenteral administration of the compound or salt. See for example In vivo Assay 4 hereinafter for one optional measurement method for anti-inflammatory effect, emetic side-effects and therapeutic index (TI) in the ferret. See also for example A. Robichaud et al., “Emesis induced by inhibitors of [PDE IV] in the ferret”, Neuropharmacology, 1999, 38, 289-297, erratum Neuropharmacology, 2001, 40, 465-465. However, optionally, emetic side-effects and therapeutic index (TI) in rats can be conveniently measured by monitoring the pica feeding behaviour of rats after administration of the compound or salt of the invention (see In Vivo Assay 2 below).

Other side effects: Some known PDE4 inhibitors can cause other side effects such as headache and other central nervous system (CNS-) mediated side effects; and/or gastrointestinal (GI) tract disturbances. Therefore, it would be preferable but not essential if a particular PDE4 inhibitory compound or salt of the invention were to cause only limited or manageable side-effects in one or more of these side-effect categories.

Other Optional In Vitro Assays:

Inhibition of TNFα (TNF-alpha) Production it Human Whole Blood

This is a useful optional supplementary test, e.g. for potentially orally-administrable PDE4 inhibitors.

Test compounds are prepared as a ca. 10 mM stock solution in DMSO and a dilution series prepared in DMSO with 8 successive 3-fold dilutions, either directly from the 10 mM stock solution or from a more dilute solution in DMSO. The compound is added to assay plates using a Biomek Fx liquid handling robot.

Heparinised blood drawn from normal volunteers is dispensed (ca. 100 ul=ca. 100 ul) into microtitre plate wells containing ca. 0.5 or ca. 1.0 μl (ul) of an appropriately diluted test compound solution. After ca. 1 hr incubation at ca. 37 ° C., 5% CO₂, ca. 25 μl (ca. 25 ul) of LPS (lipopolysaccharide) solution (S. typhosa) in RPMI 1640 (containing 1% L-glutamine and 1% Penicillin/streptomycin) is added (ca 50 ng/ml final). The samples are incubated at ca. 37° C., 5% CO₂, for ca. 20 hours, and ca. 100 μl (ca. 100 ul) physiological saline (0.138% NaCl) is added, and diluted plasma is collected using a Platemate or Biomek FX liquid handling robot after centrifugation at ca. 1300 g for ca. 10 min. Plasma TNFα content is determined by electrochemiluminescence assay using the IGEN technology (see below) or by enzyme linked immunosorbant assay (ELISA) (see below).

Inhibition of TNFα (TNF-alpha) Production in Human PBMC Assay

This is a useful optional supplementary test, e.g. for potentially inhalably-administrable PDE4 inhibitors.

Test compounds are prepared as a ca. 10 mM stock solution in DMSO and a dilution series prepared in DMSO with 8 successive 3-fold dilutions, either directly from the 10 mM stock solution or from a more dilute solution in DMSO. The compound is added to assay plates using a Biomek Fx liquid handling robot.

PBMC cells (monocytes) are prepared from heparinised human blood from normal volunteers by centrifugation on histopaque at ca. 1000 g for ca. 30 minutes. The cells are collected from the interface, washed by centrifugation (ca. 1300 g, ca. 10 minutes) and resuspended in assay buffer (RPMI1640 containing 10% foetal calf serum, 1% L-glutamine and 1% penicillin/streptomycin) at 1×10⁶ cells/ml. Ca. 50 μl (ca. 50 ul) cells are added to microtitre wells containing ca. 0.5 or ca/ 1.0 μl (ul) of an appropriately diluted compound solution. Ca. 75 μl (ul) LPS (ca. 1 ng/ml final) is added and the samples are incubated at 37° C., 5% CO₂, for 20 hours. The supernatant is removed and the concentrations of TNF are determined by electrochemiluminescence assay using the IGEN technology or by ELISA (see below).

TNFα IGEN Assay

Ca. 50 μl supernatant from either whole blood or PBMC assay plates is transferred to a 96 well polypropylene plate. Each plate also contains a TNF (standard curve (ca. 0 to 30000 pg/ml: R+D Systems, 210-TA). Ca. 50 μl (ul) of streptavidinlbiotinylated anti-TNFα antibody mix, ca. 25 μl ruthenium tagged anti-TNFα monoclonal and ca. 100 μl PBS containing 0.1% bovine serum albumin are added to each well and the plates are sealed and shaken for ca. 2 hours before being read on an IGEN instrument.

TNFα ELISA Assay

Human TNFα can be assayed using a commercial assay kit (AMS Biotechnology, 211-90-164-40) according to the manufacturers' instructions but with TNFα calibration curves prepared using Pharmingen TNFα (cat No. 555212).

In Vivo Biological Assays

The in vitro enzymatic PDE4B inhibition assay(s) described above or generally similar assays should be regarded as being the primary test(s) of biological activity. However, some additional in vivo biological tests, which are optional and which are not an essential measure of either efficacy or side-effects, and which have not necessarily been carried out, are described below.

In Vivo Assay 1. LPS-Induced Pulmonary Neutrophilia in Rats: Effect of Orally Adimistered PDE4 Inhibitors

Pulmonary neutrophil influx has been shown to be a significant component to the family of pulmonary diseases like chronic obstructive pulmonary disease (COPD) which can involve chronic bronchitis and/or emphysema (G. F. Filley, Chest. 2000; 117(5); 251s-260s). The purpose of this neutrophilia model is to study the potentially anti-inflammatory effects in vivo of orally administered PDE4 inhibitors on neutrophilia induced by inhalation of aerosolized lipopolysaccharide (LPS), modelling the neutrophil inflammatory component(s) of COPD. See the literature section below for scientific background.

Male Lewis rats (Charles River, Raleigh, N.C., USA) weighing approximately 300-400 grams are pretreated with either (a) test compound, for example suspended in ca. 0.5% methylcellulose (obtainable from Sigma-Aldrich, St Louis, Mo., USA) in water or (b) vehicle only, delivered orally in a dose volume of ca. 10 ml/kg. Generally, dose response curves can for example be generated using the following approx. doses of PDE4 inhibitors: 2.0, 0.4, 0.08, 0.016 and 0.0032 mg/kg. About thirty minutes following pretreatment, the rats are exposed to aerosolized LPS (Serotype E. Coli 026:B6 prepared by trichloroacetic acid extraction, obtainable from Sigma-Aldrich, St Louis, Mo., USA), generated from a nebulizer containing a ca. 100 μg/ml LPS solution (ca. 100 ug/ml). Rats are exposed to the LPS aerosol at a rate of ca. 4 L/min for ca. 20 minutes. LPS exposure is carried out in a closed chamber with internal dimensions of roughly 45 cm length×24 cm width×20 cm height. The nebulizer and exposure chamber are contained in a certified fume hood. At about 4 hours-post LPS exposure the rats are euthanized by overdose with pentobarbital at ca. 90 mg/kg, administered intraperitoneally. Bronchoalveolar lavage (BAL) is performed through a 14 gauge blunt needle into the exposed trachea. Five, 5 ml washes are performed to collect a total of 25 ml of BAL fluid. Total cell counts and leukocyte differentials are performed on BAL fluid in order to calculate neutrophil influx into the lung. Percent neutrophil inhibition at each dose (cf. vehicle) is calculated and a variable slope, sigmoidal dose-response curve is generated, usually using Prism Graph-Pad. The dose-response curve is used to calculate an ED50 value (in mg per kg of body weight) for inhibition by the PDE4 inhibitor of the LPS-induced neutrophilia.

Alternative method: In an alternative simpler embodiment of the procedure, a single oral dose of 10 mg/kg, or more usually 1.0 mg/kg or 0.3 mg/kg, of the PDE4 inhibitor (or vehicle) is administered to the rats, and percent neutrophil inhibition is calculated and reported for that specific dose.

Literature:

Filley G. F. Comparison of the structural and inflammatory features of COPD and asthma. Chest. 2000; 117(5) 251s-260s.

Howell R E, Jenkins L P, Fielding L E, and Grimes D. Inhibition of antigen-induced pulmonary eosinophilia and neutrophilia by selective inhibitors of phosphodiesterase types 3 and 4 in brown Norway rats. Pulmonary Pharmacology. 1995; 8: 83-89.

Spond J, Chapman R, Fine J, Jones H, Kreutner W, Kung T T, Minnicozzi M. Comparison of PDE 4 inhibitors, Rolipram and SB 207499 (Ariflo™), in a rat model of pulmonary neutrophilia. Pulmonary Pharmacology and Therapeutics. 2001; 14: 157-164.

Underwood D C, Osborn R R, Bochnowicz S, Webb E F, Rieman D J, Lee J C, Romanic A M, Adams J L, Hay D W P, and Griswold D E. SB 239063, a p38 MAPK inhibitor, reduces neutrophilia, inflammatory cytokines, MMP-9, and fibrosis in lung. Am J Physiol Lung Cell Mol Physiol. 2000; 279: L895-L902.

In Vivo Assay 2. Rat Pica Model of Enesis

Background: Selective PDE4 inhibitors have been shown to inhibit inflammation in various in vitro and in vivo models by increasing intracellular levels of cAMP of many immune cells (e.g. lymphocytes, monocytes). However, a side effect of some PDE4 inhibitors in some species is emesis. Because many rat models of inflammation are well characterized, they can be used in procedures (see e.g. In Vivo Assay 1 above) to show beneficial anti-inflammatory effects of PDE 4 inhibitors. However rats have no emetic response (they have no vomit reflex), so that the relationship between beneficial anti-inflammatory effects of PDE 4 inhibitors and emesis is difficult to study directly in rats.

However, in 1991, Takeda et al. (see Literature section below) demonstrated that the pica feeding response is analogous to emesis in rats. Pica feeding is a behavioural response to illness in rats wherein rats eat non-nutritive substances such as earth or in particular clay (e.g. kaolin) which may help to absorb toxins. Pica feeding can be induced by motion and chemicals (especially chemicals which are emetic in humans), and can be inhibited pharmacologically with drugs that inhibit emesis in humans. The Rat Pica Model, In Vivo Assay 2, can determine the level of pica response of rats to PDE 4 inhibition at pharmacologically relevant doses in parallel to in vivo anti-inflammatory Assays in (a separate set of) rats (e.g. In Vivo Assay 1 above).

Anti-inflammatory and pica assays in the same species together can provide data on the “therapeutic index” (TI) in the rat of the compounds/salts of the invention. The Rat TI can for example be calculated as the ratio of a) the potentially-emetic Pica Response ED50 dose from Assay 2 to b) the rat anti-inflammatory ED50 dose (e.g. measured by rat neutrophilia-inhibition in eg In Vivo Assay 1), with larger TI ratios possibly indicating lower emesis at many anti-inflammatory doses. This might allow a choice of a non-emetic or low-emetic pharmaceutical dose of the compounds or salts of the invention which has an anti-inflammatory effect. It is recognised however that achieving a low-emetic PDE4 inhibitory compound is not essential to the invention.

Procedure: On the first day of the experiment, the rats are housed individually in cages without bedding or “enrichment”. The rats are kept off of the cage floor by a wire screen. Pre-weighed food cups containing standard rat chow and clay pellets are placed in the cage. The clay pellets, obtainable from Languna Clay Co, City of Industry, Calif., USA, are the same size and shape as the food pellets. The rats are acclimated to the clay for 72 hours, during which time the cups and food and clay debris from the cage are weighed daily on an electronic balance capable of measuring to the nearest 0.1 grams. By the end of the 72 hour acclimation period the rats generally show no interest in the clay pellets.

At the end of 72 hours the rats are placed in clean cages and the food cups weighed. Rats that are still consuming clay regularly are removed from the study. Immediately prior to the dark cycle (the time when the animals are active and should be eating) the animals are split into treatment groups and dosed orally with a dose of the compound/salt of the invention (different doses for different treatment groups) or with vehicle alone, at a dose volume of ca. 2 ml/kg. In this oral dosing, the compound/salt can for example be in the form of a suspension in ca. 0.5% methylcellulose (obtainable Sigma-Aldrich, St. Louis, Mo., USA) in water. The food and clay cups and cage debris are weighed the following day and the total clay and food consumed that night by each individual animal is calculated.

A dose response is calculated by first converting the data into quantal response, where animals are either positive or negative for the pica response. A rat is “pica positive” if it consumes greater than or equal to 0.3 grams of clay over the mean of its control group. The D50 value is usually calculated using logistic regression performed by the Statistica software statistical package. A Pica Response ED50 value in mg per kg of body weight can then be calculated.

The Pica Response ED50 value can be compared to the neutrophilia-inhibition ED50 values for the same compound administered orally to the rat (measurable by In Vivo Assay 1 above), so that a Therapeutic Index (TI) in rats can be calculated thus:

$$Rat Therapeutic index ( TI ) ( 50 / 50 ) = \frac{Pica Response ED 50 value}{rat neutrophilia \text{-} inhibition ED 50 value}$$

In general, the Therapeutic Index (TI) calculated this way is often substantially different to, and for example can often be substantially higher than, the TI (D20/D50) calculated in the ferret (see In vivo Assay 4 below).

Alternatively, e.g. for a simpler test, the In Vivo Assay 2 (pica) can use only a single oral dose of the test compound (e.g. 10 mg/kg orally).

Literature:

Beavo J A, Contini, M., Heaslip, R. J. Multiple cyclic nucleotide phosphodiesterases. Mol Phannacol. 1994; 46:399-405.

Spond J, Chapman R, Fine J, Jones H, Kreutner W, Kung T T, Minnicozzi M. Comparison of PDE 4 inhibitors, Rolipram and SB 207499 (Ariflo™), in a rat model of pulmonary neutrophilia. Pulmonary Pharnacology and Therapeudtics. 2001; 14:157-164.

Takeda N, Hasegawa S, Morita M, and Matsunaga T. Pica in rats is analogous to emesis: an animal model in emesis research. Pharmacology, Biociemistry and Behavior. 1991; 45:817-821.

Takeda N, Hasegawa S, Morita M, Horii A, Uno A, Yamatodani A and Matsunaga T. Neuropharmacological mechanisms of emesis. I. Effects of antiemetic drugs on motion- and apomorphine-induced pica in rats. Meth Find Exp Clin Pharinacol. 1995; 17(9) 589-596.

Takeda N, Hasegawa S, Morita M, Horii A, Uno A, Yamatodani A and Matsunaga T. Neuropharmacological mechanisms of emesis. II. Effects of antiemetic drugs on cisplatin-induced pica in rats. Meth Find Exp Clin Phamacol. 1995; 17(9) 647-652.

In Vivo Assay 3. LPS Induced Pulmonary Neutrophilia in Rats: Effect of Intratracileally Adminiistered PDE4 Inhibitors

This assay is an animal model of inflammation in the lung—specifically neutrophilia induced by lipopolysaccharide (LPS)—and allows the study of putative inhibition of such neutrophilia (anti-inflammatory effect) by intratracheally (i.t.) administered PDE4 inhibitors. The PDE4 inhibitors are preferably in dry powder or wet suspension form. I.t. administration is one model of inhaled administration, allowing topical delivery to the lung.

Animals: Male CD (Sprague Dawley Derived) rats supplied by Charles River, Raleigh, NC, USA or Charles River, United Kingdom are housed in groups of 5 rats per cage, acclimatised after delivery for at least 5 days with bedding/nesting material regularly changed, fed on SDS diet R1 pelleted food given ad lib, and supplied with daily-changed pasteurised animal grade drinking water.

Device for dry powder administration: Disposable 3-way tap between dosing needle and syringe. The intratracheal dosing device (a 3-way sterile tap, Vycon 876.00; or Penn Century dry powder insufflator, DP-4) is weighed, the drug blend or inhalation grade lactose (vehicle control) is then added to the tap, the tap is closed to prevent loss of drug, and the tap is re-weighed to determine the weight of drug in the tap. After dosing, the tap is weighed again to determine the weight of drug that had left the tap. The needle, a Sigina Z21934-7 syringe needle 19-gauge 152 mm (6 inches) long with luer hub, is cut by engineering to approximately 132 mm (5.2 inches), a blunt end is made to prevent them damaging the rat's trachea, and the needle is weighed prior to and after drug delivery to confirm that no drug is retained in the needles after dosing.

Device for wet suspension administration: This is the similar to the above but a blunt dosing needle, whose forward end was slightly angled to the needle axis, is used, with a flexible plastic portex canula inserted into the needle.

Drugs and Materials: Lipopolysaccharide (LPS) (Serotype:0127:B8) (e.g. L3129 Lot 61K4075) is dissolved in phosphate-buffered saline (1BS). PDE4 inhibitors are preferably used in size-reduced (e.g. micronised) form, for example according to the Micronisation Example(s) given above.

For dry powder administration of the drug, the Dry Powder Formulation Example given above, comprising drug and inhalation-grade lactose, can optionally be used. One suitable inhalation-grade lactose that can be used (e.g. Lot E98L4675 Batch 845120) has 10% fines (10% of material under 15 um (15 micron) particle size measured by Malvern particle size).

Wet suspensions of the drug (aqueous) can be prepared by adding the required volume of vehicle to the drug; the vehicle used can for example be saline alone or a mixture of saline/tween (e.g. 0.2% tween 80). The wet suspension is usually sonicated for ca 10 minutes prior to use.

Preparation, and dosing with PDE 4 inhibitor: Rats are anaesthetised by placing the animals in a sealed Perspex chamber and exposing them to a gaseous mixture of isoflourane (4.5%), nitrous oxide (3 litres.minute⁻¹) and oxygen (1 litre.minute-⁻¹). Once anaesthetised, the animals are placed onto a stainless steel i.t. dosing support table. They are positioned on their back at approximately a 35° angle. A light is angled against the outside of the throat to highlight the trachea. The mouth is opened and the opening of the upper airway visualised. The procedure varies for wet suspension and dry powder administration of PDE4 inhibitors as follows:

Dosing with a Wet suspension: A portex cannula is introduced via a blunt metal dosing needle that has been carefully inserted into the rat trachea. The animals are intratracheally dosed with vehicle or PDE4 inhibitor via the dosing needle with a new internal canula used for each different drug group. The formulation is slowly (ca. 10 seconds) dosed into the trachea using a syringe attached to the dosing needle.

Dosing with a Dry Powder: The The intratracheal dosing device (a three-way sterile tap device, Vycon 876.00; or Penn Century dry powder insufflator, DP-4) and needle are inserted into the rat trachea up to a pre-determined point established to be located approximately 1 cm above the primary bifurcation. Another operator holds the needle at the specified position whilst 2×4 ml of air (using 3-way tap device) is delivered through the three-way tap by depressing the syringes (ideally coinciding with the animal inspiring), aiming to expel the entire drug quantity from the tap. (Alternatively, 2×3 ml of air is delevered using Penn Century dry powder insufflator device.) After dosing, the needle and tap or device are removed from the airway, and the tap closed off to prevent any retained drug leaving the tap.

After dosing with either wet suspension or dry powder, the animals are then removed from the table and observed constantly until they have recovered from the effects of anaesthesia. The animals are returned to the holding cages and given free access to food and water; they are observed and any unusual behavioural changes noted.

Exposure to LPS: About 2 hours after i.t. dosing with vehicle control or the PDE4 inhibitor, the rats are placed into sealed Perspex containers and exposed to an aerosol of LPS (nebuliser concentration ca. 150 μg.ml⁻¹=ca. 150 ug/ml) for ca. 15 minutes. Aerosols of LPS are generated by a nebuliser (DeVilbiss, USA) and this is directed into the Perspex exposure chamber. Following the 15-minute LPS-exposure period, the animals are returned to the holding cages and allowed free access to both food and water.

[In an alternative embodiment, the rats can be exposed to LPS less than 2 hours (e.g. about 30 minutes) after i.t. dosing. In another alternative embodiment, the rats can be exposed to LPS more than 2 hours (e.g. ca. 4 to ca. 24 hours) after i.t. dosing by vehicle or PDE4 inhibitor, to test whether or not the PDE4 inhibitor has a long duration of action (which is not essential).]

Bronchoalveolar ravage: About 4 hours after LPS exposure the animals are killed by overdose of sodium pentobarbitone (i.p.). The trachea is cannulated with polypropylene tubing and the lungs are ravaged (washed out) with 3×5 mls of heparinised (25 units.ml⁻¹) phosphate buffered saline (PBS).

Neutrophil cell counts: The Bronchoalveolar lavage (BAL) samples are centrifuged at ca. 1300 rpm for ca. 7 minutes. The supernatant is removed and the resulting cell pellet resuspended in ca. 1 ml PBS. A cell slide of the resuspension fluid is prepared by placing ca. 100 μl (ca. 100 ul) of resuspended BAL fluid into cytospin holders and then is spun at ca. 5000 rpm for ca. 5 minutes. The slides are allowed to air dry and then stained with Leishmans stain (ca. 20 minutes) to allow differential cell counting. The total cells are also counted from the resuspension. From these two counts, the total numbers of neutrophils in the BAL are determined. For a measure of PDE4-inhibitor-induced inhibition of neutrophilia, a comparison of the neutrophil count in rats treated with vehicle and rats treated with PDE4 inhibitors is conducted.

By varying the dose of the PDE4 inhibitor used in the dosing step (e.g. 0.2 or 0.1 mg of PDE4 inhibitor per kg of body weight, down to e.g. 0.01 mg/kg), a dose-response curve can be generated.

In Vivo Assay 4. Evaluation of Therapeutic Index of Orally-Administered PDE 4 Inhibitors in the Conscious Ferret

1.1 Materials

The following materials can be used for these studies:

PDE4 inhibitors are prepared for oral (p.o.) administration by dissolving in a fixed volume (ca. 1 ml) of acetone and then adding cremophor to ca. 20% of the final volume. Acetone is evaporated by directing a flow of nitrogen gas onto the solution. Once the acetone is removed, the solution is made up to final volume with distilled water.

LPS is dissolved in phosphate buffered saline.

1.2 Animals

Male ferrets (Mustela Pulorius Furo, weighing 1-2 kg) are transported and allowed to acclimatise for not less than 7 days. The diet comprises SDS diet C pelleted food given ad lib with Whiskers™ cat food given 3 times per week. The animals are supplied with pasteurised animal grade drinking water changed daily.

1.3 Experimental Protocol(s)

1.3.1 Dosing With PDE4 Inhibitors

PDE4 inhibitors are administered orally (p.o.), using a dose volume of ca. 1 ml/kg. Ferrets are fasted overnight but allowed free access to water. The animals are orally dosed with vehicle or PDE 4 inhibitor using a ca. 15 cm dosing needle that is passed down the back of the throat into the oesophagus. After dosing, the animals are returned to holding cages fitted with perspex doors to allow observation, and given free access to water. The animals are constantly observed and any emetic episodes (retching and vomiting) or behavioural changes are recorded. The animals are allowed access to food ca. 60-90 minutes after p.o. dosing.

1.3.2 Exposure to LPS

About thirty minutes after oral dosing with compound or vehicle control, the ferrets are placed into sealed perspex containers and exposed to an aerosol of LPS (ca. 30 μg/ml=ca. 30 ug/ml) for ca. 10 minutes. Aerosols of LPS are generated by a nebuliser (DeVilbiss, USA) and this is directed into the perspex exposure chamber. Following a 10-minute exposure period, the animals are returned to the holding cages and allowed free access to water, and at a later stage, food. General observation of the animals continues for a period of at least 2.5 hours post oral dosing. All emetic episodes and behavioural changes are recorded.

1.3.3 Bronchoalveolar Lavage and Cell Counts

About six hours after LPS exposure the animals are killed by overdose of sodium pentobarbitone administered intraperitoneally. The trachea is then cannulated with polypropylene tubing and the lungs lavaged twice with ca. 20 ml heparinised (10 units/ml) phosphate buffered saline (PBS). The bronchoalveolar lavage (BAL) samples are centrifuged at ca. 1300 rpm for ca. 7 minutes. The supernatant is removed and the resulting cell pellet re-suspended in ca. 1 ml PBS. A cell smear of re-suspended fluid is prepared and stained with Leishmans stain to allow differential cell counting. A total cell count is made using the remaining re-suspended sample. From this, the total number of neutrophils in the BAL sample is determined.

1.3.4 Pharmacodynamic Readouts

The following parameters are recorded:

a) % inhibition of LPS-induced pulmonary neutrophilia to determine the dose of PDE4 inhibitor which gives 50% inhibition (D50).

b) Emetic episodes—the number of vomits and retches are counted to determine the dose of PDE4 inhibitor that gives a 20% incidence of emesis (D20).

c) A therapeutic index (Ti), using this assay, is then calculated for each PDE4 inhibitor using the following equation:

$$Ferret Therapeutic index ( TI ) ( D 20 / D 50 ) = \frac{D 20 incidence of emesis in ferret}{D 50 inhibition of neutrophilia in ferret}$$

It is noted that the Ferret Therapeutic index (TI) (D20/D50) calculated using this in vivo Assay 4 is often substantially different to, and for example is often substantially lower than, the Rat TI (50/50) calculated using the rat oral inflammation and pica feeding Assays 1+2.

The calculation of Ferret TI using the known PDE4 inhibitor roflumilast in this Assay 4 is approximately as follows:

D20 for emesis=about 0.46 mg/kg p.o.,

D50 for ferret neutroplilia=about 0.42 mg/kg p.o.,

Ferret TI=about 1.1.

All publications, including but not limited to patents and patent applications, cited in this specification are herein incorporated by reference as if each individual publication were specifically and individually indicated to be incorporated by reference herein as though fully set forth.

### EXAMPLES

The various aspects of the invention will now be described by reference to the following examples. These examples are merely illustrative and are not to be construed as a limitation of the scope of the present invention.

In this section, “Intermediates” can represent syntheses of intermediate compounds intended for use in the synthesis of one or more of the “Examples”, or “Intermediates” can represent syntheses of intermediate compounds which can be used in the synthesis of compounds of formula (I) or salts thereof. “Examples” are generally exemplary compounds or salts of the invention, for example compounds of formula (I) or (IB) or salts thereof.

Abbreviations used herein:

AcOH acetic acid

Ac₂O acetic anhydride

BEMP 2-t-butylimino-2-diethylamino-1,3-dimethylperhydro-1,3,2-diazaphosphazine

BOC₂O di tert-butyl carbonate

DMSO dimethyl sulfoxide

DCM dichloromethane

DMF dimethyl formamide

DIPEA diisopropylethyl amine (iPr₂NEt)

EDC 1-(3-Dimethylaminopropyl)-3-ethylcarbodiimide hydrochloride

EtOAc ethyl acetate

Et₂O diethyl ether

Et₃N triethylamine

EtOH ethanol

HATU O-(7-Azabenzotriazol-1-yl)-N,N,N′,N′-tetramethyluronium hexafluorophosphate

HBTU O-(Benzotriazol-1-yl)-N,N,N′,N′-tetramethyluronium hexafluorophosphate

HOBT hydroxybenzotriazole=1-hydroxybenzotriazole

Lawesson's reagent 2,4bis(4-methoxyphenyl)-1,3-dithia-2,4-diphosphetane-2,4-disulphide

MeCN acetonitrile

MeOH methanol

THF Tetrahydrofuran

HPLC high pressure liquid chromatography

SPE solid phase extraction

NMR nuclear magnetic resonance (in which: s=singlet, d=doublet, t=triplet, q=quartet, dd=doublet of doublets, m=multiplet, H=no. of protons)

LCMS liquid chromatography/mass spectroscopy

TLC thin layer chromatography

h hours

T_{RET} retention time (from LCMS)

Room temperature this is usually in the range of about 20 to about 25° C.

General Experimental Details

Machine Methods used herein:

LCMS (liquid chromatography/mass spectroscopy)

Waters ZQ mass spectrometer operating in positive ion electrospray mode, mass range 100-1000 amu.

UV wavelength: 215-330 nM

Column: 3.3cm×4.6 mm ID, 3 μm ABZ+PLUS

Flow Rate: 3 ml/min

Injection Volume: 5

Solvent A: 95% acetonitrile+0.05% formic acid

Solvent B : 0.1% formic acid+10 mMolar ammonium acetate

Gradient: 0% A/0.7 min, 0-100% A/3.5 min, 100% A/1.1 min, 100-0% A/0.2 min

It should be noted that retention times (TmT) quoted herein may vary slightly (±0.1 min.) when samples were run on different Waters machines, even though the same type of column and identical flow rates, injection volumes, solvents and gradients were used.

Mass directed autoprep HPLC

The prep column used was a Supelcosil ABZplus (10 cm×2.12 cm) (usually 10 cm×2.12 cm×5 μm).

UV wavelength: 200-320 nM

Flow: 20 ml/min

Injection Volume: 1 ml; or more preferably 0.5 ml

Solvent A: 0.1% formic acid

Solvent B: 95% acetonitrile+5% formic acid; or more usually 99.95% acetonitrile+0.05% formic acid

Gradient: 100% A/lmin, 100-80% A/9 min, 80-1% A/3.5 min, 1% A/1.4 min, 1-100% A/0.1 min

Chiral Columns for Chromatographic Purification

ChiralPak AD, ChiralCel OD and ChiralCel OJ columns can be obtained from: Chiral Technologies Europe Sarl, Illkirch, France (Telephone: +33 (0)388795200; (cte@chiral.fr; www.chiral.fr).

Whelk-01 columns can be purchased from: Hichrom, 1, The Markham Centre, Station Road, Theale, Reading, Berks. RG7.4PE, United Kingdom (Telephone: +44 (0)1189303660; (info@hichrom.co.uk; www.hichrom.co.uk). Hichrom are agents for the manufacturers Regis Technologies Inc., 8210 Austin Avenue, Morton Grove, Ill. 60053, USA; telephone: +1-847-967-6000; www.registech.com.

Intermediates and Examples

Reagents not detailed in the text below are usually commercially available from chemicals suppliers, e.g. established suppliers such as Sigma-Aldrich. The addresses and/or contact details of the suppliers for some of the starting materials mentioned in the Intermediates and Examples below or the Assays above, or suppliers of chemicals in general, are as follows:

AB Chem, Inc., 547 Davignon, Dollard-des-Orneaux, Quebec, H9B 1Y4, Canada

ABCR GmbH & CO. KG, P.O. Box 21 01 35, 76151 Karlsruhe, Germany

ACB Blocks Ltd; Kolokolnikov Per, 9/10 Building 2, Moscow, 103045, Russia

Aceto Color Intermediates (catalogue name), Aceto Corporation, One Hollow Lane, Lake Success, N.Y., 11042-1215, USA

Acros Organics, A Division of Fisher Scientific Company, 500 American Road, Morris Plains, N.J. 07950, USA

Apin Chemicals Ltd., 82 C Milton Park, Abingdon, Oxon OX14 4RY, United Kingdom

Apollo Scientific Ltd., Unit 1A, Bingswood Industrial Estate, Whaley Bridge, Derbyshire SK23 7LY, United Kingdom

Aldrich (catalogue name), Sigma-Aldrich Company Ltd., Dorset, United Kingdom, telephone: +44 1202 733114; Fax: +44 1202 715460; ukcustsv@eurnotes.sial.com; or

Aldrich (catalogue name), Sigma-Aldrich Corp., P.O. Box 14508, St. Louis, Mo. 63178-9916, USA; telephone: +1-314-771-5765; fax: +1-314-771-5757; custser@sial.com; or

Aldrich (catalogue name), Sigma-Aldrich Chemie GmbH, Munich, Germany; telephone: +49 89 6513 0; Fax: +49 89 6513 1169; deorders@eurnotes.sial.com.

Alfa Aesar, A Johnson Matthey Company, 30 Bond Street, Ward Hill, Mass. 01835-8099, USA

Amersham Biosciences UK Ltd, Pollards Wood, Chalfont St Giles, Buckinghamshire HP8 4SP, United Kingdom

Arch Corporation, 100 Jersey Avenue, Building D, New Brunswick, N.J. 08901, USA

Array Biopharma Inc., 1885 33rd Street, Boulder, Colo. 80301, USA

AstaTech, Inc., 8301 Torresdale Ave., 19C, Philadelphia, Pa. 19136, USA

Austin Chemical Company, Inc., 1565 Barclay Blvd., Buffalo Grove, Ill. 60089, USA

Avocado Research, Shore Road, Port of Heysham Industrial Park, Heysham, Lancashire LA32XY, United Kingdom

Bayer AG, Business Group Basic and Fine Chemicals, D-51368 Leverkusen, Germany

Berk Univar plc, Berk House, P.O.Box 56, Basing View, Basingstoke, Hants RG21 2E6, United Kingdom

Bionet Research Ltd; Highfield Industrial Estate, Camelford, Comwall PL32 9QZ UK

Butt Park Ltd., Braysdown Works, Peasedown St. John, Bath BA2 8LL, United Kingdom

Chemical Building Blocks (catalogue name), Ambinter, 46 quai Louis Bleriot, Paris, F-75016, France

ChemBridge Europe, 4 Clark's Hill Rise, Hampton Wood, Evesham, Worcestershire WR11 6FW, United Kingdom

ChemService Inc., P.O.Box 3108, West Chester, Pa. 19381, USA

CiventiChem, PO Box 12041, Research Triangle Park N.C. 27709, USA

Combi-Blocks Inc., 7949 Silverton Avenue, Suite 915, San Diego, Calif. 92126, USA

Dynamit Nobel GmbH, Germany; also available from: Saville Whittle Ltd (UK agents of Dynamit Nobel), Vickers Street, Manchester M40 8EF, United Kingdom

E. Merck, Germany; or E. Merck (Merck Ltd), Hunter Boulevard, Magna Park, Lutterworth, Leicestershire LE17 4XN, United Kingdom

Esprit Chemical Company, Esprit Plaza, 7680 Matoaka Road, Sarasota, Fla. 34243, USA

Exploratory Library (catalogue name), Ambinter, 46 quai Louis Bleriot, Paris, F-75016, France

Fluka Chemie AG, Industriestrasse 25, P.O. Box 260, CH-9471 Buchs, Switzerland

Fluorochem Ltd., Wesley Street, Old Glossop, Derbyshire SKI3 7RY, United Kingdom

Heterocyclic Compounds Catalog (Florida Center for Heterocyclic Compounds, University of Florida, PO Box 117200, Gainsville, Fla. 32611-7200 USA

ICN Biomedicals, Inc., 3300 Hyland Avenue, Costa Mesa, Calif. 92626, USA

Interchim Intermediates (catalogue name), Interchim, 213 Avenue Kennedy, BP 1140, Montlucon, Cedex, 03103, France

Key Organics Ltd., 3, Highfield Indusrial Estate, Camelford, Comwall PL32 9QZ, United Kingdom

Lancaster Synthesis Ltd., Newgate, White Lund, Morecambe, Lancashire LA3 3DY, United Kingdom

Manchester Organics Ltd., Unit 2, Ashville Industrial Estate, Sutton Weaver, Runcorn, Cheshire WA7 3PF, United Kingdom

Matrix Scientific, P.O. Box 25067, Columbia, S.C. 29224-5067, USA

Maybridge Chemical Company Ltd., Trevillett, Tintagel, Cornwall PL34 0HW, United Kingdom

Maybridge Combichem (catalogue name), Maybridge Chemical Company Ltd., Trevillett, Tintagel, Cornwall PL34 0HW, United Kingdom

Maybridge Reactive Intermediates (catalogue name), Maybridge Chemical Company Ltd., Trevillett, Tintagel, Comwall PL34 0HW, United Kingdom

MicroChemistry Building Blocks (catalogue name), MicroChemistry-RadaPharma, Shosse Entusiastov 56, Moscow, 111123, Russia

Miteni S.p.A., Via Mecenate 90, Milano, 20138, Italy

Molecular Devices Corporation, Sunnydale, Calif., USA

N.D. Zelinsky Institute, Organic Chemistry, Leninsky prospect 47, 117913 Moscow B-334, Russia

Oakwood Products Inc., 1741, Old Dunbar Road, West Columbia, S.C., 29172, USA

OmegaChem Inc., 8800, Boulevard de la Rive Sud, Levis, PQ, G6V 9H1, Canada

Optimer Building Block (catalogue name), Array BioPharma, 3200 Walnut Street, Boulder, Colo. 80301, USA

Peakdale Molecular Ltd., Peakdale Science Park, Sheffield Road, Chapel-en-le-Frith, High Peak SK23 0PG, United Kingdom

Pfaltz & Bauer, Inc., 172 East Aurora Street, Waterbury, Coon. 06708, USA

Rare Chemicals (catalogue name), Rare Chemicals GmbH, Schulstrasse 6, 24214 Gettorf, Germany

SALOR (catalogue name) (Sigma Aldrich Library of Rare Chemicals), Aldrich Chemical Company Inc, 1001 West Saint Paul Avenue, Milwaukee, Wis. 53233, USA

Sigma (catalogue name), Sigma-Aldrich Corp., P.O. Box 14508, St. Louis, Mo. 63178-9916, USA; see “Aldrich” above for other non-US addresses and other contact details

SIGMA-RBI, One Strathmore Road, Natick, Mass. 01760-1312, USA

Synchem OHG Heinrich-Plett-Strasse 40, Kassel, D-34132, Germany

Syngene International Pvt Ltd, Hebbagodi, Hosur Road, Bangalore, India.

TCI America, 9211 North Harborgate Street, Portland, Oreg. 97203, USA

TimTec Building Blocks A or B, TimTec, Inc., P O Box 8941, Newark, Del. 19714-8941, USA TimTec Overseas Stock, TimTec Inc., 100 Interchange Blvd. Newark, Del. 19711, USA

TimTec Stock Library, TimTec, Inc., P O Box 8941, Newark Del. 19714-8941, USA

Trans World Chemicals, Inc., 14674 Southlawn Lane, Rockville, Md. 20850, USA

Ubichem PLC, Mayflower Close, Chandlers Ford Industrial Estate, Eastleigh, Hampshire SO53 4AR, United Kingdom

Ultrafine (UFC Ltd.), Synergy House, Guildhall Close, Manchester Science Park, Manchester M15 6SY, United Kingdom

Table of Intermediates

| Intermediate | |
|---|---|
| Number | Name |
| 1 | Ethyl 4-chloro-1-ethyl-1H-pyrazolo[3,4-b]pyridine-5-carboxylate |
| 2 | 4-Aminotetrahydropyran |
| 3 | 1-Acetyl-4-aminopiperidine |
| 4 | Ethyl 1-ethyl-4-(tetrahydro-2H-pyran-4-ylamino)-1H-pyrazolo[3,4- |
| | b]pyridine-5-carboxylate |
| 5 | ethyl 4-(cyclohexylamino)-1-ethyl-1H-pyrazolo[3,4-b]pyridine-5- |
| | carboxylate |
| 6 | Ethyl 4-[(1-acetyl-4-piperidinyl)amino]-1-ethyl-1H-pyrazolo[3,4-b]pyridine- |
| | 5-carboxylate |
| 7 | Ethyl 1-ethyl-4-[(4-hydroxycyclohexyl)amino]-1H-pyrazolo[3,4-b]pyridine- |
| | 5-carboxylate |
| 8 | Ethyl 1-ethyl-4-[(4-oxocyclohexyl)amino]-1H-pyrazolo[3,4-b]pyridine-5-carboxylate |
| 9 | Ethyl 1-ethyl-4-{[4-(hydroxyimino)cyclohexyl]amino}-1H-pyrazolo[3,4- |
| | b]pyridine-5-carboxylate |
| 10 | Ethyl 4-chloro-1-ethyl-6-methyl-1H-pyrazolo[3,4-b]pyridine-5-carboxylate |
| 11 | Ethyl 1-ethyl-6-methyl-4-(tetrahydro-2H-pyran-4-ylamino)-1H-pyrazolo[3,4- |
| | b]pyridine-5-carboxylate |
| 12 | Ethyl 1-ethyl-4-{[(1SR,3RS)-3-hydroxycyclohexyl]amino}-1H-pyrazolo[3,4- |
| | b]pyridine-5-carboxylate |
| 13 | 1-Ethyl-4-(tetrahydro-2H-pyran-4-ylamino)-1H-pyrazolo[3,4-b]pyridine-5-carboxylic acid |
| 14 | 4-(Cyclohexylamino)-1-ethyl-1H-pyrazolo[3,4-b]pyridine-5-carboxylic acid |
| 15 | 4-[(1-Acetyl-4-piperidinyl)amino]-1-ethyl-1H-pyrazolo[3,4-b]pyridine-5- |
| | carboxylic acid |
| 16 | 1-Ethyl-4-[(4-oxocyclohexyl)amino]-1H-pyrazolo[3,4-b]pyridine-5- |
| | carboxylic acid |
| 17 | 1-ethyl-4-{[4-(hydroxyimino)cyclohexyl]amino}-1H-pyrazolo[3,4- |
| | b]pyridine-5-carboxylic acid |
| 18 | 1-Ethyl-6-methyl-4-(tetrahydro-2H-pyran-4-ylamino)-1H-pyrazolo[3,4- |
| | b]pyridine-5-carboxylic acid |
| 19 | 1-Ethyl-4-{[(1SR,3RS)-3-hydroxycyclohexyl]amino}-1H-pyrazolo[3,4- |
| | b]pyridine-5-carboxylic acid |
| 20 | N-[(1E)-(2,4-dimethylphenyl)methylidene]-2-methyl-2-propanesulfinamide |
| 21 | 2-methyl-N-[(1E)-(2-methylphenyl)methylidene]-2-propanesulfinamide |
| 22 | N-[(1E)-(3-hydroxyphenyl)methylidene]-2-methyl-2-propanesulfinamide |
| 23 | 2-methyl-N-{(1E)-[3-(methyloxy)phenyl]methylidene}-2- |
| | propanesulfinamide |
| 24 | 2-methyl-N-{(1E)-[4-(methyloxy)phenyl]methylidene}-2- |
| | propanesulfinamide |
| 25 | N-[(1E)-(4-bromophenyl)methylidene]-2-methyl-2-propanesulfinamide |
| 26 | 2-methyl-N-[(1E)-(4-methylphenyl)methylidene]-2-propanesulfinamide |
| 27 | N-{(1E)-[4-(ethyloxy)phenyl]methylidene}-2-methyl-2-propanesulfinamide |
| 28 | 2-methyl-N-{(1E)-[4-(propyloxy)phenyl]methylidene}-2- |
| | propanesulfinamide |
| 29 | N-((1E)-{4-[(difluoromethyl)oxy]phenyl}methylidene)-2-methyl-2- |
| | propanesulfinamide |
| 30 | 2-methyl-N-{(1E)-[4-(trifluoromethyl)phenyl]methylidene}-2- |
| | propanesulfinamide |
| 31 | 2-methyl-N-{(1E)-[4-(1-methylethyl)phenyl]methylidene}-2- |
| | propanesulfinamide |
| 32 | N-[(1E)-(2,3-dimethylphenyl)methylidene]-2-methyl-2-propanesulfinamide |
| 33 | N-[(1E)-(4-chloro-2-fluorophenyl)methylidene]-2-methyl-2- |
| | propanesulfinamide |
| 34 | N-[(1E)-(3,4-dimethylphenyl)methylidene]-2-methyl-2-propanesulfinamide |
| 35 | N-[(1E)-(3,5-dimethylphenyl)methylidene]-2-methyl-2-propanesulfinamide |
| 36 | N-[(1E)-(3-chloro-4-methylphenyl)methylidene]-2-methyl-2- |
| | propanesulfinamide |
| 37 | N-[1-(2,4-dimethylphenyl)ethyl]-2-methyl-2-propanesulfinamide |
| 38 | 2-methyl-N-[1-(2-methylphenyl)ethyl]-2-propanesulfinamide |
| 39 | N-{1-[4-(ethyloxy)phenyl]ethyl}-2-methyl-2-propanesulfinamide |
| 40 | N-(1-{4-[(difluoromethyl)oxy]phenyl}ethyl)-2-methyl-2-propanesulfinamide |
| 41 | 2-methyl-N-{1-[4-(trifluoromethyl)phenyl]ethyl}-2-propanesulfinamide |
| 42 | N-[1-(2,3-dimethylphenyl)ethyl]-2-methyl-2-propanesulfinamide |
| 43 | N-[1-(4-chloro-2-fluorophenyl)ethyl]-2-methyl-2-propanesulfinamide |
| 44 | N-[1-(3-chloro-4-methylphenyl)ethyl]-2-methyl-2-propanesulfinamide |
| 45 | 2-methyl-N-[1-(2-methylphenyl)propyl]-2-propanesulfinamide |
| 46 | N-[1-(3-hydroxyphenyl)propyl]-2-methyl-2-propanesulfinamide |
| 47 | 2-methyl-N-{1-[3-(methyloxy)phenyl]propyl}-2-propanesulfinamide |
| 48 | 2-methyl-N-{1-[4-(methyloxy)phenyl]propyl}-2-propanesulfinamide |
| 49 | N-[1-(4-bromophenyl)propyl]-2-methyl-2-propanesulfinamide |
| 50 | 2-methyl-N-[1-(4-methylphenyl)propyl]-2-propanesulfinamide |
| 50a | 2-methyl-N-[(1S)-1-(4-methylphenyl)propyl]-2-propanesulfinamide |
| 51 | N-{1-[4-(ethyloxy)phenyl]propyl}-2-methyl-2-propanesulfinamide |
| 52 | 2-methyl-N-{1-[4-(propyloxy)phenyl]propyl}-2-propanesulfinamide |
| 53 | N-(1-{4-[(difluoromethyl)oxy]phenyl}propyl)-2-methyl-2-propanesulfinamide |
| 54 | 2-methyl-N-{1-[4-(trifluoromethyl)phenyl]propyl}-2-propanesulfinamide |
| 55 | 2-methyl-N-{1-[4-(1-methylethyl)phenyl]propyl}-2-propanesulfinamide |
| 55a | 2-methyl-N-{(1S)-1-[4-(1-methylethyl)phenyl]propyl}-2- |
| | propanesulfinamide |
| 56 | N-[1-(2,3-dimethylphenyl)propyl]-2-methyl-2-propanesulfinamide |
| 57 | N-[1-(2,4-dimethylphenyl)propyl]-2-methyl-2-propanesulfinamide |
| 58 | N-[1-(4-chloro-2-fluorophenyl)propyl]-2-methyl-2-propanesulfinamide |
| 58a | N-[(1S)-1-(4-chloro-2-fluorophenyl)propyl]-2-methyl-2-propanesulfinamide |
| 59 | N-[1-(3,4-dimethylphenyl)propyl]-2-methyl-2-propanesulfinamide |
| 60 | N-[1-(3,5-dimethylphenyl)propyl]-2-methyl-2-propanesulfinamide |
| 61 | N-[1-(3-chloro-4-methylphenyl)propyl]-2-methyl-2-propanesulfinamide |
| 62 | [1-(2,4-dimethylphenyl)ethyl]amine hydrochloride |
| 63 | [1-(2-methylphenyl)ethyl]amine hydrochloride |
| 64 | {1-[4-(ethyloxy)phenyl]ethyl}amine hydrochloride |
| 65 | (1-{4-[(difluoromethyl)oxy]phenyl}ethyl)amine hydrochloride |
| 66 | {1-[4-(trifluoromethyl)phenyl]ethyl}amine hydrochloride |
| 67 | [1-(2,4-dimethylphenyl)ethyl]amine trifluoroacetate |
| 68 | [1-(4-chloro-2-fluorophenyl)ethyl]amine hydrochloride |
| 69 | [1-(3-chloro-4-methylphenyl)ethyl]amine hydrochloride |
| 70 | [1-(2-methylphenyl)propyl]amine hydrochloride |
| 71 | 3-(1-aminopropyl)phenol hydrochloride |
| 72 | {1-[3-(methyloxy)phenyl]propyl}amine hydrochloride |
| 73 | {1-[4-(methyloxy)phenyl]propyl}amine hydrochloride |
| 74 | [1-(4-bromophenyl)propyl]amine hydrochloride |
| 75 | [1-(4-methylphenyl)propyl]amine hydrochloride |
| 75a | [(1R)-(4-methylphenyl)propyl]amine hydrochloride |
| 76 | {1-[4-(ethyloxy)phenyl]propyl}amine hydrochloride |
| 77 | {1-[4-(propyloxy)phenyl]propyl}amine hydrochloride |
| 78 | (1-{4-[(difluoromethyl)oxy]phenyl}propyl)amine hydrochloride |
| 79 | {1-[4-(trifluoromethyl)phenyl]propyl}amine hydrochloride |
| 80 | {1-[4-(1-methylethyl)phenyl]propyl}amine hydrochloride |
| 80a | {(1R)-[4-(1-methylethyl)phenyl]propyl}amine hydrochloride |
| 81 | [1-(2,3-dimethylphenyl)propyl]amine hydrochloride |
| 82 | [1-(2,4-dimethylphenyl)propyl]amine hydrochloride |
| 83 | [1-(4-chloro-2-fluorophenyl)propyl]amine hydrochloride |
| 83a | [(1R)-(4-chloro-2-fluorophenyl)propyl]amine hydrochloride |
| 84 | [1-(3,4-dimethylphenyl)propyl]amine hydrochloride |
| 85 | [1-(3,5-dimethylphenyl)propyl]amine hydrochloride |
| 86 | [1-(3-chloro-4-methylphenyl)propyl]amine hydrochloride |
| 87 | [1-(3,5-dimethylphenyl)ethyl]amine hydrochloride |
| 88 | 3-(1-aminoethyl)phenol hydrochloride |
| 89 | {1-[4-(1-methylethyl)phenyl]ethyl}amine hydrochloride |
| 90 | [1-(2,3-dihydro-1H-inden-5-yl)ethyl]amine hydrochloride |
| 91 | [1-(5,6,7,8-tetrahydro-2-naphthalenyl)ethyl]amine hydrochloride |
| 92 | (2,2,2-trifluoro-1-phenylethyl)amine hydrochloride |
| 93 | [1-(4-bromophenyl)-2,2,2-trifluoroethyl]amine hydrochloride |
| 94 | {2,2,2-trifluoro-1-[3-(methyloxy)phenyl]ethyl}amine hydrochloride |
| 95 | (1-phenylhexyl)amine hydrochloride |
| 96 | (1-phenylpentyl)amine hydrochloride |
| 97 | [cyclopropyl(phenyl)methyl]amine hydrochloride |
| 98 | (2-methyl-1-phenylpropyl)amine hydrochloride |
| 99 | (1-phenylbutyl)amine hydrochloride |
| 100 | [1-(2,4-dimethylphenyl)ethyl]amine trifluoroacetate |
| 101 | [1-(2,4-dimethylphenyl)ethyl]amine trifluoroacetate |
| 102 | Ethyl 4-[(1-{[(1,1-dimethylethyl)oxy]carbonyl}-4-piperidinyl)amino]-1- |
| | ethyl-1H-pyrazolo[3,4-b]pyridine-5-carboxylate |
| 103 | Ethyl 1-ethyl-4-(4-piperidinylamino)-1H-pyrazolo[3,4-b]pyridine-5- |
| | carboxylate hydrochloride |
| 104 | Ethyl 4-{[1-(aminocarbonyl)-4-piperidinyl]amino}-1-ethyl-1H-pyrazolo[3,4- |
| | b]pyridine-5-carboxylate |
| 105 | 4-{[1-(aminocarbonyl)-4-piperidinyl]amino}-1-ethyl-1H-pyrazolo[3,4- |
| | b]pyridine-5-carboxylic acid |
| 106 | 4-chloro-1-ethyl-1H-pyrazolo[3,4-b]pyridine-5-carboxylic acid |
| 107 | 4-chloro-1-ethyl-1H-pyrazolo[3,4-b]pyridine-5-carbonyl chloride |
| 108 | 4-chloro-1-ethyl-N-[(1R)-1-(4-methylphenyl)ethyl]-1H-pyrazolo[3,4- |
| | b]pyridine-5-carboxamide |
| 109 | 4-chloro-1-ethyl-N-[(1R)-1-phenylethyl]-1H-pyrazolo[3,4-b]pyridine-5- |
| | carboxamide |
| 110 | 1,1-dimethylethyl [1-(aminocarbonyl)-4-piperidinyl]carbamate |
| 111 | 4-amino-1-piperidinecarboxamide hydrochloride |
| 112 | 1,1-dimethylethyl [4-(aminocarbonyl)cyclohexyl]carbamate |
| 113 | 4-aminocyclohexanecarboxamide hydrochloride |
| 114 | 1,1-dimethylethyl [cis-4-(aminocarbonyl)cyclohexyl]carbamate |
| 115 | 1,1-dimethylethyl [trans-4-(aminocarbonyl)cyclohexyl]carbamate |
| 116 | cis-4-aminocyclohexanecarboxamide hydrochloride |
| 117 | trans-4-aminocyclohexanecarboxamide hydrochloride |
| 118 | ethyl 4-{[cis-4-(aminocarbonyl)cyclohexyl]amino}-1-ethyl-1H-pyrazolo[3,4- |
| | b]pyridine-5-carboxylate |
| 119 | ethyl 4-{[trans-4-(aminocarbonyl)cyclohexyl]amino}-1-ethyl-1H-pyrazolo[3,4- |
| | b]pyridine-5-carboxylate |
| 120 | 4-{[cis-4-(aminocarbonyl)cyclohexyl]amino}-1-ethyl-1H-pyrazolo[3,4-b]pyridine- |
| | 5-carboxylic acid |
| 121 | 4-{[trans-4-(aminocarbonyl)cyclohexyl]amino}-1-ethyl-1H-pyrazolo[3,4- |
| | b]pyridine-5-carboxylic acid |
| 122 | 4-chloro-N-[1-(2,4-dimethylphenyl)propyl]-1-ethyl-1H-pyrazolo[3,4-b]pyridine-5- |
| | carboxamide |
| 123 | N-[(1E)-(2-ethylphenyl)methylidene]-2-methyl-2-propanesulfinamide |
| 124 | N-[(1E)-(4-ethylphenyl)methylidene]-2-methyl-2-propanesulfinamide |
| 125 | N-[(1E)-(2,5-dimethylphenyl)methylidene]-2-methyl-2-propanesulfinamide |
| 126 | N-[(1E)-(2,6-dimethylphenyl)methylidene]-2-methyl-2-propanesulfinamide |
| 127 | 2-methyl-N-[(1E)-(2,4,6-trimethylphenyl)methylidene]-2-propanesulfinamide |
| 128 | N-[(1R)-1-(2-ethylphenyl)ethyl]-2-methyl-2-propanesulfinamide |
| 129 | N-[(1R)-1-(4-ethylphenyl)ethyl]-2-methyl-2-propanesulfinamide |
| 130 | N-[(1R)-1-(2,5-dimethylphenyl)ethyl]-2-methyl-2-propanesulfinamide |
| 131 | 2-methyl-N-[(1R)-1-(2,4,6-trimethylphenyl)ethyl]-2-propanesulfinamide |
| 132 | N-[(1S)-1-(2-ethylphenyl)propyl]-2-methyl-2-propanesulfinamide |
| 133 | N-[(1S)-1-(4-ethylphenyl)propyl]-2-methyl-2-propanesulfinamide |
| 134 | N-[1-(2,5-dimethylphenyl)propyl]-2-methyl-2-propanesulfinamide |
| 135 | N-[(1S)-1-(2,6-dimethylphenyl)propyl]-2-methyl-2-propanesulfinamide |
| 136 | 2-methyl-N-[(1S)-1-(2,4,6-trimethylphenyl)propyl]-2-propanesulfinamide |
| 137 | [(1R)-1-(2-ethylphenyl)ethyl]amine hydrochloride |
| 138 | [(1R)-1-(4-ethylphenyl)ethyl]amine hydrochloride |
| 139 | [(1R)-1-(2,5-dimethylphenyl)ethyl]amine hydrochloride |
| 140 | [(1R)-1-(2,4,6-trimethylphenyl)ethyl]amine hydrochloride |
| 141 | [(1R)-1-(2-ethylphenyl)propyl]amine hydrochloride |
| 142 | [(1R)-1-(4-ethylphenyl)propyl]amine hydrochloride |
| 143 | [(1R)-1-(2,5-dimethylphenyl)propyl]amine hydrochloride |
| 144 | [(1R)-1-(2,6-dimethylphenyl)propyl]amine hydrochloride |
| 145 | [(1R)-1-(2,4,6-trimethylphenyl)propyl]amine hydrochloride |
| 146 | ethyl 4-[((3S)-1-{[(1,1-dimethylethyl)oxy]carbonyl}-3-pyrrolidinyl)amino]-1-ethyl- |
| | 1H-pyrazolo[3,4-b]pyridine-5-carboxylate |
| 147 | ethyl 4-[((3R)-1-{[(1,1-dimethylethyl)oxy]carbonyl}-3-pyrrolidinyl)amino]-1- |
| | ethyl-1H-pyrazolo[3,4-b]pyridine-5-carboxylate |
| 148 | ethyl 1-ethyl-4-[(3S)-3-pyrrolidinylamino]-1H-pyrazolo[3,4-b]pyridine-5- |
| | carboxylate hydrochloride |
| 149 | ethyl 1-ethyl-4-[(3R)-3-pyrrolidinylamino]-1H-pyrazolo[3,4-b]pyridine-5- |
| | carboxylate hydrochloride |
| 150 | ethyl 4-{[(3S)-1-(aminocarbonyl)-3-pyrrolidinyl]amino}-1-ethyl-1H-pyrazolo[3,4- |
| | b]pyridine-5-carboxylate |
| 151 | ethyl 4-{[(3R)-1-(aminocarbonyl)-3-pyrrolidinyl]amino}-1-ethyl-1H-pyrazolo[3,4- |
| | b]pyridine-5-carboxylate |
| 152 | 4-{[(3S)-1-(aminocarbonyl)-3-pyrrolidinyl]amino}-1-ethyl-1H-pyrazolo[3,4- |
| | b]pyridine-5-carboxylic acid |
| 153 | 4-{[(3R)-1-(aminocarbonyl)-3-pyrrolidinyl]amino}-1-ethyl-1H-pyrazolo[3,4- |
| | b]pyridine-5-carboxylic acid |
| 154 | 1,1-dimethylethyl (cis-4- |
| | {[methyl(methyloxy)amino]carbonyl}cyclohexyl)carbamate |
| 155 | 1,1-dimethylethyl (cis-4-acetylcyclohexyl)carbamate |
| 156 | 1-(cis-4-aminocyclohexyl)ethanone hydrochloride |
| 157 | ethyl 4-[(4-acetylcyclohexyl)amino]-1-ethyl-1H-pyrazolo[3,4-b]pyridine-5- |
| | carboxylate (mixture of cis and trans isomers) |
| 158 | 4-[(4-acetylcyclohexyl)amino]-1-ethyl-1H-pyrazolo[3,4-b]pyridine-5-carboxylic |
| | acid (mixture of cis and trans isomers) |
| 159 | (RS)-1,1-dimethylethyl [cis-4-(1-hydroxyethyl)cyclohexyl]carbamate |
| 160 | (RS)-1-(cis-4-aminocyclohexyl)ethanol hydrochloride |
| 161 | ethyl 1-ethyl-4-{[(1S,3S)-3-hydroxycyclohexyl]amino}-1H-pyrazolo[3,4- |
| | b]pyridine-5-carboxylate and ethyl 1-ethyl-4-{[(1R,3R)-3- |
| | hydroxycyclohexyl]amino}-1H-pyrazolo[3,4-b]pyridine-5-carboxylate |
| 162 | 1-ethyl-4-{[(1R,3R)-3-hydroxycyclohexyl]amino}-1H-pyrazolo[3,4-b]pyridine-5- |
| | carboxylic acid |
| 163 | 4-[(1-{[(1,1-dimethylethyl)oxy]carbonyl}-4-piperidinyl)amino]-1-ethyl-1H- |
| | pyrazolo[3,4-b]pyridine-5-carboxylic acid |
| 164 | 1,1-dimethylethyl 4-{[1-ethyl-5-({[(1R)-1-(4-methylphenyl)ethyl]amino}carbonyl)- |
| | 1H-pyrazolo[3,4-b]pyridin-4-yl]amino}-1-piperidinecarboxylate |
| 165 | 1,1-dimethylethyl 4-{[5-({[1-(2,4-dimethylphenyl)propyl]amino}carbonyl)-1-ethyl- |
| | 1H-pyrazolo[3,4-b]pyridin-4-yl]amino}-1-piperidinecarboxylate |
| 166 | 4-Amino-4-(3-methylphenyl)butyric acid |
| 167 | 4-({[(1,1-dimethylethyl)oxy]carbonyl}amino)-4-(3-methylphenyl)butanoic acid |
| 168 | 1,1-dimethylethyl [4-(dimethylamino)-1-(3-methylphenyl)-4-oxobutyl]carbamate |
| 169 | 4-amino-N,N-dimethyl-4-(3-methylphenyl)butanamide hydrochloride |

### Intermediate 1: Ethyl 4-chloro-1-ethyl-1H-pyrazolo[3,4-b]pyridine-5-carboxylate

This can be prepared from commercially available 5-amino-1-ethyl pyrazole as described by G. Yu et. al. in J. Med Chem., 2001, 44, 1025-1027:

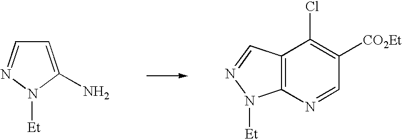

### Intermediate 1A: Ethyl 4-ethoxy-1H-pyrazolo[3,4-b]pyridine-5-carboxylate

This can be prepared by oxidative cleavage (SeO₂) of 1-furanylmethyl derivative, as described by T. M. Bare et. al. In J. Med. Chem., 1989, 32, 2561-2573, (further referenced to Zuleski, F. R., Kirkland, K. R., Melgar, M. D.; Malbica, J. Drug. Metab. Dispos., 1985, 13, 139):

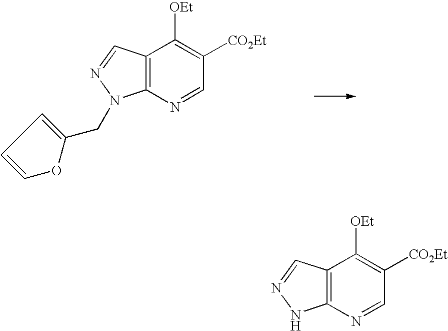

### Intermediate 2: 4-Aminotetrahydropyran

Commercially available from Combi-Blocks Inc., 7949 Silverton Avenue, Suite 915, San Diego, Calif. 92126, USA (CAS 38041-19-9)

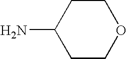

### Intermediate 2A: Tetrahydro-2H-pyran-4-amine hydrochloride=4-Aminotetrahydropyran hydrochloride

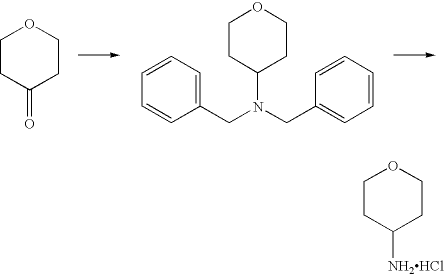

### Step 1: N,N-dibenzyltetrahydro-2H-pyran-4-amine

Dibenzylamine (34.5 g) and acetic acid (6.7 ml) were added to a stirred solution of tetrahydro-4H-pyran-4-one (16.4 g, commercially available from e.g. Aldrich) in dichloromethane (260 ml) at 0° C. to 5° C. After 2.5 h at 0° C. to 5° C., sodium triacetoxyborohydride (38.9 g) was added portionwise, and the mixture was allowed to warm to room temperature. After stirring at room temperature overnight, the reaction mixture was washed successively with 2M-sodium hydroxide (200 ml and 50 ml), water (2×50 ml) and brine (50 ml), then dried and evaporated to give a yellow oil (45 g). This oil was stirred with methanol (50 ml) at 4° C. for 30 min to give the product as a white solid (21.5 g). LCMS showed MH⁺=282; T_{RET}=1.98 min.

### Step 2: Tetrahydro-2H-pyran-4-amine hydrochloride

N,N-dibenzyltetrahydro-2H-pyran-4-amine (20.5 g) was dissolved in ethanol (210 ml) and hydrogenated over 10% palladium on carbon catalyst (4 g) at 100 psi for 72 h at room temperature. The reaction mixture was filtered and the filtrate was adjusted to pH 1 with 2M-hydrogen chloride in diethyl ether. Evaporation of solvents gave a solid which was triturated with diethyl ether to give the product as a white solid (9.23 g). ¹H NMR (400 MHz in d₆-DMSO, 27° C., δppm) 8.24 (br. s, 3H), 3.86 (dd, 12, 4Hz, 2H), 3.31 (dt, 2, 12 Hz, 2H), 3.20 (m, 1H), 1.84 (m, 2H), 1.55 (dq, 4, 12 Hz, 2H).

### Intermediate 3: 1-Acetyl-4-aminopiperidine

This can be prepared from commercially available N1-benzyl-4-aminopiperidine as described by Yamada et. al. In WO 00/42011:

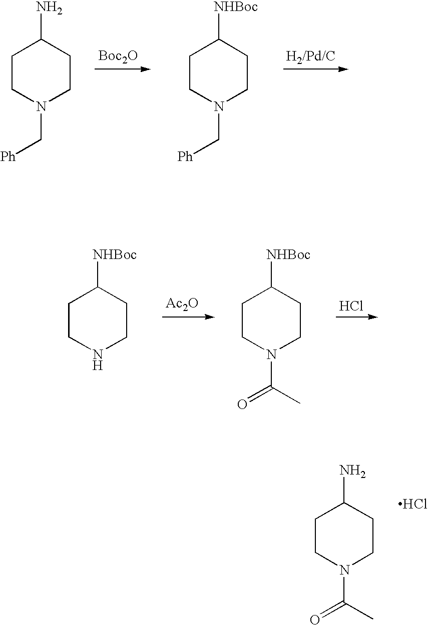

### Intermediate 4: Ethyl 1-ethyl-4-(tetrahydro-2H-pyran-4-ylamino)-1H-pyrazolo[3,4-b]pyridine-5-carboxylate

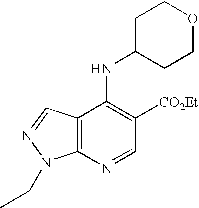

Intermediate 1 (0.20 g) and triethylamine (0.55 ml) were suspended in ethanol (8 ml) and 4-aminotetrahydropyran (Intermediate 2, 0.088 g) was added. The mixture was stirred under nitrogen and heated at 80° C. for 16 h, then concentrated in vacuo. The residue was partitioned between DCM and water. The layers were separated and the organic layer was loaded directly onto an SPE cartridge (silica, 5 g) which was eluted sequentially with; (i) DCM, (ii) DCM : Et₂O (2:1), (iii) DCM : Et₂O (1:1), (iv) Et₂O and (v) EtOAc. Fractions containing desired material were combined and concentrated in vacuo to afford Intermediate 4 (0.21 g). LCMS showed MH⁺=319; T_{RET}=2.93 min.

Similarly prepared from Intermediate 1 were the following:

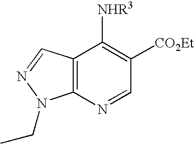

| | | | MH⁺ | T_{RET} |
|---|---|---|---|---|
| | NHR³ | Amine reagent | ion | (min) |
| Intermediate 5 | 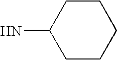 | Cyclo- hexylamine | 317 | 3.65 |
| Intermediate 6 | 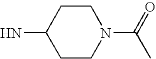 | Intermediate 3 | 360 | 2.71 |

### Intermediate 4

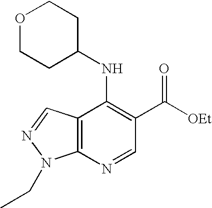

Alternative synthesis: Instead of the method shown above Intermediate 4 can also be made using the following Method B:

Method B: Intermediate 1 (2.5 g) was dissolved in acetonitrile (15 ml). 4-Aminotetrahydropyran hydrochloride (Intermediate 2A) (1.1 g) and N,N-diisopropylethylamine (9.4 ml) were added and the mixture stirred under nitrogen at 85° C. for 16 h. A trace of starting material remained, so an additional portion of 4-aminotetrahydropyran hydrochloride (0.11 g) was added and stirring continued at 85° C. for a further 16 h. The mixture was then concentrated in vacuo. The residue was partitioned between DCM and water. The layers were separated and the organic layer was washed with further water (2×20 ml) then dried (Na₂SO₄) and concentrated in vacuo. The residue was further purified by chromatography using Biotage (silica, 90 g), eluting with cyclohexane: ethyl acetate to afford Intermediate 4 (2.45 g). LCMS showed=319; T_{RET}=2.90 min.

### Intermediate 7: Ethyl 1-ethyl-4-[(4-hydroxycyclohexyl)amino]-1H-pyrazolo[3,4-b]pyridine-5-carboxylate

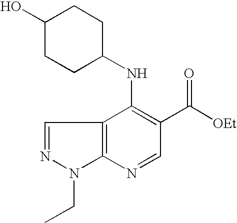

Intermediate 1 (1.5 g, 5.9 mmol) was dissolved in MeCN (80 ml). Trans-4-aminocyclohexanol (0.817 g, 7.1 mmol, commercially available from TCI-America; alternatively (e.g. as the HCl salt) from Aldrich) and DIPEA (6.18 ml, 35.5 mmol) were added and the mixture was stirred at 85° C. for 16 h. The mixture was concentrated in vacuo, and the residue was partitioned between DCM (120 ml) and water (30 ml). The phases were separated and the organic phase was dried (Na₂SO₄) and evaporated to give a pale yellow solid. The solid was dissolved in a mixture of DCM (10 ml) and chloroform (3 ml), and applied in equal portions to two SPE cartridges (silica, 20 g) which were eluted sequentially with a gradient of EtOAc:cyclohexane (1:16, then 1:8, 1:4, 1:2, 1:1 and 1:0). Fractions containing the desired material were combined and evaporated in vacuo to give Intermediate 7 (1.89 g) as a white solid. LCMS showed MH⁺=333; T_{RET}=2.79 min.

### Intermediate 8: Ethyl 1-ethyl4-[(4-oxocyclohexyl)amino]-1H-pyrazolo[3,4-b]pyridine-5-carboxylate

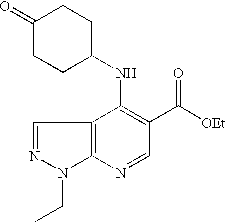

Intermediate 7 (1.893 g, 5.7 mmol) was suspended in acetone (12 ml) and the stirred suspension was treated at 0° C. with Jones reagent (1.81 ml). After 30 min, a further quantity of Jones reagent (1.81 ml) was added to the reaction mixture which was maintained at 0° C. After a further 2 h, a final portion of Jones reagent (1.44 ml) was added to the reaction mixture, and stirring at 0° C. was continued for 1 h. Isopropanol (3.8 ml) was added to the reaction mixture, followed by water (15 ml). The resulting mixture was extracted with EtOAc (2×40 ml). The combined organic extracts were washed with water (8 ml), dried (Na₂SO₄) and evaporated to a grey solid. The solid was dissolved in DCM (10 ml) and applied in equal portions to two SPE cartridges (silica, 20 g) which were eluted sequentially with a gradient of EtOAc:cyclohexane (1:16, then 1:8, 1:4, 1:2, and 1:1). Fractions containing the desired material were combined and evaporated in vacuo to give Intermediate 8 (1.893 g) as a white solid. LCMS showed MH⁺=331; T_{RET}=2.84 min.

### Intermediate 9: Ethyl 1-ethyl-4-{[4-(hydroxyimino)cyclohexyl]amino}-1H-pyrazolo[3,4-b]pyridine-5-carboxylate

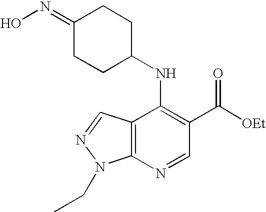

A mixture of Intermediate 8 (200 mg), hydroxylamine hydrochloride (50 mg) and anhydrous potassium carbonate (420 mg) in MeCN(10 ml) was stirred and heated at reflux for 17 hours. The solution was cooled and concentrated in vacuo. The residue was partitioned between EtOAc and water. The organic phase was separated, dried over Na₂SO₄ and concentrated in vacuo to give Intermediate 9 as a white powder (203 mg). LCMS showed MH⁺=346; T_{RET}=2.84 min.

### Intermediate 10: Ethyl 4-chloro-1-ethyl-6-methyl-1H-pyrazolo[3,4-b]pyridine-5-carboxylate

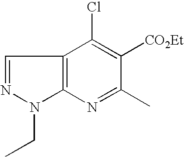

A mixture of 5-amino-1-ethylpyrazole (1.614 g, 14.5 mmol) and diethyl 2-(1-ethoxyethylidene)malonate (3.68 g, 16.0 mmol, as described by P. P. T. Sah, J. Amer. Chem. Soc., 1931, 53, 1836) was heated at 150° C. under Dean Stark conditions for 5 hours. Phosphorous oxychloride (25 ml) was carefully added to the mixture and the resulting solution was heated at 130° C. under reflux for 18 hours. The mixture was concentrated in vacuo, then the residual oil was carefully added, with cooling, to water (100 ml). The resulting mixture was extracted with DCM (3×100 ml) and the combined organic extracts were dried over anhydrous Na₂SO₄ and concentrated in vacuo. The residual oil was purified by Biotage chromatography (silica, 90 g) eluting with EtOAc-petroleum ether (1:19). Fractions containing the desired product were combined and concentrated in vacuo to afford Intermediate 10 (1.15 g). LCMS showed MH⁺=268; T_{RET}=3.18 min.

### Intermediate 11: Ethyl 1-ethyl-6-methyl-4-(tetrahydro-2H-pyran-4-ylamino)-1H-pyrazolo[3,4-b]pyridine-5-carboxylate

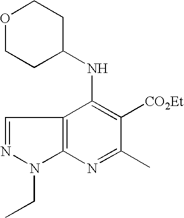

4-Aminotetrahydropyran hydrochloride (Intermediate 2A, 0.413 g, 3.0 mmol) was added to a mixture of Intermediate 10 (0.268 g, 1.0 mmol) and DIPEA (0.87 ml, 5.0 mmol) in MeCN (3 ml). The resulting mixture was heated at 85° C. for 24 hours. Volatiles were removed in vacuo and the residue was dissolved in chloroform (1.5 ml) and applied to a SPE cartridge (silica, 5 g). The cartridge was eluted successively with Et₂O, EtOAc and EtOAc-MeOH (9/1). Fractions containing the desired product were combined and concentrated in vacuo to give the desired product contaminated with starting material (Intermediate 10). Further purification using a SPE cartridge (silica, 5 g) eluting with EtOAc-cyclohexane (1:3) afforded Intermediate 11 (0.248 g). LCMS showed MH⁺=333; T_{RET}=2.75 min.

### Intermediate 12: Ethyl 1-ethyl-4-{[(1SR,3RS)-3-hydroxycyclohexyl]amino}-1H-pyrazolo [3,4-b]pyridine-5-carboxylate

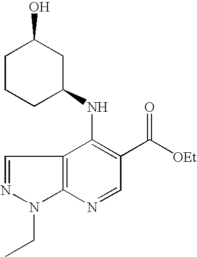

### [cis-(3-hydroxycyclohex-1-yl)amino group, racemic]

3-Aminocyclohexanol (0.677 g, 5.9 mmol, for example as described in J. Chem. Soc., Perkin Trans 1, 1994, 537 which describes the preparation of a 3.3:1 cis: trans mixture of 3-aminocyclohexanol) in MeCN(10 ml) and EtOH (1 ml) was added at room temperature to a stirred solution of Intermediate 1 (1.24 g, 4.9 mmol) and DIPEA (4.26 ml, 24.5 mmol) in MeCN (25 ml). The resulting mixture was stirred at 85° C. for 17 h. The mixture was concentrated in vacuo, and the residue was partitioned between DCM (50 ml) and water (10 ml). The phases were separated and the organic phase was dried (Na₂SO₄) and evaporated to give an orange-brown oil. The oil was purified by Biotage chromatography (silica 100 g) eluting with 30-50% EtOAc in cyclohexane to give Intermediate 12 as a white foam (0.68 g). LCMS showed MH⁺=333; T_{RET}=2.76 min.

### Intermediate 13: 1-Ethyl-4-(tetrahydro-2H-pyran-4-ylamino)-1H-pyrazolo[3,4-b]pyridine-5-carboxylic acid

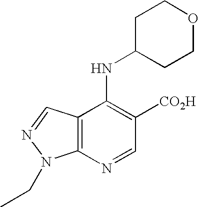

A solution of Intermediate 4 (0.21 g) in ethanol:water (95:5, 10 ml) was treated with sodium hydroxide (0.12 g). The mixture was heated at 50° C. for 8 h, then concentrated in vacuo, dissolved in water and acidified to pH 4 with acetic acid. The resultant white solid was removed by filtration and dried in vacuo to afford Intermediate 13 as an off-white solid (0.156 g). LCMS showed MH⁺=291; T_{RET}=2.11 min.

An alternative preparation of Intermediate 13 is as follows:

A solution of Intermediate 4 (37.8 g) in ethanol:water (4:1, 375 ml) was treated with sodium hydroxide (18.9 g). The mixture was heated at 50° C. for 5 hours, then concentrated in vacuo, dissolved in water and acidified to pH 2 with aqueous hydrochloric acid (2M). The resultant white solid was removed by filtration and dried in vacuo to afford Intermediate 13 as an off-white solid (29.65 g). LCMS showed MH⁺=291; T_{RET}=2.17 min.

### Intermediate 14: 4-(Cyclohexylamino)-1-ethyl-1H-pyrazolo[3,4-b]pyridine-5-carboxylic acid

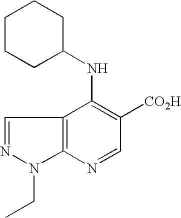

A solution of Intermediate 5 (5.37 g, 17 mmol) in EtOH (30 ml) was treated with a solution of sodium hydroxide (2.72 g, 68 mmol) in water (20 ml), and the resulting mixture was stirred at 50° C. for 3 h. The reaction mixture was concentrated in vacuo, dissolved in water (250 ml) and the cooled solution was acidified to pH 1 with 5M-hydrochloric acid. The resultant solid was collected by filtration and dried in vacuo to afford Intermediate 14 as a white solid (4.7 g). LCMS showed MH⁺=289; T_{RET}=2.83 min.

### Intermediate 15: 4-[(1-Acetyl-4-piperidinyl)amino]-1-ethyl-1H-pyrazolo[3,4-b]pyridine-5-carboxylic acid

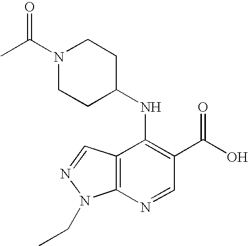

Aqueous sodium hydroxide solution (8.55 ml, 2M) was added to a solution of Intermediate 6 (1.55 g) in EtOH (13 ml). The mixture was heated at 50° C. for 18 h then neutralised using aqueous hydrochloric acid and evaporated in vacuo to afford a mixture of 1-ethyl-4-(4-piperidinylamino)-1H-pyrazolo[3,4-b]pyridine-5-carboxylic acid and 4-[(1-acetyl-4-piperidinyl)amino]-1-ethyl-1H-pyrazolo[3,4-b]pyridine-5-carboxylic acid.

Acetic acid (0.36 ml) was added to a stirred mixture of HATU (2.41 g) and DIPEA (2.21 ml) in DMF (65 ml). After stirring for 15 min the mixture was added to the mixture of 1-ethyl-4-(4-piperidinylamino)-1H-pyrazolo[3,4-b]pyridine-5-carboxylic acid and 4-[(1-acetyl-4-piperidinyl)amino]-1-ethyl-1H-pyrazolo[3,4-b]pyridine-5-carboxylic acid and the reaction mixture was stirred for 15 h. The reaction mixture was concentrated in vacuo and the residue purified by chromatography using Biotage (silica 90 g), eluting with DCM: MeOH (0% -5% MeOH) to afford Intermediate 15 (1.36 g) as a white solid. LCMS showed MH⁺=334; T_{RET}=2.06 min.

### Intermediate 16: 1-Ethyl-4-[(4-oxocyclohexyl)amino]-1H-pyrazolo[3,4-b]pyridine-5-carboxylic acid

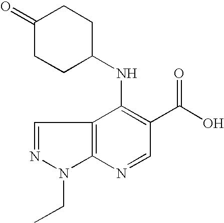

A solution of sodium hydroxide (0.053 g, 1.32 mmol) in water (0.41 ml) was added to a stirred solution of Intermediate 8 (0.1 g, 0.303 mmol) in ethanol (1 ml), and the resulting mixture was heated at 50° C. After 1 h, the cooled reaction mixture was adjusted to pH3 with 2M hydrochloric acid, and extracted with EtOAc (2×6 ml). The combined organic extracts were dried (Na₂SO₄) and evaporated to give Intermediate 16 (0.072 g) as a white solid. LCMS showed MH⁺=303; T_{RET}=2.13 min.

An alternative preparation of Intermediate 16 is as follows:

A solution of sodium hydroxide (0.792 g, 19.8 mmol) in water (6 ml) was added to a stirred solution of Intermediate 8 (1.487 g, 4.5 mmol) in EtOH (15 ml), and the resulting mixture was heated at 50° C. After 1 hour, the cooled reaction mixture was adjusted to pH4 with 2M hydrochloric acid, and extracted with EtOAc (3×30 ml). The combined organic extracts were dried (Na₂SO₄) and evaporated to give Intermediate 16 (1.188 g) as a white solid. LCMS showed MH⁺=303; T_{RET}=2.12 min.

### Intermediate 17: 1-ethyl-4-{[4-(hydroxyimino)cyclohexyl]amino}-1H-pyrazolo[3,4-b]pyridine-5-carboxylic acid

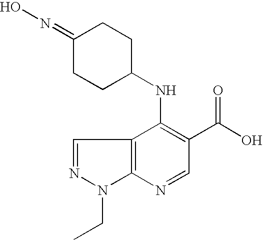

A solution of Intermediate 16 (0.58 g, 1.92 mmol), hydroxylamine hydrochloride (0.26 g, 3.74 mmol) and DIPEA (0.65 g, 5.03 mmol) in MeCN (35 ml) was stirred and heated at reflux for 3 hours, then cooled and left at room temperature overnight. Glacial AcOH (1 ml) was added, with stirring. The reaction mixture was concentrated in vacuo. EtOAc (10 ml) was added and the resultant suspension was stirred for 30 min. then applied to an SPE cartridge (silica, 20 g). The cartridge was eluted with a (250:1) mixture of EtOAc and glacial AcOH, followed by a (500:16:1) mixture of EtOAc, MeOH and glacial AcOH, to give Intermediate 17 (0.327 g) as a white solid. LCMS showed MH⁺=318; T_{RET}=2.21 min.

### Intermediate 18: 1-Ethyl-6-methyl-4-(tetrahydro-2H-pyran-4-ylamino)-1H-pyrazolo[3,4-b]pyridine-5-carboxylic acid

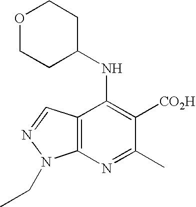

2M-Sodium hydroxide solution (0.75 ml, 1.5 mmol) was added to Intermediate 11 (0.248 g, 0.75 mmol) in EtOH (2 ml), and the mixture was heated at reflux for 16 hours. The reaction mixture was concentrated, diluted with water (1 ml) and acidified with 2M-hydrochloric acid (0.75 ml) to precipitate a solid which was collected by filtration to afford Intermediate 18 (0.168 g). LCMS showed MH⁺=305; T_{RET}=1.86 min.

### Intermediate 19: 1-Ethyl-4-{[(1SR,3RS)-3-hydroxycyclohexyl]amino}-1H-pyrazolo[3,4-b]pyridine-5-carboxylic acid

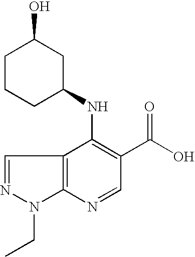

### (cis-3-hydroxycyclohex-1-ylamino group, racemic)

A solution of Intermediate 12 (0.681 g, 2.05 mmol) in EtOH (7 ml) was treated with a solution of sodium hydroxide (0.362 g, 9.05 mmol) in water (2.9 ml). The resulting mixture was stirred at 50° C. After 3 h, the reaction mixture was concentrated in vacuo to give a residual oil which was dissolved in water (3 ml), then cooled and acidified to pH 3 with 2M hydrochloric acid. After stirring at 0° C. for 1 h, the resulting precipitate was collected by filtration, washed with cooled water (0.5 ml) and dried in vacuo to afford Intermediate 19 as a white solid (0.491 g). LCMS showed MH⁺=305; T_{RET}=2.14 min.

### Intermediates 20-86

These intermediates were prepared using a modification of the procedure developed by D. A. Cogan, G. Liu and J. Ellman and described in Tetrahedron, 1999, 55, 8883-8904. In the Cogan, Liu, Ellman paper, the use of (S)-tert butyl sulphinamide in chemistry similar to that described in Intermediates 20-86 below allegedly produced an enrichment in a diastereoisomer with the general stereochemistry at the carbon atom next to the nitrogen shown here: 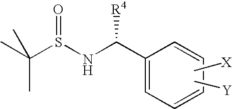
(i.e. inserted group R4 into the paper as shown, branched-benzyl is illustrative example only); this stereochemistry (R4 into the paper) was formed in the carbon-carbon bond forming reaction (i.e. before any optional separation of diastereoisomers). Therefore, compounds containing an alpha substituent on the benzylic carbon atom (Intermediates 37-86) are believed to be enriched in an enantiomer/diastereoisomer which is believed to have the (R)-stereochemistry at the benzylic carbon atom.

### Intermediate 20: N-[(1E)-(2,4-dimethylphenyl)methylidene]-2-methyl-2-propanesulfiniamide

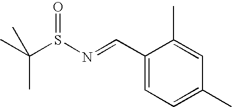

A solution of (S)-tert butyl sulphinamide (0.20 g, 1.65 mmol) in THF (2 ml) was added to 2,4-dimethylbenzaldehyde (0.22 g, 1.57 mmol) (e.g. available from Aldrich). The solution was made up to 10 ml with THF. Titanium (IV) ethoxide (0.75 g, 3.38 mmol) was added and the reaction mixture was heated at 75° for 2 hours. The reaction mixture was cooled and poured onto saturated brine, with vigorous stirring. Celite was added to the resulting suspension, which was filtered and washed with DCM. The organic phase was separated from the aqueous phase by passing through a hydrophobic frit. The DCM was evaporated. The residue was purified on a 50 g SPE cartridge, eluting first with a (9:1) mixture of cyclohexane and EtOAc and then with a (4:1) mixture of cyclohexane and EtOAc. Fractions containing the required product were combined and concentrated in vacuo to give Intermediate 20 (0.29 g) as a white solid. LCMS showed MH⁺=238; T_{RET}=3.43 min.

The following intermediates 21-36 were prepared in a similar manner from (S)-tert butyl sulphinamide and the appropriate commercially available aldehyde (substituted benzaldehyde):

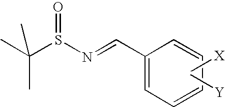

| Intermediate no. | 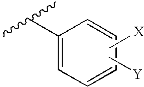 | MH⁺ ion | T_{RET} (min) | 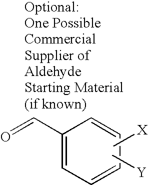 | Literature Reference to Intermediate (if known) |
|---|---|---|---|---|---|
| 21 | 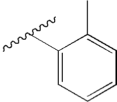 | 224 | 3.25 | Aldrich | |
| 22 | 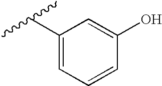 | 226 | 2.85 | Aldrich | |
| 23 | 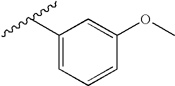 | 240 | 3.06 | Aldrich | |
| 24 | 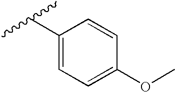 | 240 | 3.03 | Aldrich | Tetrahedron, 1999, 55, 8883-8904 |
| 25 | 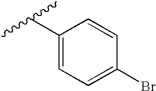 | 287 & 289 | 3.36 | Aldrich | Tetrahedron Asymm.; 2002, 13, 303-310 |
| 26 | 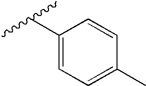 | 224 | 3.2 | Aldrich | |
| 27 | 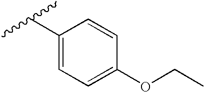 | 254 | 3.32 | Aldrich | |
| 28 | 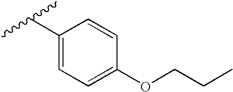 | 269 | 3.31 | Aldrich | |
| 29 | 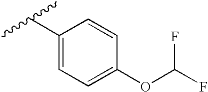 | 276 | 3.27 | Fluorochem Ltd. | |
| 30 | 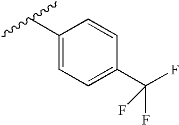 | 278 | 3.46 | Aldrich | J. Org. Chem; 2003, 68, 6894-6898 |
| 31 | 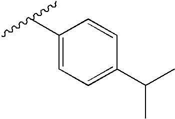 | 252 | 3.53 | Aldrich | |
| 32 | 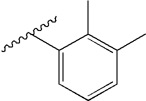 | 238 | 3.40 | Aldrich | |
| 33 | 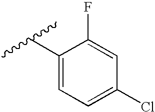 | 262 | 3.42 | Acros Organics | |
| 34 | 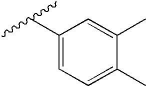 | 239 | 3.41 | Lancaster | |
| 35 | 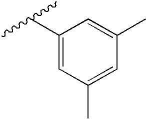 | 238 | 3.38 | Lancaster | |
| 36 | 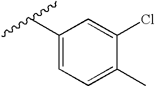 | 258 | 3.56 | Aldrich | |

### Intermediate 37: N-[-1(2,4-dimethylphenyl)ethyl]-2-methyl-2-propanesufinamide

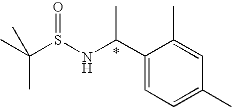

A 3.0 Molar solution of methyl magnesium bromide in Et₂O (2.6 ml) was added dropwise, with stirring, to a solution of Intermediate 20 (0.14 g, 0.59 mmol) in dry THF (5 ml) at −10° C. The reaction mixture was stirred at −10° C. for 3 hours then gradually warmed to 20° C. over 24 hours. The reaction mixture was cooled to 0° C. and treated, dropwise, with saturated ammonium chloride, with vigorous stirring. Once effervescence had ceased more ammonium chloride (5 ml) was added, followed by DCM (30 ml). The reaction mixture was stirred for 30 min. then the organic phase was filtered through a hydrophobic frit. The DCM was evaporated to leave Intermediate 37 (0.15 g) as a white solid (mixture of diastereoisomers, believed to be enriched in a diastereoisomer which is believed to have the (R)-stereochemistry at the benzylic carbon atom). LCMS showed MH⁺=254; T_{RET}=3.13 min.

The following Intermediates 38-61 were prepared in a similar manner from Intermediates 20-36, using either a 3.0 Molar solution of methylmagnesium bromide in diethyl ether (R⁴=Me) or a 3.0 Molar solution of ethylmagnesium bromide in diethyl ether (R⁴=Et):

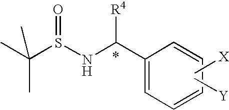

(believed to be enriched in a diastereoisomer which is believed to have the (R)-stereochemistry at the benzylic carbon atom)

| Intermediate no. | R⁴ | 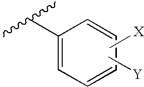 | Precursor | MH⁺ ion | T_{RET} (min) | Reference (if known) |
|---|---|---|---|---|---|---|
| 38 | Me | 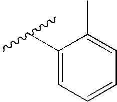 | Intermediate 21 | 240 | 2.95 | |
| 39 | Me | 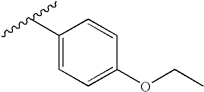 | Intermediate 27 | 270 | 2.97 | |
| 40 | Me | 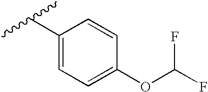 | Intermediate 29 | 292 | 3.00 | |
| 41 | Me | 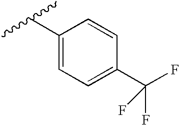 | Intermediate 30 | 294 | 3.17 | |
| 42 | Me | 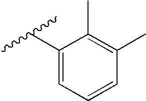 | Intermediate 32 | 254 | 3.10 | |
| 43 | Me | 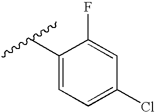 | Intermediate 33 | 278 | 3.16 | |
| 44 | Me | 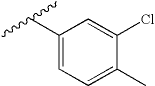 | Intermediate 34 | 274 | 3.25 | |
| 45 | Et | 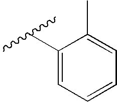 | Intermediate 21 | 254 | 3.10 | |
| 46 | Et | 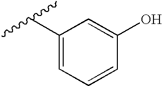 | Intermediate 22 | 256 | 2.56 & 2.69 | |
| 47 | Et | 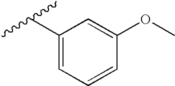 | Intermediate 23 | 270 | 2.86 & 2.94 | |
| 48 | Et | 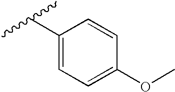 | Intermediate 24 | 270 | 2.86 & 2.93 | Tetrahedron, 1999, 55, 8883-8904 |
| 49 | Et | 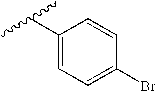 | Intermediate 25 | 317 & 319 | 3.17 | |
| 50 | Et | 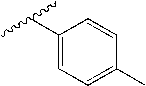 | Intermediate 26 | 254 | 3.14 | |
| 51 | Et | 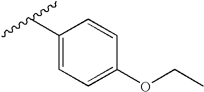 | Intermediate 27 | 284 | 3.16 | |
| 52 | Et | 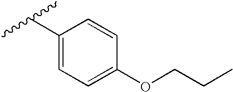 | Intermediate 28 | 298 | 3.24 & 3.28 | |
| 53 | Et | 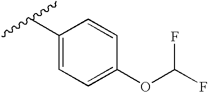 | Intermediate 29 | 306 | 3.18 | |
| 54 | Et | 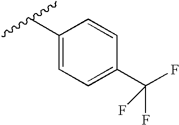 | Intermediate 30 | 308 | 3.30 | |
| 55 | Et | 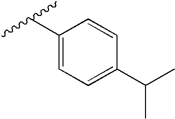 | Intermediate 31 | 282 | 3.43 | |
| 56 | Et | 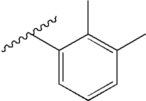 | Intermediate 32 | 268 | 3.24 | |
| 57 | Et | 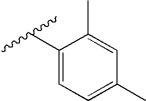 | Intermediate 20 | 268 | 3.28 | |
| 58 | Et | 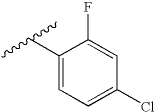 | Intermediate 33 | 292 | 3.30 | |
| 59 | Et | 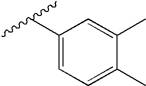 | Intermediate 34 | 268 | 3.26 & 3.31 | |
| 60 | Et | 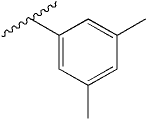 | Intermediate 35 | 268 | 3.28 & 3.33 | |
| 61 | Et | 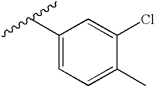 | Intermediate 36 | 288 | 3.3 | |

### Separation of the diastereoisomers of Intermediate 57

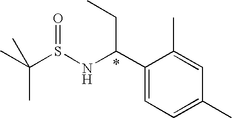

The mixture of diastereoisomers (Intermediate 57: 3 g) were purified by short path chromatography on silica, using cyclohexane containing 10-50% ethyl acetate as the eluent, to give the two diastereoisomers of Intermediate 57, as follows:

### Intermediate 57a (Diastereoisomer 1)

Isolated yield=322 mg (minor diastereomer, believed to have the (S)-stereochemistry at the benzylic carbon atom).

LCMS showed MH⁺=268; T_{RET}=3.23 min.

### Intermediate 57b (Diastereoisomer 2)

Isolated yield=1.76 g (major diastereomer, believed to have the (R)-stereochemistry at the benzylic carbon atom).

LCMS showed MH⁺=268; T^{RET}=3.23 min.

See Tim Tec Building Blocks B for the racemate of the following Intermediate 62:

### Intermediate 62: 1-(2,4-dimethylphenyl)ethyl]amine hydrochloride

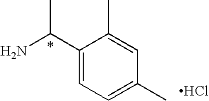

(Believed to be a mixture of enantiomers with the major enantiomer believed to have the (R)-stereochemistry)

A solution of Intermediate 37 (151 mg, 0.60 mmol) in a mixture of 4.0M hydrogen chloride in dioxan (1 ml) and MeOH (1 ml) was left to stand for 1 hour. The solvents were evaporated. The residue was triturated in Et₂O containing a few drops of MeOH to give a solid suspension. The solid was filtered off and dried to give Intermediate 62 (76 mg) as a white solid. LCMS showed MH⁺=150; T_{RET}=1.84 min.

The following Intermediates 63-86 were prepared in a similar manner from Intermediates 38-61:

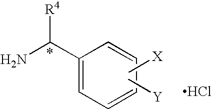

(Except for Intermediates 82a and 82b, Intermediates 63-86 are believed to be a mixture of enantiomers with the major enantiomer believed to have the
(R)-stereochemistry)

| Intermediate no. | R⁴ | 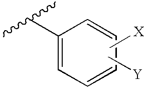 | Precursor | MH⁺ ion | T_{RET} (min) | Publication Reference to or One Possible Commercial Supplier of Intermediate (if known): reference may be made to the racemate and/or the (R)-enantiomer |
|---|---|---|---|---|---|---|
| 63 | Me | 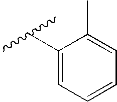 | Intermediate 38 | 136 | 1.33 | ACB Blocks Product List |
| 64 | Me | 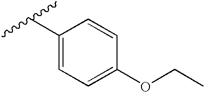 | Intermediate 39 | [MH − 16] = 149 | 1.77 | ACB Blocks Product List |
| 65 | Me | 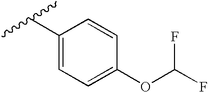 | Intermediate 40 | 188 | 1.65 | Braz. Pedido Pl; 1989, BR8804596 |
| 66 | Me | 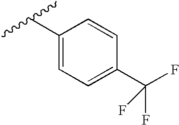 | Intermediate 41 | 190 | 1.88 | ACB Blocks Product List |
| 67 | Me | 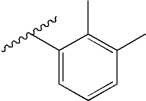 | Intermediate 42 | 150 | 1.81 | Agr. And Biol. Chem; 1973, 37, 981-988 |
| 68 | Me | 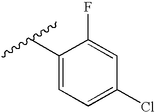 | Intermediate 43 | 174 | 1.60 | |
| 69 | Me | 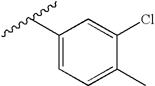 | Intermediate 44 | 169 | 1.95 | European Patent Application EP191496 A2 (1986) |
| 70 | Et | 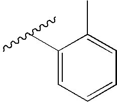 | Intermediate 45 | 150 | 1.81 | Tetrahedron Lett.; 1986, 27, 1331-1334 |
| 71 | Et | 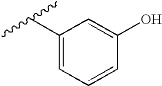 | Intermediate 46 | 152 | 1.16 | |
| 72 | Et | 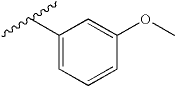 | Intermediate 47 | 166 | 1.69 | PCT Patent Appl. WO2002083624 (2002) |
| 73 | Et | 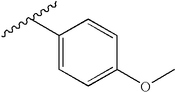 | Intermediate 48 | 166 | 1.67 | Tetrahedron Lett.; 1998, 39, 3559-3562 |
| 74 | Et | 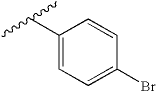 | Intermediate 49 | 214 & 216 | 1.9 | Synthesis, 1999, 930-934 |
| 75 | Et | 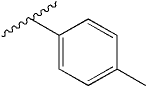 | Intermediate 50 | 150 | 1.78 | Tetrahedron Asymm.; 1999, 10, 1579-1588 |
| 76 | Et | 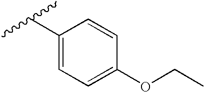 | Intermediate 51 | [M − 16] = 163 | 1.96 | |
| 77 | Et | 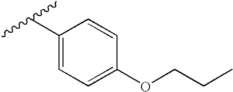 | Intermediate 52 | 194 | 2.07 | |
| 78 | Et | 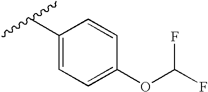 | Intermediate 53 | 202 | 1.95 | Pesticide Sci; 1998, 54, 223 |
| 79 | Et | 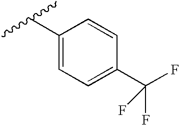 | Intermediate 54 | 204 | 2.12 | PCT Patent Appl. WO2002051809 (2002) |
| 80 | Et | 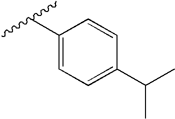 | Intermediate 55 | 178 | 2.1 | |
| 81 | Et | 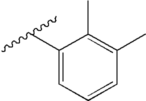 | Intermediate 56 | 164 | 2.01 | |
| 82 | Et | 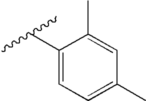 | Intermediate 57 | 164 | 2.04 | |
| 82a | Et | 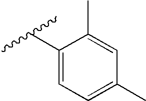 | Intermediate 57a (Diastereo- isomer 1) | | | Intermediate 82a enantiomer is believed to have the (S)- stereochemistry at the benzylic carbon atom |
| 82b | Et | 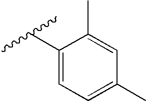 | Intermediate 57b (Diastereo- isomer 2) | | | Intermediate 82b enantiomer is believed to have the (R)- stereochemistry at the benzylic carbon atom |
| 83 | Et | 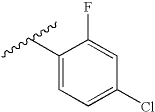 | Intermediate 58 | 188 | 1.93 | |
| 84 | Et | 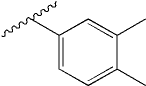 | Intermediate 59 | 164 | 2.00 | PCT Patent Appl. WO2002083624 (2002) |
| 85 | Et | 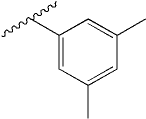 | Intermediate 60 | 164 | 2.04 | PCT Patent Appl. WO2002083624 (2002) |
| 86 | Et | 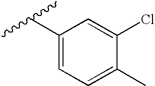 | Intermediate 61 | 185 | 2.13 | |

### Intermediate 87: [1-(3,5-dimethylphenyl)ethyl]amine hydrochloride (Jpn. Kokai Tokkyo Koho JP 62294669 (1987))

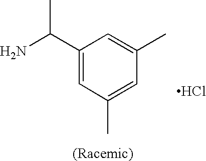

A mixture of (3,5-dimethyl)acetophenone (0.95 g, 7.0 mmol) (e.g. available from Lancaster Synthesis), formamide (1.4 ml, 1.58 g, 35.0 mmol) and formic acid (0.81 ml, 0.97 g, 21.0 mmol) was heated at 160° for 18 hours. The reaction mixture was cooled and partitioned between EtOAc and water. The organic phase was separated, washed with potassium carbonate solution and sodium chloride solution, dried over Na₂SO₄ and concentrated in vacuo. The residue was treated with 2M hydrochloric acid (10 ml) and the resultant mixture was heated at reflux for 18 hours, cooled to room temperature and washed with DCM (2×10 ml). The aqueous solution was concentrated in vacuo to leave Intermediate 87 (0.42 g) as a white solid. LCMS showed MH⁺=150; T_{RET}=1.88 min.

The following racemic Intermediates 88-99 were made in a similar manner from the appropriate acetophenone derivative, i.e. compound X—C(O)—Ar where Ar is optionally substituted phenyl or phenyl fused to C5-6cycloalkyl and X is R⁴ or R⁵ (commercially available unless stated):

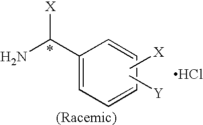

| Intermediate no. | X | 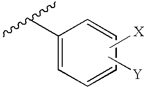 | Precursor (and one Possible Commercial Supplier- Optional) | MH⁺ion | T_{RET}(min) | Publication Reference to or One Possible Commercial Supplier of Intermediate (if known): reference |
|---|---|---|---|---|---|---|
| #may be made to the racemate and/or the (R)- enantiomer | | | | | | |
| 88 | Me | 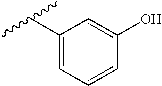 | 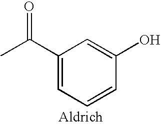 | 138 | 2.29 | Tetrahedron, 1977, 33, 489 |
| 89 | Me | 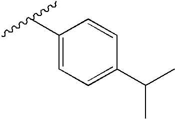 | 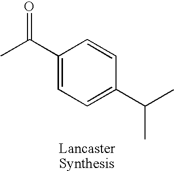 | 164 | 2.04 | Tim Tec Building Blocks B |
| 90 | Me | 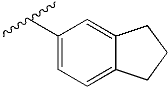 | 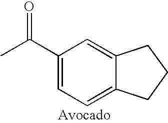 | 162 | 1.91 | Jpn. Kokai Tokkyo Koho JP 07101939 A2 (1995) |
| 91 | Me | 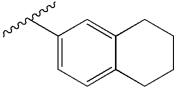 | 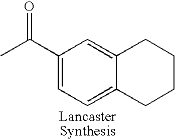 | 176 | 2.13 | Jpn. Kokai Tokkyo Koho JP 07101939 A2 (1995) |
| 92 | CF₃ | 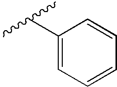 | 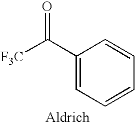 | 176 | 1.55 | Microchemistry Building Blocks |
| 93 | CF₃ | 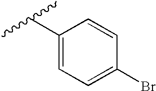 | 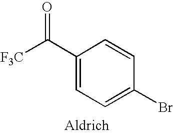 | 255 | 2.53 | Angew. Chem. Int. Ed; 2001, 40, 589-590 |
| 94 | CF₃ | 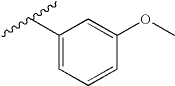 | 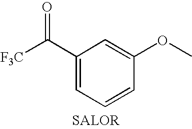 | 206 | 1.94 | |
| 95 | —(CH₂)₄CH₃ | 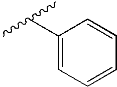 | 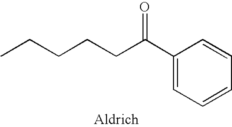 | 178 | 2.24 | J. Combinatorial Chem; 2001, 3, 71-77 |
| 96 | —(CH₂)₃CH₃ | 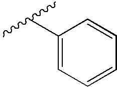 | 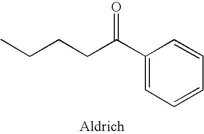 | 164 | 2.00 | Civentichem. |
| 97 | 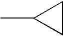 | 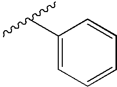 | 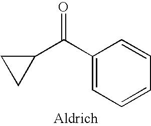 | 148 | 0.90 | ACB Blocks |
| 98 | —CH(CH₃)₂ | 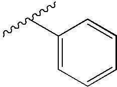 | 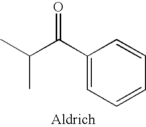 | 150 | 1.71 | Civentichem. |
| 99 | —(CH₂)₂CH₃ | 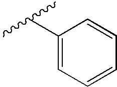 | 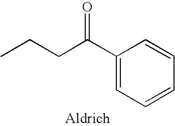 | 150 | 1.79 | Heterocyclic Compounds Catalog |

### Intermediates 100-101: [1-(2,4-dimethylphenyl)ethyl]amine trifluoroacetate

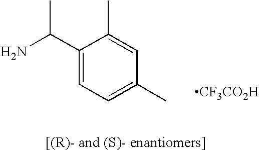

Intermediate 62 (0.40 g) was resolved by preparative chiral column chromatography, using a 2-inch×20 cm ChiralCel OJ column with a (2:98) mixture of heptane and ethanol, containing 0.1% trifluoroacetic acid, as the eluent. Intermediate 100 (first enantiomer to elute: 0.21 g) and Intermediate 101 (second enantiomer to elute: 0.12 g) were separated on the column. LCMS showed MH⁺=150; T_{RET}=1.76 min. for both enantiomers.

### Intermediate 102: Ethyl 4-[(1-{[(1,1-dimethylethyl)oxy]carbonyl}-4-piperidinyl)amino]-1-ethyl-1H-pyrazolo[3,4-b]pyridine-5-carboxylate

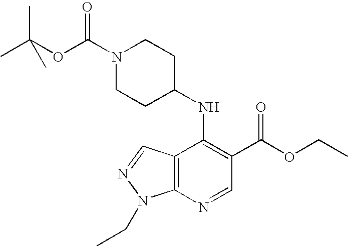

A solution of Intermediate 1 (2.3 g) in acetonitrile (50 ml) was treated with solid 1,1-dimethylethyl 4-amino-1-piperidinecarboxylate (2 g, e.g. available from AstaTech) and DIPEA (8.6 ml). The reaction mixture was heated at 90° C. for 16 h. The solvents were removed under reduced pressure and the residue was partitioned between DCM (100 ml) and water (75 ml). The organic fraction was collected through a hydrophobic frit and the solvents were removed under reduced pressure to yield Intermediate 102 as a white solid (3.9 g). LCMS showed MH⁺=418; T_{RET}=3.35 min.

### Intermediate 103: Ethyl 1-ethyl-4-(4-piperidinylamino)-1H-pyrazolo[3,4-b]pyridine-5-carboxylate hydrochloride

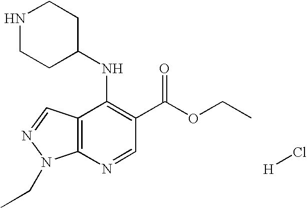

Intermediate 102 (3.9 g) was treated with 4.0 M hydrogen chloride in 1,4-dioxane (30 ml) and the reaction mixture was stirred at 22° C. for 1 h. The solvents were removed to give Intermediate 103 as a white solid (3.9 g). LCMS showed MH⁺=318; T_{RET}=2.21 min.

### Intermediate 104: Ethyl 4-{[1-(aminocarbonyl)-4-piperidinyl]amino}-1-ethyl-1H-pyrazolo[3,4-b]pyridine-5-carboxylate

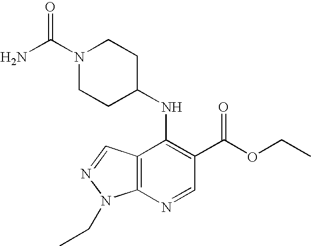

A suspension of Intermediate 103 (3.9 g) in THF (100 ml) was treated with trimethylsilyl isocyanate (1.99 ml) followed by DIPEA (2.6 ml) and the solution was stirred at 22° C. for 2 h. The volatile solvents were removed under reduced pressure and the residue was partitioned between DCM (50 ml) and water (25 ml). The organic layer was collected. The aqueous phase was re-extracted with DCM (50 ml). The organic layers were combined, separated from water by passing through a hydrophobic frit and concentrated under reduced pressure to yield Intermediate 104 as a white solid (3.9 g). LCMS showed MH⁺=361; T_{RET}=2.45 min.

### Intermediate 105: 4-{[1-(aminocarbonyl)-4-piperidinyl]amino}-1-ethyl-1H-pyrazolo[3,4-b]pyridine-5-carboxylic acid

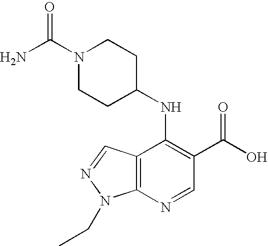

A solution of Intermediate 104 (3.9 g) in ethanol (50 ml) was treated with a solution of sodium hydroxide (1.77 g) in water (20 ml) and the reaction mixture was heated at 80° C. for 16 h. LCMS indicated that partial hydrolysis of the urea portion had occurred. The solvents were removed and the residue was dissolved in water (5 ml), the pH was adjusted to 3 (2M HCl) and the resultant white precipitate was collected by filtration and dried. This precipitate was dissolved in ethanol. The solution was treated with trimethylsilyl isocyanate (3 ml) and DIPEA (10 ml) and then stirred at 22° C. for 16 h. The solvents were removed and the residue was dissolved in water (5 ml), the pH was adjusted to 3 (2M HCl) and the resultant white precipitate was collected by filtration and dried to give Intermediate 105 as a white solid (2.66 g). LCMS showed MH⁺=333; T_{RET}=2.00 min.

### Intermediate 106: 4-chloro-1-ethyl-1H-pyrazolo[3,4-b]pyridine-5-carboxylic acid

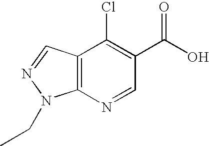

A solution of Intermediate 1 (20 g) in 1,4-dioxane (100 ml) was treated with a solution of potassium hydroxide (18 g) in water (30 ml) and the reaction mixture was stirred at 22° C. for 24 h. The solvent was evaporated and the residue was acidified to pH 3 (2M HCl). The resultant white precipitate was collected by filtration and dried to give Intermediate 106 as a white solid (16.9 g). LCMS showed MH⁺=226; T_{RET}=2.45 min.

Alternative synthesis: A solution of Intermediate 1 (3.5 g) in dioxane (28 ml) was treated with potassium hydroxide (6.3 g) as a solution in water (20 ml). The mixture was stirred for 2 h, then concentrated in vacuo, acidified to pH 3 with 2M aqueous hydrochloric acid and extracted with ethyl acetate. The layers were separated, the organic layer dried over sodium sulphate, then concentrated in vacuo to afford Intermediate 106 as a white solid (2.4 g). LCMS showed MH⁺=226; T_{RET}=2.62 min.

### Intermediate 107: 4-chloro-1-ethyl-1H-pyrazolo[3,4b]pyridine-5-carbonyl chloride

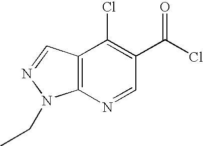

A solution of Intermediate 106 (17.8 g) in thionyl chloride (100 ml) was heated under reflux for 3.5 h. The solution was cooled to room temperature. The thionyl chloride was removed in vacuo and any remaining thionyl chloride was removed by azeotropic distillation with toluene (30 ml) to give Intermediate 107 as a beige solid (16.8 g). LCMS (MeOH solution) showed MH⁺=240 (Methyl ester); T_{RET}=2.88 min.

### Intermediate 108: 4-chloro-1-ethyl-N-[(1R)-1-(4-methylphenyl)ethyl]-1H-pyrazolo[3,4-b]pyridine-5-carboxamide

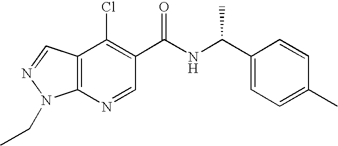

A solution of Intermediate 107 (2.0 g) in THF (20 ml) was treated with (R)-(+)-1-(4-methylphenyl) ethylamine (1.11 g) (e.g. available from Lancaster Synthesis) and DIPEA (1.06 g). The reaction mixture was stirred at 22° C. for 24 h. The solvent was evaporated and the residue was dissolved in DCM (50 ml). The solution was washed with 5% citric acid solution (50 ml) and 0.5M sodium bicarbonate solution (50 ml), dried (Na₂SO₄), filtered and concentrated to give Intermediate 108 as a white solid (1.61 g). LCMS showed MH⁺=343; T_{RET}=3.22 min.

The following Intermediate 109 was prepared in an analogous manner, suitably from (R)-(+)-1-phenylethylamine (e.g. available from Aldrich):

Intermediate 109: 4-chloro-1-ethyl-N-[(1R)-1-phenylethyl]-1H-pyrazolo[3,4-b]pyridine-5-carboxamide

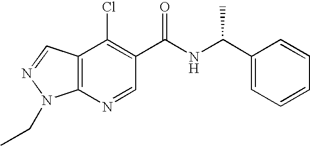

LCMS showed MH⁺=329; T_{RET}=3.0 min.

Intermediate 110: 1,1-dimethylethyl [1-(aminocarbonyl)-4-piperidinyl]carbamate

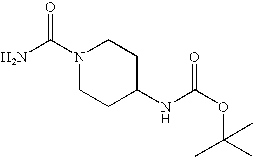

A solution of 1,1-dimethylethyl 4-piperidinylcarbamate (0.35 g, e.g. available from Syngene or AstaTech) in DCM (10 ml) was treated with trimethylsilyl isocyanate (1.1 ml). The reaction mixture was stirred at 22° C. for 72 h. The mixture was diluted with saturated NaHCO₃ solution (20 ml). The organic phase was collected through a hydrophobic frit and evaporated to give Intermediate 110 as a white foam (0.29 g). ¹H NMR (400 MHz in CDCl₃, 27° C., δ ppm) 4.45 (br. s, 3H). 3.90 (d, 2H), 3.65 (br. m, 1H), 2.9-3.0 (dt, 2H), 1.95-2.0 (br. dd, 2), 1.45 (s, 9), 1.3-1.4 (dq, 2H).

### Intermediate 111: 4-amino-1-piperidinecarboxamide hydrochloride

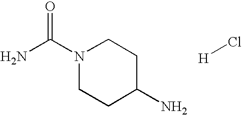

A solution of intermediate 110 (0.29 g) in 4.0M hydrogen chloride in 1,4-dioxane (5 ml) was stirred at 22° C. for 4 h. The solvent was evaporated to give Intermediate 111 as a white foam (0.27g). ¹H NMR (400 MHz in d₆-DMSO, 27° C., δ ppm) 8.1 (br. s, 2H), 3.95 (d, 2), 3.15 (m, 1H), 2.7 (dt, 2H), 1.85 (dd, 2H), 1.35 (m, 2H).

### Intermediate 112: 1,1-dimethylethyl [4-(aminocarbonyl)cyclohexyl]carbamate

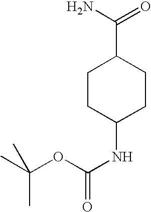

A solution of 4-({[(1,1-dimethylethyl)oxy]carbonyl}amino)cyclohexanecarboxylic acid (from Fluka, 1 g) in DMF (30 ml) was treated with HATU (1.72 g) and DIPEA (5.4 ml). The reaction mixture was stirred at 22° C. for 10 min. A 0.5M solution of ammonia in 1,4-dioxane (40 ml) was added and the reaction mixture was stirred at 22° C. for 72 h. The solvents were evaporated and the residue was purified by loading the crude mixture onto a 50 g aminopropyl SPE cartridge and eluting with ethyl acetate (100 ml), then methanol (100 ml). Intermediate 112 was isolated by evaporation of the methanol fraction as a yellow oil (0.99 g). LCMS showed MH⁺=242; T_{RET}=2.2 min.

### Intermediate 113: 4-aminocyclohexanecarboxamide hydrochloride

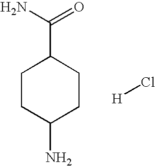

4.0M hydrogen chloride in 1,4-dioxane (14 ml) was added to Intermediate 112 (0.99 g) and the reaction mixture was stirred at 22° C. for 30 min. The solvent was evaporated to give Intermediate 113 as a yellow gum (1.03 g). ¹H NMR (400 MHz in d₆-DMSO, 27° C., δppm) 7.9 (br. S, 2H), 3.9 (br. S, 2H), 3.10 (m, 1H), 1.92 (m, 2H), 1.68 (m, 4H), 1.50 (m, 2H).

### Intermediate 114: 1,1-dimethylethyl [cis-4-(aminocarbonyl)cyclohexyl]-carbamate

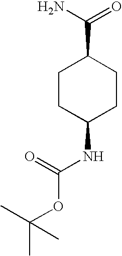

A solution of cis-4-({[(1,1-dimethylethyl)oxy]carbonyl}amino)cyclohexane-carboxylic acid (5.0 g) (e.g. available from Fluka), EDC (5.9 g) and HOBT (4.17 g) was stirred for 20 min. Ammonia solution (Specific Gravity=0.88; 8 ml) was added. The reaction mixture was stirred at room temperature overnight, concentrated in vacuo and partitioned between DCM and saturated sodium bicarbonate solution. The aqueous phase was separated and washed with DCM. The combined organics were dried over MgSO₄ and concentrated in vacuo to give Intermediate 114 (4.84 g) as a white solid. LCMS showed MH⁺=243; T_{RET}=2.3 min.

The following Intermediate 115 was prepared in a similar manner from trans-4-({[(1,1-dimethylethyl)oxy]carbonyl}amino)cyclohexanecarboxylic acid (e.g. available from Fluka):

### Intermediate 115: 1,1-dimethylethyl [trans-4-(aminocarbonyl)cyclohexyl]-carbamate

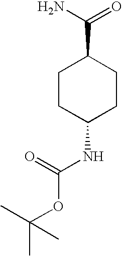

LCMS showed MNH₄⁺=260; T_{RET}=2.24 min.

### Intermediate 116: cis-4-aminocyclohexanecarboxamide hydrochloride

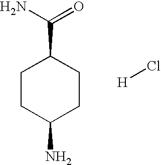

4.0M HCl in dioxan (50 ml) was added to a stirred solution of Intermediate 114 (4.84 g) in dioxan (100 ml). The reaction mixture was stirred for 1 hour at room temperature and then left at 0° C. for 3 days. The reaction mixture was concentrated in vacuo to give Intermediate 116 (4.1 g) as a white solid. LCMS showed MH⁺=143; T_{RET}=0.31 min.

The following Intermediate 117 was prepared in a similar manner from Intermediate 115:

### Intermediate 117: trans-4-aminocyclohexanecarboxamide hydrochloride

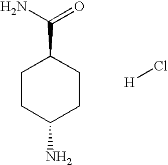

LCMS showed MH⁺=143; T_{RET}=0.30 min.

### Intermediate 118: ethyl 4-{[cis-4-(aminocarbonyl)cyclohexyl]amino}-1-ethyl-1H-pyrazolo[3,4-b]pyridine-5-carboxylate

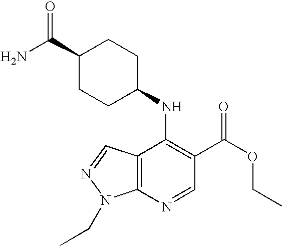

A solution of Intermediate 1 (2.0 g), Intermediate 116 (1.55 g) and DIPEA (6.9 ml) in ethanol (140 ml) was stirred and heated at reflux overnight. More of Intermediate 116 (420 mg) and DIPEA (3.5 ml) were added. The reaction mixture was stirred and heated at reflux overnight, cooled and concentrated in vacuo. The residue was partitioned between DCM and saturated sodium bicarbonate solution. The organic phase was concentrated in vacuo. The residue was triturated in a mixture of DCM and cyclohexane to give a solid. The solid was filtered off and dried to give Intermediate 118 (2.16 g) as a yellow solid. LCMS showed MH⁺=360; T_{RET}=2.56 min.

The following Intermediate 119 was prepared in a similar manner from Intermediate 1 and Intermediate 117:

### Intermediate 119: ethyl 4-{[trans4-(aminocarbonyl)cyclohexyl]amino}-1-ethyl-1H-pyrazolo[3,4-b]pyridine-5-carboxylate

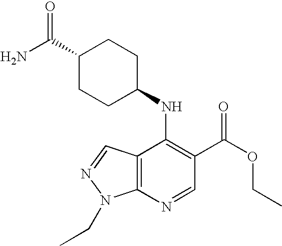

LCMS showed MH⁺=360; T_{RET}=2.84 min.

### Intermediate 120: 4-{[cis-4-(aminocarbonyl)cyclohexyl]amino)-1-ethyl-1H-pyrazolo[3,4-b]pyridine-5-carboxylic acid

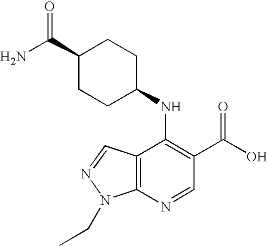

A mixture of Intermediate 118 (1.54 g) and sodium hydroxide (0.68 g) in 95% aqueous EtOH (EtOH containing 5% water) (60 ml) was stirred and heated at 50° C. overnight. The solvent was removed in vacuo. The residue was dissolved in water. The solution was cooled to 0-5° C., with stirring, and acidified with 2M HCl. The resultant suspension was refrigerated for 3 days then filtered under suction. The residue was dried in a vacuum oven to give Intermediate 120 (1.58 g) as a yellow solid. LCMS showed MH⁺=332; T_{RET}=2.06 min.

The following Intermediate 121 was prepared in an analogous manner from Intermediate 1 and Intermediate 119:

### Intermediate 121: 4-{[trans-4-(aminocarbonyl)cyclohexyl]amino}-1-ethyl-1H-pyrazolo[3,4-b]pyridine-5-carboxylic acid

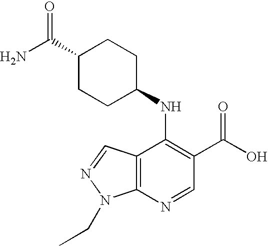

LCMS showed MH⁺=332; T_{RET}=2.06 min.

### Intermediate 122: 4-chloro-N-[1-(2,4-dimethylphenyl)propyl]-1-ethyl-1H-pyrazolo[3,4-b]pyridine-5-carboxamide

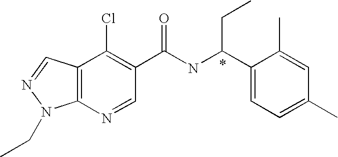

(Believed to be a mixture of enantiomers with the major enantiomer believed to have the (R)-stereochemistry)

Prepared from Intermediates 82 and 107 using a method analogous to that used to make Intermediate 108.

LCMS showed MH⁺=371; T_{RET}=3.32 min.

### Intermediates 123 to 145, 50a, 55a, 58a. 75a, 80a and 83a

Like Intermediates 20-86, these intermediates were prepared using a modification of the procedure developed by D. A. Cogan, G. Liu and J. Elhman and described in Tetrahedron, 1999, 55, 8883-8904. In the Cogan, Liu, Eliman paper, the use of (S)-tert butyl sulphinamide in chemistry similar to that described in Intermediates 123-127 and 128-136 below allegedly produced an enrichment in a diastereoisomer with the general stereochemistry at the carbon atom next to the nitrogen shown here: 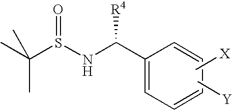
(i.e. inserted group R4 into the paper as shown, branched-benzyl is illustrative example only); this stereochemistry (R4 into the paper) was formed in the carbon-carbon bond forming reaction (i.e. before any optional separation of diastereoisomers). As the process of Intermediates 128-136, 50a, 55a and 58a herein includes an additional step separating the diastereomers, the compounds containing an alpha substituent on the benzylic carbon atom (Intermediates 128 to 136, 50a, 55a and 58a, and Intermediates 137 to 145, 75a, 80a and 83a) are believed to consist essentially of an enantiomer/diastereoisomer which is believed to have the (R)-stereochemistry at the benzylic carbon atom.

### Intermediates 123 to 127

The following Intermediates 123 to 127 were prepared from (S)-tert butyl sulphinamide and the appropriate commercially available aldehyde (substituted benzaldehyde), by adopting a similar method to that used to prepare Intermediate 20:

| Inter- medi- ate no. | 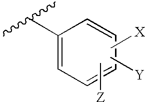 | MH⁺ion | T_{RET}(min) | 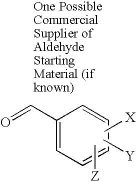 |
|---|---|---|---|---|
| 123 | 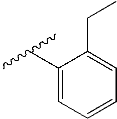 | | | Aldrich |
| 124 | 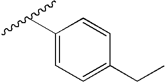 | 238 | 3.43 | Aldrich |
| 125 | 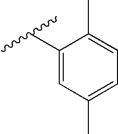 | 238 | 3.31 | Aldrich |
| 126 | 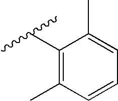 | 238 | 3.27 | Aldrich |
| 127 | 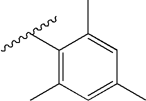 | 252 | 3.55 | Avocado Research |

### Intermediates 128 to 136, 50a, 55a and 58a

### Intermediate 128 Synthesis

A 3.0 Molar solution of methylmagnesium bromide in diethyl ether (3.8 ml) was added to a stirred solution of Intermediate 123 (0.91 g) in dry DCM (20 ml) at −78° C. The reaction mixture was stirred at −78° C. for 1 hour, warmed to room temperature and stirred at room temperature for 24 h. The reaction mixture was cooled again to −78° C. More 3.0 Molar methylmagnesium bromide solution in diethyl ether (1.9 ml) was added. The reaction mixture was stirred at −78° C. for 1 hour, warmed to room temperature and stirred at room temperature for 2 h, then cooled to 0° C. and treated dropwise with stirring with saturated ammonium chloride solution (10 ml) followed by DCM (20 ml). The organic phase was filtered through a hydrophobic frit. The DCM was evaporated. The residue was purified on a 50 g silica SPE cartridge, using cyclohexane containing a gradient of 0% to 100% ethyl acetate. The fractions containing the major diastereoisomer (e.g. can be eluted using 100% ethyl acetate) were combined and evaporated to give Intermediate 128 as a solid. LCMS showed MH⁺=254, T_{RET}=3.07 or 3.12.

The following Intermediates 129 to 136, 50a, 55a and 58a were prepared from Intermediates 124 to 127, 26, 31 or 33 in the same or a similar manner to that described above for Intermediate 128, using either a 3.0 Molar solution of methylmagnesium bromide in diethyl ether (R⁴=Me) or a 3.0 Molar solution of ethylmagnesium bromide in diethyl ether (R⁴=Et):

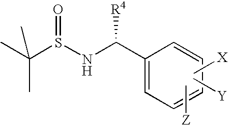

(Intermediates 128 to 136, 50a, 55a and 58a are believed to consist
essentially of an isomer believed to have the (R)-stereochemistry at the
benzylic carbon atom.)

| Inter- mediate no. | R⁴ | 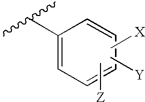 | Precursor | MH⁺ion | T_{RET}(min) |
|---|---|---|---|---|---|
| 128 | Me | 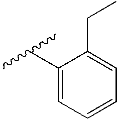 | Intermediate 123 | 254 | 3.12 |
| 129 | Me | 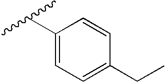 | Intermediate 124 | 254 | 3.15 |
| 130 | Me | 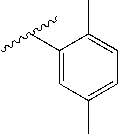 | Intermediate 125 | 254 | 3.11 |
| 131 | Me | 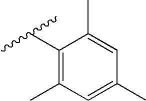 | Intermediate 127 | 268 | 3.21 |
| 132 | Et | 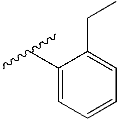 | Intermediate 123 | | |
| 133 | Et | 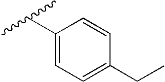 | Intermediate 124 | 268 | 3.27 |
| 134 | Et | 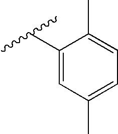 | Intermediate 125 | 268 | 3.17 |
| 135 | Et | 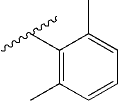 | Intermediate 126 | | |
| 136 | Et | 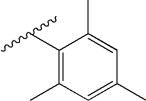 | Intermediate 127 | 282 | 3.33 |
| 50a | Et | 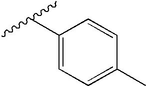 | Intermediate 26 | | |
| 55a | Et | 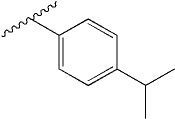 | Intermediate 31 | | |
| 58a | Et | 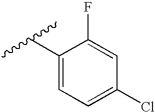 | Intermediate 33 | | |

### Intermediates 137 to 145, 75a, 80a and 83a

The following Intermediates 137 to 145, 75a, 80a and 83a were prepared, in a similar manner that described for the synthesis of Intermediate 62, from Intermediates 128 to 136, 50a 55a or 58a:

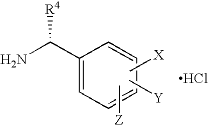

(Intermediates 137 to 145, 75a, 80a and 83a are believed to consist essentially of an enantiomer believed to have the
(R)-stereochemistry at the benzylic carbon atom.)

| Intermediate no. | R⁴ | 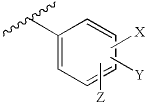 | Precursor | MH⁺ ion | T_{RET}(min) | Publication Reference to, or a Possible Commercial Supplier of, Intermediate (if known): reference may be made to the racemate and/or the (R)- enantiomer |
|---|---|---|---|---|---|---|
| 137 | Me | 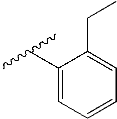 | Intermediate 128 | | | CAS 104338-67-2 (Chem. Abs. Service) |
| 138 | Me | 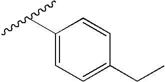 | Intermediate 129 | | | Tim Tec Overseas Stock Chembridge |
| 139 | Me | 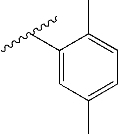 | Intermediate 130 | 150 | 1.84 | Tim Tec Overseas Stock |
| 140 | Me | 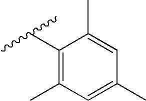 | Intermediate 131 | | | T. Kohara et. Al; Tetrahedron Asymmetry, 1999, 10, 4831-4840 |
| 141 | Et | 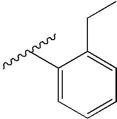 | Intermediate 132 | | | |
| 142 | Et | 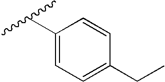 | Intermediate 133 | | | |
| 143 | Et | 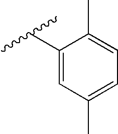 | Intermediate 134 | | | |
| 144 | Et | 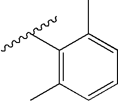 | Intermediate 135 | | | |
| 145 | Et | 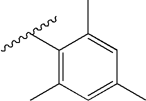 | Intermediate 136 | | | |
| 75a | Et | 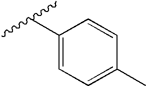 | Intermediate 50a | | | Tetrahedron Asymm.; 1999, 10, 1579-1588 |
| 80a | Et | 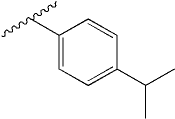 | Intermediate 55a | | | |
| 83a | Et | 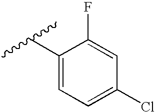 | Intermediate 58a | | | |

### Intermediate 146: ethyl 4-[((3S)-1-{[(1,1-dimethylethyl)oxylcarbonyl}-3-pyrrolidinyl)amino]-1-ethyl-1H-pyrazolo [3,4-b]pyridine-5-carboxylate

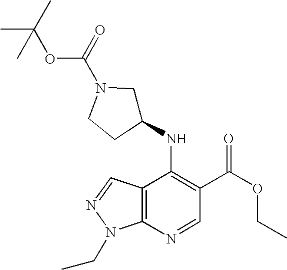

A solution of Intermediate 1 (680 mg), DIPEA (2.3 ml) and 1,1-dimethylethyl (3S)-3-amino-1-pyrrolidinecarboxylate (500 mg) (e.g. available from Aldrich) in MeCN (15 ml) was stirred and heated at reflux for 16 h. The solvent was evaporated and the residue was partitioned between DCM and water. The organic phase was isolated by passage through a hydrophobic frit. The solvent was evaporated and the residue was purified on a 100 g “flashmaster” cartridge (e.g. available from Jones Chromatography Ltd., United Kingdom), using a mixture of EtOAc and cyclohexane as the eluent, to give Intermediate 146 (720 mg) as a solid. LCMS showed MH⁺=404; T_{RET}=3.20 min.

The following Intermediate 147 was prepared in a similar manner from Intermediate 1 and 1,1-dimethylethyl (3R)-3-amino-1-pyrrolidinecarboxylate (e.g. available from Aldrich):

### Intermediate 147: ethyl 4-[((3R)-1-{[(1,1-dimethylethyl)oxy]carbonyl}-3-pyrrolidinyl)amino]-1-ethyl-1H-pyrazolo [3,4-b]pyridine-5-carboxylate

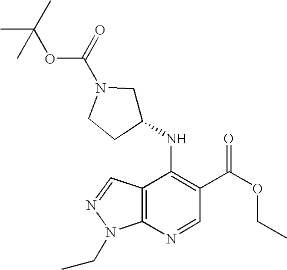

LCMS showed MH⁺=404; T_{RET}=3.20 min.

### Intermediate 148: ethyl 1-ethyl-4-[(3S)-3-pyrrolidinylamino]-1H-pyrazolo[3,4-b]pyridine-5-carboxylate hydrochloride

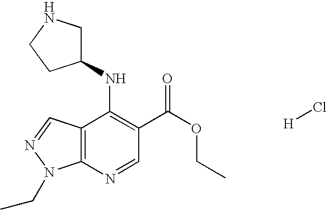

A solution of Intermediate 146 (720 mg) in 4.0 M hydrogen chloride in dioxan (30 ml) was stirred at 22° C. for 3 h. The solvent was evaporated to give Intermediate 148 (606 mg) as a white solid. LCMS showed MH⁺=304; T_{RET}=2.00 min.

The following Intermediate 149 was prepared in a similar manner from Intermediate 147:

### Intermediate 149: ethyl 1-ethyl4-[(3R)-3-pyrrolidinylamino]-1H-pyrazolo[3,4-b]pyridine-5-carboxylate hydrochloride

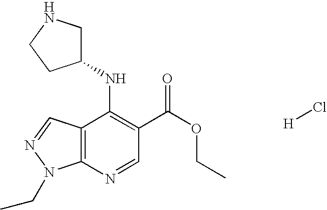

LCMS showed MH⁺=304; T^{RET}=2.00 min.

### Intermediate 150: ethyl 4-{[(3S)-1-(aminocarbonyl)-3-pyrrolidinyl]amino}-1-ethyl-1H-pyrazolo[3,4-b]pyridine-5-carboxylate

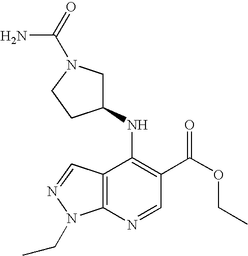

A solution of Intermediate 148 (606 mg) in DCM (30 ml) was stirred and treated with DIPEA (1.15 ml) followed by trimethylsilyl isocyanate (1.03 ml). The reaction mixture was stirred at 22° C. for 2 h. The solution was washed with water. The aqueous phase was extracted with dichloromethane. The combined organics were passed through a hydrophobic frit and then concentrated to give Intermediate 150 (660 mg) as a solid. LCMS showed MH⁺=347; T_{RET}=2.40 min.

The following Intermediate 151 was prepared in a similar manner from Intermediate 149:

### Intermediate 151: ethyl 4-{[(3R)-1-(aminocarbonyl)-3-pyrrolidinyl]amino}-1-ethyl-1H-pyrazolo[3,4-b]pyridine-5-carboxylate

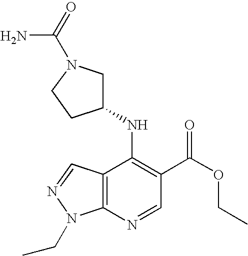

LCMS showed MH⁺=347; T_{RET}=2.40 min.

### Intermediate 152: 4-{[(3S)-1-(aminocarbonyl)-3-pyrrolidinyl]amino}-1-ethyl-1H-pyrazolo[3,4-b]pyridine-5-carboxylic acid

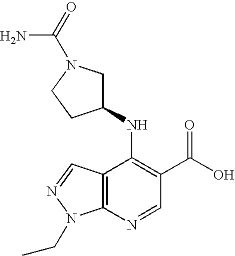

A mixture of Intermediate 150 (660 mg) and sodium hydroxide (300 mg) in ethanol (15 ml) and water (8 ml) was stirred and heated at 60° C. for 2 h. The solvents were removed in vacuo. Water (8 ml) was added to the residue and the resultant solution was acidified with 2M hydrochloric acid. The resultant suspension was filtered under suction. The residue was dried in vacuo to give Intermediate 152 (270 mg) as a solid. LCMS showed MH⁺=319; T_{RET}=1.90 min.

The following Intermediate 153 was prepared in a similar manner from Intermediate 151:

### Intermediate 153: 4-{[(3R)-1-(aminocarbonyl)-3-pyrrolidinyl]amino}-1-ethyl-1H-pyrazolo[3,4-b]pyridine-5-carboxylic acid

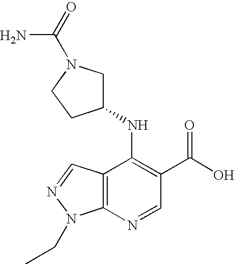

LCMS showed MH⁺=319; T_{RET}=1.90 min.

### Intermediate 154: 1,1-dimethylethyl (cis-4-{[methyl(methyloxy)amino]carbonyl}cyclohexyl)carbamate

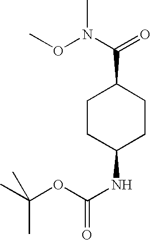

A solution of cis-4-({[(1,1-dimethylethyl)oxy]carbonyl}amino)cyclohexanecarboxylic acid (1.0 g) (e.g. available from Fluka), EDC (0.95 g), HOBT (0.61 g) and DIPEA (2.1 ml) in THF (60 ml) was stirred at 22° C. for 30 min then N,O-dimethylhydroxylamine hydrochloride (0.5 g) was added. The reaction mixture was stirred for 7 h. The solvent was removed and the residue was partitioned between DCM and saturated sodium bicarbonate solution. The organic phase was separated and the solvent was evaporated. The residue was applied to a 20 g SPE cartridge. The cartridge was eluted with cyclohexane containing 10-50% EtOAc to give Intermediate 154 (768 mg).

### Intermediate 155: 1,1-dimethylethyl (cis-4-acetylcyclohexyl)carbamate

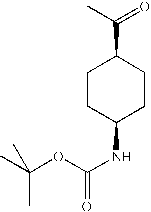

A solution of Intermediate 154 (768 mg) in THF (25 ml) was cooled to 0° C. A 3.0 Molar solution of methyhnagnesium bromide in diethyl ether (2.2 ml) was added rapidly dropwise over 5 min. The reaction mixture was stirred at 0-5° C. for 3 hours. More 3.0 Molar methylmagnesium bromide in diethyl ether (0.9 ml) was added. The reaction mixture was stirred at 0-5° C. overnight. 1M hydrochloric acid (20 ml) was added, dropwise. The reaction mixture was extracted with EtOAc. The organic extracts were dried over Na₂SO₄ and concentrated in vacuo. The residue was applied to a 10 g SPE cartridge. The cartridge was eluted with a (1:1) mixture of cyclohexane and EtOAc to give Intermediate 155 (340 mg).

### Intermediate 156: 1-(cis-4-aminocyclohexyl)ethanone hydrochloride

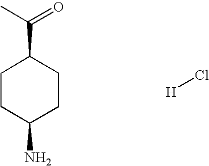

A stirred solution of Intermediate 155 (115 mg) in dioxan (1 ml) was treated with a 4M solution of hydrogen chloride in dioxan (240 μl). The reaction mixture was stirred at room temperature for 4 h then refrigerated overnight. The reaction mixture was concentrated i71 vacuo to give Intermediate 156 (72 mg) as a solid.

### Intermediate 157: ethyl 4-[(4-acetylcyclohexyl)amino]-1-ethyl-1H-pyrazolo[3,4-b]pyridine-5-carboxylate (mixture of cis and traits isomers)

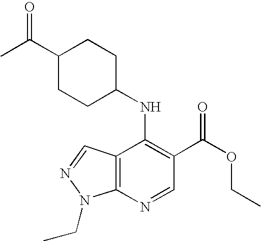

A solution of Intermediate 1 (93 mg), Intermediate 156 (72 mg) and DIPEA (0.32 ml) in EtOH (10 ml) was stirred and heated at reflux overnight. The solvent was evaporated and the residue was partitioned between DCM and saturated sodium bicarbonate solution. The organic phase was separated and concentrated. The residue was purified by mass directed autoprep HPLC to give Intermediate 157 (102 mg) as a mixture of cis and trans isomers. LCMS showed MH⁺=359; T_{RET}=3.05 min.

### Intermediate 158: 4-[(4-acetylcyclobexyl)amino]-1-ethyl-1H-pyrazolo[3,4-b]pyridine-5-carboxylic acid (mixture of cis and trans isomers)

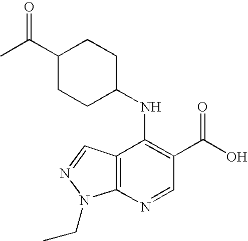

A solution of Intermediate 157 (102 mg) and sodium hydroxide (45 mg) in 95% aqueous EtOH was stirred and heated at 50° C. overnight. The solvents were removed in vacuo. Water was added to the residue and the resultant solution was acidified with 2M hydrochloric acid. The resultant suspension was filtered. The residue was dried in vacuo to give Intermediate 158. The aqueous filtrate was extracted with EtOAc and DCM. The organic extracts were combined and concentrated to give a further quantity of Intermediate 158. The overall yield of Intermediate 158 was 70 mg. LCMS showed MH⁺=331; T_{RET}=2.46 min.

### Intermediate 159: (RS)-1,1-dimethylethyl [cis-4-(1-hydroxyethyl)cyclohexyl]carbamate

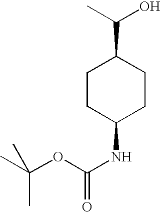

A 1.5 Molar solution of diisobutylaluminium hydride in toluene (0.77 ml) was added, dropwise, to a stirred solution of Intermediate 155 (112 mg) in TBF (5 ml) at 0-5° C. The reaction mixture was stirred and warmed to room temperature overnight. More diisobutylaluminium hydride in toluene (0.31 ml) was added. The reaction mixture was left at 22° C. over the weekend., then treated with saturated sodium potassium tartrate solution (15 ml). The mixture was stirred for 0.75 h, then extracted with EtOAc. The combined extracts were washed with saturated sodium chloride solution, dried over MgSO₄ and concentrated. The residue was applied to a 2 g SPE cartridge. The cartridge was eluted with cyclohexane containing 0-20% EtOAc to give Intermediate 159 (10 mg).

### Intermediate 160: (RS)-1-(cis-4-aminocyclohexyl)ethanol hydrochloride

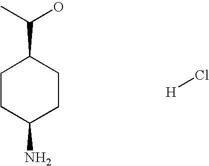

A solution of Intermediate 159 (10 mg) in dioxan (0.5 ml) was treated with a 4M solution of hydrogen chloride in dioxan (240 μl). The reaction mixture was stirred at room temperature for 5 h then left to stand overnight. The solvent was removed to give Intermediate 160 as a solid (7 mg).

### Intermediate 161: ethyl 1-ethyl-4-{[(1SR,3SR)-3-hydroxycyclohexyl]amino}-1H-pyrazolo[3,4-b]pyridine-5-carboxylate

### [traizs-(3-hydroxycyclohex-1-yl)amino group, racemic]

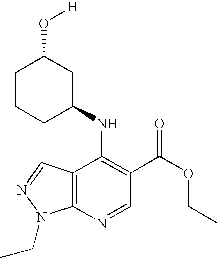

A solution of 3-aminocyclohexanol (mixture of cis and trans isomers, 4.25 g) (e.g. such a mixture is available from AB Chem, Inc., Canada; or see for example J. Chem. Soc., Perkin Trans 1, 1994, 537 for a 3.3: 1 cis : trans mixture of 3-aminocyclohexanol), Intermediate 1 (7.8 g) and DIPEA (25 ml) in MeCN(50 ml) and EtOH (5 ml) was stirred and heated at reflux for 16 h. The solvents were removed under reduced pressure and the residue was partitioned between DCM and water. The organic phase was concentrated and the residue was applied to a 100 g SPE cartridge. The cartridge was eluted with a (1:2) mixture of EtOAc and cyclohexane to give Intermediate 161 (trans isomer: 326 mg). LCMS showed MH⁺=333; T_{RET}=2.90 min.

### Intermediate 162: 1-ethyl-4-{[(1SR,3SR)-3-hydroxycyclohexyl]amino}-1H-pyrazolo[3,4-b]pyridine-5-carboxylic acid

### [trans-(3-hydroxycyclohex-1-yl)amino group, racemic]

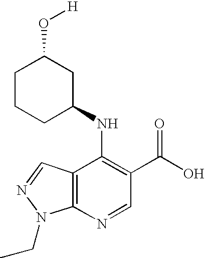

A mixture of Intermediate 161 (326 mg) and sodium hydroxide (156 mg) in water (2 ml) and EtOH (4.6 ml) was stirred and heated at 60° C. for 5 h then cooled and concentrated under reduced pressure. The residue was dissolved in water. The solution was acidified with 2M hydrochloric acid. The resultant suspension was filtered. The residue was dried in vacuo to give Intermediate 162 (270 mg) as a white solid. LCMS showed MH⁺=305; T_{RET}=2.21 min.

### Intermediate 163: 4-[(1-{[(1,1-dimethylethyl)oxy]carbonyl}-4-piperidinyl)amino]-1-ethyl-1H-pyrazolo[3,4-b]pyridine-5-carboxylic acid

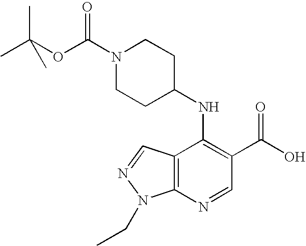

A mixture of Intermediate 102 (750 mg) and sodium hydroxide (290 mg) in EtOH (20 ml) and water (5 ml) was stirred and heated at 50° C. for 2.5 h then cooled and concentrated under reduced pressure. A solution of the residue in water (20 ml) was cooled to 0-5° C., with stirring, and acidified to pH=5 with 2M hydrochloric acid. The resultant solid suspension was filtered. The solid residue was washed with water and dried to give Intermediate 163 (575 mg) as a white solid. LCMS showed MH⁺=390; T_{RET}=2.86 min.

### Intermediate 164: 1,1-dimethylethyl 4-{[1-ethyl-5-({[(1R)-1-(4-methylphenyl)ethyl]amino}carbonyl)-1H-pyrazolo[3,4-b]pyridin-4-yl]amino}-1-piperidinecarboxylate

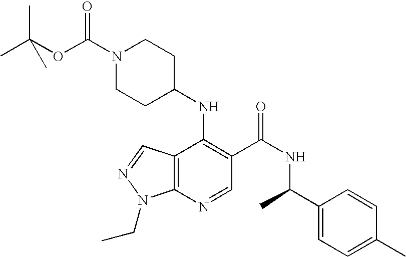

A solution of Intermediate 163 (100 mg), EDC (54 mg), HOBT (38 mg) and DIPEA (0.11 ml) in DMF (5 ml) was added to [(1R)-1-(4-methylphenyl)ethyl]amine (38 mg) (e.g. available from Lancaster). The solution was left to stand overnight. The solvent was evaporated. The residue was partitioned between DCM and saturated sodium bicarbonate solution. The organic phase was separated and evaporated. The residue was purified by passing through a 10 g SPE cartridge, using a gradient of ethyl acetate and cyclohexane (0-100% EtOAc) as the eluent, to give Intermediate 164 (125 mg). LCMS showed MH⁺=507; T_{REF}=3.85 min.

The following Intermediate 165 was prepared in a similar manner from Intermediate 163 and Intermediate 82:

### Intermediate 165: 1,1-dimethylethyl 4-{[5-({[1-(2,4-dimethylphenyl)propyl]amino}carbonyl)-1-ethyl-1H-pyrazolo[3,4-b]pyridin-4-yl]amino}-1-piperidinecarboxylate

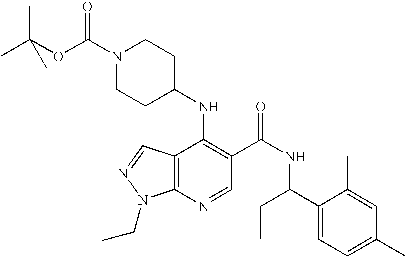 (believed to be a mixture of isomers with the major isomer believed to have the (R)-stereochemistry at the benzylic carbon atom). LCMS showed MH⁺=535; T_{RET}=3. min.

### Intermediate 166: 4-Amino-4-(3-methylphenyl)butyric acid

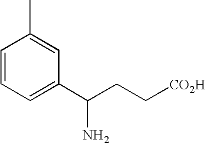

Triethylamine (6.3 g) was added to a cooled (0-5° C.) solution of 4-(3-methylphenyl)-4-oxobutyric acid (e.g. available from Oakwood Products Inc., 8 g) in DCM (100 ml). Hydroxylamine hydrochloride (3.47 g) was added slowly over 15 min. and the reaction mixture was stirred at room temperature overnight. The reaction mixture was extracted with 10% w/v sodium bicarbonate solution (2×75 ml). The aqueous extracts were combined, washed with diethyl ether, acidified to pH=2 with concentrated hydrochloric acid and extracted with ethyl acetate (3×100 ml). The combined ethyl acetate extracts were washed with water and brine, dried over Na₂SO₄ and concentrated in vacuo to give the intermediate oxime (8 g). A solution of the oxime (4 g) in methanol (50 ml) was hydrogenated overnight at room temperature and 4-Kg hydrogen pressure, using 10% palladium on carbon as the catalyst. The reaction mixture was filtered through celite. The celite was washed with methanol and the combined filtrate and washings were concentrated. The residue was slurried in ethyl acetate. The resultant suspension was filtered. The residue was dried to give Intermediate 166 as a white solid (3.5 g).

### Intermediate 167: 4-({[(1,1-dimethylethyl)oxy]carbonyl}amino)-4-(3-methylphenyl)butanoic acid

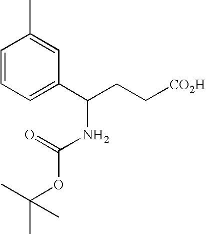

“BOC Anhydride” (di-tert-butyl carbonate, 4 g) was added to a solution of Intermediate 166 (3.3 g),and triethylamine (2.6 g) in methanol (50 ml) at 0-5° C. The reaction mixture was stirred at room temperature for 2 hours. 10% w/v Sodium bicarbonate solution (100 ml) was added. The reaction mixture was washed with diethyl ether, acidified to pH=3 with 20% w/v citric acid solution and extracted with ethyl acetate (3×50 ml). The combined organics were washed with water and brine, dried over Na₂SO₄ and concentrated in vacuo to give Intermediate 167 (5.6 g) as a white solid.

### Intermediate 168: 1,1-dimethylethyl [4-(dimethylamino)-1-(3-methylphenyl)-4-oxobutyl]carbamate

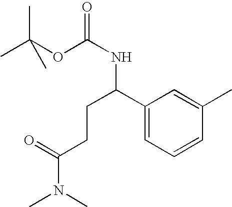

A 30% w/v solution of dinethylamine in EtOH (0.46 ml) was added to a stirred solution of Intermediate 167 (250 mg), HOBT (126 mg), EDC (180 mg) and DIPEA (0.37 ml) in MeCN. The reaction mixture was stirred for 24 h. The solvent was removed in vacuo and the residue was partitioned between EtOAc and 0.5M sodium bicarbonate solution. The organic phase was washed with saturated brine and dried by passing through a 10 g cartridge of MgSO₄ under suction. The solution was concentrated in vacuo. The residue was purified by passing through a 10 g SPE cartridge, using a (1:1) mixture of cyclohexane and EtOAc as the eluent, to give Intermediate 168 (109 mg) as a white solid. LCMS showed MH⁺=321; T_{RET}=2.88 min.

### Intermediate 169: 4-amino-N,N-dimethyl-4-(3-methylphenyl)butanamide hydrochloride

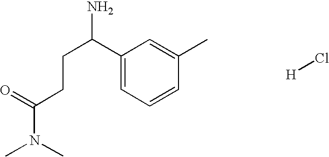

Intermediate 168 (108 mg) was treated with a 4M solution of hydrogen chloride in dioxan (2 ml). The reaction mixture was stirred for 6.5 h then concentrated in vacuo. The residue was triturated in diethyl ether. The diethyl ether was decanted. The residue was purified by passing through a 5 g SPE silica cartridge, using a gradient of 10-50% methanol in ethyl acetate as the eluent, to give Intermediate 169 (56 mg) as a white solid. LCMS showed MH⁺=221; T_{RET}=1.74 min.

### Intermediate 170

### Intermediate 170 can be synthesised according to the following reaction scheme:

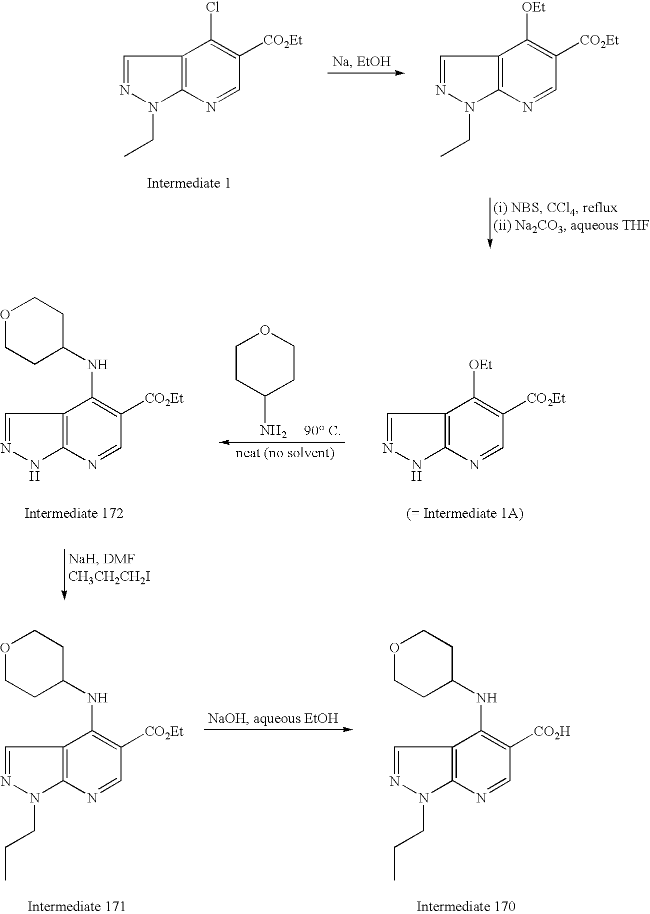

The final step in the above Intermediate 170 reaction scheme can optionally be performed as follows:

### Intermediate 170: 1-n-Propyl4-(tetrahydro-2H-pyran-4-ylamino)-1H-pyrazolo[3,4-b]pyridine-5-carboxylic acid

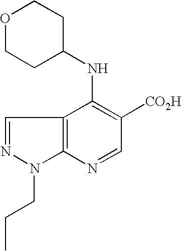

Optional synthesis: 2M-Sodium hydroxide solution (0.7 ml) was added to a stirred suspension of the corresponding ethyl ester (Intermediate 171) (0.23 g) in ethanol (5 ml) and water (1.5 ml). After stirring overnight at room temperature, a further quantity of 2M-sodium hydroxide solution (0.7 ml) was added, and the reaction mixture was heated at 43° C. for 2.5 h. The reaction solution was concentrated, diluted with water (5 ml) and acidified with 2M-hydrochloric acid. The resulting precipitate was collected by filtration, washed with water and dried to give Intermediate 170 as a white solid (0.14 g). LCMS showed MH⁺=305; T_{RET}=2.42 min.

The penultimate step in the above Intermediate 170 reaction scheme (to make Intermediate 171) can optionally be performed as follows:

### Intermediate 171: Ethyl 1-n-propyl4-(tetrahydro-2H-pyran-4-ylamino)-1H-pyrazolo[3,4-b]pyridine-5-carboxylate

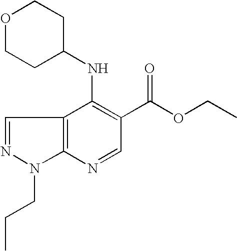

Optional synthesis: Sodium hydride (0.067 g, 60% dispersion in oil) was added to a stirred solution of Intermediate 172 (0.47 g) in DMF (19 ml), followed by n-propyl iodide (0.17 ml). The mixture was stirred at 23° C. for 16 hours, then concentrated, diluted with chloroform (30 ml) and washed with 1:1 water:brine solution (30 ml), separated and the organic layer concentrated. The residue was purified on a SPE catridge (silica, 10 g) eluting with 10 ml volumes of dichloromethane, 1:1 diethyl ether:cyclohexane, and diethyl ether. The combined 1:1 diethyl ether:cyclohexane, and diethyl ether, fractions were concentrated to give Intermediate 171 as a clear gum (0.23 g). LCMS showed M⁺=333; T_{RET}=3.14 min.

The ante-penultimate step in the above Intermediate 170 reaction scheme (to make Intermediate 172) can optionally be performed as follows:

### Intermediate 172: Ethyl 4-(tetrahydro-2H-pyran-4-ylamino)-1H-pyrazolo[3,4-b]pyridine-5carboxylate

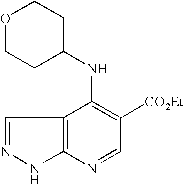

Optional Synthesis No. 1:

Intermediate 1A (0.035 g) was placed in a Reactivial™ and treated with 4-aminotetrahydropyran (0.05 ml). The mixture was heated at 90° C. for 1.5 h, then allowed to cool to room temperature and partitioned between chloroform (2 ml) and water (1 ml). The layers were separated and the organic phase was concentrated. The crude product was purified by mass directed autoprep HPLC to afford Intermediate 172 as an off-white solid (0.011 g). LCMS showed MH⁺=291; T_{RET}=2.08 min.

Alternative Optional Synthesis No. 2:

Intermediate 1A (2 g) was suspended in 4-aminotetrahydropyran (2 g), and the mixture was heated at 90° C. for 6 h. The residual mixture was allowed to cool to room temperature and partitioned between chloroform (50 ml) and water (50 ml). The phases were separated and the organic phase was evaporated to dryness. The residue was triturated with Et₂O (30 ml) and the insoluble solid was collected and dried to afford Intermediate 172 as a cream solid (2.24 g). LCMS showed MH⁺=291; T_{RET}=2.19 min.

### Intermediate 173: Ethyl 1-(2-hydroxyethyl)-4-(tetrahydro-2H-pyran-4-ylamino)-1H-pyrazolo[3,4-b]pyridine-5-carboxylate

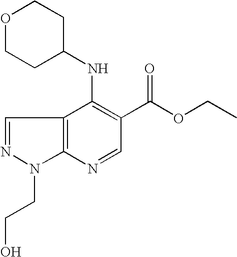

2-Bromoethanol (0.008 ml) was added to a solution of Intermediate 172 (0.03 g) in anhydrous DMF (1.5 ml), with 2-tert-butylimino-2-diethylamino-1,3-dimethyl-perhydro-1,3,2-diazaphosphorine (polymer bound, 2.3 mmol/g loading, 0.045 g). The mixture was shaken at 23° C. for 16 hours, then the solution drained from the resin, and the resin was washed with DMF. The combined organics were concentrated, and the residue purified on a SPE cartridge (silica, 1 g) eluting with 70-100% ethyl acetate in cyclohexane. The combined fractions were concentrated to give Intermediate 173 as a white solid (0.011 g). LCMS showed MH⁺=335; T_{RET}=2.47 min.

### Intermediate 175: (R)-(+)-3-Amino tetrahydrofuran 4-toluenesulphonate

Commercially available from Fluka Chemie AG, Germany (CAS 111769-27-8)

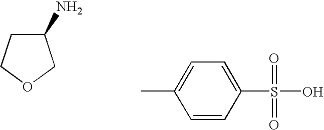

### Intermediate 176: (S)-(−)-3-Amino tetrahydrofuran 4-toluenesulphonate

Commercially available from E. Merck, Germany; or from E. Merck (Merck Ltd), Hunter Boulevard, Magna Park, Lutterworth, Leicestershire LE17 4XN, United Kingdom (CAS 104530-80-5)

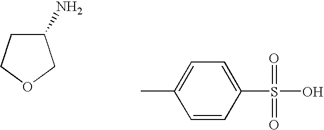

### Intermediate 177: Tetrahydro-2H-thiopyran-4-amine

This can be prepared from commercially available tetrahydrothiopyran-4-one as described by Subramanian et. al., J. Org. Chem., 1981, 46, 4376-4383. Subsequent preparation of the hydrochloride salt can be achieved by conventional means.

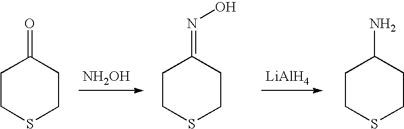

### Intermediate 178: Tetrahydro-3-thiopheneamine

This can be prepared in an analogous manner to Intermediate 177 from commercially available tetrahydrothiophene-4-one. The oxime formation is described by Grigg et. al., Tetrahedron, 1991, 47, 4477-4494 and the oxime reduction by Unterhalt et. al., Arch. Pharm., 1990,317-318.

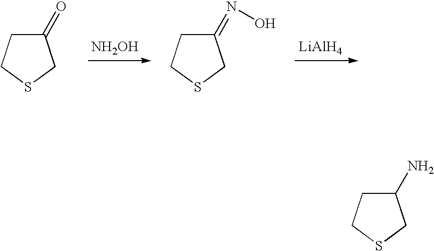

### Intermediate 179: Tetrahydro-3-thiopheneamine 1,1-dioxide hydrochloride

Commercially available from Sigma Aldrich Library of Rare Chemicals (SALOR) (CAS-6338-70-1). Preparation of the hydrochloride salt of the amine can be achieved by conventional means.

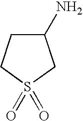

### Intermediate 180: Tetrahydro-2H-thiopyran-4-amine-1,1-dioxide hydrochloride

This can be prepared in an analogous manner to Intermediate 177 from commercially available tetrahydrothiopyran-4-one. Oxidation to 1,1-dioxo-tetrahydro-1λ⁶-thiopyran-4-one is described by Rule et. al., in J. Org. Chem., 1995, 60, 1665-1673. Oxime formation is described by Truce et. al., in J. Org. Chem., 1957, 617, 620 and oxime reduction by Barkenbus et. al., J. Am. Chem. Soc., 1955, 77, 3866. Subsequent preparation of the hydrochloride salt of the amine can be achieved by conventional means.

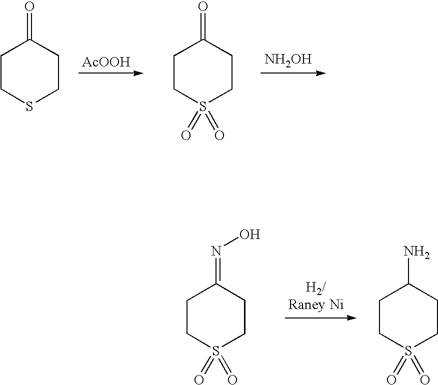

### Intermediate 181: Ethyl 1-methyl-4-ethoxy-1H-pyrazolo[3,4-b]pyridine-5-carboxylate

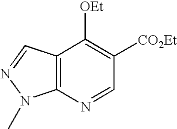

A mixture of Intermediate 1A (0.47 g) and anhydrous potassium carbonate (0.83 g) (previously dried by heating at 100° C.) in anhydrous dimethylformamide (DMF) (4 ml) was treated with iodomethane (0.26 ml) and stirred vigorously for 3 h. The mixture was then filtered and the filtrate concentrated in vacuo to afford a residual oil, which was partitioned between dichloromethane (DCM) (25 ml) and water (25 ml). The layers were separated and the aqueous phase was extracted with further DCM (2×25 ml). The combined organic extracts were dried over anhydrous sodium sulphate and evaporated to an orange solid which was applied to an SPE cartridge (silica, 20 g). The cartridge was eluted sequentially with EtOAc:petrol (1:4, 1:2 and 1:1), then chloroform:methanol (49:1, 19:1 and 9:1). Fractions containing desired material were combined and concentrated in vacuo to afford Intermediate 181 (0.165 g). LCMS showed MH⁺=250; T_{RET}=2.59 min.

### Intermediate 182: Ethyl 4-chloro-1-methyl-1H-pyrazolo[3,4-b]pyridine-5-carboxylate

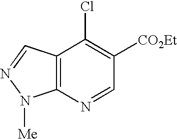

A mixture of 5-amino-1-methyl pyrazole (4.0 g) and diethylethoxymethylene malonate (9.16 ml) was heated at 150° C. under Dean Stark conditions for 5 h. Phosphorous oxychloride (55 ml) was carefully added to the mixture and the resulting solution heated at 130° C. under reflux for 18 h. The mixture was concentrated in vacuo, then the residual oil cooled in an ice bath and treated carefully with water (100 ml) (caution:exotherm). The resulting mixture was extracted with DCM (3×100 ml) and the combined organic extracts were dried over anhydrous sodium sulphate and concentrated in vacuo. The residual solid was purified by Biotage chromatography (silica, 90 g), eluting with Et₂O:petrol (1:3). Fractions containing desired material were combined and concentrated in vacuo to afford Intermediate 182 (4.82 g). LCMS showed MH⁺=240; T_{RET}=2.98 min

### Intermediate 183: 4-Chloro-1-methyl-1H-pyrazolo[3,4-b]pyridine-5-carboxylic acid

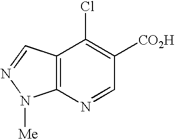

A solution of Intermediate 182 (4.0 g) in dioxane (30 ml) was treated with potassium hydroxide (7.54 g) as a solution in water (20 ml). The mixture was stirred for 16 h, then diluted with water (150 ml) and acidified to pH 3 with 5M aqueous hydrochloric acid. The mixture was stirred in an ice bath for 15 min, then collected by filtration, washed with ice-cold water and dried in vacuo over phosphorous pentoxide to afford Intermediate 183 as a white solid (2.83 g). LCMS showed MH⁺212; T_{RET}=2.26 min.

### Intermediate 184: Ethyl 1-ethyl-4-[(3S)-tetrahydrofuran-3-ylamino]-1H-pyrazolo[3,4-b]pyridine-5-carboxylate

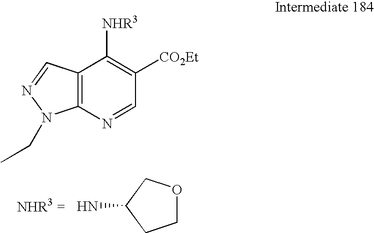

Intermediate 1 (0.05 g) and (S)-(−)-3-aminotetrahydrofuran 4-toluenesulphonate (Intermediate 176) (0.052 g) were suspended in ethanol (1 ml) and triethylamine (0.14 ml) was added. The mixture was stirred under nitrogen and heated at 80° C. for 24 h. After cooling to room temperature, ethanol was removed by evaporation under a stream of nitrogen and the residue partitioned between DCM (2 ml) and water (1.5 ml). The layers were separated and the organic layer concentrated to dryness. Purification was carried out using an SPE cartridge (silica, 5 g), eluting with a gradient of EtOAc:cyclohexane; (1:16 then, 1:8, 1:4, 1:2, 1:1 and 1:0). Fractions containing desired material were combined and concentrated in vacuo to afford Intermediate 184 (0.052 g). LCMS showed MH⁺=305; T_{RET}=2.70 min.

Similarly prepared were the following:

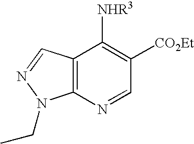

| | | | MH⁺ | |
|---|---|---|---|---|
| | NHR³ | Amine Reagent | ion | T_{RET}(min) |
| Intermediate 185 | 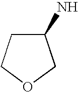 | (R)-(+)-3-Amino- tetrahydrofuran 4-toluene- sulphonate (Intermediate 175) | 305 | 2.73 |
| Intermediate 186 | 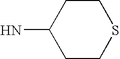 | Intermediate 177 | 335 | 3.21 |
| Intermediate 187 | 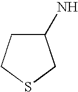 | Intermediate 178 | 321 | 3.10 |
| Intermediate 188 | 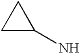 | Cyclo- propylamine | 275 | 2.98 |

### Intermediate 189: Ethyl 4-[(1,1-dioxidotetrahydrothien-3-yl)amino]-1-ethyl-1H-pyrazolo [3,4-b]pyridine-5-carboxylate

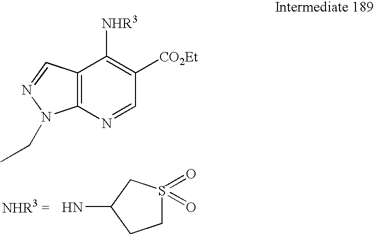

Intermediate 1 (0.05 g) and Intermediate 179 (0.027 g) were suspended in ethanol (1 ml) and triethylamine (0.14 ml) was added. The mixture was stirred under nitrogen and heated at 80° C. for 24 h. After cooling to room temperature, ethanol was removed by evaporation under a stream of nitrogen and the residue partitioned between DCM (2 ml) and water (1.5 ml). The layers were separated and the organic layer concentrated to dryness. Purification was carried out using an SPE cartridge (silica, 5 g), eluting with a gradient of EtOAc:cyclohexane; (1:8 then 1:4, 1:2, 1:1 and 1:0). Fractions containing desired material were combined and concentrated in vacuo to afford Intermediate 189 (0.045 g) as a mixture of enantiomers. LCMS showed MH⁺=353; T_{RET}=2.60 min.

Similarly prepared was the following:

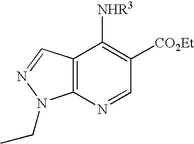

| | | | MH⁺ | |
|---|---|---|---|---|
| | NHR³ | Amine Reagent | ion | T_{RET}(min) |
| Inter- mediate 190 | 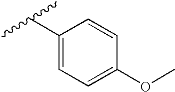 | Intermediate 180 | 367 | 2.64 |

### Intermediate 191: 1-Ethyl4-[(3S)-tetrahydrofuran-3-ylamino]-1H-pyrazolo[3,4-b]pyridine-5-carboxylic acid

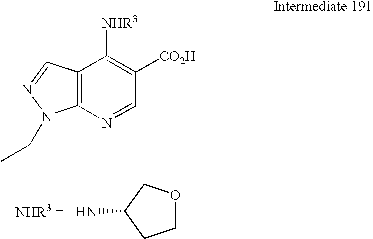

A solution of Intermediate 184 (0.037 g) in ethanol:water (95:5, 3 ml) was treated with sodium hydroxide (0.019 g). The mixture was heated at 50° C. for 16 h, then concentrated in vacuo. The residue was dissolved in water (1.5 ml) and acidified to pH 4 with acetic acid. The resultant white solid precipitate was removed by filtration and dried under vacuum. The filtrate was extracted with ethyl acetate and the organic layer collected and concentrated in vacuo to afford a further portion of white solid. The two solids were combined to afford Intermediate 191 (0.033 g). LCMS showed MH⁺=277; T_{RET}=2.05 min.

Similarly prepared were the following:

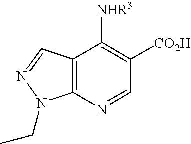

| | | | MH⁺ | |
|---|---|---|---|---|
| | NHR³ | Starting material | ion | T_{RET}(min) |
| Inter- mediate 192 | 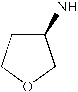 | Intermediate 185 | 277 | 2.05 |
| Inter- mediate 193 | 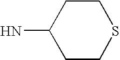 | Intermediate 186 | 307 | 2.40 |
| Inter- mediate 194 | 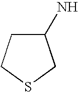 | Intermediate 187 | 293 | 2.59 |
| Inter- mediate 195 | 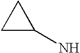 | Intermediate 188 | 247 | 2.24 |
| Inter- mediate 196 | 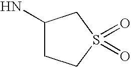 | Intermediate 189 | 325 | 2.05 |
| Inter- mediate 197 | 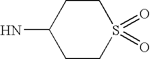 | Intermediate 190 | 339 | 2.05 |

### Intermediate 198: Ethyl 4-(cyclohexylamino)-1H-pyrazolo[3,4-b]pyridine-5-carboxylate

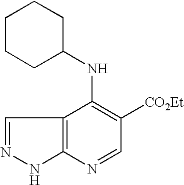

Intermediate 1A (0.69 g) was suspended in cyclohexylamine (1.01 ml), and the mixture was heated at 90° C. for 3 h. The residual mixture was allowed to cool to room temperature and partitioned between chloroform (25 ml) and water (25 ml). The phases were separated and the organic phase was evaporated to dryness. The residue was triturated with Et₂O (25 ml) and the insoluble solid was collected and dried to afford Intermediate 198 as a beige solid (0.58 g). LCMS showed MH⁺=289; T_{RET}=2.91 min.

### Intermediate 199: 4-(Cyclohexylamino)-1H-pyrazolo[3,4-b]pyridine-5-carboxylic acid

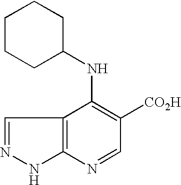

2M-Sodium hydroxide solution (0.5 ml) was added to a stirred suspension of Intermediate 198 (0.2 g) in dioxan (4 ml) and water (0.5 ml). After stirring overnight at room temperature, the reaction mixture was heated at 40° C. for 8 h. A further quantity of 2M-sodium hydroxide solution (1.5 ml) was added, and the reaction mixture was heated at 40° C. for 48 h. The reaction solution was concentrated, diluted with water (10 ml) and acidified with glacial acetic acid. The resulting precipitate was collected by filtration, washed with water and dried to give Intermediate 199 (0.18 g). LCMS showed MH⁺=261; T_{RET}=2.09 min.

### Intermediate 200: Ethyl 4-(cyclohexylamino)-1-ethyl-6-methyl-1H-pyrazolo[3,4-b]pyridine-5-carboxylate

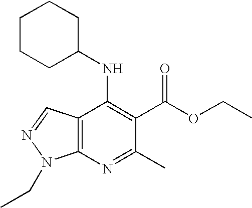

Cyclohexylamine (0.149 g, 1.5 mmol) was added to a mixture of Intermediate 10 (0.201 g, 0.75 mmol) and N,N-diisopropylethylamine (0.65 ml, 3.73 mmol) in acetonitrile (3 ml). The resulting mixture was heated at 85° C. for 40 hours. Volatiles were removed in vacuo and the residue was dissolved in chloroform (1.5 ml) and applied to a SPE cartridge (silica, 5 g). The cartridge was eluted successively with Et₂O, EtOAc and MeOH. Fractions containing the desired product were combined and concentrated in vacuo to afford Intermediate 200 (0.128 g). LCMS showed MH⁺=331; T_{RET}=3.64 min.

### Intermediate 201: 4-(Cyclohexylamino)-1-ethyl-6-methyl-1H-pyrazolo[3,4-b]pyridine-5-carboxylic acid

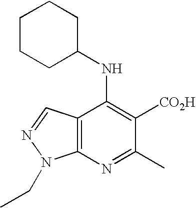

2M-Sodium hydroxide solution (0.39 ml, 0.78 mmol) was added to the corresponding ethyl ester (Intermediate 200) (0.128 g, 0.39 mmol) in ethanol (1.5 ml), and the mixture was heated at 50° C. for 16 hours. The reaction mixture was concentrated, and the resulting aqueous solution was neutralised with 2M-hydrochloric acid to precipitate a solid which was collected by filtration. The filtrate was applied to an OASIS ® hydrophilic-lipophilic balance (HLB) Extraction cartridge*(1 g) which was eluted with water followed by methanol. Evaporation of the methanol fraction gave a solid which was combined with the initial precipitated solid to afford Intermediate 201 (0.083 g) as a white solid, presumed to be the carboxylic acid.

OASIS ® HLB Extraction cartridges are available from Waters Corporation, 34 Maple Street, Milford, Mass. 01757, USA. The cartridges include a column containing a copolymer sorbent having a HLB such that when an aqueous solution is eluted through the column, the solute is absorbed or adsorbed into or onto the sorbent, and such that when organic solvent (e.g. methanol) is eluted the solute is released as an organic (e.g. methanol) solution. This is a way to separate the solute from aqueous solvent.

### Intermediate 202: 1-Ethyl-6-methyl-4-(tetrahydro-2H-pyran-4-ylamino)-1H-pyrazolo[3,4-b]pyridine-5-carboxylic acid

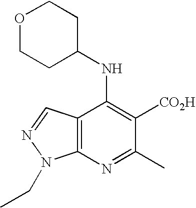

2M-Sodium hydroxide solution (0.75 ml, 1.5 mmol) was added to Intermediate 11 (0.248 g, 0.75 mmol) in ethanol (2 ml), and the mixture was heated at reflux for 16 hours. The reaction mixture was concentrated, diluted with water (1 ml) and acidified with 2M-hydrochloric acid (0.75 ml) to precipitate a solid which was collected by filtration to afford Intermediate 202 (0.168 g). LCMS showed MH⁺=305; T_{RET}=1.86 min.

### Intermediate 203: 4-Aminocyclohexanone hydrochloride

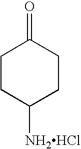

A solution of hydrogen chloride in dioxan (0.5 ml, 2.0 mmol, 4M) was added to a stirred solution of tert-butyl 4-oxocyclohexylcarbamate (0.043 g, 0.20 mmol, commercially available from AstaTech Inc., Philadelphia, USA) in dioxan (0.5 ml) and the mixture was stirred at room temperature. After 1 h, the reaction mixture was evaporated to give Intermediate 203 as a cream solid (34 mg). ¹H NMR (400 MHz in d₆-DMSO, 27° C., δ ppm) 8.09 (br. s, 3H), 3.51 (tt, 11, 3.5Hz, 1H), 2.45 (m, 2H, partially obscured), 2.29 (m, 2H), 2.16 (m, 2H), 1.76 (m, 2H).

### Intermediate 204: Ethyl 1-ethyl-4-(tetrahydro-2H-pyran-3-ylamino)-1H-pyrazolo[3,4-b]pyridine-5-carboxylate

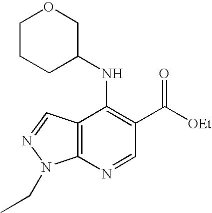

Intermediate 1 (0.76 g, 3.0 mmol)) was dissolved in acetonitrile (10 ml). Tetrahydro-2H-pyran-3-amine hydrochloride (0.5 g, 3.6 mmol, Anales De Quimica, 1988, 84, 148) and N,N-diisopropylethylamine (3.14 ml, 18.0 mmol) were added and the mixture was stirred at 85° C. for 24 h. After 24 h a further portion of tetrahydro-2H-pyran-3-amine hydrochloride (0.14 g, 1.02 mmol) was added and stirring was continued at 85° C. After a further 8 h, the mixture was concentrated in vacuo. The residue was partitioned between DCM (20 ml) and water (12 ml). The layers were separated and the aqueous layer was extracted with further DCM (12 ml). The combined organic extracts were dried (Na₂SO₄), and concentrated in vacuo to give a brown solid which was purified on a SPE cartridge (silica, 20 g) eluting with a gradient of ethyl acetate:cyclohexane (1:16, 1:8, 1:4, 1:2, 1:1, 1:0). Fractions containing the desired material were combined and evaporated to afford Intermediate 204 (0.89 g). LCMS showed MH⁺=319; T_{RET}=2.92 min.

### Intermediate 205: 1-Ethyl4-(tetrahydro-2H-pyran-3-ylamino)-1H-pyrazolo[3,4b]pyridine-5-carboxylic acid

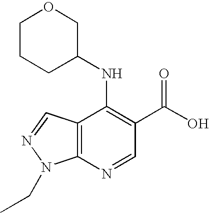

A solution of Intermediate 204 (0.89 g, 2.79 mmol) in ethanol (16.7 ml) was treated with sodium hydroxide (0.47 g, 11.7 mmol) as a solution in water (3.1 ml). The mixture was stirred at 50° C. After 12 h, the reaction mixture was concentrated in vacuo to give a residual oil which was dissolved in water (16 ml), then cooled and acidified to pH 3 with 2M hydrochloric acid. After stirring at 0° C. for 30 min, the resulting precipitate was collected by filtration, washed with cooled water (2 ml) and dried in vacuo to afford Intermediate 205 as a white solid (0.73 g). LCMS showed MH⁺=291; T_{RET}=2.19 min.

### Intermediate 206: 1,1-Dimethylethyl (4,4-difluorocyclohexyl)carbamate

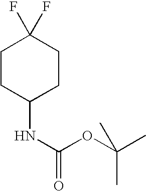

(Diethylamino)sulphur trifluoride (DAST), (0.06 ml, 0.47 mmol), was added to a stirred solution of 1,1-dimethylethyl(4-oxocyclohexyl)carbamate, (250 mg, 1.17 mmol, commercially available from AstaTech Inc., Philadelphia, USA) in anhydrous dichloromethane (5 ml) and the mixture was stirred under nitrogen at 20° C. After 22 h, the reaction mixture was cooled to 0° C., treated with saturated sodium hydrogen carbonate solution (4 ml), and then allowed to warm to ambient temperature. The phases were separated by passage through a hydrophobic frit and the aqueous phase was further extracted with DCM (5 ml). The combined organic phases were concentrated in vacuo to give an orange solid (369 mg) which was further purified by-chromatography using a SPE cartridge (silica, 10 g), eluting with DCM to afford Intermediate 206 (140 mg) containing 20% of 1,1-dimethylethyl (4-fluoro-3-cyclohexen-1-yl)carbamate. ¹H NMR (400 MHz in CDCl₃, 27° C., δ ppm).

Minor component: 85.11 (dm, 16 Hz, 1H), 4.56 (br, 1H), 3.80 (br, 1H) 2.45-1.45 (m's, 6H excess), 1.43 (s, 9H). Major component: δ4.43 (br, 1H), 3.58 (br, 1H), 2.45-1.45 (m's, 8H excess), 1.45 (s, 9H).

### Intermediate 207: (4,4-Difluorocyclohexyl)amine hydrochloride

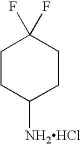

A solution of hydrogen chloride in dioxane (4M, 1.6 ml) was added at 20° C. to a stirred solution of Intermediate 206 (140 mg, 0.6 mmol), in dioxane (1.6 ml). After 3 h, the reaction mixture was concentrated in vacuo to afford Intermediate 207 (96.5 mg) containing 4-fluoro-3-cyclohexen-1-amine. ¹H NMR (400 MHz in d₆-DMSO, 27° C., δ ppm) Minor component: 68.22 (br, 3H excess), 5.18 (dm, 16 Hz, 1H), 3.28-3.13 (m, 1H excess), 2.41-1.53 (m's, 6H excess). Major component: δ8.22 (br, 3H excess), 3.28-3.13 (m, 1H excess), 2.41-1.53 (m's, 8H excess). Impurities are also present.

### Intermediates 208 to 229: different types of R³NH₂

| Inter- | | One Possible Source of, |
|---|---|---|
| mediate | | and/or a Reference to, |
| Number | R³NH₂ | R³NH₂ |
| 208 | 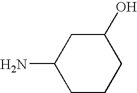 | AB Chem, Inc., Canada (mixture of cis and trans); or J. Chem. Soc., Perkin Trans. 1, 1994, 537 |
| 208A | as Intermediate 208, but | J. Chem. Soc., Perkin |
| | racemic cis-isomer, i.e. | Trans 1, 1994, 537 |
| | racemic cis-(3-hydroxy- | (discloses a 3.3:1 cis:trans |
| | cyclohex-1-yl)-amine | mixture) |
| 209 | 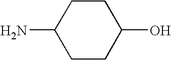 | Aldrich; or TCI-America |
| 210 | 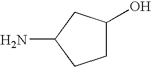 | U.S. Pat. No. 4219660 |
| 211 | 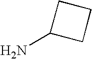 | Aldrich |
| 212 | 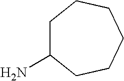 | Aldrich |
| 213 | 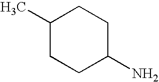 | Aldrich |
| 214 | 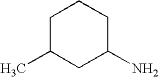 | Pfaltz-Bauer |
| 215 | 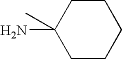 | J. Org. Chem., 1985, 50(11), 1859 |
| 216 | 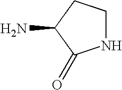 | WO 99/12933 |
| 217 | 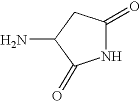 | EP 1188744 |
| 218 | 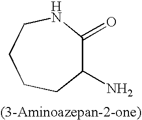 | Sigma-Aldrich Company Ltd |
| 219* | 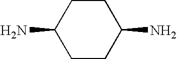 | J. Med. Chem., 1994, 37(17), 2360 |
| 220* | 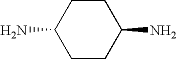 | Aldrich |
| 221* | 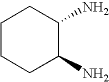 | Aldrich |
| 222* | 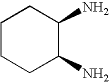 | Aldrich |
| 223* | 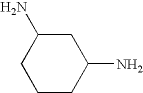 | Peakdale Molecular Ltd |
| 224 | 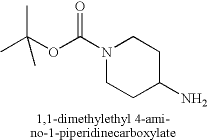 | AstaTech |
| 225 | 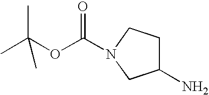 | |
| 226 | 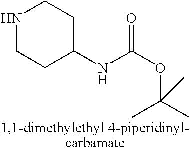 | Syngene or AstaTech |
| 227 | 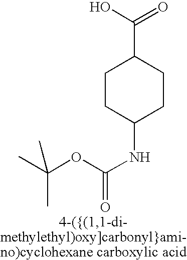 | Fluka |
| 228 | 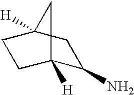 | Aldrich |
| 229 | 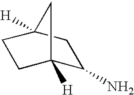 | Aldrich |

*For R³NH₂ in Intermediates 219-223, R³NH₂ is the cis or trans isomer, if shown. For Intermediates 221-223, R³NH₂ is usually the 3-amino- or 2-amino- cyclohex-1-ylamine in a racemic form.

Many of Intermediates 208 to 229, either as they are or after deprotection, protection and/or functional group interconversion(s), can optionally be used as R³NH₂ amines in the preparation of compounds of formula (I) or precursors thereto, e.g. as described in

Processes A or B and/or Process D hereinabove; optionally followed by deprotection, protection and/or functional group interconversion(s) e.g. in the 4-(R³NH) group of the pyrazolopyridine compound prepared.

Table of Examples

| Example | |
|---|---|
| Number | Name |
| 1 | 1-ethyl-N-[(1R)-1-phenylpropyl]-4-(tetrahydro-2H-pyran-4-ylamino)-1H- |
| | pyrazolo[3,4-b]pyridine-5-carboxamide |
| 2 | 1-ethyl-N-(1-methyl-1-phenylethyl)-4-(tetrahydro-2H-pyran-4-ylamino)- |
| | 1H-pyrazolo[3,4-b]pyridine-5-carboxamide |
| 3 | 1-ethyl-N-{1-[4-(methylsulfonyl)phenyl]ethyl}-4-(tetrahydro-2H-pyran-4- |
| | ylamino)-1H-pyrazolo[3,4-b]pyridine-5-carboxamide |
| 4 | N-(diphenylmethyl)-1-ethyl-4-(tetrahydro-2H-pyran-4-ylamino)-1H- |
| | pyrazolo[3,4-b]pyridine-5-carboxamide |
| 5 | 1-ethyl-N-[1-(3-pyridinyl)ethyl]-4-(tetrahydro-2H-pyran-4-ylamino)-1H- |
| | pyrazolo[3,4-b]pyridine-5-carboxamide |
| 6 | 1-ethyl-N-[(1S)-1-phenylpropyl]-4-(tetrahydro-2H-pyran-4-ylamino)-1H- |
| | pyrazolo[3,4-b]pyridine-5-carboxamide |
| 7 | 1-ethyl-N-[(1S)-1-phenylethyl]-4-(tetrahydro-2H-pyran-4-ylamino)-1H- |
| | pyrazolo[3,4-b]pyridine-5-carboxamide |
| 8 | 1-ethyl-N-[(1R)-1-phenylethyl]-4-(tetrahydro-2H-pyran-4-ylamino)-1H- |
| | pyrazolo[3,4-b]pyridine-5-carboxamide |
| 9 | 1-ethyl-N-[1-methyl-1-(4-pyridinyl)ethyl]-4-(tetrahydro-2H-pyran-4- |
| | ylamino)-1H-pyrazolo[3,4-b]pyridine-5-carboxamide |
| 10 | 1-ethyl-N-[(1R)-1-phenylethyl]-4-(tetrahydro-2H-pyran-4-ylamino)-1H- |
| | pyrazolo[3,4-b]pyridine-5-carboxamide |
| 11 | N-[1-(4-chlorophenyl)propyl]-1-ethyl-4-(tetrahydro-2H-pyran-4-ylamino)- |
| | 1H-pyrazolo[3,4-b]pyridine-5-carboxamide |
| 12 | 1-ethyl-N-{1-[4-(ethyloxy)phenyl]ethyl}-4-(tetrahydro-2H-pyran-4- |
| | ylamino)-1H-pyrazolo[3,4-b]pyridine-5-carboxamide |
| 13 | 1-ethyl-N-(3-hydroxy-1-phenylpropyl)-4-(tetrahydro-2H-pyran-4-ylamino)- |
| | 1H-pyrazolo[3,4-b]pyridine-5-carboxamide |
| 14 | 1-ethyl-N-[1-(3-hydroxyphenyl)ethyl]-4-(tetrahydro-2H-pyran-4-ylamino)- |
| | 1H-pyrazolo[3,4-b]pyridine-5-carboxamide |
| 15 | N-[2-(dimethylamino)-1-phenylethyl]-1-ethyl-4-(tetrahydro-2H-pyran-4- |
| | ylamino)-1H-pyrazolo[3,4-b]pyridine-5-carboxamide |
| 16 | 1-ethyl-N-[1-phenyl-2-(1-pyrrolidinyl)ethyl]-4-(tetrahydro-2H-pyran-4- |
| | ylamino)-1H-pyrazolo[3,4-b]pyridine-5-carboxamide |
| 17 | 1-ethyl-N-[1-(hydroxymethyl)-1-phenylpropyl]-4-(tetrahydro-2H-pyran-4- |
| | ylamino)-1H-pyrazolo[3,4-b]pyridine-5-carboxamide |
| 18 | 1-ethyl-N-{1-[4-(propyloxy)phenyl]ethyl}-4-(tetrahydro-2H-pyran-4- |
| | ylamino)-1H-pyrazolo[3,4-b]pyridine-5-carboxamide |
| 19 | methyl 3-({[1-ethyl-4-(tetrahydro-2H-pyran-4-ylamino)-1H-pyrazolo[3,4- |
| | b]pyridin-5-yl]carbonyl}amino)-3-phenylpropanoate |
| 20 | 1-ethyl-N-[1-(4-fluorophenyl)ethyl]-4-(tetrahydro-2H-pyran-4-ylamino)- |
| | 1H-pyrazolo[3,4-b]pyridine-5-carboxamide |
| 21 | N-[1-(4-chlorophenyl)ethyl]-1-ethyl-4-(tetrahydro-2H-pyran-4-ylamino)- |
| | 1H-pyrazolo[3,4-b]pyridine-5-carboxamide |
| 22 | ethyl ({[1-ethyl-4-(tetrahydro-2H-pyran-4-ylamino)-1H-pyrazolo[3,4- |
| | b]pyridin-5-yl]carbonyl}amino)(phenyl)acetate |
| 23 | 1-ethyl-N-{(1R)-1-[3-(methyloxy)phenyl]ethyl}-4-(tetrahydro-2H-pyran-4- |
| | ylamino)-1H-pyrazolo[3,4-b]pyridine-5-carboxamide |
| 24 | 1-ethyl-N-[(1S)-2-(methyloxy)-1-phenylethyl]-4-(tetrahydro-2H-pyran-4- |
| | ylamino)-1H-pyrazolo[3,4-b]pyridine-5-carboxamide |
| 25 | N-[(1R)-2-amino-2-oxo-1-phenylethyl]-1-ethyl-4-(tetrahydro-2H-pyran-4- |
| | ylamino)-1H-pyrazolo[3,4-b]pyridine-5-carboxamide |
| 26 | 1-ethyl-N-[(1R)-2-hydroxy-1-phenylethyl]-4-(tetrahydro-2H-pyran-4- |
| | ylamino)-1H-pyrazolo[3,4-b]pyridine-5-carboxamide |
| 27 | 1-ethyl-N-[(1R)-1-(4-nitrophenyl)ethyl]-4-(tetrahydro-2H-pyran-4- |
| | ylamino)-1H-pyrazolo[3,4-b]pyridine-5-carboxamide |
| 28 | 1-ethyl-N-[(1S)-2-hydroxy-1-phenylethyl]-4-(tetrahydro-2H-pyran-4- |
| | ylamino)-1H-pyrazolo[3,4-b]pyridine-5-carboxamide |
| 29 | 1-ethyl-N-[(1R)-2-(methyloxy)-1-phenylethyl]-4-(tetrahydro-2H-pyran-4- |
| | ylamino)-1H-pyrazolo[3,4-b]pyridine-5-carboxamide |
| 30 | 1-ethyl-N-(2-hydroxy-1,1-diphenylethyl)-4-(tetrahydro-2H-pyran-4- |
| | ylamino)-1H-pyrazolo[3,4-b]pyridine-5-carboxamide |
| 31 | N-[1-(3-cyanophenyl)ethyl]-1-ethyl-4-(tetrahydro-2H-pyran-4-ylamino)- |
| | 1H-pyrazolo[3,4-b]pyridine-5-carboxamide |
| 32 | N-[cyano(phenyl)methyl]-1-ethyl-4-(tetrahydro-2H-pyran-4-ylamino)-1H- |
| | pyrazolo[3,4-b]pyridine-5-carboxamide |
| 33 | N-{cyclopropyl[4-(methyloxy)phenyl]methyl}-1-ethyl-4-(tetrahydro-2H- |
| | pyran-4-ylamino)-1H-pyrazolo[3,4-b]pyridine-5-carboxamide |
| 34 | 1-ethyl-N-[1-(1-naphthalenyl)ethyl]-4-(tetrahydro-2H-pyran-4-ylamino)- |
| | 1H-pyrazolo[3,4-b]pyridine-5-carboxamide |
| 35 | N-(1,2-diphenylethyl)-1-ethyl-4-(tetrahydro-2H-pyran-4-ylamino)-1H- |
| | pyrazolo[3,4-b]pyridine-5-carboxamide |
| 36 | 1-ethyl-N-{1-[4-(methyloxy)phenyl]butyl}-4-(tetrahydro-2H-pyran-4- |
| | ylamino)-1H-pyrazolo[3,4-b]pyridine-5-carboxamide |
| 37 | 1-ethyl-N-[(1R)-1-(1-naphthalenyl)ethyl]-4-(tetrahydro-2H-pyran-4- |
| | ylamino)-1H-pyrazolo[3,4-b]pyridine-5-carboxamide |
| 38 | 1-ethyl-N-[(1S)-1-(1-naphthalenyl)ethyl]-4-(tetrahydro-2H-pyran-4- |
| | ylamino)-1H-pyrazolo[3,4-b]pyridine-5-carboxamide |
| 39 | N-[1-(aminocarbonyl)-1-phenylpropyl]-1-ethyl-4-(tetrahydro-2H-pyran-4- |
| | ylamino)-1H-pyrazolo[3,4-b]pyridine-5-carboxamide |
| 40 | 1-ethyl-N-(1-phenylcyclopentyl)-4-(tetrahydro-2H-pyran-4-ylamino)-1H- |
| | pyrazolo[3,4-b]pyridine-5-carboxamide |
| 41 | 1-ethyl-N-(4-phenyltetrahydro-2H-pyran-4-yl)-4-(tetrahydro-2H-pyran-4- |
| | ylamino)-1H-pyrazolo[3,4-b]pyridine-5-carboxamide |
| 42 | 1-ethyl-N-(1-phenylcyclopropyl)-4-(tetrahydro-2H-pyran-4-ylamino)-1H- |
| | pyrazolo[3,4-b]pyridine-5-carboxamide |
| 43 | N-{1-[4-(cyclohexyloxy)-3-methylphenyl]ethyl}-1-ethyl-4-(tetrahydro-2H- |
| | pyran-4-ylamino)-1H-pyrazolo[3,4-b]pyridine-5-carboxamide |
| 44 | N-{1-[3-(cyclohexyloxy)-4-(methyloxy)phenyl]ethyl}-1-ethyl-4- |
| | (tetrahydro-2H-pyran-4-ylamino)-1H-pyrazolo[3,4-b]pyridine-5- |
| | carboxamide |
| 45 | N-[1-(2,3-dichlorophenyl)ethyl]-1-ethyl-4-(tetrahydro-2H-pyran-4- |
| | ylamino)-1H-pyrazolo[3,4-b]pyridine-5-carboxamide |
| 46 | N-{1-[4-(cyclohexyloxy)-3-hydroxyphenyl]ethyl}-1-ethyl-4-(tetrahydro- |
| | 2H-pyran-4-ylamino)-1H-pyrazolo[3,4-b]pyridine-5-carboxamide |
| 47 | N-{1-[4-(cyclopentyloxy)phenyl]ethyl}-1-ethyl-4-(tetrahydro-2H-pyran-4- |
| | ylamino)-1H-pyrazolo[3,4-b]pyridine-5-carboxamide |
| 48 | 1-ethyl-N-[1-(4-methylphenyl)ethyl]-4-(tetrahydro-2H-pyran-4-ylamino)- |
| | 1H-pyrazolo[3,4-b]pyridine-5-carboxamide |
| 49 | N-{1-[4-(1,1-dimethylethyl)phenyl]cycloheptyl}-1-ethyl-4-(tetrahydro-2H- |
| | pyran-4-ylamino)-1H-pyrazolo[3,4-b]pyridine-5-carboxamide |
| 50 | N-[1-(4-bromophenyl)ethyl]-1-ethyl-4-(tetrahydro-2H-pyran-4-ylamino)- |
| | 1H-pyrazolo[3,4-b]pyridine-5-carboxamide |
| 51 | 1-ethyl-N-[(1S)-1-(4-iodophenyl)ethyl]-4-(tetrahydro-2H-pyran-4-ylamino)- |
| | 1H-pyrazolo[3,4-b]pyridine-5-carboxamide |
| 52 | N-{1-[4-(aminosulfonyl)phenyl]ethyl}-1-ethyl-4-(tetrahydro-2H-pyran-4- |
| | ylamino)-1H-pyrazolo[3,4-b]pyridine-5-carboxamide |
| 53 | 1-ethyl-N-(1-methyl-1-phenylpropyl)-4-(tetrahydro-2H-pyran-4-ylamino)- |
| | 1H-pyrazolo[3,4-b]pyridine-5-carboxamide |
| 54 | N-[1-(1,3-benzodioxol-5-yl)cyclohexyl]-1-ethyl-4-(tetrahydro-2H-pyran-4- |
| | ylamino)-1H-pyrazolo[3,4-b]pyridine-5-carboxamide |
| 55 | 1-ethyl-N-{1-[4-(methyloxy)phenyl]cyclohexyl}-4-(tetrahydro-2H-pyran-4- |
| | ylamino)-1H-pyrazolo[3,4-b]pyridine-5-carboxamide |
| 56 | 1-ethyl-N-[1-(4-fluorophenyl)cyclohexyl]-4-(tetrahydro-2H-pyran-4- |
| | ylamino)-1H-pyrazolo[3,4-b]pyridine-5-carboxamide |
| 57 | N-[1-(3-chlorophenyl)cyclopentyl]-1-ethyl-4-(tetrahydro-2H-pyran-4- |
| | ylamino)-1H-pyrazolo[3,4-b]pyridine-5-carboxamide |
| 58 | N-[1-(2-chlorophenyl)cyclopentyl]-1-ethyl-4-(tetrahydro-2H-pyran-4- |
| | ylamino)-1H-pyrazolo[3,4-b]pyridine-5-carboxamide |
| 59 | N-{1-[4-(1,1-dimethylethyl)phenyl]cyclohexyl}-1-ethyl-4-(tetrahydro-2H- |
| | pyran-4-ylamino)-1H-pyrazolo[3,4-b]pyridine-5-carboxamide |
| 60 | 1-ethyl-N-{1-[4-(1-methylethyl)phenyl]ethyl}-4-(tetrahydro-2H-pyran-4- |
| | ylamino)-1H-pyrazolo[3,4-b]pyridine-5-carboxamide |
| 61 | N-[1-(3,5-dimethylphenyl)ethyl]-1-ethyl-4-(tetrahydro-2H-pyran-4- |
| | ylamino)-1H-pyrazolo[3,4-b]pyridine-5-carboxamide |
| 62 | 1-ethyl-N-[(1S,2R)-2-hydroxy-1-phenylpropyl]-4-(tetrahydro-2H-pyran-4- |
| | ylamino)-1H-pyrazolo[3,4-b]pyridine-5-carboxamide |
| 63 | 1-ethyl-N-{(1R)-1-[4-(methyloxy)phenyl]ethyl}-4-(tetrahydro-2H-pyran-4- |
| | ylamino)-1H-pyrazolo[3,4-b]pyridine-5-carboxamide |
| 64 | 1-ethyl-N-{(1S)-1-[4-(methyloxy)phenyl]ethyl}-4-(tetrahydro-2H-pyran-4- |
| | ylamino)-1H-pyrazolo[3,4-b]pyridine-5-carboxamide |
| 65 | 1-ethyl-N-(1-phenylhexyl)-4-(tetrahydro-2H-pyran-4-ylamino)-1H- |
| | pyrazolo[3,4-b]pyridine-5-carboxamide |
| 66 | 1-ethyl-N-(1-phenylpentyl)-4-(tetrahydro-2H-pyran-4-ylamino)-1H- |
| | pyrazolo[3,4-b]pyridine-5-carboxamide |
| 67 | 1-ethyl-N-(2-methyl-1-phenylpropyl)-4-(tetrahydro-2H-pyran-4-ylamino)- |
| | 1H-pyrazolo[3,4-b]pyridine-5-carboxamide |
| 68 | 1-ethyl-N-(1-phenylbutyl)-4-(tetrahydro-2H-pyran-4-ylamino)-1H- |
| | pyrazolo[3,4-b]pyridine-5-carboxamide |
| 69 | 1-ethyl-4-(tetrahydro-2H-pyran-4-ylamino)-N-(2,2,2-trifluoro-1- |
| | phenylethyl)-1H-pyrazolo[3,4-b]pyridine-5-carboxamide |
| 70 | N-[cyclopropyl(phenyl)methyl]-1-ethyl-4-(tetrahydro-2H-pyran-4- |
| | ylamino)-1H-pyrazolo[3,4-b]pyridine-5-carboxamide |
| 71 | 1-ethyl-N-[1-(4-fluorophenyl)propyl]-4-(tetrahydro-2H-pyran-4-ylamino)- |
| | 1H-pyrazolo[3,4-b]pyridine-5-carboxamide |
| 72 | N-[1-(2,3-dichlorophenyl)propyl]-1-ethyl-4-(tetrahydro-2H-pyran-4- |
| | ylamino)-1H-pyrazolo[3,4-b]pyridine-5-carboxamide |
| 73 | 1-ethyl-N-[(1R)-1-(4-methylphenyl)ethyl]-4-(tetrahydro-2H-pyran-4- |
| | ylamino)-1H-pyrazolo[3,4-b]pyridine-5-carboxamide |
| 74 | 1-ethyl-N-(1-phenylethyl)-4-(tetrahydro-2H-pyran-4-ylamino)-1H- |
| | pyrazolo[3,4-b]pyridine-5-carboxamide |
| 75 | N-[(1R)-1-(4-bromophenyl)ethyl]-1-ethyl-4-(tetrahydro-2H-pyran-4- |
| | ylamino)-1H-pyrazolo[3,4-b]pyridine-5-carboxamide |
| 76 | N-[1-(4-chlorophenyl)-2-hydroxyethyl]-1-ethyl-4-(tetrahydro-2H-pyran-4- |
| | ylamino)-1H-pyrazolo[3,4-b]pyridine-5-carboxamide |
| 77 | N-[1-(3,4-dichlorophenyl)-2-hydroxyethyl]-1-ethyl-4-(tetrahydro-2H-pyran- |
| | 4-ylamino)-1H-pyrazolo[3,4-b]pyridine-5-carboxamide |
| 78 | 1-ethyl-N-{1-[3-(methyloxy)phenyl]propyl}-4-(tetrahydro-2H-pyran-4- |
| | ylamino)-1H-pyrazolo[3,4-b]pyridine-5-carboxamide |
| 79 | 1-ethyl-N-{1-[4-(methyloxy)phenyl]propyl}-4-(tetrahydro-2H-pyran-4- |
| | ylamino)-1H-pyrazolo[3,4-b]pyridine-5-carboxamide |
| 80 | N-[1-(4-bromophenyl)propyl]-1-ethyl-4-(tetrahydro-2H-pyran-4-ylamino)- |
| | 1H-pyrazolo[3,4-b]pyridine-5-carboxamide |
| 81 | 1-ethyl-N-{1-[4-(propyloxy)phenyl]propyl}-4-(tetrahydro-2H-pyran-4- |
| | ylamino)-1H-pyrazolo[3,4-b]pyridine-5-carboxamide |
| 82 | N-[1-(3,5-dimethylphenyl)propyl]-1-ethyl-4-(tetrahydro-2H-pyran-4- |
| | ylamino)-1H-pyrazolo[3,4-b]pyridine-5-carboxamide |
| 83 | 1-ethyl-N-[1-(4-methylphenyl)propyl]-4-(tetrahydro-2H-pyran-4-ylamino)- |
| | 1H-pyrazolo[3,4-b]pyridine-5-carboxamide |
| 84 | 1-ethyl-N-{1-[4-(1-methylethyl)phenyl]propyl}-4-(tetrahydro-2H-pyran-4- |
| | ylamino)-1H-pyrazolo[3,4-b]pyridine-5-carboxamide |
| 85 | 1-ethyl-N-[1-(2-methylphenyl)ethyl]-4-(tetrahydro-2H-pyran-4-ylamino)- |
| | 1H-pyrazolo[3,4-b]pyridine-5-carboxamide |
| 86 | N-(1-{4-[(difluoromethyl)oxy]phenyl}ethyl)-1-ethyl-4-(tetrahydro-2H- |
| | pyran-4-ylamino)-1H-pyrazolo[3,4-b]pyridine-5-carboxamide |
| 87 | 1-ethyl-4-(tetrahydro-2H-pyran-4-ylamino)-N-{1-[4- |
| | (trifluoromethyl)phenyl]ethyl}-1H-pyrazolo[3,4-b]pyridine-5-carboxamide |
| 88 | 1-ethyl-N-[1-(2-methylphenyl)propyl]-4-(tetrahydro-2H-pyran-4-ylamino)- |
| | 1H-pyrazolo[3,4-b]pyridine-5-carboxamide |
| 89 | 1-ethyl-N-{1-[4-(ethyloxy)phenyl]propyl}-4-(tetrahydro-2H-pyran-4- |
| | ylamino)-1H-pyrazolo[3,4-b]pyridine-5-carboxamide |
| 90 | N-(1-{4-[(difluoromethyl)oxy]phenyl}propyl)-1-ethyl-4-(tetrahydro-2H- |
| | pyran-4-ylamino)-1H-pyrazolo[3,4-b]pyridine-5-carboxamide |
| 91 | 1-ethyl-4-(tetrahydro-2H-pyran-4-ylamino)-N-{1-[4- |
| | (trifluoromethyl)phenyl]propyl}-1H-pyrazolo[3,4-b]pyridine-5- |
| | carboxamide |
| 92 | N-[1-(3,4-dimethylphenyl)propyl]-1-ethyl-4-(tetrahydro-2H-pyran-4- |
| | ylamino)-1H-pyrazolo[3,4-b]pyridine-5-carboxamide |
| 93 | N-[1-(2,3-dimethylphenyl)ethyl]-1-ethyl-4-(tetrahydro-2H-pyran-4- |
| | ylamino)-1H-pyrazolo[3,4-b]pyridine-5-carboxamide |
| 94 | N-[1-(2,4-dimethylphenyl)ethyl]-1-ethyl-4-(tetrahydro-2H-pyran-4- |
| | ylamino)-1H-pyrazolo[3,4-b]pyridine-5-carboxamide |
| 95 | N-[1-(4-chloro-2-fluorophenyl)ethyl]-1-ethyl-4-(tetrahydro-2H-pyran-4- |
| | ylamino)-1H-pyrazolo[3,4-b]pyridine-5-carboxamide |
| 96 | N-[1-(3-chloro-4-methylphenyl)ethyl]-1-ethyl-4-(tetrahydro-2H-pyran-4- |
| | ylamino)-1H-pyrazolo[3,4-b]pyridine-5-carboxamide |
| 97 | N-[1-(2,3-dimethylphenyl)propyl]-1-ethyl-4-(tetrahydro-2H-pyran-4- |
| | ylamino)-1H-pyrazolo[3,4-b]pyridine-5-carboxamide |
| 98 | N-[1-(2,4-dimethylphenyl)propyl]-1-ethyl-4-(tetrahydro-2H-pyran-4- |
| | ylamino)-1H-pyrazolo[3,4-b]pyridine-5-carboxamide |
| 99 | N-[1-(4-chloro-2-fluorophenyl)propyl]-1-ethyl-4-(tetrahydro-2H-pyran-4- |
| | ylamino)-1H-pyrazolo[3,4-b]pyridine-5-carboxamide |
| 100 | N-[1-(3-chloro-4-methylphenyl)propyl]-1-ethyl-4-(tetrahydro-2H-pyran-4- |
| | ylamino)-1H-pyrazolo[3,4-b]pyridine-5-carboxamide |
| 101 | 1-ethyl-N-[1-(3-hydroxyphenyl)propyl]-4-(tetrahydro-2H-pyran-4- |
| | ylamino)-1H-pyrazolo[3,4-b]pyridine-5-carboxamide |
| 102 | N-[1-(2,3-dihydro-1H-inden-5-yl)ethyl]-1-ethyl-4-(tetrahydro-2H-pyran-4- |
| | ylamino)-1H-pyrazolo[3,4-b]pyridine-5-carboxamide |
| 103 | 1-ethyl-N-[1-(5,6,7,8-tetrahydro-2-naphthalenyl)ethyl]-4-(tetrahydro-2H- |
| | pyran-4-ylamino)-1H-pyrazolo[3,4-b]pyridine-5-carboxamide |
| 104 | N-[1-(4-bromophenyl)-2,2,2-trifluoroethyl]-1-ethyl-4-(tetrahydro-2H- |
| | pyran-4-ylamino)-1H-pyrazolo[3,4-b]pyridine-5-carboxamide |
| 105 | 1-ethyl-4-(tetrahydro-2H-pyran-4-ylamino)-N-{2,2,2-trifluoro-1-[3- |
| | (methyloxy)phenyl]ethyl}-1H-pyrazolo[3,4-b]pyridine-5-carboxamide |
| 106 | 4-(cyclohexylamino)-1-ethyl-N-{1-[4-(methylsulfonyl)phenyl]ethyl}-1H- |
| | pyrazolo[3,4-b]pyridine-5-carboxamide |
| 107 | 4-(cyclohexylamino)-1-ethyl-N-[(1R)-1-phenylpropyl]-1H-pyrazolo[3,4- |
| | b]pyridine-5-carboxamide |
| 108 | 4-(cyclohexylamino)-N-(diphenylmethyl)-1-ethyl-1H-pyrazolo[3,4- |
| | b]pyridine-5-carboxamide |
| 109 | 4-(cyclohexylamino)-1-ethyl-N-[(1R)-1-phenylethyl]-1H-pyrazolo[3,4- |
| | b]pyridine-5-carboxamide |
| 110 | ethyl ({[4-(cyclohexylamino)-1-ethyl-1H-pyrazolo[3,4-b]pyridin-5- |
| | yl]carbonyl}amino)(phenyl)acetate |
| 111 | N-[1-(4-chlorophenyl)ethyl]-4-(cyclohexylamino)-1-ethyl-1H-pyrazolo[3,4- |
| | b]pyridine-5-carboxamide |
| 112 | 4-(cyclohexylamino)-1-ethyl-N-(1-methyl-1-phenylethyl)-1H-pyrazolo[3,4- |
| | b]pyridine-5-carboxamide |
| 113 | 4-(cyclohexylamino)-1-ethyl-N-[1-(4-fluorophenyl)ethyl]-1H-pyrazolo[3,4- |
| | b]pyridine-5-carboxamide |
| 114 | N-[1-(4-chlorophenyl)propyl]-4-(cyclohexylamino)-1-ethyl-1H- |
| | pyrazolo[3,4-b]pyridine-5-carboxamide |
| 115 | 4-(cyclohexylamino)-N-(1,2-diphenylethyl)-1-ethyl-1H-pyrazolo[3,4- |
| | b]pyridine-5-carboxamide |
| 116 | 4-(cyclohexylamino)-1-ethyl-N-{1-[4-(propyloxy)phenyl]ethyl}-1H- |
| | pyrazolo[3,4-b]pyridine-5-carboxamide |
| 117 | methyl 3-({[4-(cyclohexylamino)-1-ethyl-1H-pyrazolo[3,4-b]pyridin-5- |
| | yl]carbonyl}amino)-3-phenylpropanoate |
| 118 | 4-(cyclohexylamino)-1-ethyl-N-[1-(hydroxymethyl)-1-phenylpropyl]-1H- |
| | pyrazolo[3,4-b]pyridine-5-carboxamide |
| 119 | 4-(cyclohexylamino)-1-ethyl-N-(3-hydroxy-1-phenylpropyl)-1H- |
| | pyrazolo[3,4-b]pyridine-5-carboxamide |
| 120 | 4-(cyclohexylamino)-1-ethyl-N-{1-[4-(ethyloxy)phenyl]ethy}-1H- |
| | pyrazolo[3,4-b]pyridine-5-carboxamide |
| 121 | 4-(cyclohexylamino)-1-ethyl-N-[1-(3-hydroxyphenyl)ethyl]-1H- |
| | pyrazolo[3,4-b]pyridine-5-carboxamide |
| 122 | 4-(cyclohexylamino)-1-ethyl-N-[1-phenyl-2-(1-pyrrolidinyl)ethyl]-1H- |
| | pyrazolo[3,4-b]pyridine-5-carboxamide |
| 123 | 4-(cyclohexylamino)-N-[2-(dimethylamino)-1-phenylethyl]-1-ethyl-1H- |
| | pyrazolo[3,4-b]pyridine-5-carboxamide |
| 124 | 4-(cyclohexylamino)-1-ethyl-N-[(1R)-2-(methyloxy)-1-phenylethyl]-1H- |
| | pyrazolo[3,4-b]pyridine-5-carboxamide |
| 125 | N-[(1R)-2-amino-2-oxo-1-phenylethyl]-4-(cyclohexylamino)-1-ethyl-1H- |
| | pyrazolo[3,4-b]pyridine-5-carboxamide |
| 126 | 4-(cyclohexylamino)-1-ethyl-N-[(1R)-2-hydroxy-1-phenylethyl]-1H- |
| | pyrazolo[3,4-b]pyridine-5-carboxamide |
| 127 | 4-(cyclohexylamino)-1-ethyl-N-[(1S)-2-hydroxy-1-phenylethyl]-1H- |
| | pyrazolo[3,4-b]pyridine-5-carboxamide |
| 128 | 4-(cyclohexylamino)-1-ethyl-N-{(1R)-1-[3-(methyloxy)phenyl]ethyl}-1H- |
| | pyrazolo[3,4-b]pyridine-5-carboxamide |
| 129 | 4-(cyclohexylamino)-1-ethyl-N-[(1S)-2-(methyloxy)-1-phenylethyl]-1H- |
| | pyrazolo[3,4-b]pyridine-5-carboxamide |
| 130 | 4-(cyclohexylamino)-1-ethyl-N-[(1R)-1-(4-nitrophenyl)ethyl]-1H- |
| | pyrazolo[3,4-b]pyridine-5-carboxamide |
| 131 | 4-(cyclohexylamino)-1-ethyl-N-[(1S)-1-(1-naphthalenyl)ethyl]-1H- |
| | pyrazolo[3,4-b]pyridine-5-carboxamide |
| 132 | 4-(cyclohexylamino)-1-ethyl-N-[phenyl(4-phenyl-1,3-thiazol-2-yl)methyl]- |
| | 1H-pyrazolo[3,4-b]pyridine-5-carboxamide |
| 133 | N-[cyano(phenyl)methyl]-4-(cyclohexylamino)-1-ethyl-1H-pyrazolo[3,4- |
| | b]pyridine-5-carboxamide |
| 134 | 4-(cyclohexylamino)-1-ethyl-N-[1-(1-naphthalenyl)ethyl]-1H-pyrazolo[3,4- |
| | b]pyridine-5-carboxamide |
| 135 | 4-(cyclohexylamino)-1-ethyl-N-(2-hydroxy-1,1-diphenylethyl)-1H- |
| | pyrazolo[3,4-b]pyridine-5-carboxamide |
| 136 | 4-(cyclohexylamino)-1-ethyl-N-{(1R)-1-[4-(methyloxy)phenyl]ethyl}-1H- |
| | pyrazolo[3,4-b]pyridine-5-carboxamide |
| 137 | 4-(cyclohexylamino)-1-ethyl-N-[1-(4-fluorophenyl)propyl]-1H- |
| | pyrazolo[3,4-b]pyridine-5-carboxamide |
| 138 | 4-(cyclohexylamino)-N-[1-(2,3-dichlorophenyl)propyl]-1-ethyl-1H- |
| | pyrazolo[3,4-b]pyridine-5-carboxamide |
| 139 | 4-(cyclohexylamino)-1-ethyl-N-[(1R)-1-(4-methylphenyl)ethyl]-1H- |
| | pyrazolo[3,4-b]pyridine-5-carboxamide |
| 140 | 4-(cyclohexylamino)-1-ethyl-N-(1-phenylethyl)-1H-pyrazolo[3,4- |
| | b]pyridine-5-carboxamide |
| 141 | N-[(1R)-1-(4-bromophenyl)ethyl]-4-(cyclohexylamino)-1-ethyl-1H- |
| | pyrazolo[3,4-b]pyridine-5-carboxamide |
| 142 | 4-(cyclohexylamino)-N-[1-(2,3-dichlorophenyl)ethyl]-1-ethyl-1H- |
| | pyrazolo[3,4-b]pyridine-5-carboxamide |
| 143 | 4-(cyclohexylamino)-1-ethyl-N-{1-[3-(methyloxy)phenyl]propyl}-1H- |
| | pyrazolo[3,4-b]pyridine-5-carboxamide |
| 144 | 4-(cyclohexylamino)-1-ethyl-N-{1-[4-(methyloxy)phenyl]propyl}-1H- |
| | pyrazolo[3,4-b]pyridine-5-carboxamide |
| 145 | N-[1-(4-bromophenyl)propyl]-4-(cyclohexylamino)-1-ethyl-1H- |
| | pyrazolo[3,4-b]pyridine-5-carboxamide |
| 146 | 4-(cyclohexylamino)-1-ethyl-N-{1-[4-(propyloxy)phenyl]propyl}-1H- |
| | pyrazolo[3,4-b]pyridine-5-carboxamide |
| 147 | 4-(cyclohexylamino)-N-[1-(3,5-dimethylphenyl)propyl]-1-ethyl-1H- |
| | pyrazolo[3,4-b]pyridine-5-carboxamide |
| 148 | 4-(cyclohexylamino)-1-ethyl-N-[1-(4-methylphenyl)propyl]-1H- |
| | pyrazolo[3,4-b]pyridine-5-carboxamide |
| 149 | 4-(cyclohexylamino)-1-ethyl-N-{1-[4-(1-methylethyl)phenyl]propyl}-1H- |
| | pyrazolo[3,4-b]pyridine-5-carboxamide |
| 150 | 4-(cyclohexylamino)-1-ethyl-N-[1-(2-methylphenyl)ethyl]-1H- |
| | pyrazolo[3,4-b]pyridine-5-carboxamide |
| 151 | 4-(cyclohexylamino)-N-(1-{4-[(difluoromethyl)oxy]phenyl}ethyl)-1-ethyl- |
| | 1H-pyrazolo[3,4-b]pyridine-5-carboxamide |
| 152 | 4-(cyclohexylamino)-1-ethyl-N-{1-[4-(trifluoromethyl)phenyl]ethyl}-1H- |
| | pyrazolo[3,4-b]pyridine-5-carboxamide |
| 153 | 4-(cyclohexylamino)-1-ethyl-N-[1-(2-methylphenyl)propyl]-1H- |
| | pyrazolo[3,4-b]pyridine-5-carboxamide |
| 154 | 4-(cyclohexylamino)-1-ethyl-N-{1-[4-(ethyloxy)phenyl]propyl}-1H- |
| | pyrazolo[3,4-b]pyridine-5-carboxamide |
| 155 | 4-(cyclohexylamino)-N-(1-{4-[(difluoromethyl)oxy]phenyl}propyl)-1- |
| | ethyl-1H-pyrazolo[3,4-b]pyridine-5-carboxamide |
| 156 | 4-(cyclohexylamino)-1-ethyl-N-{1-[4-(trifluoromethyl)phenyl]propyl}-1H- |
| | pyrazolo[3,4-b]pyridine-5-carboxamide |
| 157 | 4-(cyclohexylamino)-N-[1-(3,4-dimethylphenyl)propyl]-1-ethyl-1H- |
| | pyrazolo[3,4-b]pyridine-5-carboxamide |
| 158 | 4-(cyclohexylamino)-N-[1-(2,3-dimethylphenyl)ethyl]-1-ethyl-1H- |
| | pyrazolo[3,4-b]pyridine-5-carboxamide |
| 159 | 4-(cyclohexylamino)-N-[1-(2,4-dimethylphenyl)ethyl]-1-ethyl-1H- |
| | pyrazolo[3,4-b]pyridine-5-carboxamide |
| 160 | N-[1-(4-chloro-2-fluorophenyl)ethyl]-4-(cyclohexylamino)-1-ethyl-1H- |
| | pyrazolo[3,4-b]pyridine-5-carboxamide |
| 161 | N-[1-(3-chloro-4-methylphenyl)ethyl]-4-(cyclohexylamino)-1-ethyl-1H- |
| | pyrazolo[3,4-b]pyridine-5-carboxamide |
| 162 | 4-(cyclohexylamino)-N-[1-(2,3-dimethylphenyl)propyl]-1-ethyl-1H- |
| | pyrazolo[3,4-b]pyridine-5-carboxamide |
| 163 | 4-(cyclohexylamino)-N-[1-(2,4-dimethylphenyl)propyl]-1-ethyl-1H- |
| | pyrazolo[3,4-b]pyridine-5-carboxamide |
| 164 | N-[1-(4-chloro-2-fluorophenyl)propyl]-4-(cyclohexylamino)-1-ethyl-1H- |
| | pyrazolo[3,4-b]pyridine-5-carboxamide |
| 165 | N-[1-(3-chloro-4-methylphenyl)propyl]-4-(cyclohexylamino)-1-ethyl-1H- |
| | pyrazolo[3,4-b]pyridine-5-carboxamide |
| 166 | 4-(cyclohexylamino)-1-ethyl-N-[1-(3-hydroxyphenyl)propyl]-1H- |
| | pyrazolo[3,4-b]pyridine-5-carboxamide |
| 167 | N-[1-(4-chlorophenyl)-2-hydroxyethyl]-4-(cyclohexylamino)-1-ethyl-1H- |
| | pyrazolo[3,4-b]pyridine-5-carboxamide |
| 168 | 4-(cyclohexylamino)-N-[1-(2,3-dihydro-1H-inden-5-yl)ethyl]-1-ethyl-1H- |
| | pyrazolo[3,4-b]pyridine-5-carboxamide |
| 169 | 4-(cyclohexylamino)-1-ethyl-N-[1-(5,6,7,8-tetrahydro-2- |
| | naphthalenyl)ethyl]-1H-pyrazolo[3,4-b]pyridine-5-carboxamide |
| 170 | 4-[(1-acetyl-4-piperidinyl)amino]-1-ethyl-N-[(1S)-1-phenylpropyl]-1H- |
| | pyrazolo[3,4-b]pyridine-5-carboxamide |
| 171 | 4-[(1-acetyl-4-piperidinyl)amino]-1-ethyl-N-[(1R)-1-phenylethyl]-1H- |
| | pyrazolo[3,4-b]pyridine-5-carboxamide |
| 172 | 4-[(1-acetyl-4-piperidinyl)amino]-N-(diphenylmethyl)-1-ethyl-1H- |
| | pyrazolo[3,4-b]pyridine-5-carboxamide |
| 173 | 4-[(1-acetyl-4-piperidinyl)amino]-1-ethyl-N-{1-[4- |
| | (methylsulfonyl)phenyl]ethyl}-1H-pyrazolo[3,4-b]pyridine-5-carboxamide |
| 174 | 4-[(1-acetyl-4-piperidinyl)amino]-1-ethyl-N-[(1R)-1-phenylpropyl]-1H- |
| | pyrazolo[3,4-b]pyridine-5-carboxamide |
| 175 | N-[1-(4-chlorophenyl)ethyl]-1-ethyl-4-[(4-oxocyclohexyl)amino]-1H- |
| | pyrazolo[3,4-b]pyridine-5-carboxamide |
| 176 | N-[1-(4-chlorophenyl)propyl]-1-ethyl-4-[(4-oxocyclohexyl)amino]-1H- |
| | pyrazolo[3,4-b]pyridine-5-carboxamide |
| 177 | 1-ethyl-N-[(1S)-1-(4-nitrophenyl)ethyl]-4-[(4-oxocyclohexyl)amino]-1H- |
| | pyrazolo[3,4-b]pyridine-5-carboxamide |
| 178 | 1-ethyl-N-[(1R)-1-(4-nitrophenyl)ethyl]-4-[(4-oxocyclohexyl)amino]-1H- |
| | pyrazolo[3,4-b]pyridine-5-carboxamide |
| 179 | 1-ethyl-N-{1-[4-(ethyloxy)phenyl]ethyl}-4-[(4-oxocyclohexyl)amino]-1H- |
| | pyrazolo[3,4-b]pyridine-5-carboxamide |
| 180 | 1-ethyl-4-[(4-oxocyclohexyl)amino]-N-{1-[4-(propyloxy)phenyl]ethyl}-1H- |
| | pyrazolo[3,4-b]pyridine-5-carboxamide |
| 181 | 1-ethyl-N-[1-(4-fluorophenyl)ethyl]-4-[(4-oxocyclohexyl)amino]-1H- |
| | pyrazolo[3,4-b]pyridine-5-carboxamide |
| 182 | 1-ethyl-N-[(1R)-2-hydroxy-1-phenylethyl]-4-[(4-oxocyclohexyl)amino]- |
| | 1H-pyrazolo[3,4-b]pyridine-5-carboxamide |
| 183 | 1-ethyl-4-[(4-oxocyclohexyl)amino]-N-(1-phenylpropyl)-1H-pyrazolo[3,4- |
| | b]pyridine-5-carboxamide |
| 184 | (2R)-[({1-ethyl-4-[(4-oxocyclohexyl)amino]-1H-pyrazolo[3,4-b]pyridin-5- |
| | yl}carbonyl)amino][3-(methyloxy)phenyl]ethanoic acid |
| 185 | 1-ethyl-N-{1-[4-(1-methylethyl)phenyl]ethyl}-4-[(4-oxocyclohexyl)amino]- |
| | 1H-pyrazolo[3,4-b]pyridine-5-carboxamide |
| 186 | 1-ethyl-N-[1-(2-methylphenyl)ethyl]-4-[(4-oxocyclohexyl)amino]-1H- |
| | pyrazolo[3,4-b]pyridine-5-carboxamide |
| 187 | N-[1-(3,5-dimethylphenyl)ethyl]-1-ethyl-4-[(4-oxocyclohexyl)amino]-1H- |
| | pyrazolo[3,4-b]pyridine-5-carboxamide |
| 188 | 1-ethyl-N-{(1R)-1-[4-(methyloxy)phenyl]ethyl}-4-[(4- |
| | oxocyclohexyl)amino]-1H-pyrazolo[3,4-b]pyridine-5-carboxamide |
| 189 | 1-ethyl-N-[1-(4-fluorophenyl)propyl]-4-[(4-oxocyclohexyl)amino]-1H- |
| | pyrazolo[3,4-b]pyridine-5-carboxamide |
| 190 | N-[1-(2,3-dichlorophenyl)propyl]-1-ethyl-4-[(4-oxocyclohexyl)amino]-1H- |
| | pyrazolo[3,4-b]pyridine-5-carboxamide |
| 191 | 1-ethyl-N-[(1R)-1-(4-methylphenyl)ethyl]-4-[(4-oxocyclohexyl)amino]-1H- |
| | pyrazolo[3,4-b]pyridine-5-carboxamide |
| 192 | 1-ethyl-4-[(4-oxocyclohexyl)amino]-N-(1-phenylethyl)-1H-pyrazolo[3,4- |
| | b]pyridine-5-carboxamide |
| 193 | N-[(1R)-1-(4-bromophenyl)ethyl]-1-ethyl-4-[(4-oxocyclohexyl)amino]-1H- |
| | pyrazolo[3,4-b]pyridine-5-carboxamide |
| 194 | 1-ethyl-N-[(1S)-2-hydroxy-1-phenylethyl]-4-[(4-oxocyclohexyl)amino]-1H- |
| | pyrazolo[3,4-b]pyridine-5-carboxamide |
| 195 | N-[1-(4-chlorophenyl)-2-hydroxyethyl]-1-ethyl-4-[(4- |
| | oxocyclohexyl)amino]-1H-pyrazolo[3,4-b]pyridine-5-carboxamide |
| 196 | N-(1-{4-[(difluoromethyl)oxy]phenyl}ethyl)-1-ethyl-4-[(4- |
| | oxocyclohexyl)amino]-1H-pyrazolo[3,4-b]pyridine-5-carboxamide |
| 197 | 1-ethyl-4-[(4-oxocyclohexyl)amino]-N-{1-[4- |
| | (trifluoromethyl)phenyl]ethyl}-1H-pyrazolo[3,4-b]pyridine-5-carboxamide |
| 198 | 1-ethyl-N-[1-(2-methylphenyl)propyl]-4-[(4-oxocyclohexyl)amino]-1H- |
| | pyrazolo[3,4-b]pyridine-5-carboxamide |
| 199 | 1-ethyl-N-{1-[4-(ethyloxy)phenyl]propyl}-4-[(4-oxocyclohexyl)amino]-1H- |
| | pyrazolo[3,4-b]pyridine-5-carboxamide |
| 200 | N-(1-{4-[(difluoromethyl)oxy]phenyl}propyl)-1-ethyl-4-[(4- |
| | oxocyclohexyl)amino]-1H-pyrazolo[3,4-b]pyridine-5-carboxamide |
| 201 | 1-ethyl-4-[(4-oxocyclohexyl)amino]-N-{1-[4- |
| | (trifluoromethyl)phenyl]propyl}-1H-pyrazolo[3,4-b]pyridine-5- |
| | carboxamide |
| 202 | N-[1-(3,4-dimethylphenyl)propyl]-1-ethyl-4-[(4-oxocyclohexyl)amino]-1H- |
| | pyrazolo[3,4-b]pyridine-5-carboxamide |
| 203 | 1-ethyl-4-[(4-oxocyclohexyl)amino]-N-[(1R)-1-phenylpropyl]-1H- |
| | pyrazolo[3,4-b]pyridine-5-carboxamide |
| 204 | 1-ethyl-N-{(1R)-1-[3-(methyloxy)phenyl]ethyl}-4-[(4- |
| | oxocyclohexyl)amino]-1H-pyrazolo[3,4-b]pyridine-5-carboxamide |
| 205 | N-[1-(2,3-dimethylphenyl)ethyl]-1-ethyl-4-[(4-oxocyclohexyl)amino]-1H- |
| | pyrazolo[3,4-b]pyridine-5-carboxamide |
| 206 | N-[1-(2,4-dimethylphenyl)ethyl]-1-ethyl-4-[(4-oxocyclohexyl)amino]-1H- |
| | pyrazolo[3,4-b]pyridine-5-carboxamide |
| 207 | N-[1-(4-chloro-2-fluorophenyl)ethyl]-1-ethyl-4-[(4-oxocyclohexyl)amino]- |
| | 1H-pyrazolo[3,4-b]pyridine-5-carboxamide |
| 208 | N-[1-(3-chloro-4-methylphenyl)ethyl]-1-ethyl-4-[(4-oxocyclohexyl)amino]- |
| | 1H-pyrazolo[3,4-b]pyridine-5-carboxamide |
| 209 | N-[1-(2,3-dimethylphenyl)propyl]-1-ethyl-4-[(4-oxocyclohexyl)amino]-1H- |
| | pyrazolo[3,4-b]pyridine-5-carboxamide |
| 210 | N-[1-(2,4-dimethylphenyl)propyl]-1-ethyl-4-[(4-oxocyclohexyl)amino]-1H- |
| | pyrazolo[3,4-b]pyridine-5-carboxamide |
| 211 | N-[1-(4-chloro-2-fluorophenyl)propyl]-1-ethyl-4-[(4- |
| | oxocyclohexyl)amino]-1H-pyrazolo[3,4-b]pyridine-5-carboxamide |
| 212 | N-[1-(3-chloro-4-methylphenyl)propyl]-1-ethyl-4-[(4- |
| | oxocyclohexyl)amino]-1H-pyrazolo[3,4-b]pyridine-5-carboxamide |
| 213 | 1-ethyl-N-[1-(3-hydroxyphenyl)ethyl]-4-[(4-oxocyclohexyl)amino]-1H- |
| | pyrazolo[3,4-b]pyridine-5-carboxamide |
| 214 | 1-ethyl-N-[1-(3-hydroxyphenyl)propyl]-4-[(4-oxocyclohexyl)amino]-1H- |
| | pyrazolo[3,4-b]pyridine-5-carboxamide |
| 215 | N-[1-(2,3-dichlorophenyl)ethyl]-1-ethyl-4-[(4-oxocyclohexyl)amino]-1H- |
| | pyrazolo[3,4-b]pyridine-5-carboxamide |
| 216 | 1-ethyl-N-{1-[3-(methyloxy)phenyl]propyl}-4-[(4-oxocyclohexyl)amino]- |
| | 1H-pyrazolo[3,4-b]pyridine-5-carboxamide |
| 217 | 1-ethyl-N-{1-[4-(methyloxy)phenyl]propyl}-4-[(4-oxocyclohexyl)amino]- |
| | 1H-pyrazolo[3,4-b]pyridine-5-carboxamide |
| 218 | N-[1-(4-bromophenyl)propyl]-1-ethyl-4-[(4-oxocyclohexyl)amino]-1H- |
| | pyrazolo[3,4-b]pyridine-5-carboxamide |
| 219 | 1-ethyl-4-[(4-oxocyclohexyl)amino]-N-{1-[4-(propyloxy)phenyl]propyl}- |
| | 1H-pyrazolo[3,4-b]pyridine-5-carboxamide |
| 220 | N-[1-(3,5-dimethylphenyl)propyl]-1-ethyl-4-[(4-oxocyclohexyl)amino]-1H- |
| | pyrazolo[3,4-b]pyridine-5-carboxamide |
| 221 | 1-ethyl-N-[1-(4-methylphenyl)propyl]-4-[(4-oxocyclohexyl)amino]-1H- |
| | pyrazolo[3,4-b]pyridine-5-carboxamide |
| 222 | 1-ethyl-N-{1-[4-(1-methylethyl)phenyl]propyl}-4-[(4- |
| | oxocyclohexyl)amino]-1H-pyrazolo[3,4-b]pyridine-5-carboxamide |
| 223 | 1-ethyl-N-(1-{4-[(1-methylethyl)oxy]phenyl}ethyl)-4-[(4- |
| | oxocyclohexyl)amino]-1H-pyrazolo[3,4-b]pyridine-5-carboxamide |
| 224 | 1-ethyl-4-[(4-oxocyclohexyl)amino]-N-[1-(5,6,7,8-tetrahydro-2- |
| | naphthalenyl)ethyl]-1H-pyrazolo[3,4-b]pyridine-5-carboxamide |
| 225 | N-[1-(4-bromophenyl)-2,2,2-trifluoroethyl]-1-ethyl-4-[(4- |
| | oxocyclohexyl)amino]-1H-pyrazolo[3,4-b]pyridine-5-carboxamide |
| 226 | 1-ethyl-4-[(4-oxocyclohexyl)amino]-N-{2,2,2-trifluoro-1-[3- |
| | (methyloxy)phenyl]ethyl}-1H-pyrazolo[3,4-b]pyridine-5-carboxamide |
| 227 | 1-ethyl-4-{[4-(hydroxyimino)cyclohexyl]amino}-N-[1-(5,6,7,8-tetrahydro- |
| | 2-naphthalenyl)ethyl]-1H-pyrazolo[3,4-b]pyridine-5-carboxamide |
| 228 | 1-ethyl-4-{[4-(hydroxyimino)cyclohexyl]amino}-N-[(1S)-2-hydroxy-1- |
| | phenylethyl]-1H-pyrazolo[3,4-b]pyridine-5-carboxamide |
| 229 | N-[1-(2,3-dihydro-1H-inden-5-yl)ethyl]-1-ethyl-4-{[4- |
| | (hydroxyimino)cyclohexyl]amino}-1H-pyrazolo[3,4-b]pyridine-5- |
| | carboxamide |
| 230 | N-[1-(4-chlorophenyl)-2-hydroxyethyl]-1-ethyl-4-{[4- |
| | (hydroxyimino)cyclohexyl]amino}-1H-pyrazolo[3,4-b]pyridine-5- |
| | carboxamide |
| 231 | 1-ethyl-N-{1-[4-(ethyloxy)phenyl]ethyl}-4-{[4- |
| | (hydroxyimino)cyclohexyl]amino}-1H-pyrazolo[3,4-b]pyridine-5- |
| | carboxamide |
| 232 | 1-ethyl-4-{[4-(hydroxyimino)cyclohexyl]amino}-N-{1-[4- |
| | (propyloxy)phenyl]ethyl}-1H-pyrazolo[3,4-b]pyridine-5-carboxamide |
| 233 | 1-ethyl-N-[1-(4-fluorophenyl)ethyl]-4-{[4- |
| | (hydroxyimino)cyclohexyl]amino}-1H-pyrazolo[3,4-b]pyridine-5- |
| | carboxamide |
| 234 | 1-ethyl-4-{[4-(hydroxyimino)cyclohexyl]amino}-N-[(1R)-2-hydroxy-1- |
| | phenylethyl]-1H-pyrazolo[3,4-b]pyridine-5-carboxamide |
| 235 | 1-ethyl-4-{[4-(hydroxyimino)cyclohexyl]amino}-N-(1-phenylpropyl)-1H- |
| | pyrazolo[3,4-b]pyridine-5-carboxamide |
| 236 | 1-ethyl-4-{[4-(hydroxyimino)cyclohexyl]amino}-N-{1-[4-(1- |
| | methylethyl)phenyl]ethyl}-1H-pyrazolo[3,4-b]pyridine-5-carboxamide |
| 237 | N-[1-(3,5-dimethylphenyl)ethyl]-1-ethyl-4-{[4- |
| | (hydroxyimino)cyclohexyl]amino}-1H-pyrazolo[3,4-b]pyridine-5- |
| | carboxamide |
| 238 | 1-ethyl-4-{[4-(hydroxyimino)cyclohexyl]amino}-N-{(1R)-1-[4- |
| | (methyloxy)phenyl]ethyl}-1H-pyrazolo[3,4-b]pyridine-5-carboxamide |
| 239 | 1-ethyl-N-[1-(4-fluorophenyl)propyl]-4-{[4- |
| | (hydroxyimino)cyclohexyl]amino}-1H-pyrazolo[3,4-b]pyridine-5- |
| | carboxamide |
| 240 | N-[1-(2,3-dichlorophenyl)propyl]-1-ethyl-4-{[4- |
| | (hydroxyimino)cyclohexyl]amino}-1H-pyrazolo[3,4-b]pyridine-5- |
| | carboxamide |
| 241 | 1-ethyl-4-{[4-(hydroxyimino)cyclohexyl]amino}-N-[(1R)-1-(4- |
| | methylphenyl)ethyl]-1H-pyrazolo[3,4-b]pyridine-5-carboxamide |
| 242 | 1-ethyl-4-{[4-(hydroxyimino)cyclohexyl]amino}-N-(1-phenylethyl)-1H- |
| | pyrazolo[3,4-b]pyridine-5-carboxamide |
| 243 | N-[(1R)-1-(4-bromophenyl)ethyl]-1-ethyl-4-{[4- |
| | (hydroxyimino)cyclohexyl]amino}-1H-pyrazolo[3,4-b]pyridine-5- |
| | carboxamide |
| 244 | N-[1-(2,3-dichlorophenyl)ethyl]-1-ethyl-4-{[4- |
| | (hydroxyimino)cyclohexyl]amino}-1H-pyrazolo[3,4-b]pyridine-5- |
| | carboxamide |
| 245 | N-[1-(4-chlorophenyl)propyl]-1-ethyl-4-{[4- |
| | (hydroxyimino)cyclohexyl]amino}-1H-pyrazolo[3,4-b]pyridine-5- |
| | carboxamide |
| 246 | N-[1-(4-chlorophenyl)ethyl]-1-ethyl-4-{[4- |
| | (hydroxyimino)cyclohexyl]amino}-1H-pyrazolo[3,4-b]pyridine-5- |
| | carboxamide |
| 247 | 1-ethyl-4-{[4-(hydroxyimino)cyclohexyl]amino}-N-{1-[3- |
| | (methyloxy)phenyl]propyl}-1H-pyrazolo[3,4-b]pyridine-5-carboxamide |
| 248 | 1-ethyl-4-{[4-(hydroxyimino)cyclohexyl]amino}-N-{1-[4- |
| | (methyloxy)phenyl]propyl}-1H-pyrazolo[3,4-b]pyridine-5-carboxamide |
| 249 | N-[1-(4-bromophenyl)propyl]-1-ethyl-4-{[4- |
| | (hydroxyimino)cyclohexyl]amino}-1H-pyrazolo[3,4-b]pyridine-5- |
| | carboxamide |
| 250 | 1-ethyl-4-{[4-(hydroxyimino)cyclohexyl]amino}-N-{1-[4- |
| | (propyloxy)phenyl]propyl}-1H-pyrazolo[3,4-b]pyridine-5-carboxamide |
| 251 | N-[1-(3,5-dimethylphenyl)propyl]-1-ethyl-4-{[4- |
| | (hydroxyimino)cyclohexyl]amino}-1H-pyrazolo[3,4-b]pyridine-5- |
| | carboxamide |
| 252 | 1-ethyl-4-{[4-(hydroxyimino)cyclohexyl]amino}-N-[1-(4- |
| | methylphenyl)propyl]-1H-pyrazolo[3,4-b]pyridine-5-carboxamide |
| 253 | 1-ethyl-4-{[4-(hydroxyimino)cyclohexyl]amino}-N-{1-[4-(1- |
| | methylethyl)phenyl]propyl}-1H-pyrazolo[3,4-b]pyridine-5-carboxamide |
| 254 | 1-ethyl-4-{[4-(hydroxyimino)cyclohexyl]amino}-N-[1-(2- |
| | methylphenyl)ethyl]-1H-pyrazolo[3,4-b]pyridine-5-carboxamide |
| 255 | N-(1-{4-[(difluoromethyl)oxy]phenyl}ethyl)-1-ethyl-4-{[4- |
| | (hydroxyimino)cyclohexyl]amino}-1H-pyrazolo[3,4-b]pyridine-5- |
| | carboxamide |
| 256 | 1-ethyl-4-{[4-(hydroxyimino)cyclohexyl]amino}-N-{1-[4- |
| | (trifluoromethyl)phenyl]ethyl}-1H-pyrazolo[3,4-b]pyridine-5-carboxamide |
| 257 | 1-ethyl-4-{[4-(hydroxyimino)cyclohexyl]amino}-N-[1-(2- |
| | methylphenyl)propyl]-1H-pyrazolo[3,4-b]pyridine-5-carboxamide |
| 258 | 1-ethyl-N-{1-[4-(ethyloxy)phenyl]propyl}-4-{[4- |
| | (hydroxyimino)cyclohexyl]amino}-1H-pyrazolo[3,4-b]pyridine-5- |
| | carboxamide |
| 259 | N-(1-{4-[(difluoromethyl)oxy]phenyl}propyl)-1-ethyl-4-{[4- |
| | (hydroxyimino)cyclohexyl]amino}-1H-pyrazolo[3,4-b]pyridine-5- |
| | carboxamide |
| 260 | 1-ethyl-4-{[4-(hydroxyimino)cyclohexyl]amino}-N-{1-[4- |
| | (trifluoromethyl)phenyl]propyl}-1H-pyrazolo[3,4-b]pyridine-5- |
| | carboxamide |
| 261 | N-[1-(3,4-dimethylphenyl)propyl]-1-ethyl-4-{[4- |
| | (hydroxyimino)cyclohexyl]amino}-1H-pyrazolo[3,4-b]pyridine-5- |
| | carboxamide |
| 262 | 1-ethyl-4-{[4-(hydroxyimino)cyclohexyl]amino}-N-[(1R)-1-phenylpropyl]- |
| | 1H-pyrazolo[3,4-b]pyridine-5-carboxamide |
| 263 | 1-ethyl-4-{[4-(hydroxyimino)cyclohexyl]amino}-N-{(1R)-1-[3- |
| | (methyloxy)phenyl]ethyl}-1H-pyrazolo[3,4-b]pyridine-5-carboxamide |
| 264 | N-[1-(2,3-dimethylphenyl)ethyl]-1-ethyl-4-{[4- |
| | (hydroxyimino)cyclohexyl]amino}-1H-pyrazolo[3,4-b]pyridine-5- |
| | carboxamide |
| 265 | N-[1-(2,4-dimethylphenyl)ethyl]-1-ethyl-4-{[4- |
| | (hydroxyimino)cyclohexyl]amino}-1H-pyrazolo[3,4-b]pyridine-5- |
| | carboxamide |
| 266 | N-[1-(4-chloro-2-fluorophenyl)ethyl]-1-ethyl-4-{[4- |
| | (hydroxyimino)cyclohexyl]amino}-1H-pyrazolo[3,4-b]pyridine-5- |
| | carboxamide |
| 267 | N-[1-(3-chloro-4-methylphenyl)ethyl]-1-ethyl-4-{[4- |
| | (hydroxyimino)cyclohexyl]amino}-1H-pyrazolo[3,4-b]pyridine-5- |
| | carboxamide |
| 268 | N-[1-(2,3-dimethylphenyl)propyl]-1-ethyl-4-{[4- |
| | (hydroxyimino)cyclohexyl]amino}-1H-pyrazolo[3,4-b]pyridine-5- |
| | carboxamide |
| 269 | N-[1-(2,4-dimethylphenyl)propyl]-1-ethyl-4-{[4- |
| | (hydroxyimino)cyclohexyl]amino}-1H-pyrazolo[3,4-b]pyridine-5- |
| | carboxamide |
| 270 | N-[1-(4-chloro-2-fluorophenyl)propyl]-1-ethyl-4-{[4- |
| | (hydroxyimino)cyclohexyl]amino}-1H-pyrazolo[3,4-b]pyridine-5- |
| | carboxamide |
| 271 | N-[1-(3-chloro-4-methylphenyl)propyl]-1-ethyl-4-{[4- |
| | (hydroxyimino)cyclohexyl]amino}-1H-pyrazolo[3,4-b]pyridine-5- |
| | carboxamide |
| 272 | 1-ethyl-4-{[4-(hydroxyimino)cyclohexyl]amino}-N-[1-(3- |
| | hydroxyphenyl)ethyl]-1H-pyrazolo[3,4-b]pyridine-5-carboxamide |
| 273 | 1-ethyl-4-{[4-(hydroxyimino)cyclohexyl]amino}-N-[1-(3- |
| | hydroxyphenyl)propyl]-1H-pyrazolo[3,4-b]pyridine-5-carboxamide |
| 274 | N-[1-(2,4-dimethylphenyl)ethyl]-1-ethyl-4-{[4- |
| | (hydroxyimino)cyclohexyl]amino}-1H-pyrazolo[3,4-b]pyridine-5- |
| | carboxamide |
| 275 | N-[1-(2,4-dimethylphenyl)ethyl]-1-ethyl-4-{[4- |
| | (hydroxyimino)cyclohexyl]amino}-1H-pyrazolo[3,4-b]pyridine-5- |
| | carboxamide |
| 276 | N-[1-(3,5-dimethylphenyl)ethyl]-1-ethyl-4-{[4- |
| | (hydroxyimino)cyclohexyl]amino}-1H-pyrazolo[3,4-b]pyridine-5- |
| | carboxamide |
| 277 | N-[1-(3,5-dimethylphenyl)ethyl]-1-ethyl-4-{[4- |
| | (hydroxyimino)cyclohexyl]amino}-1H-pyrazolo[3,4-b]pyridine-5- |
| | carboxamide |
| 278 | 1-ethyl-4-{[4-(hydroxyimino)cyclohexyl]amino}-N-(1-{4-[(1- |
| | methylethyl)oxy]phenyl}ethyl)-1H-pyrazolo[3,4-b]pyridine-5-carboxamide |
| 279 | 1-ethyl-4-{[4-(hydroxyimino)cyclohexyl]amino}-N-(1-{4-[(1- |
| | methylethyl)oxy]phenyl}ethyl)-1H-pyrazolo[3,4-b]pyridine-5-carboxamide |
| 280 | 1-ethyl-N-[1-(4-fluorophenyl)ethyl]-4-{[4- |
| | (hydroxyimino)cyclohexyl]amino}-1H-pyrazolo[3,4-b]pyridine-5- |
| | carboxamide |
| 281 | 1-ethyl-N-[1-(4-fluorophenyl)ethyl]-4-{[4- |
| | (hydroxyimino)cyclohexyl]amino}-1H-pyrazolo[3,4-b]pyridine-5- |
| | carboxamide |
| 282 | N-[1-(4-chlorophenyl)propyl]-1-ethyl-4-{[(1S,3R)- and/or (1R,3S)-3- |
| | hydroxycyclohexyl]amino}-1H-pyrazolo[3,4-b]pyridine-5-carboxamide |
| 283 | 1-ethyl-4-{[(1S,3R)- and/or (1R,3S)-3-hydroxycyclohexyl]amino}-N-[(1R)-1- |
| | (4-methylphenyl)ethyl]-1H-pyrazolo[3,4-b]pyridine-5-carboxamide |
| 284 | N-[1-(2,4-dimethylphenyl)ethyl]-1-ethyl-4-{[(1S,3R)- and/or (1R,3S)-3- |
| | hydroxycyclohexyl]amino}-1H-pyrazolo[3,4-b]pyridine-5-carboxamide |
| | (Isomer 1) |
| 285 | N-[1-(2,4-dimethylphenyl)ethyl]-1-ethyl-4-{[(1S,3R)- and/or (1R,3S)-3- |
| | hydroxycyclohexyl]amino}-1H-pyrazolo[3,4-b]pyridine-5-carboxamide |
| | (Isomer 2) |
| 286 | N-[1-(3,4-dimethylphenyl)propyl]-1-ethyl-4-{[(1S,3R)- and/or (1R,3S)-3- |
| | hydroxycyclohexyl]amino}-1H-pyrazolo[3,4-b]pyridine-5-carboxamide |
| 287 | N-[1-(4-chlorophenyl)propyl]-1-ethyl-6-methyl-4-(tetrahydro-2H-pyran-4- |
| | ylamino)-1H-pyrazolo[3,4-b]pyridine-5-carboxamide |
| 288 | N-[1-(4-chlorophenyl)ethyl]-1-ethyl-6-methyl-4-(tetrahydro-2H-pyran-4- |
| | ylamino)-1H-pyrazolo[3,4-b]pyridine-5-carboxamide |
| 289 | N-[1-(4-chlorophenyl)ethyl]-1-ethyl-4-(tetrahydro-2H-pyran-4-ylamino)- |
| | 1H-pyrazolo[3,4-b]pyridine-5-carboxamide (Enantiomer 1) |
| 290 | N-[1-(4-chlorophenyl)ethyl]-1-ethyl-4-(tetrahydro-2H-pyran-4-ylamino)- |
| | 1H-pyrazolo[3,4-b]pyridine-5-carboxamide (Enantiomer 2) |
| 291 | N-[1-(4-chlorophenyl)propyl]-1-ethyl-4-(tetrahydro-2H-pyran-4-ylamino)- |
| | 1H-pyrazolo[3,4-b]pyridine-5-carboxamide (Enantiomer 1) |
| 292 | N-[1-(4-chlorophenyl)propyl]-1-ethyl-4-(tetrahydro-2H-pyran-4-ylamino)- |
| | 1H-pyrazolo[3,4-b]pyridine-5-carboxamide (Enantiomer 2) |
| 293 | 1-ethyl-N-{1-[4-(ethyloxy)phenyl]ethyl}-4-(tetrahydro-2H-pyran-4- |
| | ylamino)-1H-pyrazolo[3,4-b]pyridine-5-carboxamide (Enantiomer 1) |
| 294 | 1-ethyl-N-{1-[4-(ethyloxy)phenyl]ethyl}-4-(tetrahydro-2H-pyran-4- |
| | ylamino)-1H-pyrazolo[3,4-b]pyridine-5-carboxamide (Enantiomer 2) |
| 295 | N-[1-(2,4-dimethylphenyl)ethyl]-1-ethyl-4-[(4-oxocyclohexyl)amino]-1H- |
| | pyrazolo[3,4-b]pyridine-5-carboxamide (Enantiomer 1) |
| 296 | N-[1-(2,4-dimethylphenyl)ethyl]-1-ethyl-4-[(4-oxocyclohexyl)amino]-1H- |
| | pyrazolo[3,4-b]pyridine-5-carboxamide (Enantiomer 2) |
| 297 | N-[1-(3,5-dimethylphenyl)ethyl]-1-ethyl-4-[(4-oxocyclohexyl)amino]-1H- |
| | pyrazolo[3,4-b]pyridine-5-carboxamide (Enantiomer 1) |
| 298 | N-[1-(3,5-dimethylphenyl)ethyl]-1-ethyl-4-[(4-oxocyclohexyl)amino]-1H- |
| | pyrazolo[3,4-b]pyridine-5-carboxamide (Enantiomer 2) |
| 299 | 1-ethyl-N-(1-{4-[(1-methylethyl)oxy]phenyl}ethyl)-4-[(4- |
| | oxocyclohexyl)amino]-1H-pyrazolo[3,4-b]pyridine-5-carboxamide |
| | (Enantiomer 1) |
| 300 | 1-ethyl-N-(1-{4-[(1-methylethyl)oxy]phenyl}ethyl)-4-[(4- |
| | oxocyclohexyl)amino]-1H-pyrazolo[3,4-b]pyridine-5-carboxamide |
| | (Enantiomer 2) |
| 301 | 1-ethyl-N-[1-(4-fluorophenyl)ethyl]-4-[(4-oxocyclohexyl)amino]-1H- |
| | pyrazolo[3,4-b]pyridine-5-carboxamide (Enantiomer 1) |
| 302 | 1-ethyl-N-[1-(4-fluorophenyl)ethyl]-4-[(4-oxocyclohexyl)amino]-1H- |
| | pyrazolo[3,4-b]pyridine-5-carboxamide (Enantiomer 2) |
| 303 | N-[1-(2,4-dimethylphenyl)propyl]-1-ethyl-4-(tetrahydro-2H-pyran-4- |
| | ylamino)-1H-pyrazolo[3,4-b]pyridine-5-carboxamide (Enantiomer 1) |
| 304 | N-[1-(2,4-dimethylphenyl)propyl]-1-ethyl-4-(tetrahydro-2H-pyran-4- |
| | ylamino)-1H-pyrazolo[3,4-b]pyridine-5-carboxamide (Enantiomer 2) |
| 305 | 1-ethyl-4-{[(1S,3R)- and/or (1R,3S)-3-hydroxycyclohexyl]amino}-N-[(1R)- |
| | 1-(4-methylphenyl)ethyl]-1H-pyrazolo[3,4-b]pyridine-5-carboxamide |
| | (Diastereoisomer 1) |
| 306 | 1-ethyl-4-{[(1S,3R)- and/or (1R,3S)-3-hydroxycyclohexyl]amino}-N-[(1R)- |
| | 1-(4-methylphenyl)ethyl]-1H-pyrazolo[3,4-b]pyridine-5-carboxamide |
| | (Diastereoisomer 2) |
| 307 | N-[1-(2,4-dimethylphenyl)propyl]-1-ethyl-4-(tetrahydro-2H-pyran-4- |
| | ylamino)-1H-pyrazolo[3,4-b]pyridine-5-carboxamide (Enantiomer 2) |
| | hydrochloride |
| 308 | 4-{[1-(aminocarbonyl)-4-piperidinyl]amino}-1-ethyl-N-[(1R)-1-(4- |
| | methylphenyl)ethyl]-1H-pyrazolo[3,4-b]pyridine-5-carboxamide |
| 309 | 4-{[1-(aminocarbonyl)-4-piperidinyl]amino}-1-ethyl-N-[(1R)-1- |
| | phenylethyl]-1H-pyrazolo[3,4-b]pyridine-5-carboxamide |
| 310 | 4-{[1-(aminocarbonyl)-4-piperidinyl]amino}-N-[(1R)-1-(4- |
| | bromophenyl)ethyl]-1-ethyl-1H-pyrazolo[3,4-b]pyridine-5-carboxamide |
| 311 | 4-{[1-(aminocarbonyl)-4-piperidinyl]amino}-N-[1-(2,4- |
| | dimethylphenyl)propyl]-1-ethyl-1H-pyrazolo[3,4-b]pyridine-5-carboxamide |
| 312 | 4-{[1-(aminocarbonyl)-4-piperidinyl]amino}-N-[1-(3-chloro-4- |
| | methylphenyl)propyl]-1-ethyl-1H-pyrazolo[3,4-b]pyridine-5-carboxamide |
| 313 | 4-{[1-(aminocarbonyl)-4-piperidinyl]amino}-N-[1-(4-chloro-2- |
| | fluorophenyl)propyl]-1-ethyl-1H-pyrazolo[3,4-b]pyridine-5-carboxamide |
| 314 | 4-{[4-(aminocarbonyl)cyclohexyl]amino}-1-ethyl-N-[(1R)-1-phenylethyl]- |
| | 1H-pyrazolo[3,4-b]pyridine-5-carboxamide |
| 314A | 4-{cis-[4-(aminocarbonyl)cyclohexyl]amino}-1-ethyl-N-[(1R)-1- |
| | phenylethyl]-1H-pyrazolo[3,4-b]pyridine-5-carboxamide |
| 315 | N-[(1S)-1-(2,4-dimethylphenyl)propyl]-1-ethyl-4-(tetrahydro-2H-pyran-4- |
| | ylamino)-1H-pyrazolo[3,4-b]pyridine-5-carboxamide |
| 316 | N-[(1R)-1-(2,4-dimethylphenyl)propyl]-1-ethyl-4-(tetrahydro-2H-pyran-4- |
| | ylamino)-1H-pyrazolo[3,4-b]pyridine-5-carboxamide |
| 317 | N-[(1R)-1-(2,5-dimethylphenyl)ethyl]-1-ethyl-4-(tetrahydro-2H-pyran-4- |
| | ylamino)-1H-pyrazolo[3,4-b]pyridine-5-carboxamide |
| 318 | 1-ethyl-4-(tetrahydro-2H-pyran-4-ylamino)-N-[(1R)-1-(2,4,6- |
| | trimethylphenyl)ethyl]-1H-pyrazolo[3,4-b]pyridine-5-carboxamide |
| 319 | 1-ethyl-N-[(1R)-1-(2-ethylphenyl)ethyl]-4-(tetrahydro-2H-pyran-4- |
| | ylamino)-1H-pyrazolo[3,4-b]pyridine-5-carboxamide |
| 320 | 1-ethyl-N-[(1R)-1-(4-ethylphenyl)ethyl]-4-(tetrahydro-2H-pyran-4- |
| | ylamino)-1H-pyrazolo[3,4-b]pyridine-5-carboxamide |
| 321 | 1-ethyl-N-[(1R)-1-(4-methylphenyl)propyl]-4-(tetrahydro-2H-pyran-4- |
| | ylamino)-1H-pyrazolo[3,4-b]pyridine-5-carboxamide |
| 322 | 1-ethyl-N-[(1R)-1-(4-ethylphenyl)propyl]-4-(tetrahydro-2H-pyran-4- |
| | ylamino)-1H-pyrazolo[3,4-b]pyridine-5-carboxamide |
| 323 | 1-ethyl-N-{(1R)-1-[4-(1-methylethyl)phenyl]propyl}-4-(tetrahydro-2H- |
| | pyran-4-ylamino)-1H-pyrazolo[3,4-b]pyridine-5-carboxamide |
| 324 | N-[(1R)-1-(4-chloro-2-fluorophenyl)propyl]-1-ethyl-4-(tetrahydro-2H- |
| | pyran-4-ylamino)-1H-pyrazolo[3,4-b]pyridine-5-carboxamide |
| 325 | N-[(1R)-1-(2,6-dimethylphenyl)propyl]-1-ethyl-4-(tetrahydro-2H-pyran-4- |
| | ylamino)-1H-pyrazolo[3,4-b]pyridine-5-carboxamide |
| 326 | N-[(1R)-1-(2,5-dimethylphenyl)propyl]-1-ethyl-4-(tetrahydro-2H-pyran-4- |
| | ylamino)-1H-pyrazolo[3,4-b]pyridine-5-carboxamide |
| 327 | 1-ethyl-N-[(1R)-1-(2-ethylphenyl)propyl]-4-(tetrahydro-2H-pyran-4- |
| | ylamino)-1H-pyrazolo[3,4-b]pyridine-5-carboxamide |
| 328 | 1-ethyl-4-(tetrahydro-2H-pyran-4-ylamino)-N-[(1R)-1-(2,4,6- |
| | trimethylphenyl)propyl]-1H-pyrazolo[3,4-b]pyridine-5-carboxamide |
| 329 | 4-{[1-(aminocarbonyl)-4-piperidinyl]amino}-N-[(1R)-1-(2,5- |
| | dimethylphenyl)ethyl]-1-ethyl-1H-pyrazolo[3,4-b]pyridine-5-carboxamide |
| 330 | 4-{[1-(aminocarbonyl)-4-piperidinyl]amino}-1-ethyl-N-[(1R)-1-(4- |
| | ethylphenyl)ethyl]-1H-pyrazolo[3,4-b]pyridine-5-carboxamide |
| 331 | 4-{[1-(aminocarbonyl)-4-piperidinyl]amino}-1-ethyl-N-[(1R)-1-(2- |
| | ethylphenyl)ethyl]-1H-pyrazolo[3,4-b]pyridine-5-carboxamide |
| 332 | 4-{[1-(aminocarbonyl)-4-piperidinyl]amino}-1-ethyl-N-[(1R)-1-(2,4,6- |
| | trimethylphenyl)ethyl]-1H-pyrazolo[3,4-b]pyridine-5-carboxamide |
| 333 | 4-{[1-(aminocarbonyl)-4-piperidinyl]amino}-N-[(1R)-1-(2,4- |
| | dimethylphenyl)propyl]-1-ethyl-1H-pyrazolo[3,4-b]pyridine-5-carboxamide |
| 334 | 4-{[1-(aminocarbonyl)-4-piperidinyl]amino}-N-[1-(4-chlorophenyl)ethyl]- |
| | 1-ethyl-1H-pyrazolo[3,4-b]pyridine-5-carboxamide |
| 335 | 4-{[1-(aminocarbonyl)-4-piperidinyl]amino}-1-ethyl-N-[(1R)-1- |
| | phenylpropyl]-1H-pyrazolo[3,4-b]pyridine-5-carboxamide |
| 336 | 4-{[1-(aminocarbonyl)-4-piperidinyl]amino}-N-[1-(4- |
| | chlorophenyl)propyl]-1-ethyl-1H-pyrazolo[3,4-b]pyridine-5-carboxamide |
| 337 | 4-{[1-(aminocarbonyl)-4-piperidinyl]amino}-1-ethyl-N-[1-(4- |
| | fluorophenyl)propyl]-1H-pyrazolo[3,4-b]pyridine-5-carboxamide |
| 338 | 4-{[1-(aminocarbonyl)-4-piperidinyl]amino}-1-ethyl-N-[(1R)-1-(4- |
| | methylphenyl)propyl]-1H-pyrazolo[3,4-b]pyridine-5-carboxamide |
| 339 | 4-{[1-(aminocarbonyl)-4-piperidinyl]amino}-1-ethyl-N-[(1R)-1-(4- |
| | ethylphenyl)propyl]-1H-pyrazolo[3,4-b]pyridine-5-carboxamide |
| 340 | 4-{[1-(aminocarbonyl)-4-piperidinyl]amino}-1-ethyl-N-{(1R)-1-[4-(1- |
| | methylethyl)phenyl]propyl}-1H-pyrazolo[3,4-b]pyridine-5-carboxamide |
| 341 | 4-{[1-(aminocarbonyl)-4-piperidinyl]amino}-N-[(1R)-1-(4-chloro-2- |
| | fluorophenyl)propyl]-1-ethyl-1H-pyrazolo[3,4-b]pyridine-5-carboxamide |
| 342 | 4-{[1-(aminocarbonyl)-4-piperidinyl]amino}-N-[(1R)-1-(2,6- |
| | dimethylphenyl)propyl]-1-ethyl-1H-pyrazolo[3,4-b]pyridine-5-carboxamide |
| 343 | 4-{[1-(aminocarbonyl)-4-piperidinyl]amino}-N-[(1R)-1-(2,5- |
| | dimethylphenyl)propyl]-1-ethyl-1H-pyrazolo[3,4-b]pyridine-5-carboxamide |
| 344 | 4-{[1-(aminocarbonyl)-4-piperidinyl]amino}-1-ethyl-N-[(1R)-1-(2- |
| | ethylphenyl)propyl]-1H-pyrazolo[3,4-b]pyridine-5-carboxamide |
| 345 | 4-{[1-(aminocarbonyl)-4-piperidinyl]amino}-1-ethyl-N-[(1R)-1-(2,4,6- |
| | trimethylphenyl)propyl]-1H-pyrazolo[3,4-b]pyridine-5-carboxamide |
| 346 | 4-{[4-(aminocarbonyl)cyclohexyl]amino}-N-[1-(4-chlorophenyl)propyl]-1- |
| | ethyl-1H-pyrazolo[3,4-b]pyridine-5-carboxamide |
| 347 | 4-{[4-(aminocarbonyl)cyclohexyl]amino}-1-ethyl-N-[(1R)-1- |
| | phenylpropyl]-1H-pyrazolo[3,4-b]pyridine-5-carboxamide |
| 348 | 4-{[4-(aminocarbonyl)cyclohexyl]amino}-N-(1-{4- |
| | [(difluoromethyl)oxy]phenyl}ethyl)-1-ethyl-1H-pyrazolo[3,4-b]pyridine-5- |
| | carboxamide |
| 349 | 4-{[4-(aminocarbonyl)cyclohexyl]amino}-N-[1-(4-chlorophenyl)ethyl]-1- |
| | ethyl-1H-pyrazolo[3,4-b]pyridine-5-carboxamide |
| 350 | 4-{[4-(aminocarbonyl)cyclohexyl]amino}-1-ethyl-N-[1-(4- |
| | fluorophenyl)propyl]-1H-pyrazolo[3,4-b]pyridine-5-carboxamide |
| 351 | 4-{[4-(aminocarbonyl)cyclohexyl]amino}-N-[(1R)-1-(4- |
| | bromophenyl)ethyl]-1-ethyl-1H-pyrazolo[3,4-b]pyridine-5-carboxamide |
| 352 | 4-{[cis-4-(aminocarbonyl)cyclohexyl]amino}-N-[(1R)-1-(2,4- |
| | dimethylphenyl)propyl]-1-ethyl-1H-pyrazolo[3,4-b]pyridine-5-carboxamide |
| 353 | 4-{[cis-4-(aminocarbonyl)cyclohexyl]amino}-1-ethyl-N-[(1R)-1-(4- |
| | methylphenyl)ethyl]-1H-pyrazolo[3,4-b]pyridine-5-carboxamide |
| 354 | 4-{[cis-4-(aminocarbonyl)cyclohexyl]amino}-1-ethyl-N-[(1R)-1- |
| | phenylethyl]-1H-pyrazolo[3,4-b]pyridine-5-carboxamide |
| 355 | 4-{[cis-4-(aminocarbonyl)cyclohexyl]amino}-N-[(1R)-1-(4- |
| | bromophenyl)ethyl]-1-ethyl-1H-pyrazolo[3,4-b]pyridine-5-carboxamide |
| 356 | 4-{[trans-4-(aminocarbonyl)cyclohexyl]amino}-N-[(1R)-1-(2,4- |
| | dimethylphenyl)propyl]-1-ethyl-1H-pyrazolo[3,4-b]pyridine-5-carboxamide |
| 357 | 4-{[trans-4-(aminocarbonyl)cyclohexyl]amino}-1-ethyl-N-[(1R)-1-(4- |
| | methylphenyl)ethyl]-1H-pyrazolo[3,4-b]pyridine-5-carboxamide |
| 358 | 4-{[trans-4-(aminocarbonyl)cyclohexyl]amino}-1-ethyl-N-[(1R)-1- |
| | phenylethyl]-1H-pyrazolo[3,4-b]pyridine-5-carboxamide |
| 359 | 4-{[trans-4-(aminocarbonyl)cyclohexyl]amino}-N-[(1R)-1-(4- |
| | bromophenyl)ethyl]-1-ethyl-1H-pyrazolo[3,4-b]pyridine-5-carboxamide |
| 360 | 4-{[(3S)-1-(aminocarbonyl)pyrrolidin-3-yl]amino}-N-[1-(2,4- |
| | dimethylphenyl)propyl]-1-ethyl-1H-pyrazolo[3,4-b]pyridine-5-carboxamide |
| 361 | 4-{[(3S)-1-(aminocarbonyl)pyrrolidin-3-yl]amino}-1-ethyl-N-[(1R)-1-(4- |
| | methylphenyl)ethyl]-1H-pyrazolo[3,4-b]pyridine-5-carboxamide |
| 362 | 4-{[(3S)-1-(aminocarbonyl)pyrrolidin-3-yl]amino}-N-[1-(3,4- |
| | dimethylphenyl)propyl]-1-ethyl-1H-pyrazolo[3,4-b]pyridine-5-carboxamide |
| 363 | 4-{[(3S)-1-(aminocarbonyl)pyrrolidin-3-yl]amino}-N-[(1R)-1-(4- |
| | bromophenyl)ethyl]-1-ethyl-1H-pyrazolo[3,4-b]pyridine-5-carboxamide |
| 364 | 4-{[(3R)-1-(aminocarbonyl)pyrrolidin-3-yl]amino}-N-[1-(2,4- |
| | dimethylphenyl)propyl]-1-ethyl-1H-pyrazolo[3,4-b]pyridine-5-carboxamide |
| 365 | 4-{[(3R)-1-(aminocarbonyl)pyrrolidin-3-yl]amino}-1-ethyl-N-[(1R)-1-(4- |
| | methylphenyl)ethyl]-1H-pyrazolo[3,4-b]pyridine-5-carboxamide |
| 366 | 4-{[(3R)-1-(aminocarbonyl)pyrrolidin-3-yl]amino}-N-[1-(3,4- |
| | dimethylphenyl)propyl]-1-ethyl-1H-pyrazolo[3,4-b]pyridine-5-carboxamide |
| 367 | 4-{[(3R)-1-(aminocarbonyl)pyrrolidin-3-yl]amino}-N-[(1R)-1-(4- |
| | bromophenyl)ethyl]-1-ethyl-1H-pyrazolo[3,4-b]pyridine-5-carboxamide |
| 368 | 4-{[cis-3-(aminocarbonyl)cyclobutyl]amino}-1-ethyl-N-[(1R)-1-(4- |
| | methylphenyl)ethyl]-1H-pyrazolo[3,4-b]pyridine-5-carboxamide |
| 369 | 4-{[cis-3-(aminocarbonyl)cyclobutyl]amino}-N-[1-(2,4- |
| | dimethylphenyl)propyl]-1-ethyl-1H-pyrazolo[3,4-b]pyridine-5-carboxamide |
| 370 | 4-[(trans-4-acetylcyclohexyl)amino]-1-ethyl-N-[(1R)-1-(4- |
| | methylphenyl)ethyl]-1H-pyrazolo[3,4-b]pyridine-5-carboxamide |
| 371 | 4-[(4-acetylcyclohexyl)amino]-N-[(1R)-1-(2,4-dimethylphenyl)propyl]-1- |
| | ethyl-1H-pyrazolo[3,4-b]pyridine-5-carboxamide |
| 372 | 4-[(cis-4-acetylcyclohexyl)amino]-1-ethyl-N-[(1R)-1-(4- |
| | methylphenyl)ethyl]-1H-pyrazolo[3,4-b]pyridine-5-carboxamide |
| 373 | 4-{[cis-4-(1-hydroxyethyl)cyclohexyl]amino}-N-[1-(2,4- |
| | dimethylphenyl)propyl]-1-ethyl-1H-pyrazolo[3,4-b]pyridine-5-carboxamide |
| 374 | 1-ethyl-4-{[trans-3-hydroxycyclohexyl]amino}-N-[(1R)-1-(4- |
| | methylphenyl)ethyl]-1H-pyrazolo[3,4-b]pyridine-5-carboxamide |
| 375 | N-[(1S)-1-(2,4-dimethylphenyl)ethyl]-1-ethyl-4-{[trans-3- |
| | hydroxycyclohexyl]amino}-1H-pyrazolo[3,4-b]pyridine-5-carboxamide |
| 376 | N-[(1R)-1-(2,4-dimethylphenyl)ethyl]-1-ethyl-4-{[trans-3- |
| | hydroxycyclohexyl]amino}-1H-pyrazolo[3,4-b]pyridine-5-carboxamide |
| 377 | N-[(1R)-1-(4-bromophenyl)ethyl]-1-ethyl-4-{[trans-3- |
| | hydroxycyclohexyl]amino}-1H-pyrazolo[3,4-b]pyridine-5-carboxamide |
| 378 | N-[1-(3,4-dimethylphenyl)propyl]-1-ethyl-4-{[trans-3- |
| | hydroxycyclohexyl]amino}-1H-pyrazolo[3,4-b]pyridine-5-carboxamide |
| 379 | N-[4-(dimethylamino)-1-(3-methylphenyl)-4-oxobutyl]-1-ethyl-4- |
| | (tetrahydro-2H-pyran-4-ylamino)-1H-pyrazolo[3,4-b]pyridine-5- |
| | carboxamide |
| 380 | 4-{[1-(aminocarbonyl)-4-piperidinyl]amino}-N-[4-(dimethylamino)-1-(3- |
| | methylphenyl)-4-oxobutyl]-1-ethyl-1H-pyrazolo[3,4-b]pyridine-5- |
| | carboxamide |
| 381 | 1-ethyl-N-[(1R)-1-(4-methylphenyl)ethyl]-4-(4-piperidinylamino)-1H- |
| | pyrazolo[3,4-b]pyridine-5-carboxamide hydrochloride |
| 382 | N-[1-(2,4-dimethylphenyl)propyl]-1-ethyl-4-(4-piperidinylamino)-1H- |
| | pyrazolo[3,4-b]pyridine-5-carboxamide hydrochloride |

### Examples 1 to 105

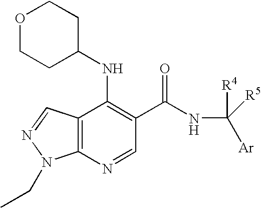

General Procedure:

A mixture of Intermediate 13 (0.1 mmol), HATU (0.1 mmol) and DIPEA (0.4 mmol) in DMF (0.4 ml) was shaken at room temperature for 10 min. A solution of the amine reagent Ar—C(R⁴)(R⁵)—NH₂ (0.1 mmol) in DMF (0.2 ml) was then added and the mixture was agitated for several minutes to give a solution. The solution was stored at room temperature for 16 hours then concentrated in vacuo. The residue was dissolved in chloroform (0.5 ml) and applied to a SPE cartridge (aminopropyl, 0.5 g). The cartridge was eluted successively with chloroform (1.5 ml), EtOAc (1.5 ml) and EtOAc:MeOH (9:1, 1.5 ml). Fractions containing the desired product were concentrated in vacuo and the residue purified by mass directed autoprep HPLC.

The following Examples 1 to 105 were prepared from Intermediate 13 and the appropriate amine reagent Ar-C(R⁴)(R⁵)-NH₂ using the above or a similar procedure:

| Example Number | 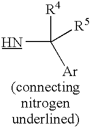 | 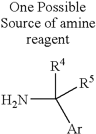 | MH⁺ Ion | LC-MS retention time |
|---|---|---|---|---|
| 1 | 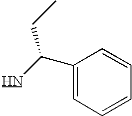 | Lancaster | 408 | 3.05 |
| 2 | 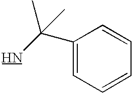 | Fluorochem. Ltd. | 408 | 2.69 |
| 3 | 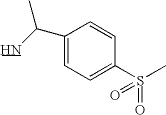 | Peakdale Molecular Ltd. | 472 | 2.44 |
| 4 | 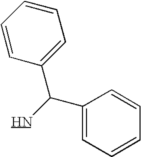 | Aldrich | 456 | 3.06 |
| 5 | 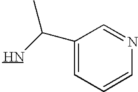 | | 395 | 1.83 |
| 6 | 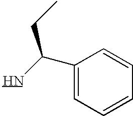 | Lancaster | 408 | 2.81 |
| 7 | 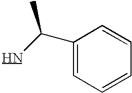 | Aldrich | 394 | 2.64 |
| 8 | 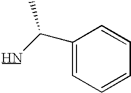 | Aldrich | 394 | 2.89 |
| 9 | 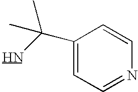 | | 409 | 1.89 |
| 10 | 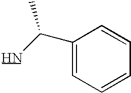 | Aldrich | 394 | 2.91 |
| 11 | 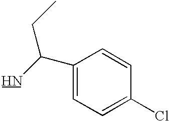 | J. Pharm. Pharmacol; 1997, 49(1), 10-15 | 442 +444 | 3.22 |
| 12 | 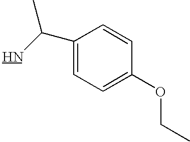 | Tim Tec Building Blocks Inc. (Intermediate 64) | 438 | 2.98 |
| 13 | 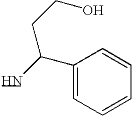 | Acros | 424 | 2.71 |
| 14 | 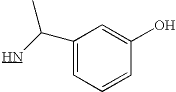 | Tetrahedron, 1977, 33(5), 489-495 (Intermediate 88) | 410 | 2.70 |
| 15 | 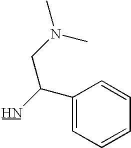 | MicroChemistry Building Blocks | 437 | 2.34 |
| 16 | 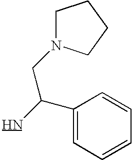 | MicroChemistry Building Blocks | 463 | 2.37 |
| 17 | 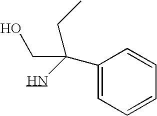 | EP 534553 Al (1993) | 438 | 2.83 |
| 18 | 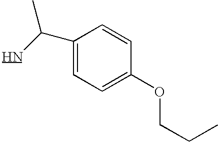 | Biochem. Pharm. 1959, 2, 264-9 (no ref. To preparation) | 452 | 3.22 |
| 19 | 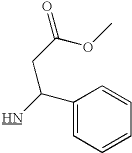 | Chembridge Europe | 452 | 2.95 |
| 20 | 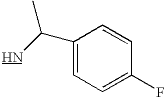 | Aldrich | 412 | 3.06 |
| 21 | 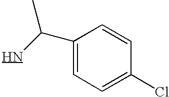 | Bionet Research | 428 +430 | 3.24 |
| 22 | 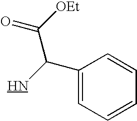 | Maybridge Combichem. | 452 | 3.10 |
| 23 | 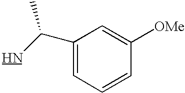 | Lancaster | 424 | 3.01 |
| 24 | 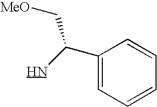 | OmegaChem | 424 | 2.90 |
| 25 | 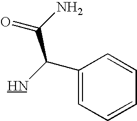 | Acros | 423 | 2.57 |
| 26 | 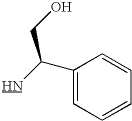 | Aldrich | 410 | 2.67 |
| 27 | 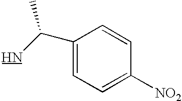 | Aldrich (hydrochloride) | 439 | 3.07 |
| 28 | 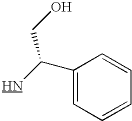 | Aldrich | 410 | 2.67 |
| 29 | 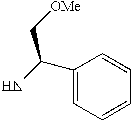 | Omega Chem | 424 | 2.90 |
| 30 | 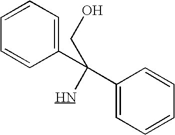 | Org. Lett; 2001, 3 (2), 299-302 | 486 | 3.09 |
| 31 | 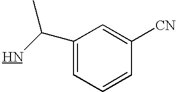 | J. Amer. Chem. Soc; 1990, 112, 5741-5747 | 419 | 2.98 |
| 32 | 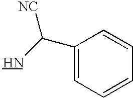 | Aldrich | 405 | 3.06 |
| 33 | 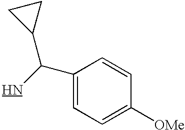 | Interchim Intermediates | 450 | 3.15 |
| 34 | 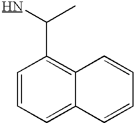 | Fluka | 444 | 3.36 |
| 35 | 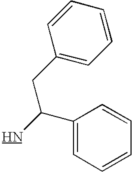 | Aldrich | 470 | 3.40 |
| 36 | 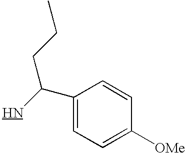 | Gaodeng Xuexiao Huaxue Xuebao, 2001, 22(10, Suppl.), 89-91 | 452 | 3.29 |
| 37 | 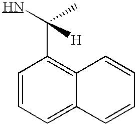 | Fluka | 444 | 3.36 |
| 38 | 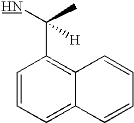 | Fluka | 444 | 3.36 |
| 39 | 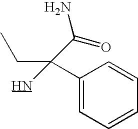 | Tim Tec Stock Library | 451 | 2.36 |
| 40 | 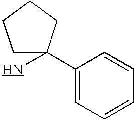 | Synthesis, 1978, 1, 24-6. | 434 | 2.80 |
| 41 | 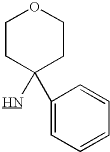 | J. Med. Chem; 1967, 10 (1), 128-9 | 450 | 2.44 |
| 42 | 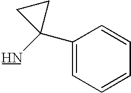 | Org. Lett; 2003, 5 (5), 753-755 | 406 | 2.99 |
| 43 | 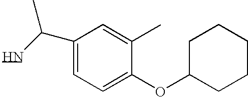 | Biochem. Pharmacol., 1959, 2, 264-9 (Prep. Not given) | 506 | 3.75 |
| 44 | 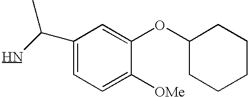 | Not known | 522 | 3.32 |
| 45 | 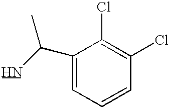 | Sigma | 462 +464 | 3.38 |
| 46 | 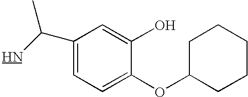 | | 508 | 3.28 |
| 47 | 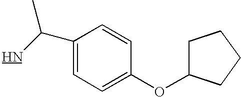 | | 478 | 3.39 |
| 48 | 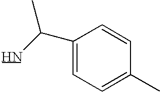 | Aldrich | 408 | 3.09 |
| 49 | 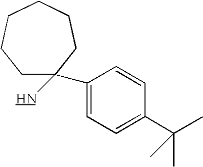 | | 518 | 3.88 |
| 50 | 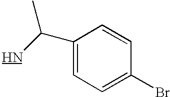 | Aldrich | 472 +474 | 3.22 |
| 51 | 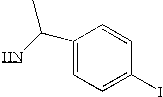 | | 520 | 3.30 |
| 52 | 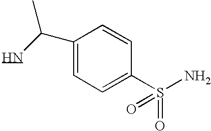 | | 473 | 2.57 |
| 53 | 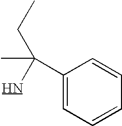 | SALOR | 422 | 3.12 |
| 54 | 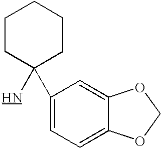 | | 491 | 3.26 |
| 55 | 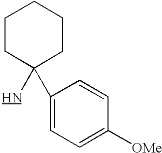 | | 478 | 3.30 |
| 56 | 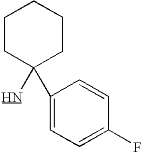 | | 466 | 3.31 |
| 57 | 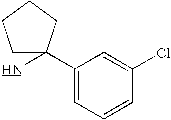 | | 468 +470 | 3.38 |
| 58 | 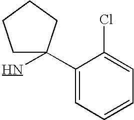 | | 468 +470 | 3.22 |
| 59 | 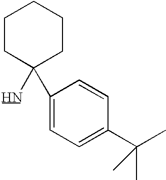 | | 504 | 3.74 |
| 60 | 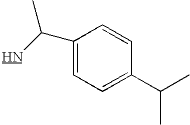 | Tim Tec Building Blocks B (Intermediate 90) | 436 | 3.36 |
| 61 | 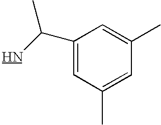 | Intermediate 87 | 422 | 3.23 |
| 62 | 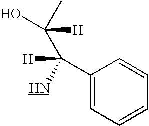 | | 424 | 2.58 |
| 63 | 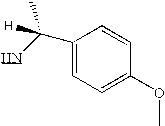 | Lancaster | 424 | 2.87 |
| 64 | 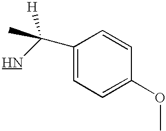 | Lancaster | 424 | 2.98 |
| 65 | 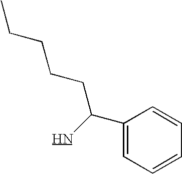 | Intermediate 95 | 450 | 3.54 |
| 66 | 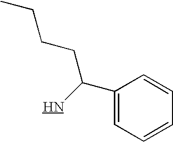 | Intermediate 96 | 436 | 3.39 |
| 67 | 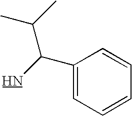 | Intermediate 98 | 422 | 3.19 |
| 68 | 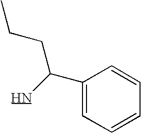 | Intermediate 99 | 422 | 3.17 |
| 69 | 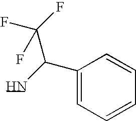 | Intermediate 92 | 448 | 3.21 |
| 70 | 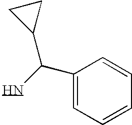 | Intermediate 97 | 420 | 3.09 |
| 71 | 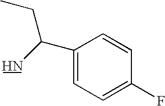 | U.S. Pat. No. 4154599 (1980) | 426 | 3.18 |
| 72 | 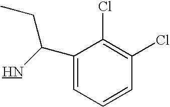 | | 476 | 3.53 |
| 73 | 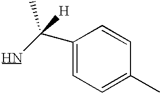 | Lancaster | 408 | 3.14 |
| 74 | 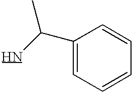 | Aldrich | 394 | 2.99 |
| 75 | 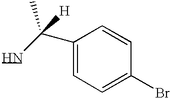 | Lancaster | 472 | 3.28 |
| 76 | 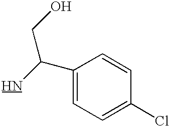 | Ger. Offen DE4443892 (1996) | 445 | 2.85 |
| 77 | 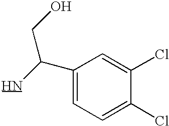 | WO 9709335 (1997) | 478 | 2.95 |
| 78 | 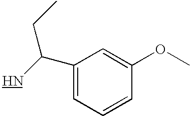 | Intermediate 72 | 438 | 3.12 |
| 79 | 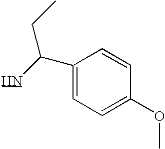 | Intermediate 73 | 438 | 3.10 |
| 80 | 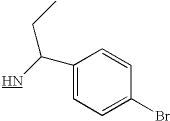 | Intermediate 74 | 486 | 3.39 |
| 81 | 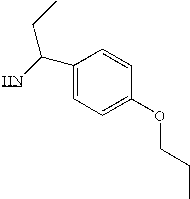 | Intermediate 77 | 466 | 3.41 |
| 82 | 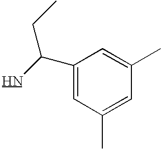 | Intermediate 85 | 436 | 3.39 |
| 83 | 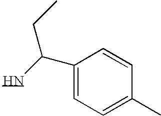 | Intermediate 75 | 422 | 3.26 |
| 84 | 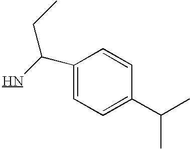 | Intermediate 80 | 450 | 3.51 |
| 85 | 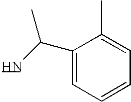 | Intermediate 63 | 408 | 3.13 |
| 86 | 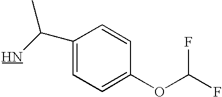 | Intermediate 65 | 460 | 3.17 |
| 87 | 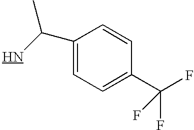 | Intermediate 66 | 462 | 3.67 |
| 88 | 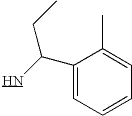 | Intermediate 70 | 422 | 3.40 |
| 89 | 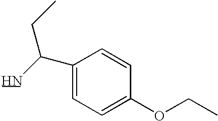 | Intermediate 76 | 452 | 3.24 |
| 90 | 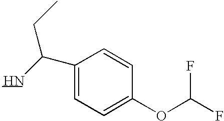 | Intermediate 78 | 474 | 3.28 |
| 91 | 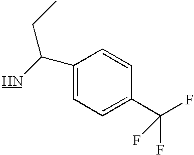 | Intermediate 79 | 476 | 3.81 |
| 92 | 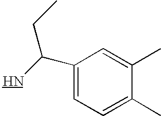 | Intermediate 84 | 436 | 3.37 |
| 93 | 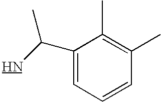 | Intermediate 67 | 422 | 3.46 |
| 94 | 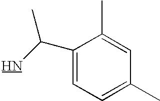 | Intermediate 62 | 422 | 3.28 |
| 95 | 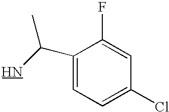 | Intermediate 68 | 446 | 3.31 |
| 96 | 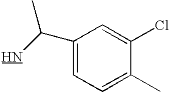 | Intermediate 69 | 442 | 3.36 |
| 97 | 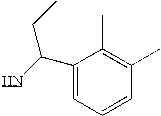 | Intermediate 81 | 436 | 3.58 |
| 98 | 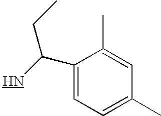 | Intermediate 82 | 436 | 3.41 |
| 99 | 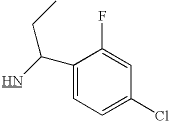 | Intermediate 83 | 460 | 3.43 |
| 100 | 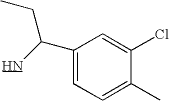 | Intermediate 86 | 456 | 4.02 |
| 101 | 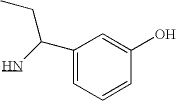 | Intermediate 71 | 424 | 2.87 |
| 102 | 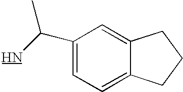 | Intermediate 90 | 433 | 3.18 |
| 103 | 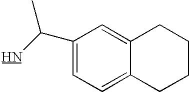 | Intermediate 91 | 447 | 3.29 |
| 104 | 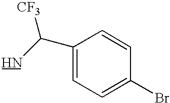 | Intermediate 93 | 527 | 3.35 |
| 105 | 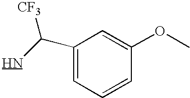 | Intermediate 94 | 478 | 3.14 |

When Examples 78 to 101 are made from an amine reagent Ar-C(R⁴)(R⁵)-NH₂ which is an appropriate one of Intermediates 62 to 86 (excluding Intermediates 75a, 80a, 82a, 82b, and 83a) as disclosed in the Examples 1-105 table above, then Examples 78 to 101 are believed to be a mixture of enantiomers with the major enantiomer believed to have the (R)-stereochemistry (i.e. at the benzylic carbon atom).

### Alternative Preparation of Example 73

A solution of Intermediate 13 (2.0 g) in thionyl chloride (20 ml) was stirred and heated at reflux for 2.5 hours. The solution was cooled and the thionyl chloride was removed in vacuo to leave the intermediate acid chloride (2.1 g). A solution of the acid chloride (2.1 g), (R)-1-(4-methylphenyl)ethylamine (1.0 g) and DIPEA (1.4 g) in THF (100 ml) was stirred for 18 hours. The reaction mixture was concentrated in vacuo. The residue was partitioned between 0.5M sodium bicarbonate (250 ml) and ethyl acetate (250 ml). The organic phase was separated, washed with water (250 ml), dried over Na₂SO₄ and concentrated in vacuo to give a foam. The foam was crystallised from a (5:1) mixture of cyclohexane and Et₂O. One recrystallisation from a (5:1) mixture of cyclohexane and Et₂O gave Example 73 (0.96 g) as white needles. LC-MS showed MH⁺=408; T_{RET}=3.05 min.

### Examples 106 to 169

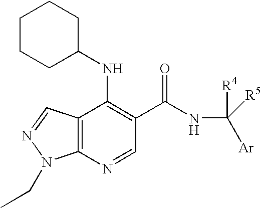

General Procedure:

A mixture of Intermediate 14 (0.1 mmol), HATU (0.1 mmol) and DIPEA (0.4 mmol) in DMF (0.4 ml) was shaken at room temperature for 10 min. A solution of the amine Ar-C(R⁴)(R⁵)-NH₂ (0.1 mmol) in DMF (0.2 ml) was then added and the mixture was agitated for several minutes to give a solution. The solution was stored at room temperature for 16 hours then concentrated in vacuo. The residue was dissolved in chloroform (0.5 ml) and applied to a SPE cartridge (aminopropyl, 0.5 g). The cartridge was eluted successively with chloroform (1.5 ml), EtOAc (1.5 ml) and EtOAc:MeOH (9:1, 1.5 ml). Fractions containing the desired product were concentrated in vacuo and the residue purified by mass directed autoprep HPLC.

The following Examples 106 to 169 were prepared from Intermediate 14 and the appropriate amine Ar-C(R⁴)(R⁵)-NH₂ using the above or a similar procedure:

| Example Number | 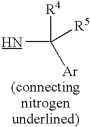 | 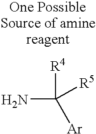 | MH⁺ Ion | LC-MS retention time |
|---|---|---|---|---|
| 106 | 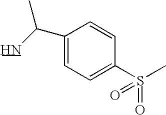 | Peakdale Molecular Ltd. | 470 | 3.25 |
| 107 | 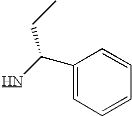 | Lancaster | 406 | 3.72 |
| 108 | 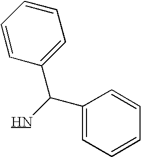 | Aldrich | 454 | 3.88 |
| 109 | 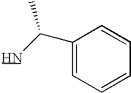 | Aldrich | 392 | 3.60 |
| 110 | 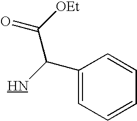 | Maybridge Combichem | 450 | 3.65 |
| 111 | 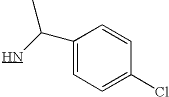 | Bionet Research | 426 | 3.82 |
| 112 | 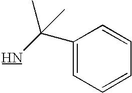 | Fluorochem. Ltd. | 406 | 3.64 |
| 113 | 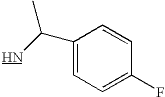 | Aldrich | 410 | 3.64 |
| 114 | 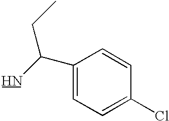 | | 440 | 3.93 |
| 115 | 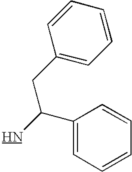 | Aldrich | 468 | 3.90 |
| 116 | 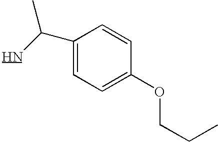 | | 450 | 3.78 |
| 117 | 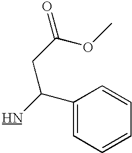 | Chembridge Europe | 450 | 3.49 |
| 118 | 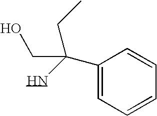 | | 436 | 3.39 |
| 119 | 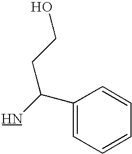 | Acros | 422 | 2.81 |
| 120 | 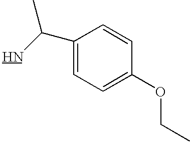 | Tim Tec Building Blocks Inc. (Intermediate 64) | 436 | 3.22 |
| 121 | 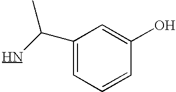 | Intermediate 88 | 408 | 2.87 |
| 122 | 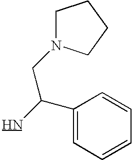 | MicroChemistry Building Blocks | 461 | 2.26 |
| 123 | 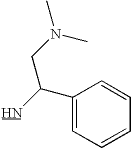 | MicroChemistry Building Blocks | 436 | 2.23 |
| 124 | 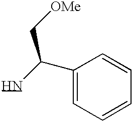 | Omega Chem | 422 | 3.47 |
| 125 | 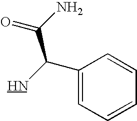 | | 421 | 3.08 |
| 126 | 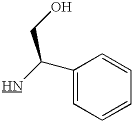 | Aldrich | 408 | 3.21 |
| 127 | 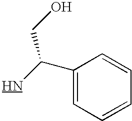 | Aldrich | 408 | 3.21 |
| 128 | 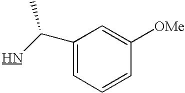 | Lancaster | 422 | 4.97 |
| 129 | 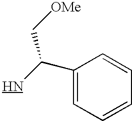 | Omega Chem | 422 | 3.02 |
| 130 | 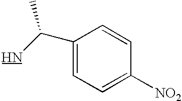 | Aldrich (hydrochloride) | 437 | 3.20 |
| 131 | 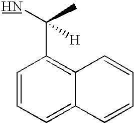 | Fluka | 442 | 3.45 |
| 132 | 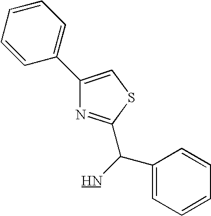 | | 537 | 4.01 |
| 133 | 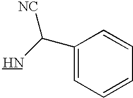 | Aldrich (hydrochloride) | 403 | 3.60 |
| 134 | 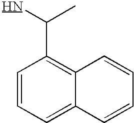 | Fluka | 442 | 3.90 |
| 135 | 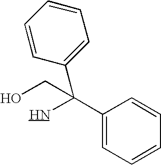 | | 484 | 3.57 |
| 136 | 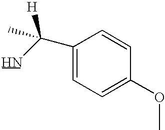 | Lancaster | 422 | 3.54 |
| 137 | 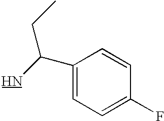 | U.S. Pat. No. 4154599 (1980) | 424 | 3.75 |
| 138 | 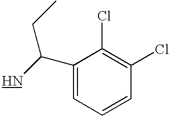 | | 474 | 4.13 |
| 139 | 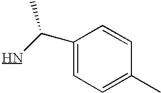 | Lancaster | 406 | 3.71 |
| 140 | 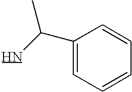 | Aldrich | 392 | 3.58 |
| 141 | 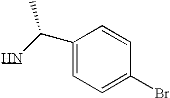 | Lancaster | 470 | 3.85 |
| 142 | 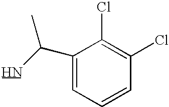 | Sigma | 460 | 4.03 |
| 143 | 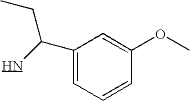 | Intermediate 72 | 436 | 3.68 |
| 144 | 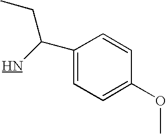 | Intermediate 73 | 436 | 3.65 |
| 145 | 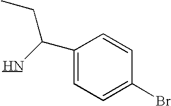 | Intermediate 74 | 484 | 3.97 |
| 146 | 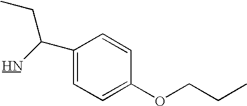 | Intermediate 77 | 464 | 3.94 |
| 147 | 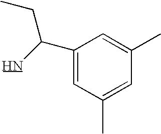 | Intermediate 85 | 434 | 3.95 |
| 148 | 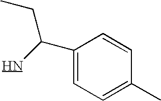 | Intermediate 75 | 420 | 3.83 |
| 149 | 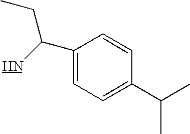 | Intermediate 80 | 448 | 4.05 |
| 150 | 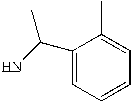 | Intermediate 63 | 406 | 3.74 |
| 151 | 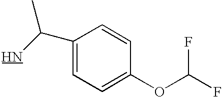 | Intermediate 65 | 458 | 3.84 |
| 152 | 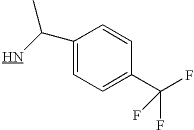 | Intermediate 66 | 460 | 3.84 |
| 153 | 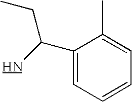 | Intermediate 70 | 420 | 3.87 |
| 154 | 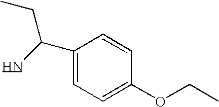 | Intermediate 76 | 450 | 4.34 |
| 155 | 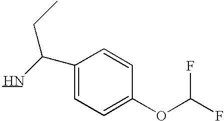 | Intermediate 78 | 472 | 4.00 |
| 156 | 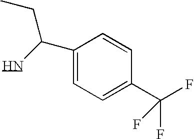 | Intermediate 79 | 474 | 3.95 |
| 157 | 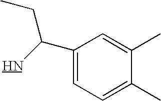 | Intermediate 84 | 434 | 3.93 |
| 158 | 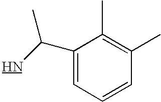 | Intermediate 67 | 420 | 3.85 |
| 159 | 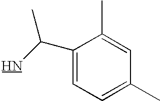 | Intermediate 62 | 420 | 3.86 |
| 160 | 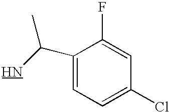 | Intermediate 68 | 444 | 4.39 |
| 161 | 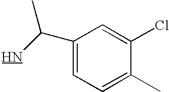 | Intermediate 69 | 440 | 4.10 |
| 162 | 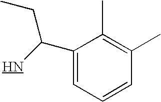 | Intermediate 81 | 434 | 3.96 |
| 163 | 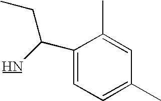 | Intermediate 82 | 434 | 3.99 |
| 164 | 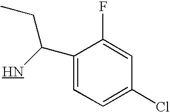 | Intermediate 83 | 458 | 4.37 |
| 165 | 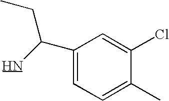 | Intermediate 86 | 454 | 4.26 |
| 166 | 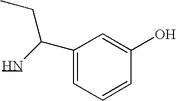 | Intermediate 71 | 422 | 3.43 |
| 167 | 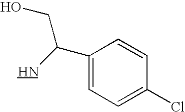 | Ger. Offen DE4443892 (1996) | 442 | 3.38 |
| 168 | 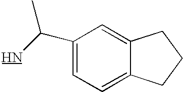 | Intermediate 90 | 431 | 3.76 |
| 169 | 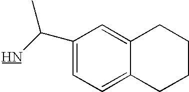 | Intermediate 91 | 445 | 3.96 |

When Examples 143 to 166 are made from an amine reagent Ar-C(R⁴)(R⁵)-NH₂ which is an appropriate one of Intermediates 62 to 86 (excluding Intermediates 75a, 80a, 82a, 82b, and 83a) as disclosed in the Examples 106-169 table above, then Examples 143 to 166 are believed to be a mixture of enantiomers with the major enantiomer believed to have the (R)-stereochemistry (i.e. at the benzylic carbon atom).

### Examples 170 to 174

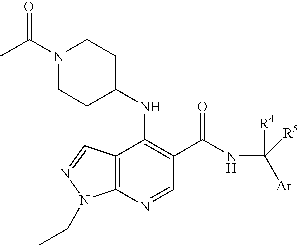

General Procedure:

A mixture of Intermediate 15 (0.1 mmol), HATU (0.1 mmol) and DIPEA (0.4 mmol) in DMF (0.4 ml) was shaken at room temperature for 10 min. A solution of the amine Ar-C(R⁴)(R⁵)-NH₂ (0.1 mmol) in DMF (0.2 ml) was then added and the mixture was agitated for several minutes to give a solution. The solution was stored at room temperature for 16 hours then concentrated in vacuo. The residue was dissolved in chloroform (0.5 ml) and applied to a SPE cartridge (aminopropyl, 0.5 g). The cartridge was eluted successively with chloroform (1.5 ml), EtOAc (1.5 ml) and EtOAc:MeOH (9:1, 1.5 ml). Fractions containing the desired product were concentrated in vacuo and the residue purified by mass directed autoprep HPLC.

The following Examples 170 to 174 were prepared from Intermediate 15 and the appropriate amine Ar-C(R⁴)(R⁵)-NH₂ using the above or a similar procedure:

| Ex- ample Number | 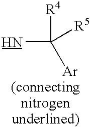 | 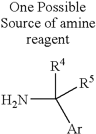 | MH⁺Ion | LC-MS retention time |
|---|---|---|---|---|
| 170 | 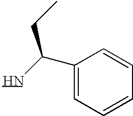 | Lancaster | 449 | 2.94 |
| 171 | 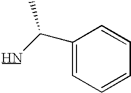 | Aldrich | 435 | 2.84 |
| 172 | 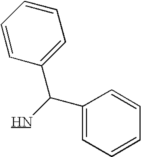 | Aldrich | 497 | 3.16 |
| 173 | 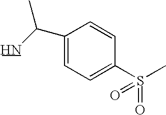 | Peakdale Molecular Ltd. | 513 | 2.63 |
| 174 | 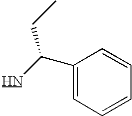 | Lancaster | 449 | 2.95 |

### Examples 175 to 226

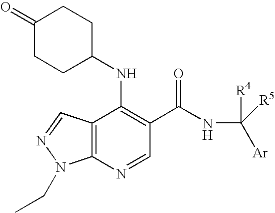

General Procedure:

A mixture of Intermediate 16 (0.1 mmol), HATU (0.1 mmol) and DIPEA (0.4 mmol) in DMF (0.4 ml) was shaken at room temperature for 10 min. A solution of the amine Ar-C(R⁴)(R⁵)-NH₂ (0.1 mmol) in DMF (0.2 ml) was then added and the mixture was agitated for several minutes to give a solution. The solution was stored at room temperature for 16 hours then concentrated in vacuo. The residue was dissolved in chloroform (0.5 ml) and applied to a SPE cartridge (aminopropyl, 0.5 g). The cartridge was eluted successively with chloroform (1.5 ml), EtOAc (1.5 ml) and EtOAc:MeOH (9:1, 1.5 ml). Fractions containing the desired product were concentrated in vacuo and the residue purified by mass directed autoprep HPLC.

The following Examples 175 to 226 were prepared from Intermediate 16 and the appropriate amine Ar-C(R⁴)(R⁵)-NH₂ using the above or a similar procedure:

| Example Number | 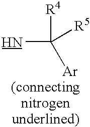 | 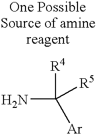 | MH⁺ Ion | LC-MS retention time |
|---|---|---|---|---|
| 175 | 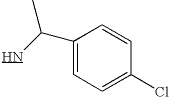 | Bionet Research | 440 | 3.22 |
| 176 | 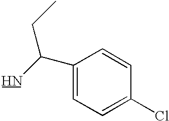 | | 454 | 3.20 |
| 177 | 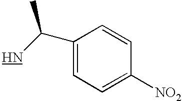 | Aldrich (hydrochloride) | 451 | 3.02 |
| 178 | 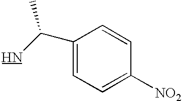 | Aldrich (hydrochloride) | 451 | 3.02 |
| 179 | 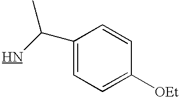 | Tim Tec Building Blocks Inc. Intermediate 64 | 450 | 3.06 |
| 180 | 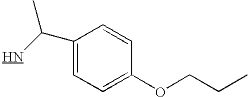 | GR87015X/A | 464 | 3.26 |
| 181 | 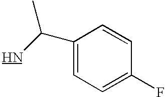 | Aldrich | 424 | 3.02 |
| 182 | 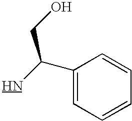 | Aldrich | 422 | 2.64 |
| 183 | 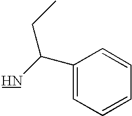 | Aldrich | 420 | 3.06 |
| 184 | 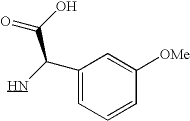 | | 466 | 2.76 |
| 185 | 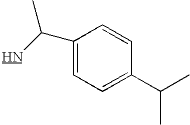 | Tim Tec Building Blocks B Intermediate 89 | 448 | 3.36 |
| 186 | 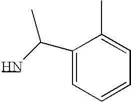 | Tim Tec Building Blocks B | 420 | 2.79 |
| 187 | 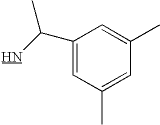 | Intermediate 87 | 434 | 3.25 |
| 188 | 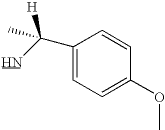 | Lancaster | 436 | 2.99 |
| 189 | 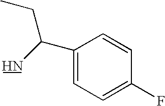 | | 438 | 3.19 |
| 190 | 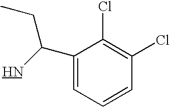 | | 488 | 3.52 |
| 191 | 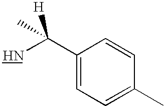 | Lancaster | 420 | 3.15 |
| 192 | 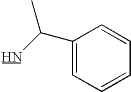 | Aldrich | 406 | 3.01 |
| 193 | 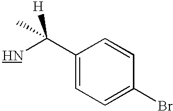 | Lancaster | 484 | 3.28 |
| 194 | 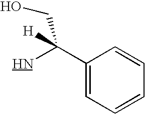 | Aldrich | 422 | 2.54 |
| 195 | 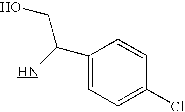 | Ger. Offen DE4443892 (1996) | 456 | 2.86 |
| 196 | 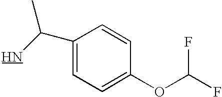 | Intermediate 65 | 472 | 2.85 |
| 197 | 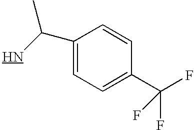 | Intermediate 66 | 474 | 3.00 |
| 198 | 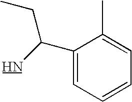 | Intermediate 70 | 434 | 2.92 |
| 199 | 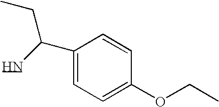 | Intermediate 76 | 464 | 2.90 |
| 200 | 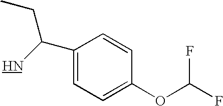 | Intermediate 78 | 486 | 2.96 |
| 201 | 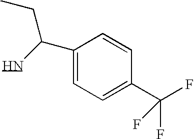 | Intermediate 79 | 488 | 3.11 |
| 202 | 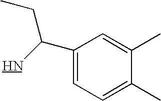 | Intermediate 84 | 448 | 3.02 |
| 203 | 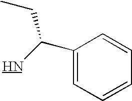 | Lancaster | 420 | 2.79 |
| 204 | 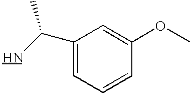 | Lancaster | 436 | 2.67 |
| 205 | 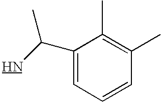 | Intermediate 67 | 434 | 2.90 |
| 206 | 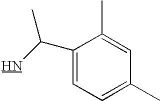 | Intermediate 62 | 434 | 2.93 |
| 207 | 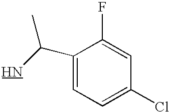 | Intermediate 68 | 458 | 2.98 |
| 208 | 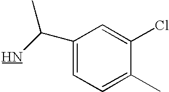 | Intermediate 69 | 454 | 3.03 |
| 209 | 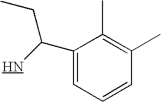 | Intermediate 81 | 448 | 3.03 |
| 210 | 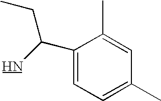 | Intermediate 82 | 448 | 3.05 |
| 211 | 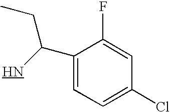 | Intermediate 83 | 472 | 3.10 |
| 212 | 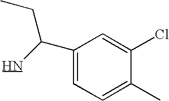 | Intermediate 86 | 468 | 3.14 |
| 213 | 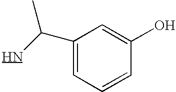 | Intermediate 88 | 422 | 2.44 |
| 214 | 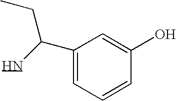 | Intermediate 71 | 436 | 2.56 |
| 215 | 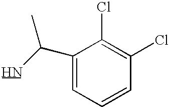 | Sigma | 474 | 3.41 |
| 216 | 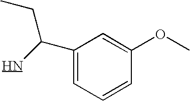 | Intermediate 72 | 450 | 3.13 |
| 217 | 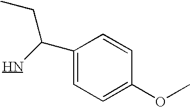 | Intermediate 73 | 450 | 3.12 |
| 218 | 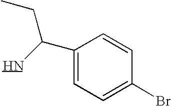 | Intermediate 74 | 498 | 3.39 |
| 219 | 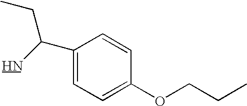 | Intermediate 77 | 478 | 3.42 |
| 220 | 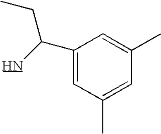 | Intermediate 85 | 448 | 3.39 |
| 221 | 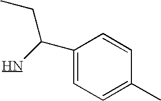 | Intermediate 75 | 434 | 3.48 |
| 222 | 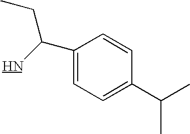 | Intermediate 80 | 462 | 3.54 |
| 223 | 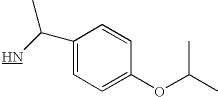 | J. Chem. Soc. Abstracts 1951, 3430-3 | 464 | 3.19 |
| 224 | 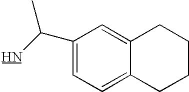 | Intermediate 91 | 460 | 3.39 |
| 225 | 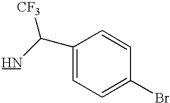 | Intermediate 93 | 539 | 3.45 |
| 226 | 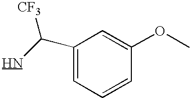 | Intermediate 94 | 490 | 3.24 |

When Examples 196 to 202, 205 to 212, 214, and 216 to 222 are made from an amine reagent Ar-C(R⁴)(R⁵)-NH₂ which is an appropriate one of Intermediates 62 to 86 (excluding Intermediates 75a, 80a, 82a, 82b, and 83a) as disclosed in the Examples 175-226 table above, then Examples 196 to 202, 205 to 212, 214, and 216 to 222 are believed to be a mixture of enantiomers with the major enantiomer believed to have the (R)-stereochemistry (i.e. at the benzylic carbon atom).

### Example 227

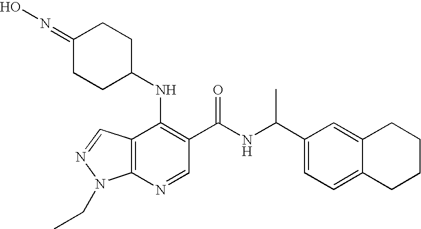

A mixture of Intermediate 17 (25 mg, 0.079 mmol), HATU (35 mg, 0.092 mmol) and DIPEA (50 mg, 0.387 mmol) in MeCN (2.0 ml) was stirred at room temperature for 10 min. Intermediate 91 (30 mg, 0.142 mmol) was then added and the mixture was stirred for 2.5 hours then left to stand overnight. The solution was concentrated in vacuo. The residue was dissolved in EtOAc and applied to a SPE cartridge (silica, 5 g). The cartridge was eluted with EtOAc. Fractions containing the desired product were concentrated in vacuo to give Example 227 as a white solid. LCMS showed MH⁺=475; T_{RET}=3.32 min.

### Examples 228 to 230

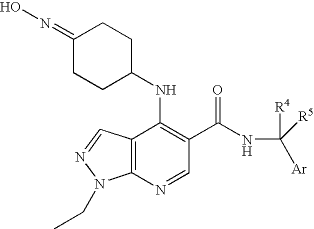

The following Examples 228 to 230 were prepared from Intermediate 17 and the appropriate amine Ar-C(R⁴)(R⁵)-NH₂ using a similar procedure to that used for the preparation of Example 227:

| Ex- ample Number | 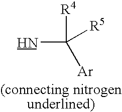 | 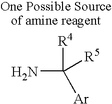 | MH⁺Ion | LC-MS re- tention time |
|---|---|---|---|---|
| 228 | 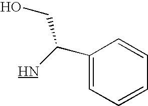 | Aldrich | 438 | 2.59 |
| 229 | 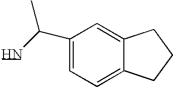 | Intermediate 90 | 461 | 3.19 |
| 230 | 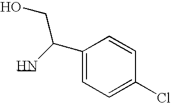 | Ger. Offen DE4443892 (1996) | 471 | 2.78 +2.81 |

### Examples 231 to 281

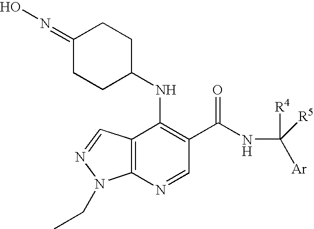

General Procedure:

A mixture of the appropriate ketone (0.05 mmol), hydroxylamine hydrochloride (0.07 mmol) and DIPEA (0.05 ml) in MeCN (1.0 ml) was heated at reflux for 5 hours. The solvent was removed. The residue was dissolved in chloroform and applied to a SPE cartridge (silica, 0.5 g). The cartridge was eluted with EtOAc. Fractions containing the desired product were concentrated in vacuo to give the appropriate oxime.

The following Examples 231 to 281 were prepared in the above or a similar manner:

| Ex- am- ple Num- ber | 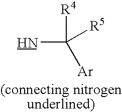 | Starting Ketone | MH⁺Ion | LC-MS re- tention time |
|---|---|---|---|---|
| 231 | 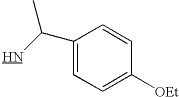 | Example 179 | 465 | 2.92 |
| 232 | 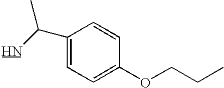 | Example 180 | 479 | 3.09 |
| 233 | 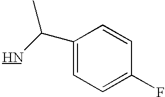 | Example 181 | 439 | 2.87 |
| 234 | 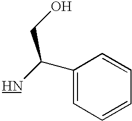 | Example 182 | 437 | 2.47, 2.51 |
| 235 | 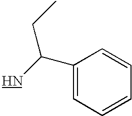 | Example 183 | 435 | 3.02 |
| 236 | 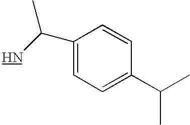 | Example 185 | 463 | 3.28 |
| 237 | 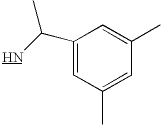 | Example 187 | 449 | 3.15 |
| 238 | 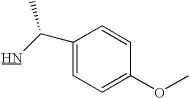 | Example 188 | 451 | 2.58 |
| 239 | 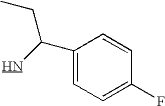 | Example 189 | 453 | 2.78 |
| 240 | 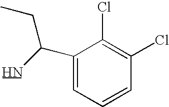 | Example 190 | 503 | 3.11 |
| 241 | 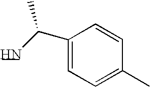 | Example 191 | 435 | 2.72 |
| 242 | 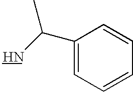 | Example 192 | 421 | 2.58 |
| 243 | 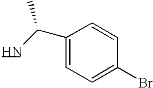 | Example 193 | 499 | 2.86 |
| 244 | 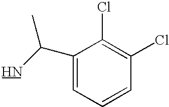 | Example 215 | 489 | 3.01 |
| 245 | 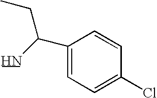 | Example 176 | 469 | 2.94 |
| 346 | 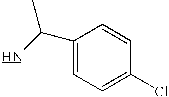 | Example 175 | 455 | 2.82 |
| 247 | 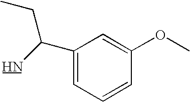 | Example 216 | 465 | 2.72 |
| 248 | 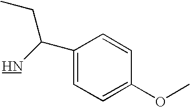 | Example 217 | 465 | 2.70 |
| 249 | 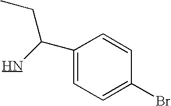 | Example 218 | 513 | 2.98 |
| 250 | 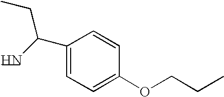 | Example 219 | 493 | 2.99 |
| 251 | 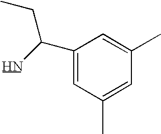 | Example 220 | 463 | 2.96 |
| 252 | 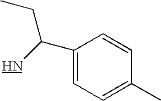 | Example 221 | 449 | 2.84 |
| 253 | 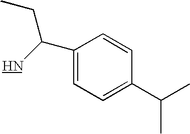 | Example 222 | 477 | 3.08 |
| 254 | 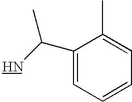 | Example 186 | 435 | 2.72 |
| 255 | 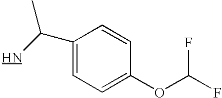 | Example 196 | 487 | 2.77 |
| 256 | 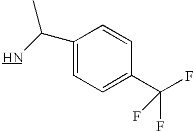 | Example 197 | 489 | 2.92 |
| 257 | 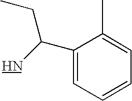 | Example 198 | 449 | 2.83 |
| 258 | 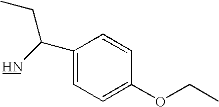 | Example 199 | 479 | 2.82 |
| 259 | 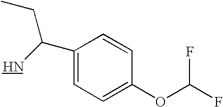 | Example 200 | 501 | 2.88 |
| 260 | 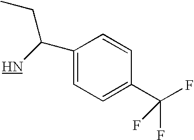 | Example 201 | 503 | 3.02 |
| 261 | 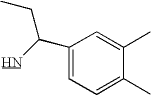 | Example 202 | 463 | 2.99 |
| 262 | 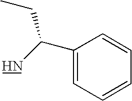 | Example 203 | 435 | 2.71 |
| 263 | 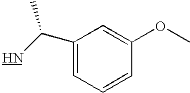 | Example 204 | 451 | 2.60 |
| 264 | 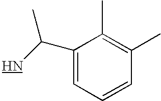 | Example 205 | 449 | 2.82 |
| 265 | 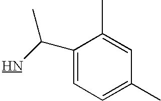 | Example 206 | 449 | 2.84 |
| 266 | 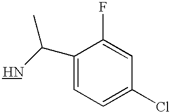 | Example 207 | 473 | 2.90 |
| 267 | 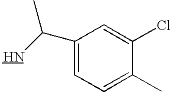 | Example 208 | 469 | 2.94 |
| 268 | 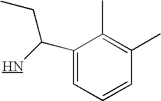 | Example 209 | 463 | 2.93 |
| 269 | 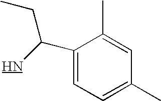 | Example 210 | 463 | 2.95 |
| 270 | 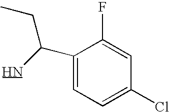 | Example 211 | 487 | 3.01 |
| 271 | 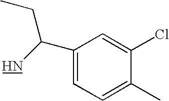 | Example 212 | 483 | 3.05 |
| 272 | 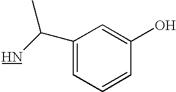 | Example 213 | 437 | 2.40 |
| 273 | 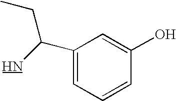 | Example 214 | 451 | 2.52 |
| 274 | 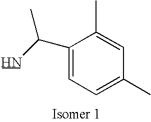 | Example 295 | 449 | 3.05 |
| 275 | 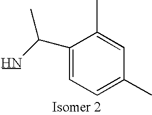 | Example 296 | 449 | 3.05 |
| 276 | 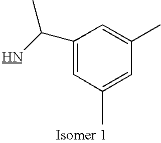 | Example 297 | 449 | 3.06 |
| 277 | 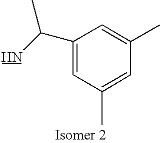 | Example 298 | 449 | 3.06 |
| 278 | 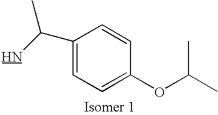 | Example 299 | 479 | 3.01 |
| 279 | 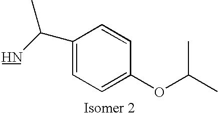 | Example 300 | 479 | 3.01 |
| 280 | 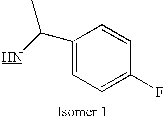 | Example 301 | 439 | 2.90 |
| 281 | 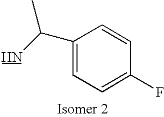 | Example 302 | 439 | 2.90 |

When Examples 196 to 202, 205 to 212, 214, and 216 to 222 are made from an amine reagent Ar-C(R⁴)(R⁵)-NH₂ which is an appropriate one of Intermediates 62 to 86 (excluding Intermediates 75a, 80a, 82a, 82b, and 83a) as disclosed in the Examples 175-226 table above, then the derived Examples 247 to 253, 255 to 261, 264 to 271, and 273 disclosed in the Examples 231-281 table above are generally believed to be a mixture of isomers with the major isomer(s) believed to have the (R)-stereochemistry (i.e. at the benzylic carbon atom).

### Examples 282 to 286

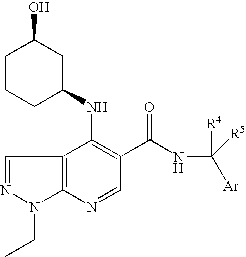 [cis-(3-hydroxycyclohex-1-yl)amino group; (1:1) mixture of cis-stereoisomers]

General Procedure:

A mixture of Intermediate 19 (0.075 mmol), HATU (0.09 mmol) and DIPEA (0.19 mmol) in MeCN (2.0 ml) was stirred at room temperature for 10 min. then added to the amine reagent Ar-C(R⁴)(R⁵)-NH₂ (0.075 mmol). The reaction mixture was stirred at room temperature for 7 h. The solvent was removed by blowing nitrogen over the reaction mixture. The residue was partitioned between EtOAc (5 ml) and 0.5M sodium bicarbonate (5 ml). The organic phase was separated, washed with water (5 ml) and dried over MgSO₄. The solvent was blown off and the residue dried in vacuo to leave the desired product.

The following Examples 282-286 were prepared from Intermediate 19 and the appropriate amine Ar-C(R⁴)(R⁵)-NH₂ using this or a similar procedure:

| Ex- ample Number | 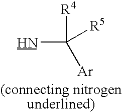 | 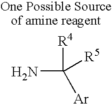 | MH⁺Ion | LC-MS retention time |
|---|---|---|---|---|
| 282 | 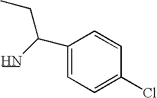 | | 456 | 3.19 |
| 283 | 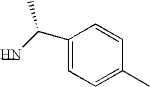 | Lancaster | 422 | 2.91 |
| 284 | 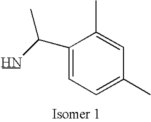 | Intermediate 100 | 436 | 3.12 |
| 285 | 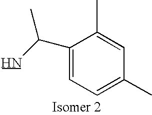 | Intermediate 101 | 436 | 3.14 |
| 286 | 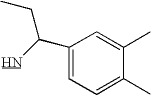 | Intermediate 84 | 450 | 3.15 |

When Example 286 is made from an amine reagent Ar-C(R⁴)(R5)-NH₂ which is Intermediates 84 as disclosed in the table above, then Example 286 is believed to be a mixture of isomers with the major isomer(s) believed to have the (R)-stereochemistry (i.e. at the benzylic carbon atom).

### Examples 287 to 288

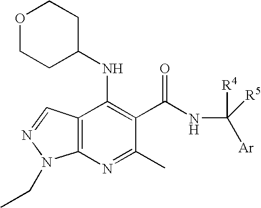

General Procedure:

A mixture of Intermediate 18 (0.1 mmol), HATU (0.1 mmol) and DIPEA (0.4 mmol) in DMF (0.4 ml) was shaken at room temperature for 10 min. A solution of the amine reagent Ar-C(R⁴)(R⁵)-NH₂ (0.1 mmol) in DMF (0.2 ml) was then added and the mixture was agitated for several minutes to give a solution. The solution was stored at room temperature for 16 hours then concentrated in vacuo. The residue was dissolved in chloroform (0.5 ml) and applied to a SPE cartridge (aminopropyl, 0.5 g). The cartridge was eluted successively with chloroform (1.5 ml), EtOAc (1.5 ml) and EtOAc:MeOH (9:1, 1.5 ml). Fractions containing the desired product were concentrated in vacuo and the residue purified by mass directed autoprep HPLC.

The following Examples 287-288 were prepared from Intermediate 18 and the appropriate amine Ar-C(R⁴)(R⁵)-NH₂ using this or a similar procedure:

| Ex- ample Number | 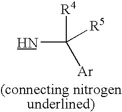 | 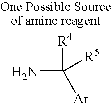 | MH⁺Ion | LC-MS retention time |
|---|---|---|---|---|
| 287 | 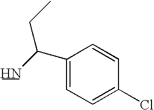 | | 456 +458 | 2.88 |
| 288 | 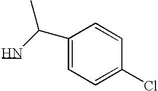 | Bionet Research | 442 +444 | 2.73 |

### Examples 289 to 306

### Separation of isomers of Examples on Chiral Columns

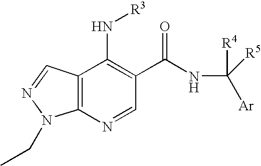

General Procedure:

The Examples below, which were generally either believed to be racemic or believed to be a mixture of isomers generally enriched in major isomer(s) believed to have the (R)-stereochemistry (i.e. at the benzylic carbon atom), were resolved by preparative chiral column chromatography, using either a 2-inch×20 cm Whelk 0-1 chiral column with 100% EtOH or a mixture of EtOH and n-heptane as the eluent or a 2-inch ChiralPak AD chiral column with 100% ethanol as the eluent. In the Table, “Isomer 1” relates to the first enantiomer to be eluted from the column and “Isomer 2” relates to the second enantiomer.

Example 283 (mixture of diastereoisomers) was also separated into its component isomers by preparative chiral column chromatography, using a 2-inch ChiralCel OD chiral column with a (95:5) mixture of heptane and ethanol as the eluent. In the Table, “Isomer 1” relates to the first enantiomer to be eluted from the column and “Isomer 2” relates to the second enantiomer.

| Example Number | NHR³ | 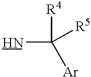 | Starting Material | MH⁺ Ion | LC-MS retention time |
|---|---|---|---|---|---|
| 289 | 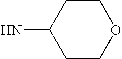 | 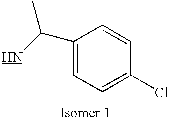 | Example 21 | 428 | 3.18 |
| 290 | 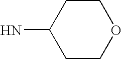 | 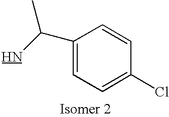 | Example 21 | 428 | 3.18 |
| 291 | 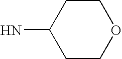 | 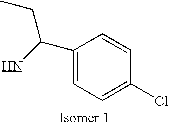 | Example 11 | 442 | 3.30 |
| 292 | 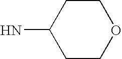 | 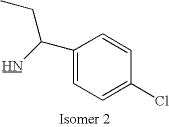 | Example 11 | 442 | 3.30 |
| 293 | 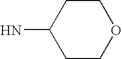 | 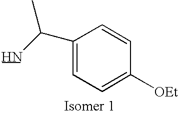 | Example 12 | 438 | 3.07 |
| 294 | 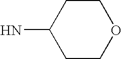 | 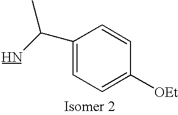 | Example 12 | 438 | 3.07 |
| 295 | 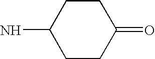 | 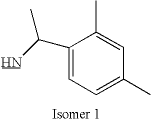 | Example 206 | 434 | 3.25 |
| 296 | 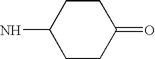 | 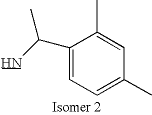 | Example 206 | 434 | 3.25 |
| 297 | 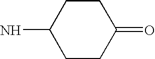 | 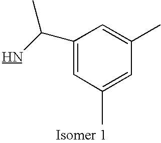 | Example 187 | 434 | 3.25 |
| 298 | 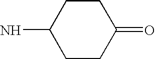 | 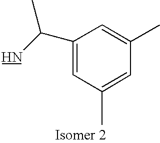 | Example 187 | 434 | 3.26 |
| 299 | 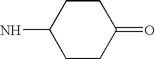 | 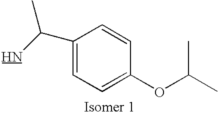 | Example 223 | 464 | 3.21 |
| 300 | 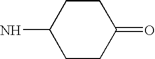 | 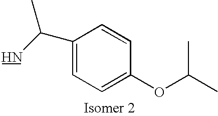 | Example 223 | 464 | 3.19 |
| 301 | 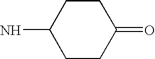 | 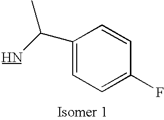 | Example 181 | 424 | 2.93 |
| 302 | 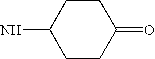 | 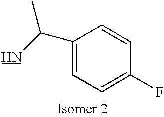 | Example 181 | 424 | 2.93 |
| 303 | 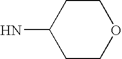 | 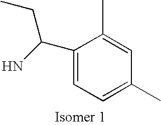 | Example 98 | 436 | 3.36 |
| 304 | 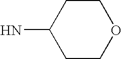 | 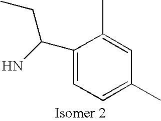 | Example 98 | 436 | 3.36 |
| 305 | 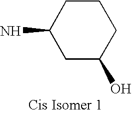 | 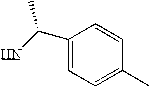 | Example 283 | 422 | 2.90 |
| 306 | 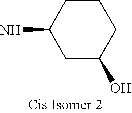 | 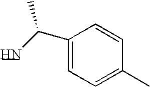 | Example 283 | 422 | 2.90 |

### Example 307 Preparation of the Hydrochloride of Example 304

### N-[1-(2,4-dimethylphenyl)propyl]-[-ethyl-4-(tetrahydro-2H-pyran-4-ylamino)-1H-pyrazolo]3,4-b]pyridine-5-carboxamide (Enantiomer 2) hydrochloride

A solution of Example 304 (1.3 g) in Et₂O (30 ml) was treated, rapidly dropwise with stirring, with a molar excess (relative to Example 304, i.e. more than 1 mole equivalent cf. Example 304) of 1.0 M hydrogen chloride in Et₂O. The resultant suspension was left to stand for 2 hours. The solvent was removed in vacuo. The residual solid was recrystallised from ethanol to give the hydrochloride (0.64 g) as white needles. LC-MS showed MH⁺=436; T_{RET}=3.35 min.

### Example 308

### 4-{[1-(aminocarbonyl)-4-piperidinyl]amino}-1-ethyl-N-[(1R)-1-(4-methylphenyl)ethyl]-1H-pyrazolo[3,4b]pyridine-5-carboxamide

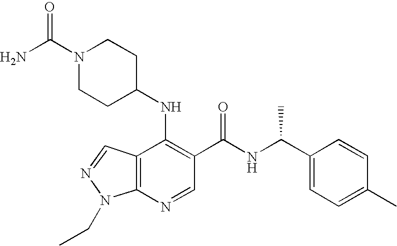

A solution of Intermediate 105 (0.066 mmol) in DMF (1 ml) was treated with EDC (0.066 mmol), HOBT (0.066 mmol) and DIPEA (0.151 mmol) followed by 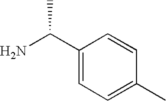
(0.066 mmol) (e.g. available from Lancaster Synthesis), for example at room temperature. The reaction mixture was left to stand at 22° C. for 16 h. The DMF was evaporated and the residue was partitioned between DCM (5 ml) and saturated aqueous sodium bicarbonate (2 ml). The organic layer was collected through a hydrophobic frit and evaporated. The residue was purified by mass directed autoprep. HPLC to give the title compound as a gum (8.9 mg). LCMS showed MH⁺=450; T_{RET}=2.76 min.

The following Examples 309 to 313 were prepared from Intermediate 105 and the appropriate amine Ar-C(R⁴)(R⁵)-NH₂ using substantially the above procedure:

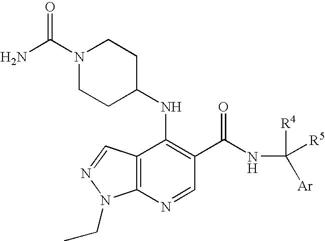

| Ex- ample Number | 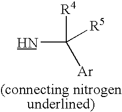 | 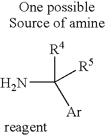 | MH⁺Ion | LC-MS retention time |
|---|---|---|---|---|
| 309 | 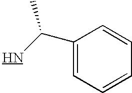 | Aldrich | 436 | 2.62 |
| 310 | 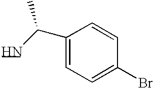 | Lancaster | 516 | 2.8 |
| 311 | 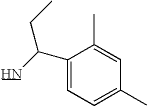 | Intermediate 82 | 478 | 2.96 |
| 312 | 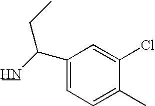 | Intermediate 86 | 498 | 2.9 |
| 313 | 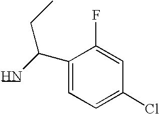 | Intermediate 83 | 502 | 2.88 |

When Examples 311, 312 and 313 are, made from Intermediates 82, 86 and 83 respectively, as disclosed in the table above, then Examples 311, 312 and 313 are believed to be a mixture of enantiomers with the major enantiomer believed to have the (R)-stereochemistry (i.e. at the benzylic carbon atom).

### Alternative Preparation of Example 309

### 4-{[1-(aminocarbonyl)-4-piperidinyl]amino}-1-ethyl-N-[(1R)-1-phenylethyl]-1H-pyrazolo[3,4-b]pyridine-5-carboxamide

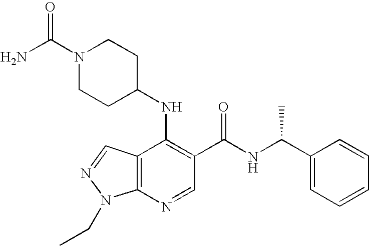

A mixture of Intermediate 109 (27 mg) and Intermediate 111 (16 mg) in MeCN (2 ml) was treated with DIPEA (35 μL). The reaction mixture was heated under reflux for 72 h. The solvent was evaporated and the residue was partitioned between DCM (5 ml) and saturated aqueous sodium bicarbonate (2 ml). The organic layer was collected through a hydrophobic frit and evaporated. The residue was purified by mass directed autoprep. HPLC to give Example 309 as a white solid (5.0 mg). LCMS showed MH⁺=436; T_{RET}=2.62 min.

### Example 314

### 4-{[4-(aminocarbonyl)cyclohexyl]amino}-1-ethyl-N-[(1R)-1-phenylethyl]-1H-pyrazolo[3,4-b]pyridine-5-carboxamide

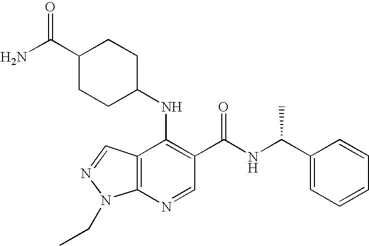

A solution of Intermediate 109 (0.08 mmol) in MeCN (1 ml) was treated with Intermediate 113 (0.088 mmol) and DIPEA (0.2 mmol). The reaction mixture was heated at reflux for 20 h. The solvents were evaporated and the residue was partitioned between DCM (5 ml) and water (2 ml). The organic phase was collected through a hydrophobic frit and evaporated. The residue was purified by mass directed autoprep. HPLC to give Example 314 as a white solid (12.2 mg). LCMS showed MH⁺=435; T_{RET}=2.7 min.

In Example 314, the R³NH group, i.e. the [4-(aminocarbonyl)cyclohexyl]amino group, is preferably in the cis configuration. In this case, (Example 314A), it is 4-{cis-[4-(aminocarbonyl)cyclohexyl]amino}-1-ethyl-N-[(1R)-1-phenylethyl]-1H-pyrazolo[3,4-b]pyridine-5-carboxamide.

### Examples 315 to 328

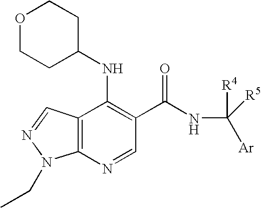

General Procedure:

A mixture of Intermediate 13 (0.1 mmol), HATU (0.1 mmol) and DIPEA (0.4 mmol) in DMF (0.4 ml) was shaken at room temperature for 10 min. A solution of the amine reagent Ar-C(R⁴)(R⁵)-NH₂ (0.1 mmol) in DMF (0.2 ml) was then added and the mixture was agitated for several minutes to give a solution. The solution was stored at room temperature for 16 hours then concentrated in vacuo. The residue was dissolved in chloroform (0.5 ml) and applied to a SPE cartridge (aminopropyl, 0.5 g). The cartridge was eluted successively with chloroform (1.5 ml), EtOAc (1.5 ml) and EtOAc:MeOH (9:1, 1.5 ml). Fractions containing the desired product were concentrated in vacuo and the residue purified by mass directed autoprep HPLC.

The following Examples 315 to 328 were prepared from Intermediate 13 and the appropriate amine reagent Ar-C(R⁴)(R⁵)-NH₂ using this or a similar procedure:

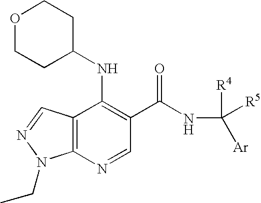

(of which, Examples 316 to 328 are believed to consist essentially of an
enantiomer having the (R)-stereochemistry at the benzylic carbon atom, as
shown below)

| Ex- ample Number | 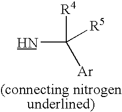 | 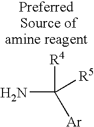 | MH⁺Ion | LC-MS re- tention time |
|---|---|---|---|---|
| 315 | 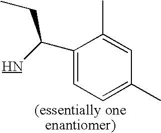 | Intermediate 82a | 436 | 3.31 |
| 316 | 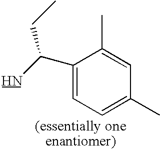 | Intermediate 82b | 436 | 3.31 |
| 317 | 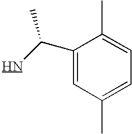 | Intermediate 139 | 422 | 3.21 |
| 318 | 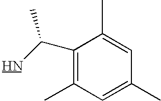 | Intermediate 140 | 436 | 3.34 |
| 319 | 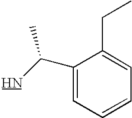 | Intermediate 137 | 422 | 3.23 |
| 320 | 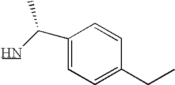 | Intermediate 138 | 422 | 3.23 |
| 321 | 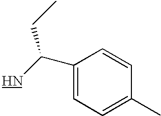 | Intermediate 75a | 422 | 3.04 |
| 322 | 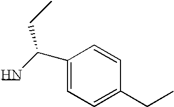 | Intermediate 142 | 436 | 3.19 |
| 323 | 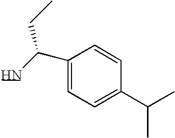 | Intermediate 80a | 450 | 3.32 |
| 324 | 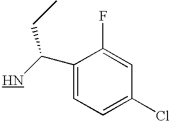 | Intermediate 83a | 460 | 3.24 |
| 325 | 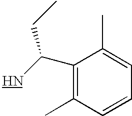 | Intermediate 144 | 436 | 3.17 |
| 326 | 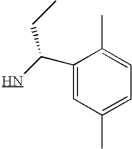 | Intermediate 143 | 436 | 3.19 |
| 327 | 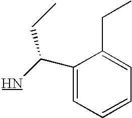 | Intermediate 141 | 436 | 3.19 |
| 328 | 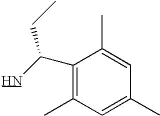 | Intermediate 145 | 450 | 3.31 |

### Example 329

### 4-{[1-(aminocarbonyl)-4-piperidinyl]amino}-N-[(1R)-1-(2,5-dimethylphenyl)ethyl -1-ethyl-1H-pyrazolo[3,4-b]pyridine-5-carboxamide

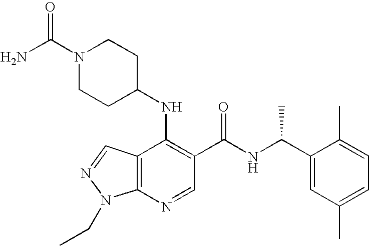 (believed to consist essentially of an enantiomer believed to have the (R)-stereochemistry at the benzylic carbon atom, as shown above)

A solution of Intermediate 105 (29 mg), HATU (36 mg) and DIPEA (0.037 ml) in acetonitrile (5 ml) was stirred at room temperature for 10 min. Intermediate 139 (18 mg) was added. The reaction mixture was left to stand at 22° C. for 16 h. The solvent was evaporated. The residue was dissolved in chloroform and applied to an SPE cartridge (aminopropyl, 2 g). The cartridge was eluted initially with chloroform and then with 20% methanol in ethyl acetate, to give Example 329 (23 mg) as an amorphous solid. LCMS showed MH⁺=464; T_{RET}=2.87 min.

### Examples 330 to 345

The following Examples 330 to 345 were prepared from Intermediate 105 and the appropriate amine Ar-C(R⁴)(R⁵)-NH₂ using the same or a similar procedure to that used for Example 329 e.g. with the same or similar numbers of moles of reagents:

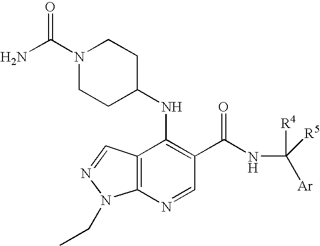

(of which, Examples 330 to 333, Example 335 and Examples 338 to 345, are believed to consist essentially of an enantiomer believed to have the (R)-stereochemistry at the benzylic carbon atom, as shown below)

| Example Number | 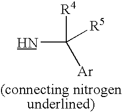 | 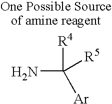 | MH⁺ Ion | LC-MS retention time |
|---|---|---|---|---|
| 330 | 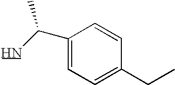 | Intermediate 138 | 464 | 2.9 |
| 331 | 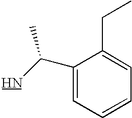 | Intermediate 137 | 464 | 2.88 |
| 332 | 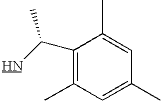 | Intermediate 140 | 478 | 2.96 |
| 333 | 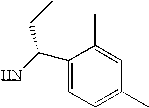 | Intermediate 82b | 478 | 3 |
| 334 | 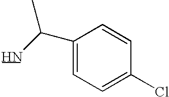 | Bionet Research | 470 | 2.87 |
| 335 | 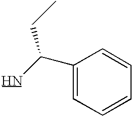 | Lancaster | 450 | 2.78 |
| 336 | 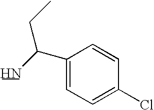 | J. Pharm. Pharmacol; 1997, 49 (1), 10-15 | 484 | 2.98 |
| 337 | 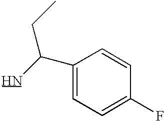 | U.S. Pat. No. 4154599 (1980) | 468 | 2.84 |
| 338 | 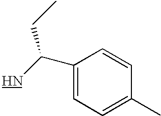 | Intermediate 75a | 464 | 2.74 |
| 339 | 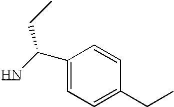 | Intermediate 142 | 478 | 2.88 |
| 340 | 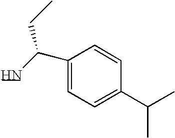 | Intermediate 80a | 492 | 2.99 |
| 341 | 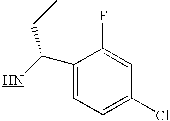 | Intermediate 83a | 502 | 2.9 |
| 342 | 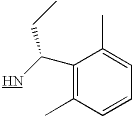 | Intermediate 144 | 478 | 2.83 |
| 343 | 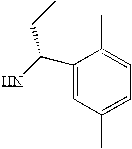 | Intermediate 143 | 478 | 2.85 |
| 344 | 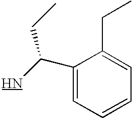 | Intermediate 141 | 478 | 2.85 |
| 345 | 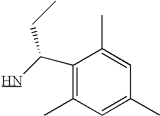 | Intermediate 145 | 492 | 2.95 |

### Examples 346 to 351

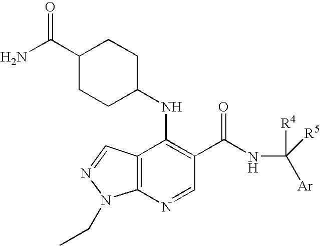 (of which, Example 348 is believed to be a mixture of isomers enriched in a major isomer believed to have the (R)-stereochemistry at the benzylic carbon atom)

General Procedure:

A mixture of Intermediate 120 (0.1 mmol), HATU (0.1 mmol) and DIPEA (0.4 mmol) in DMF (0.4 ml) was shaken at room temperature for 10 min. A solution of the amine reagent Ar-C(R⁴)(R⁵)-NH₂ (0.1 mmol) in DMF (0.2 ml) was then added and the mixture was agitated for several minutes to give a solution. The solution was stored at room temperature for 16-64 hours then concentrated in vacuo. The residue was dissolved in chloroform (0.5 ml) and applied to a SPE cartridge (aminopropyl, 0.5 g). The cartridge was eluted successively with chloroform (1.5 ml), EtOAc (1.5 ml) and EtOAc:MeOH (9:1, 1.5 ml). Fractions containing the desired product were concentrated in vacuo and the residue purified by mass directed autoprep HPLC.

The following Examples 346 to 351 were prepared from Intermediate 120 and the appropriate amine reagent Ar-C(R⁴)(R⁵)-NH₂ using this or a similar procedure. The Examples were isolated as a mixture of cis and trains isomers (at the cyclohexane ring), with the cis isomer predominating.

| Example Number | 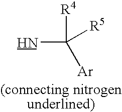 | 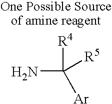 | MH⁺ Ion | LC-MS retention time |
|---|---|---|---|---|
| 346 | 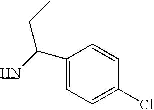 | J. Pharm. Pharmacol; 1997, 49(1), 10-15 | 483 | 3.09 |
| 347 | 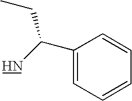 | Lancaster | 449 | 2.88 |
| 348 | 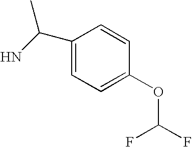 | Intermediate 65 | 501 | 2.95 |
| 349 | 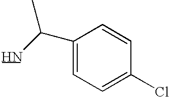 | Bionet Research | 469 | 2.98 |
| 350 | 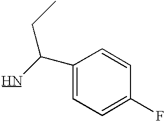 | U.S. Pat. No. 4154599 (1980) | 467 | 2.94 |
| 351 | 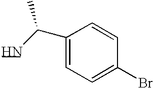 | Lancaster | 513 | 3.02 |

### Examples 352 to 355

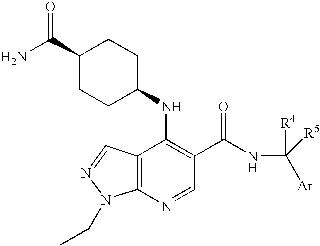 (of which, at least Example 352 is believed to consist essentially of isomer(s) believed to have the (R)-stereochemistry at the benzylic carbon atom, as shown below)

General Procedure:

A mixture of Intermediate 120 (0.09 mmol), EDC (0.1 mmol) and HOBT (0.1 mmol) in DMF (1 ml) was stirred at room temperature for 30 min. DIPEA (0.23 mmol) was added and the solution was added to the amine reagent Ar-C(R⁴)(R⁵)-NH₂ (0.12 mmol) in DMF. The mixture was stirred for 30 min. then left to stand at room temperature for 16 hours. The solvent was evaporated. The residue was partitioned between DCM and saturated sodium bicarbonate solution. The organic phase was separated and evaporated. The residue was purified by mass directed autoprep HPLC to obtain the desired product.

The following Examples 352 to 355 were prepared from Intermediate 120 and the appropriate amine reagent Ar-C(R⁴)(R⁵)-NH₂ using this or a similar procedure:

| Ex- ample Number | 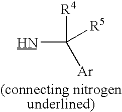 | 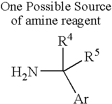 | MH⁺Ion | LC-MS retention time |
|---|---|---|---|---|
| 352 | 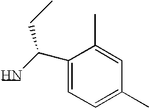 | Intermediate 82b | 477 | 2.92 |
| 353 | 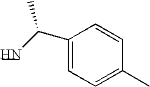 | Lancaster | 449 | 2.72 |
| 354 | 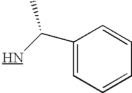 | Aldrich | 435 | 2.63 |
| 355 | 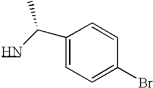 | Lancaster | 513 | 2.90 |

### Examples 356 to 359

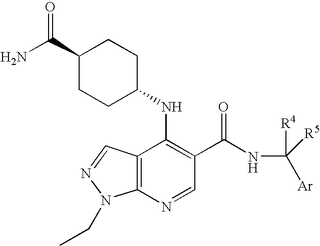 (of which, at least Example 356 is believed to consist essentially of isomer(s) believed to have the (R)-stereochemistry at the benzylic carbon atom, as shown below)

General Procedure:

A mixture of Intermediate 121 (0.09 mmol), EDC (0.1 mmol) and HOBT (0.1 mmol) in DMF (1 ml) was stirred at room temperature for 30 min. DIPEA (0.23 mmol) was added and the solution was added to the amine reagent Ar-C(R⁴)(R⁵)-NH₂ (0.12 mmol) in DMF. The mixture was stirred for 30 min. then left to stand at room temperature for 16 hours. The solvent was evaporated. The residue was partitioned between DCM and saturated sodium bicarbonate solution. The organic phase was separated and evaporated. The residue was purified by mass directed autoprep HPLC to obtain the desired product.

The following Examples 356 to 359 were prepared from Intermediate 121 and the appropriate amine reagent Ar-C(R⁴)(R⁵)-NH₂ using this or a similar procedure:

| Example Number | 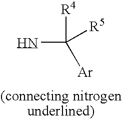 | One Possible Source of amine reagent 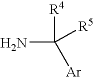 | MH⁺Ion | LC-MS reten- tion time |
|---|---|---|---|---|
| 356 | 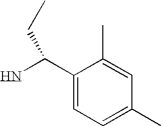 | Intermediate 82b | 477 | 2.98 |
| 357 | 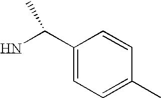 | Lancaster | 449 | |
| 358 | 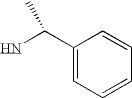 | Aldrich | 435 | 2.65 |
| 359 | 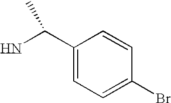 | Lancaster | 513 | 2.90 |

### Examples 360 to 363

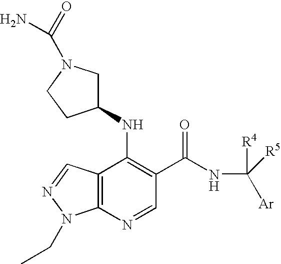 (of which, Examples 360 and possibly Example 362 are believed to be mixtures of diastereoisomers enriched in a major diastereoisomer believed to have the (R)-stereochemistry at the benzylic carbon atom)

General Procedure:

A mixture of Intermediate 152 (30 mg), HATU (120 mg) and DIPEA (0.09 ml) in acetonitrile (2ml) was added to the amine reagent Ar-C(R⁴)(R⁵)-NH₂ (0.09 mmol). The mixture was left to stand at room temperature for 16 hours. The solvent was evaporated. The residue was partitioned between DCM and saturated sodium bicarbonate solution. The organic phase was separated and evaporated. The residue was purified by mass directed autoprep HPLC to obtain the desired product.

The following Examples 360 to 363 were prepared from Intermediate 152 and the appropriate amine reagent Ar-C(R⁴)(R⁵)-NH₂ using this or a similar procedure:

| Example Number | 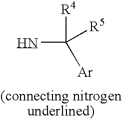 | One Possible Source of amine reagent 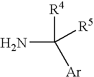 | MH⁺Ion | LC-MS reten- tion time |
|---|---|---|---|---|
| 360 | 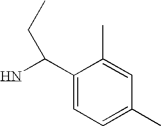 | Intermediate 82 | 464 | 2.8 |
| 361 | 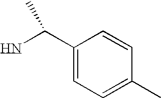 | Lancaster | 436 | 2.6 |
| 362 | 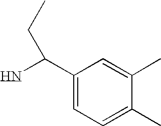 | Intermediate 84 | 464 | 2.8 |
| 363 | 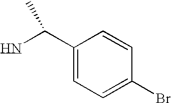 | Lancaster | 500 +502 | 2.7 |

### Examples 364 to 367

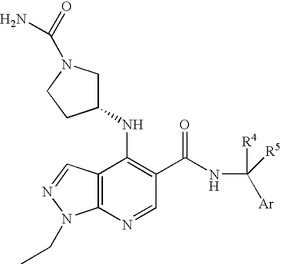 (of which, Examples 364 and possibly 366 are believed to be mixtures of diastereoisomers enriched in a major diastereoisomer believed to have the (R)-stereochemistry at the benzylic carbon atom)

General Procedure:

A mixture of Intermediate 153 (30 mg), HATU (120 mg) and DIPEA (0.09 ml) in acetonitrile (2ml) was added to the amine reagent Ar-C(R⁴)(R⁵)-NH₂ (0.09 mmol). The mixture was left to stand at room temperature for 16 hours. The solvent was evaporated. The residue was partitioned between DCM and saturated sodium bicarbonate solution. The organic phase was separated and evaporated. The residue was purified by mass directed autoprep HPLC to obtain the desired product.

The following Examples 364 to 367 were prepared from Intermediate 153 and the appropriate amine reagent Ar-C(R⁴)(R⁵)-NH₂ using this or a similar procedure:

| Example Number | 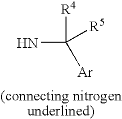 | One Possible Source of amine reagent 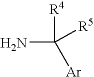 | MH⁺Ion | LC-MS reten- tion time |
|---|---|---|---|---|
| 364 | 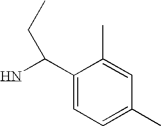 | Intermediate 82 | 464 | 2.81 |
| 365 | 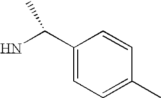 | Lancaster | 436 | 2.62 |
| 366 | 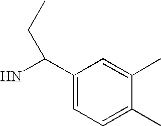 | Intermediate 84 | 464 | 2.82 |
| 367 | 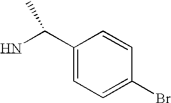 | Lancaster | 500 +502 | 2.74 |

### Examples 368 to 369

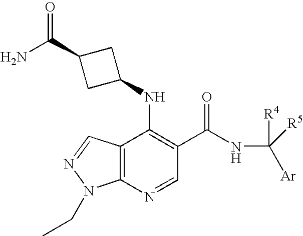

### Example 368

A mixture of Intermediate 108 (25 mg), cis-3-aminocyclobutanecarboxarnde (Chemical Abstracts Service, CAS 84182-57-0) (10 mg) and DIPEA (23 mg) in acetonitrile (4 ml) was heated at reflux for 24 h. The reaction mixture was cooled and the solvent was evaporated. The residue was purified by mass directed autoprep HPLC to give Example 368 (19 mg) as a white solid.

### Example 369

Example 369 was prepared from cis-3-aminocyclobutanecarboxamide and Intermediate 122 using a procedure similar to that used for the preparation of Example 368. Example 369 is believed to be a mixture of isomers enriched in a major isomer believed to have the (R)-stereochemistry at the benzylic carbon atom.

| Example Number | 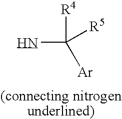 | Source of aryl chloride | MH⁺Ion | LC-MS retention time |
|---|---|---|---|---|
| 368 | 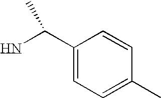 | Intermediate 108 | 421 | 2.78 |
| 369 | 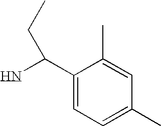 | Intermediate 122 | 449 | 3.01 |

### Examples 370 to 372

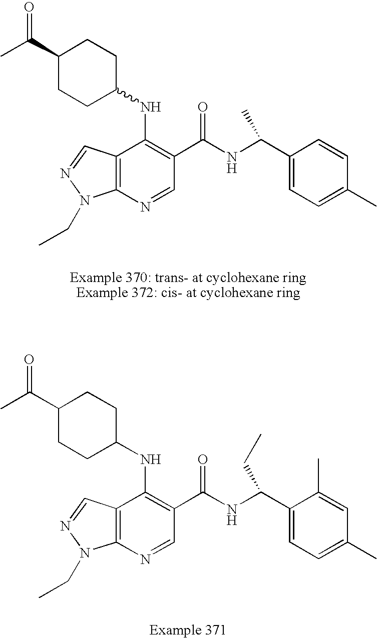 (of which, Example 371 is a mixture of isomers enriched in a major isomer(s) believed to have the (R)-stereochemistry at the benzylic carbon atom)

A mixture of Intermediate 158 (23 mg), EDC (15 mg), HOBT (10.5 mg) and DIPEA (27 ul) in DMF (1 ml) was stirred at room temperature for 30 min. then added to [(1R)-1-(4-methylphenyl)ethyl]amine (10.5 mg) (e.g. available from Lancaster). The mixture was stirred for 3 h. and then left to stand at room temperature for 16 hours. More EDC (7.5 mg) and HOBT (5.3 mg) were added and the mixture was left to stand for 3 h. More [(1R)-1-(4-methylphenyl)ethyl]amine (5.3 mg) was added and the mixture was left to stand overnight. The solvent was evaporated. The residue was partitioned between DCM and saturated sodium bicarbonate. The organic phase was separated and evaporated. The residue was purified by mass directed autoprep HPLC to obtain Example 370 (10.1 mg; major component, contains 4-(trans-4-acetylcyclohexyl)amino group).

The isomeric ketone, Example 372, was isolated as a minor component (3.7 mg, contains 4-(cis-4-acetylcyclohexyl)amino group) from the purification of Example 370.

The following Example 371 (mixture of cis and trans isomers at cyclohexane ring, and believed to consist essentially of isomers believed to have the (R)-stereochemistry at the benzylic carbon atom) was prepared from Intermediate 158 and the appropriate amine reagent (preferably Intermediate 82b) using the above procedure or a similar procedure:

| Example Number | 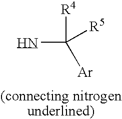 | One Possible Source of amine reagent 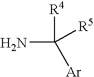 | MH⁺Ion | LC-MS reten- tion time |
|---|---|---|---|---|
| 370 | 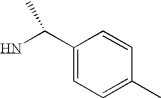 | Lancaster | 448 | 3.17 |
| 371 | 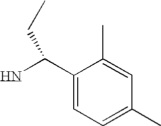 | Intermediate 82b | 476 | 3.39, 3.41 |
| 372 | 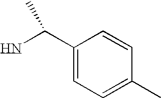 | Lancaster | 448 | 3.14 |

### Example 373

### 4-{[cis-4-(1-hydroxyethyl)cyclohexyl]amino}-N-[1-(2,4-dimethylphenyl)propyl]-1-ethyl-1H-pyrazolo[3,4-b]pyridine-5carboxamide

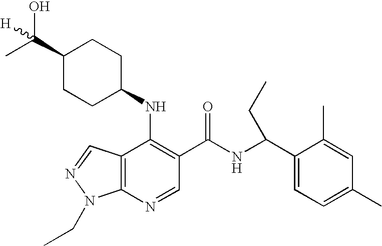 (believed to be a mixture of isomers enriched in a major isomer(s) believed to have the (R)-stereochemistry at the benzylic carbon atom)

A mixture of Intermediate 122 (13 mg), Intermediate 160 (7 mg) and DIPEA (0.3 ml) in ethanol (1 ml) was stirred and heated at reflux overnight. The mixture was cooled and the solvent was evaporated. The residue was partitioned between DCM and sodium bicarbonate solution. The organic phase was concentrated. The residue was passed through a silica column, using a mixture of cyclohexane and EtOAc as the eluent, to give Example 373 (3 mg). LCMS showed MH⁺=478; T_{RET}=3.35min.

### Examples 374 to 378

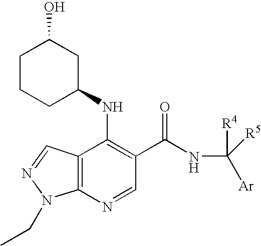 relative stereochemistry at cyclohexane ring as drawn, racemic; i.e. trans-(3-hydroxycyclohex-1-yl)amino, racemic =(trans-3-hydroxycyclohexyl)amino group, racemic
(of which Example 378 is believed to be a mixture of isomers enriched in a major isomer(s) believed to have the (R)-stereochemistry at the benzylic carbon atom; and of which Examples 375 and 376 are believed to consist essentially of isomer(s) believed to have the stereochemistry at the benzylic carbon atom shown below)

General Procedure:

A mixture of Intermediate 162 (25 mg), HATU (32 mg) and DIPEA (68 ul) in acetonitrile (2 ml) was added to the amine reagent Ar-C(R⁴)(R⁵)-NH₂ (0.08 mmol). The mixture was left to stand at room temperature for 72 hours. The solvent was evaporated. The residue was purified by mass directed autoprep HPLC to obtain the desired product.

The following Examples 374-378 were prepared from Intermediate 162 and the appropriate amine reagent Ar-C(R⁴)(R5)-NH₂ using this or a similar procedure:

| Example Number | 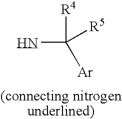 | One Possible Source of amine reagent Ar—C(R⁴)(R⁵)—NH₂ | MH⁺Ion | LC-MS retention time |
|---|---|---|---|---|
| 374 | 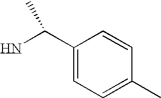 | Lancaster | 422 | 3.10 |
| 375 | 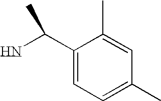 | Intermediate 101 | 436 | 3.23 |
| 376 | 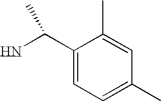 | Intermediate 100 | 436 | 3.24 |
| 377 | 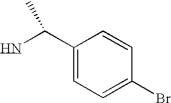 | Lancaster | 487 | 3.24 |
| 378 | 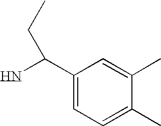 | Intermediate 84 | 450 | 3.32 |

### Example 379

### N-[4-(dimethylamino)-1-(3-methylphenyl)-4-oxobutyl]-1-ethyl4-(tetrahydro-2H-pyran4-ylamino)-1H-pyrazolo[3,4-b]pyridine-5-carboxamide

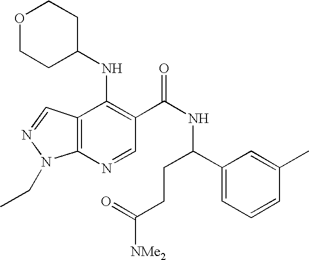

A mixture of Intermediate 13 (19 mg), HOBT (10 mg), EDC (14 mg) and DIPEA (26 mg) in acetonitrile (2.5 ml) was stirred for 10 min then added to Intermediate 169 (20 mg). The solution was stirred for 3 h then left to stand overnight at room temperature. More DIPEA (53 mg) was added. The reaction mixture was stirred for 6 h then left to stand for 3 days at room temperature. The solvent was removed in vacuo. The residue was partitioned between DCM and 1M sodium bicarbonate solution. The organic phase was separated, washed with water and concentrated in vacuo. The residue was purified by passing through a 1 g SPE cartridge, using ethyl acetate containing 50-0% cyclohexane as the eluent, to give Example 379 (18 mg) as a colourless gum. LCMS showed MH⁺=493; T_{RRT}=2.83 min.

### Example 380

### 4-{[1-(aminocarbonyl)4-piperidinyl]amino}-N-[4-(dimethylamino)-1-(3-methylphenyl)4-oxobutyl]-1ethyl-1H-pyrazolo[3,4-b]pyridine-5-carboxamide

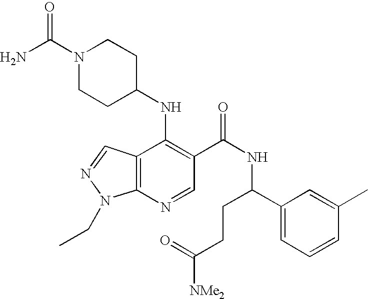

Example 380 was prepared from Intermediate 105 and Intermediate 169 using a procedure similar to that used to prepare Example 379. LCMS showed MH⁺=535; T_{RET}=2.61 min.

### Examples 381 to 382

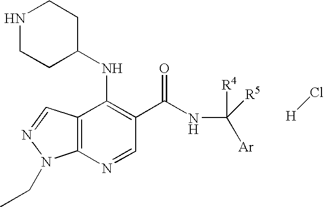

General Procedure:

A solution of the appropriate intermediate carbamate (Intermediate 164 or 165; 0.2 to 0.25 mmol) in a 4M solution of hydrogen chloride in dioxan (5 ml) was stirred for 1 h at room temperature. The solution was concentrated in vacuo to leave the product as a solid.

The following Examples 381 and 382 were prepared in this manner:

| Example Number | 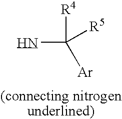 | Starting material | MH⁺Ion | LC-MS retention time |
|---|---|---|---|---|
| 381 (as hydro- chloride) | 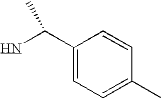 | Intermediate 164 | 407 | 2.34 |
| 382 (as hydro- chloride) | 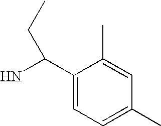 | Intermediate 165 | 435 | 2.51 |

Example 382 is believed to be a mixture of isomers with the major isomer believed to have the (R)-stereochemistry at the benzylic carbon atom.

1-72. (canceled)

73. A compound of formula (I) or a salt thereof: 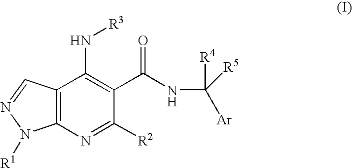
wherein Ar is (x) or (z): 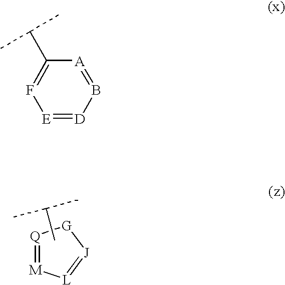
wherein:
R¹ is C₁₋₃alkyl, C₁₋₃fluoroalkyl, or —CH₂CH₂OH;
R² is hydrogen, methyl or C₁ fluoroalkyl;
R³ is unsubstituted or substituted C₃₋₈cycloalkyl, unsubstituted or substituted mono-unsaturated-C₅₋₇cycloalkenyl, or an unsubstituted or substituted heterocyclic ring selected from the group consisting of formula (aa), (bb) and (cc); 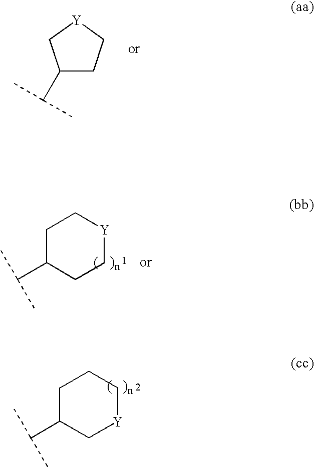
in which n¹ and n² independently are 1 or 2; and in which Y is O, S, SO₂, or NR¹⁰; where R¹⁰ is hydrogen, C₁₋₂alkyl, C₁₋₂fluoroalkyl, C(O)NH₂, C(O)—C₁₋₂alkyl, C(O)—C₁fluoroalkyl or —C(O)—CH₂O—C₁ alkyl;
and wherein in R³ the C₃₋₈cycloalkyl is unsubstituted or substituted on a ring carbon with one or two substituents selected independently from the group consisting of oxo; OH; methoxy; fluoromethoxy; NHR²¹ wherein R²¹ is hydrogen; methyl; ethyl; fluoromethyl; —CH₂OH; —CH₂NHR²² wherein R²² is H; —C(O)OR²³ wherein R²³ is H; —C(O)NHR²⁴ wherein R²⁴ is H or methyl; —C(O)R²⁵ wherein R²⁵ is methyl, fluoro, hydroxyimino, and (C₁₋₄alkoxy)imino; and wherein any OH, methoxy, fluoromethoxy, or NHR²¹ substituent is not substituted at the R³ ring carbon attached to the —NH— group of formula (I);
and wherein in R³ the heterocyclic group of sub-formula (aa), (bb) or (cc) is substituted on a ring carbon with one or two oxo groups;
and wherein when R³ is substituted mono-unsaturated-C₅₋₇cycloalkenyl, then the cycloalkenyl is substituted with a fluoro or C₁₋₂alkyl group or two substituents which are, independently, fluoro or methyl; and the R³ ring carbon bonded to the —NH— group of formula (I) does not partake in the cycloalkenyl double bond;
or R³ is a bicyclic group of sub-formula (ee): 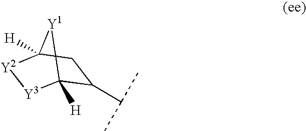
Y¹, Y² and Y³ are CH₂;
R⁴ is hydrogen, methyl, ethyl, n-propyl, isopropyl, C₁₋₂fluoroalkyl, cyclopropyl, —CH₂OR^{4a}, —CH(Me)OR^{4a}, or —CH₂CH₂OR^{4a}; wherein R^{4a} is hydrogen, methyl, or C₁fluoroalkyl;
R⁵ is hydrogen, C₁₋₈alkyl, C₁₋₃fluoroalkyl, C₃₋₈-cycloalkyl unsubstituted or substituted by a C₁₋₂alkyl group, or —(CH₂)ₙ⁴—C₃₋₈cycloalkyl unsubstituted or substituted in the —(CH₂)ₙ⁴— moiety or in the C₃₋₈cycloalkyl moiety by a C₁₋₂alkyl group, and n⁴ is 1 or 2;
or R⁵ is C₁₋₄alkyl substituted by one substituent R¹¹ wherein R¹¹ is hydroxyl, C₁₋₆alkoxy, C₁₋₂fluoroalkoxy, phenyloxy, monofluoro- or difluoro-phenyloxy, monomethyl- or dimethyl-phenyloxy, benzyloxy, —NR¹²R¹³, —NR¹⁵—C(O)R¹⁶, —NR¹⁵—C(O)—NH—R¹⁵, or —NR¹⁵—S(O)₂R¹⁶;
or R⁵ is C₂₋₄alkyl substituted on different carbon atoms by two hydroxy substituents;
or R⁵ is —(CH₂)ₙ¹¹—C(O)R¹⁶, —(CH₂)ₙ¹¹—C(O)NR¹²R¹³, —CHR¹⁹—C(O)NR¹²R¹³, —(CH₂)ₙ¹¹—C(O)OR¹⁶, —(CH₂)ₙ¹¹—C(O)OH, —CHR¹⁹—C(O)OR¹⁶, —CHR¹⁹—C(O)OH, —(CH₂)ₙ¹¹—S(O)₂—NR¹²R¹³, —(CH₂)ₙ¹¹—S(O)₂R¹⁶, or —(CH₂)ₙ¹¹—CN; wherein n¹¹ is 0, 1, 2 or 3 wherein for each R⁵ group n¹¹ is independent of the value of n¹¹ in other R⁵ groups; and wherein R¹⁹ is C₁₋₂alkyl;
or R⁵ is —(CH₂)ₙ¹³-Het wherein n¹³ is 0, 1 or 2 and Het is a 4-, 5-, 6- or 7-membered saturated or unsaturated heterocyclic ring other than —NR¹²R¹³ containing one or two ring-hetero-atoms independently selected from the group consisting of O, S, and N; wherein any ring-hetero-atoms present are not bound to the —(CH₂)ₙ¹³-moiety when n¹³ is 0, wherein any ring-nitrogen which is present and which is not unsaturated and which is not a connecting nitrogen is present as NR¹⁷; and wherein one or two of the carbon ring-atoms are unsubstituted or independently substituted by C₁₋₂alkyl;
or R⁵ is phenyl, —CH₂—Ph, —CHMe—Ph, —CHEt—Ph, —CMe₂Ph, or —CH₂CH₂—Ph; wherein the phenyl ring Ph is optionally substituted with one or two substituents selected independently from the group consisting of halo, C₁₋₄alkyl, C₁₋₂fluoroalkyl, C₁₋₄alkoxy, C₁₋₂fluoroalkoxy, cyclopropyl, cyclopropyloxy, —C(O)—C₁₋₄alkyl, —C(O)OH, —C(O)—OC₁₋₄alkyl, C₁₋₄alkyl-S(O)₂—, C₁₋₄alkyl-S(O)₂—NR^{8a}—, R^{7a}R^{8a}N—S(O)₂—, R^{7a}R^{8a}N—C(O)—, —NR^{8a}—C(O)—C₁₋₄alkyl, R^{7a}R^{8a}N, OH, nitro (—NO₂), or cyano;
or R⁴ and R⁵ taken together are —(CH₂)ₚ¹— or —(CH₂)ₚ³—X⁵—(CH₂)ₚ⁴—, in which X⁵ is O or NR^{17a}, p¹ is 2, 3, 4, 5 or 6, and p³ and p⁴ independently are 1, 2 or 3 provided that if p³ is 3 then p⁴ is 1 or 2 and if p⁴ is 3 then p³ is 1 or 2; provided that at least one of R⁴ and R⁵ is not hydrogen;
and in sub-formula (x) is:
A is C—R^{6A}, nitrogen or N⁺—O—;
B is C—R^{6B}, nitrogen or N⁺—O—;
D is C—R^{6D}, nitrogen or N⁺—O—;
E is C—R^{6E}, nitrogen or N⁺—O—; or
F is C—R^{6F}, nitrogen or N⁺—O—;
wherein R^{6A}, R^{6B}, R^{6D}, R^{6E} and R^{6F} independently are hydrogen, halo, C₁₋₆alkyl, C₁₋₄fluoroalkyl, C₃₋₆cycloalkyl, C₁₋₄alkoxy, C₁₋₂fluoroalkoxy, C₃₋₆cycloalkyloxy, —C(O)R^{16a}, —C(O)OR³⁰, —S(O)₂—R^{16a}, R^{16a}—S(O)₂—NR^{15a}—, R⁷R⁸N—S(O)₂—, C₁₋₂alkyl—C(O)—R^{15a}N—S(O)₂—, C₁₋₄alkyl—S(O)—, Ph-S(O)—, R⁷R⁸N—CO—, —NR^{15a}—C(O)R^{16a}, R⁷R⁸N, nitro, OH, C₁₋₄alkoxymethyl, C₁₋₄alkoxyethyl, C₁₋₂alkyl—S(O)₂—CH₂—, R⁷R⁸N—S(O)₂—CH₂—, C₁₋₂alkyl—S(O)₂—NR^{15a}—CH₂—, —CH₂—OH, —CH₂CH₂—OH, —CH₂—NR⁷R⁸, —CH₂—CH₂—NR⁷R⁸, —CH₂—C(O)OR³⁰, —CH₂—C(O)—NR⁷R⁸, —CH2—NR^{15a}—C(O)—C₁₋₃alkyl, —(CH₂)ₙ¹⁴—Het¹ where n¹⁴ is 0 or 1, cyano, Ar^{5b}, phenyl, pyridinyl or pyrimidinyl; wherein phenyl, pyridinyl or pyrimidinyl independently are unsubstituted or substituted by one or two groups selected from the group consisting of fluoro, chloro, C₁₋₂alkyl, fluoromethyl, C₁₋₂alkoxy and fluoromethoxy;
and/or two adjacent groups selected from the group consisting of R^{6A}, R^{6B}, R^{6D}, R^{6E} and R^{6F} are taken together and are: —CH═CH—CH═CH—; —(CH₂)ₙ^{14a}— where n^{14a} is 3, 4 or 5; —O—(CMe₂)—O—; —O—(CH₂)ₙ^{14b}—O— where n^{14b} is 1 or 2; —CH═CH—NR^{15b}—; —N═CH—NR^{15b}; —CH═N—NR^{15b}; —N═N—NR^{15b}—; —CH═CH—O—; —N═CH—O—; —CH═CH—S—; or —N═CH—S—; wherein R^{15b} is H or C₁₋₂alkyl;
provided that:
two or more of A, B, D, E and F are independently C—H, C—F, nitrogen, or N⁺—O—;
and no more than two of A, B, D, E and F are independently nitrogen or N⁺—O—,
and no more than one of A, B, D, E and F is N⁺—O—;
and wherein, in sub-formula (z):
G is O, S or NR⁹ wherein R⁹ is hydrogen, C₁₋₄alkyl, or
C₁₋₂fluoroalkyl;
J is C—R^{6J}, C-[connection point to formula (I)], or nitrogen;
L is C—R^{6L}, C-[connection point to formula (I)], or nitrogen;
M is C—R^{6M}, C-[connection point to formula (I)], or nitrogen;
Q is C—R^{6Q}, C-[connection point to formula (I)], or nitrogen;
wherein R^{6J}, R^{6L}, R^{6M} and R^{6Q} independently are hydrogen; halo; C₁₋₄alkyl; C₁₋₃fluoroalkyl; C₃₋₆cycloalkyl; C₁₋₄alkoxy; C₁₋₂fluoroalkoxy; C₃₋₆cycloalkyloxy; OH; or phenyl unsubstituted or substituted by one or two substituents independently selected from the group consisting of fluoro, chloro, C₁₋₂alkyl, C₁fluoroalkyl, C₁₋₂alkoxy and fluoromethoxy;
provided that:
two or more of J, L, M and Q independently are C—H, C—F, C—C₁₋₂alkyl, C-[connection point to formula (I)], or nitrogen;
and no more than three of J, L, M and Q are nitrogen;
and wherein:
R⁷ and R⁸ are independently hydrogen; C₁₋₄alkyl; C₃₋₆cycloalkyl; or phenyl unsubstituted or substituted by one or two substituents independently selected from the group consisting of fluoro, chloro, C₁₋₂alkyl, flurormethyl, C₁₋₂alkoxy and fluoromethoxy;
or R⁷ and R⁸ together are —(CH₂)ₙ⁶—, —C(O)—(CH₂)ₙ⁷—, —C(O)—(CH₂)ₙ¹⁰—C(O)—, —(CH₂)ₙ⁸—X⁷—(CH₂)ₙ⁹—, or —C(O)—X⁷—(CH₂)ₙ¹⁰—; in which n⁶ is 3, 4, 5 or 6; n⁷ is 2, 3, 4, or 5; n⁸ and n⁹ and n¹⁰ independently are 2 or 3; and X⁷ is O or NR¹⁴;
R^{7a} is hydrogen or C₁₋₄alkyl;
R^{8a} is hydrogen or methyl;
R¹² and R¹³ independently are H; C₁₋₄alkyl; C₃₋₆cycloalkyl; or phenyl unsubstituted or substituted by one or two substituents independently from the group consisting of fluoro, chloro, C₁₋₂alkyl, fluoromethyl, C₁₋₂alkoxy and fluoromethoxy;
or R¹² and R¹³ together are -(CH₂)ₙ^{6a}—, —C(O)—(CH₂)ₙ^{7a}—, —C(O)—(CH₂)ₙ^{10a}—C(O)—, —(CH₂)ₙ^{8a}—X¹²—(CH₂)ₙ^{9a}—, or —C(O)—X¹²—(CH₂)ₙ^{10a}—; in which n^{6a} is 3, 4, 5 or 6; n^{7a} is 2, 3, 4, or 5; n^{8a} and n^{9a} and n^{10a} independently are 2 or 3; and X¹² is O or NR^{14a};
R¹⁴, R^{14a}, R¹⁷ and R^{17a} independently are hydrogen, C₁₋₄alkyl, C₁₋₂fluoroalkyl, cyclopropyl, —C(O)—C₁₋₄alkyl, —C(O)NR^{7a}R^{8a}, or —S(O)₂—C₁₋₄alkyl;
R¹⁵, independent of another R¹⁵, is hydrogen; C₁₋₄alkyl; C₃₋₆cycloalkyl; or phenyl unsubstituted or substituted by one or two groups selected from the group consisting of halo, C₁₋₂alkyl, fluoromethyl, C₁₋₂alkoxy and fluoromethoxy;
R^{15a}, independent of other R^{15a}, is hydrogen or C₁₋₄alkyl;
R¹⁶ is C₁₋₄alkyl, C₃₋₆cycloalkyl, C₃₋₆cycloalkyl-CH₂—, phenyl or benzyl; wherein the phenyl and benzyl are unsubstituted or independently substituted on their ring by one or two substituents which are independently fluoro, chloro, methyl, C₁ fluoroalkyl, methoxy or C₁ fluoroalkoxy;
R^{16a} is C₁₋₆alkyl; C₃₋₆cycloalkyl unsubstituted or substituted by one oxo, OH or C₁₋₂alkyl substituent; C₃₋₆cycloalkyl-CH₂—; pyridinyl unsubstituted or substituted on a ring carbon atom by one halo, C₁₋₂alkyl, fluoromethyl, C₁₋₂alkoxy or flurormethoxy; Ar^{5c}; phenyl unsubstituted or substituted by one or two substituents which are independently halo, C₁₋₂alkyl, fluoromethyl, C₁₋₂alkoxy, fluoromethoxy, or benzyl unsubstituted or substituted by one or two substituents which are independently halo, C₁₋₂alkyl, C₁fluoroalkyl, C₁₋₂alkoxy or C₁fluoroalkoxy; or a 4-, 5-, 6- or 7-membered saturated heterocyclic ring connected at a ring-carbon and containing one or two ring-hetero-atoms independently selected from the group consisting of O, S, and N; wherein any ring-nitrogen which is present is present as NR²⁷ where R²⁷ is H, C₁₋₂alkyl or —C(O)Me; and wherein the ring is optionally substituted at carbon by one C₁₋₂alkyl or oxo substituent, provided that any oxo substituent is substituted at a ring-carbon atom bonded to a ring-nitrogen;
R³⁰, independent of other R³⁰, is hydrogen, C₁₋₄alkyl or C₃₋₆cycloalkyl;
Ar^{5b} and Ar^{5c} independently are a 5-membered aromatic heterocyclic ring containing one O, S or NR^{15a} in the 5-membered ring, wherein the 5-membered ring can contain one or two N atoms and wherein the heterocyclic ring is unsubstituted, or substituted on a ring carbon atom by a group selected from the group consisting or halo, C₁₋₂alkyl, C₁fluoroalkyl, —CH₂OH, —CH₂—OC₁₋₂alkyl, OH and —CH₂—NR²⁸R²⁹ wherein R²⁸ and R²⁹ independently are H or methyl;
Het¹ is a 4-, 5-, 6- or 7-membered saturated heterocyclic ring connected at a ring-carbon and containing one or two ring-hetero-atoms which are independently O, S, and N wherein any ring-nitrogen which is present is present as NR³¹ where R³¹ is H, C₁₋₂alkyl or —C(O)Me; and wherein the ring is unsubstituted or substituted at carbon by C₁₋₂alkyl or oxo provided that any oxo group is substituted at a ring-carbon atom bonded to a ring-nitrogen;
provided that:
when R³ is (bb) n¹ is 1 and Y is NR¹⁰, then R¹⁰ is other than C₁₋₂alkyl or C₁₋₂fluoroalkyl; and
when R³ is (aa) and Y is NR¹⁰ then R¹⁰ is other than C(O)—C₁₋₂alkyl, C(O)—C₁ fluoroalkyl or —C(O)—CH₂O—C₁ alkyl; and
when R³ is (cc) then Y is O, S, SO₂ or NR¹⁰ wherein R¹⁰ is H;
and provided that:
when R³ is substituted C₃₋₈cycloalkyl or substituted C₅₋₇cycloalkenyl, then any —C(O)OR²³, —C(O)NHR²⁴, —C(O)R²⁵, —CH₂OH or fluoro substituent is at the 3-position of a R³ cyclobutyl ring; or at the 3- or 4-position of a R³ C₅cycloalkyl (cyclopentyl) or cyclopentenyl ring; or at the 4-position of a R³ C₆cycloalkyl (cyclohexyl) or cyclohexenyl ring; or at the 3-, 4-, 5- or 6-position of a R³ cycloheptyl or cycloheptenyl ring; or at the 3-, 4-, 5-, 6- or 7-position of a R³ cyclooctyl ring;
and provided that:
when R³ is substituted C₃₋₈cycloalkyl, then any OH, alkoxy, fluoroalkoxy or —CH₂NHR²² substituent is at the 3-position of a R³ cyclobutyl ring; or at the 3- or 4-position of a R³ cyclopentyl ring; or at the 3-, 4- or 5-position of a R³ cyclohexyl ring; or at the 3-, 4-, 5- or 6-position of
a R³ cycloheptyl ring; or at the 3-, 4-, 5-, 6- or 7-position of a R³ cyclooctyl ring.

74. A compound as claimed in claim 73 wherein R¹ is ethyl, n-propyl or —CH₂CH₂OH.

75. A compound as claimed in claim 74 wherein R¹ is ethyl.

76. A compound as claimed in claim 73 wherein R² is hydrogen or methyl.

77. A compound as claimed in claim 76 wherein R² is hydrogen.

78. A compound as claimed in claim 73 wherein in R³ there is one substituent or no substituent.

79. A compound as claimed in claim 73 wherein R³ is the unsubstituted or substituted C₃₋₈cycloalkyl or the unsubstituted or substituted heterocyclic group of sub-formula (aa), (bb) or (cc).

80. A compound as claimed in claim 73 wherein R³ is unsubstituted or unsubstituted or substituted cyclobutyl.

81. A compound as claimed in claim 73 wherein R³ is C₃₋₈cycloalkyl unsubstituted or substituted with one or two substituents which are independently oxo; OH; NHR²¹ wherein R²¹ is hydrogen; methyl; —CH₂F; —CHF₂; —C(O)OR²³ wherein R²³ is H; —C(O)NHR²⁴ wherein R²⁴ is H; fluoro; hydroxyimino (═N—OH); or methoxyimino.

82. A compound as claimed in claim 81 wherein R³ is C₃₋₈cycloalkyl substituted with one substituent selected from the group consisting of OH; —C(O)NHR²⁴ wherein R²⁴ is H; oxo, and hydroxyimino.

83. A compound as claimed in claim 81 wherein, R³ is substituted at other than the ring atom connecting to the —NH— in formula (I), and R³ is substituted at other than the two ring atoms on either side of the connecting atom.

84. A compound as claimed in claim 73 wherein R³ is substituted by one or two substituents
(a) at the 3-position of a R³ cyclobutyl ring; or
(b) at the 3- and/or 4-position(s) of a R³ cyclopentyl or cyclopentenyl ring; or
(c) at the 3-, 4- and/or 5-position(s) of a R³ cyclohexyl or cyclohexenyl ring; or;
(d) at the 3-, 4-, 5- and/or 6-position(s) of a R³ cycloheptyl or cycloheptenyl ring; or
(e) at the 3-, 4-, 5-, 6- and/or 7-position(s) of a R³ cyclooctyl ring; or
(f) at the 1-, 2- and/or highest-numbered position(s) of a R³ cycloalkyl or cycloalkenyl ring, for alkyl or fluoroalkyl substituent(s); or
(g) at the 2- and/or highest-numbered-position(s) of a R³ cycloalkyl or cycloalkenyl ring, for NHR²¹ substituent(s).

85. A compound as claimed in claim 73 wherein R³ is substituted mono-unsaturated-C₆cycloalkenyl wherein the R³ cyclohexenyl is substituted with one fluoro or methyl group.

86. A compound as claimed in claim 73 wherein R³ is selected from the group (aa), (bb) and (cc); and Y is O or NR¹⁰ wherein R¹⁰ is H, C(O)NH₂ or C(O)methyl.

87. A compound as claimed in claim 73 wherein R¹⁰ is H, C(O)NH₂ or C(O)methyl.

88. A compound as claimed in claim 73 wherein R³ is sub-formula (bb) and n¹ is 1.

89. A compound as claimed in claim 73 wherein R³ is (aa), (bb) or (cc), and has an oxo group on a carbon atom bonded to Y.

90. A compound as claimed in claim 73 wherein R³ is unsubstituted (aa), (bb) or (cc).

91. A compound as claimed in claim 73 wherein NHR³ is sub-formula (a), (a1), (b), (c), (c1), (c2), (c3), (c4), (c5), (c6), (c7), (d), (e), (f), (g), (g1), (g2) (g3), (g4), (h), (i), (j), (k), (k1), (k2), (L), (m), (m1), (m2), (m3), (n), (o), (o1), (o2), (o3), (p), (p1), (p2), (p3), (p5), (p6), (p9), (p10), (p11) or (q):

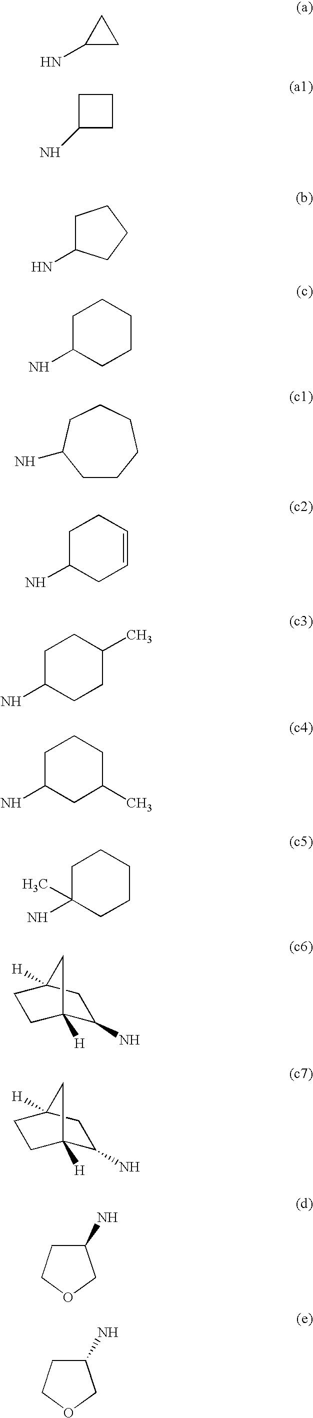

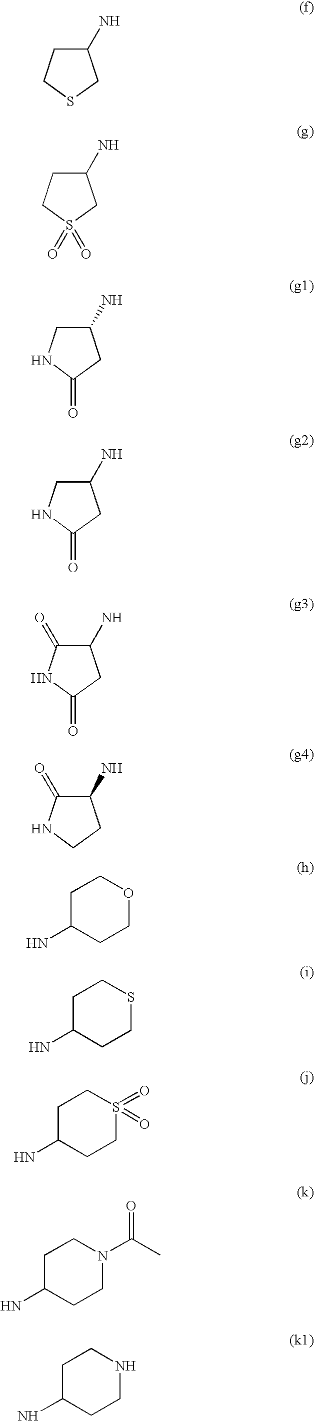

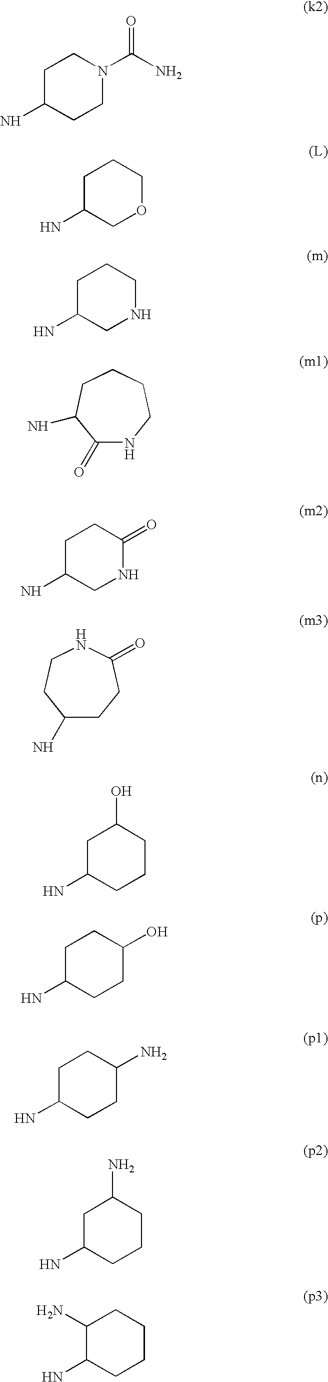

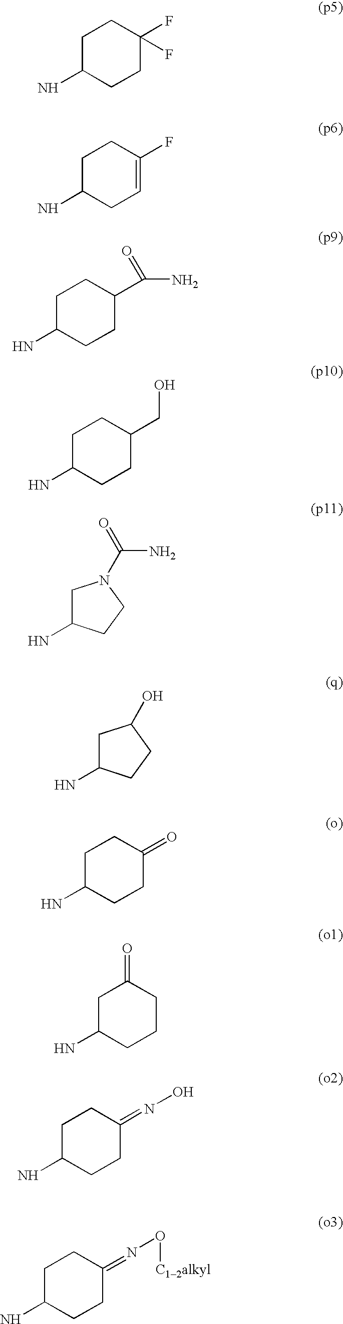

92. A compound as claimed in claim 91 wherein NHR³ is (c), (c1), (c4), (c5), (h), (i), (j), (k), (k2), (m1), (n), (o), (o2), (o3), (p2), (p5), (p6), (p9), (p11) or (q).

93. A compound as claimed in claim 91 wherein NHR³ is (c), (h), (k2), (n), (o), (o2), (p9) or (p11).

94. A compound as claimed in claim 93 wherein:
when NHR³ is formula (n), then it is in the cis configuration; and
when NHR³ is formula (p9), then it is in the cis configuration.

95. A compound as claimed in claim 91 wherein NHR³ (h) or (k2).

96. A compound as claimed in claim 73 wherein R⁴ is hydrogen, methyl, ethyl, fluoromethyl, —CH₂OH, —CH(Me)OH, —CH₂CH₂OH, or —CH₂OMe.

97. A compound as claimed in claim 96, wherein R⁴ is hydrogen, methyl, ethyl, CF₃, —CH₂OH, or —CH₂OMe.

98. A compound as claimed in claim 73 wherein:
R⁵ is hydrogen; C₁₋₅alkyl; C₁₋₂fluoroalkyl; unsubstituted C₃₋₆cycloalkyl, or unsubstituted —(CH₂)ₙ⁴—C₃₋₆cycloalkyl, wherein n⁴ is 1 or 2;
or R⁵ is C₁₋₃alkyl substituted by one R¹¹ which is hydroxyl, C₁₋₄alkoxy, fluoromethoxy, —NR¹²R¹³, —NR¹⁵—C(O)R¹⁶, or —NR¹⁵—S(O)₂R¹⁶;
or R⁵ is —(CH₂)ₙ¹¹—C(O)NR¹²R¹³, —(CH₂)ₙ¹¹—C(O)OR¹⁶, —(CH₂)ₙ¹¹—C(O)OH, or —(CH₂)ₙ¹¹—CN; wherein n¹¹ is 0, 1 or 2 and wherein for each R⁵ group n¹¹ is independent of the value of n¹¹ in other R⁵ groups;
or R⁵ is —(CH₂)ₙ¹³-Het wherein n¹³ is 0 or 1 and Het is: 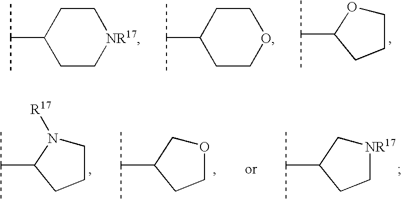
or R⁵ is phenyl or —CH₂-phenyl wherein the phenyl ring is unsubstituted or substituted with one or two substituents which are independently fluoro, chloro, C₁₋₂alkyl, flyoromethyl, C₁₋₂alkoxy, or fluoromethoxy;
or R⁴ and R⁵ taken together are —(CH₂)₂—O—(CH₂)₂— or —(CH₂)ₚ¹— in which: p¹ is 2, 4 or 5.

99. A compound as claimed in claim 98, wherein R¹¹ is OH, ethoxy, methoxy, NH₂, NHMe, NHEt, NMe₂, pyrrolidin-1-yl or piperidin-1-yl.

100. A compound as claimed in claim 98, wherein:
R^{7a} is H or methyl;
R^{8a} is H;
R⁷ and R⁸ are independently hydrogen; C₁₋₂alkyl; C₃₋₆cycloalkyl; or phenyl optionally substituted by one substituent which is fluoro, chloro, C₁₋₂alkyl, fluoromethyl, C₁₋₂alkoxy or fluoromethoxy; and when R⁷ is cycloalkyl or unsubstituted or substituted phenyl then R⁸ is other than cycloalkyl, phenyl or substituted phenyl;
or R⁷ and R⁸ together are —(CH₂)ₙ⁶— or —(CH₂)ₙ⁸—X⁷—(CH₂)ₙ⁹—, wherein X⁷ is NR¹⁴ or O, n⁶ is 4 or 5, and n⁸ and n⁹ are 2;
R¹² and R¹³ independently are H; C₁₋₂alkyl; C₃₋₆cycloalkyl; or phenyl unsubstituted or substituted by one substituent which is fluoro, chloro, C₁₋₂alkyl, fluoromethyl, C₁₋₂alkoxy or fluoromethoxy; and wherein when R¹² is cycloalkyl or optionally substituted phenyl then R¹³ is other than cycloalkyl, phenyl or substituted phenyl;
or R¹² and R¹³ together are —(CH₂)ₙ^{6a}— or —(CH₂)ₙ^{8a}—X¹²—(CH₂)ₙ^{9a}—, wherein X¹² is NR^{14a} or O, n^{6a} is 4 or 5, and n^{8a} and n^{9a} are 2;
R¹⁴, R^{14a}, R¹⁷ and R^{17a} independently are H, C₁₋₂alkyl, or —C(O)Me;
R¹⁵ is hydrogen or methyl;
R^{15a}, independent of other R^{15a}, is H or C₁₋₂alkyl;
R^{15b} is H;
R¹⁶ is C₁₋₄alkyl;
R^{16a} is C₁₋₄alkyl; unsubstituted C₃₋₆cycloalkyl; phenyl; phenyl substituted by one or two substituents which are independently halo, C₁₋₂alkyl, fluoromethyl, C₁₋₂alkoxy or fluoromethoxy; benzyl or benzyl substituted on its ring by one or two substituents which are independently halo, C₁₋₂alkyl, fluoromethyl, C₁₋₂alkoxy or fluoromethyl; and
R³⁰, independent of other R³⁰, is hydrogen or C₁₋₄alkyl.

101. A compound as claimed in claim 100 wherein
R⁷ and R⁸ independently are hydrogen or C₁₋₂alkyl;
R¹² and R¹³ independently are hydrogen or C₁₋₂alkyl; and
R^{16a} is C₁₋₄alkyl.

102. A compound as claimed in claim 73 wherein Ar has the sub-formula (x).

103. A compound as claimed in claim 103 wherein, in (x):
two or more of A, B, D, E and F are C—H; and one or more others of A, B, D, E and F are independently C—H, C—F, C—Cl, C—Me, C—OMe, or nitrogen;
no more than one of A, B, D, E and F is nitrogen; and
A, B, D, E and F are other than N⁺—O—.

104. A compound as claimed in claim 102 wherein Ar is (x) comprising a sub-formula selected from the group consisting of (x1), (x2), (x3), (x4), (x5), (x6), (x7), (x8), (x9), (x10), (x11), (x12), (x13), (x14), (x15) or (x16):

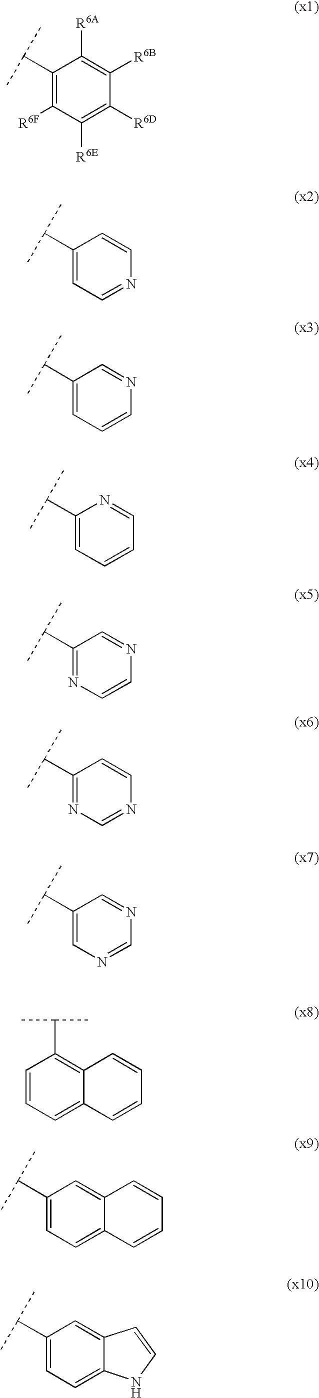

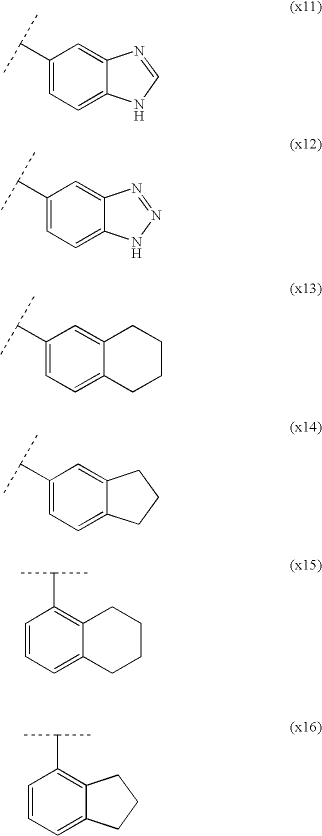

105. A compound as claimed in claim 104 wherein Ar is (x1), (x8), (x13), or (x14).

106. A compound as claimed in claim 105 wherein Ar is (x1).

107. A compound as claimed in claim 106, (x1) is monoalkyl-phenyl-, mono(fluoroalkyl)-phenyl-, monohalo-phenyl-, monoalkoxy-phenyl-, mono(fluoroalkoxy)-phenyl-, dialkyl-phenyl-, monoalkyl-monohalo-phenyl-, dihalo-phenyl- or dihalo-monoalkyl-phenyl-.

108. A compound as claimed in claim 107 wherein Ar is monoC₁₋₃alkyl-phenyl; monofluoromethyl-phenyl-; monoC -3alkoxy-phenyl-; mono(fluoromethoxy)-phenyl-; diC₁₋₂alkyl-phenyl-; mono C₁₋₃ alkyl-monohalo-phenyl-; dihalo-phenyl-; or dihalo-monoC₁₋₂alkyl-phenyl-.

109. A compound as claimed in claim 106 wherein in sub-formula (x), R^{6A}, R^{6B}, R^{6D}, R^{6E} and R^{6F}, independently of each other, are hydrogen, fluoro, chloro, bromo, iodo, methyl, ethyl, n-propyl, isopropyl, C₄alkyl, trifluoromethyl, —CH₂OH, methoxy, ethoxy, n-propoxy, isopropoxy, fluoromethoxy, cyclohexyloxy; cyclopentyloxy, nitro, OH, C₁₋₃alkylS(O)₂—, C₁₋₃alkylS(O)₂—NH—, Me₂N—S(O)₂—, H₂N—S(O)₂—, —CONH₂, —CONHMe, —C(O)OH, cyano, NMe₂, or C₁₋₂alkyl-S(O)₂—CH₂—.

110. A compound as claimed in claim 109 wherein R^{6A}, R^{6B}, R^{6D}, R^{6E} and R^{6F}, independently of each other, are hydrogen, fluror, chloro, bormo, methyl, ethyl, n-propyl, isopropyl, trifluoromethyl, —CH₂OH, methoxy, ethoxy, n-propoxy, difluoromethoxy, OH or MeS(O)₂—.

111. A compound as claimed in claim 73 wherein
R⁹ is hydrogen or methyl;
R^{6J}, R^{6L}, R^{6M} and R^{6Q} independently are H, OH, C₁₋₂alkyl or C₁fluoroalkyl; and
when Ar has the sub-formula (z), then sub-formula (z) is selected from the group consisting of:

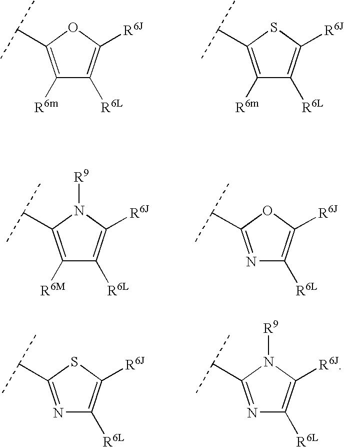

112. A compound as claimed in claim 73 wherein the compound of formula (I) or the salt thereof is racemic at the carbon atom bearing the R⁴ and R⁵ groups, or the compound of formula (I) is a compound of formula (IA): 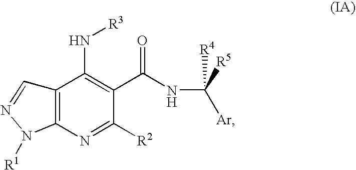
wherein Formula (IA) means that more than 50% of the compound present has the stereochemistry shown at the carbon atom bearing the R⁴ and R⁵ groups.

113. A compound as claimed in claim 112 which is formula (IA) or a salt thereof.

114. A compound as claimed in claim 113 wherein in Formula (IA) the stereochemistry at the carbon atom bearing the R⁴ and R⁵ groups is such that there is an enantiomeric excess of 50% or more at the carbon atom bearing the R⁴ and R⁵ groups and wherein enantiomeric excess is defined as the percentage of the major isomer present minus the percentage of the minor isomer present.

115. A compound as claimed in claim 114 wherein in formula (IA) R⁵ is hydrogen and R⁴ is other than hydrogen.

116. A compound as claimed in claim 115 wherein in formula (IA) R⁵ is hydrogen; and R⁴ is methyl, ethyl, C₁fluoroalkyl, —CH₂OH, or —CH₂OMe.

117. A compound as claimed in claim 116 wherein, in formula (IA), R⁵ is hydrogen and R⁴ is methyl or ethyl.

118. A compound as claimed in claim 117 wherein Ar is (x) or (z) wherein
A is C—R^{6A}, nitrogen or N⁺—O—;
B is C—R^{6B}, nitrogen or N⁺—O—;
D is C—R^{6D}, nitrogen or N⁺—O—;
E is C—R^{6E}, nitrogen or N⁺—O—; and
F is C—R^{6F}, nitrogen or N⁺—O—;
wherein:
R^{6A}, R^{6B}, R^{6D}, R^{6E} and R^{6F} independently are hydrogen, halo, C₁₋₆alkyl, C₁₋₄fluoroalkyl, C₃₋₆cycloalkyl, C₁₋₄alkoxy, C₁₋₂fluoroalkoxy, C₃₋₆cycloalkyloxy, —C(O)R^{16a}, —C(O)OR³⁰, —S(O)₂—R^{16a}, R^{16a}—S(O)₂—NR^{15a}—, R⁷R⁸N—S(O)₂—, C₁₋₂alkyl-C(O)-R^{15a}N—S(O)₂—, C₁₋₄alkyl-S(O)—, Ph-S(O)—, R⁷R⁸N—CO—, —NR^{15a}—C(O)R^{16a}, R⁷R⁸N, nitro, OH, C₁₋₄alkoxymethyl, C₁₋₄alkoxyethyl, C₁₋₂alkyl-S(O)₂—CH₂—, R⁷R⁸N—S(O)₂—CH₂—, C₁₋₂alkyl-S(O)₂—NR^{15a}—CH₂—, —CH₂—OH, —CH₂CH₂—OH, —CH₂—NR⁷R⁸, —CH₂—CH₂—NR⁷R⁸, —CH₂—C(O)OR³⁰, —CH₂—C(O)—NR⁷R⁸, —CH₂—NR15a—C(O)—C₁₋₃alkyl, —(CH₂)ₙ¹⁴-Het¹where n¹⁴ is 0 or 1, cyano, Ar^{5b}b, phenyl, pyridinyl or pyrimidinyl; wherein phenyl, pyridinyl or pyrimidinyl independently are unsubstituted or substituted by one or two groups selected from the group consisting of fluoro, chloro, C₁₋₂alkyl, fluoromethyl, C₁₋₂alkoxy and fluoromethoxy;

119. A compound as claimed in claim 117 which is a compound of Formula (XXVIII) or a salt thereof: 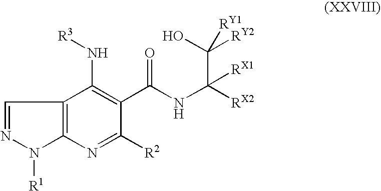
wherein:
R^{X1} is hydrogen, C₁₋₂alkyl or fluoromethyl;
R^{Y1} is hydrogen or C₁₋₂alkyl;
R^{Y2} is hydrogen; C₁₋₃alkyl; or —(CH₂)ₙ^{7aa}—OH; wherein n^{7aa} is 1, 2 or 3; and
R^{X2} is Ar^{A}, wherein:
(i) Ar^{A} is phenyl unsubstituted or substituted by one or two substituents which are independently fluoro, chloro, bromo, C₁₋₂alkyl, C₁₋₂fluoroalkyl, C₁₋₂alkoxy, C₁₋₂fluoroalkoxy; OH; —NR^{11aa}R^{11bb} (wherein R^{11aa} is H or C₁₋₂alkyl and R^{11bb} is H, C₁₋₂alkyl, —C(O)—C₁₋₂alkyl or —S(O)₂-C₁₋₂alkyl); cyano; —C(O)—NR^{11cc}R^{11dd} (wherein R^{11cc} and R^{11dd} independently are H or C₁₋₂alkyl); —C(O)—OR^{11ee} wherein R^{11ee} is H or C₁₋₂alkyl; or —S(O)₂—R^{11ff} (wherein R^{11ff} is C₁₋₂alkyl, NH₂, NHMe or NMe₂); or the phenyl Ar^{A} is substituted at two adjacent Ar ring atoms by the two ends of a chain which is: —(CH₂)₄—, —(CH₂)₃—, or —CH═CH—CH═CH—; or
(ii) Ar^{A} is an unsubstituted or substituted 5-membered heterocyclic aromatic ring containing 1, 2, 3 or 4 heteroatoms selected from the group consisting of O, N or S; and wherein when the heterocyclic aromatic ring Ar^{A} contains 2, 3 or 4 heteroatoms, one is O, N or S and the remaining heteroatom(s) are N; and wherein the heterocyclic aromatic ring Ar^{A} is optionally substituted by one or two groups independently being C₁₋₄alkyl or OH.

120. A compound of formula (I) according to claim 73 which is:
1-ethyl-N-[(1R)-1-phenylpropyl]-4-(tetrahydro-2H-pyran-4-ylamino)-1H-pyrazolo[3,4-b]pyridine-5-carboxamide;
1-ethyl-N-(1-methyl-1-phenylethyl)-4-(tetrahydro-2H-pyran-4-ylamino)-1H-pyrazolo[3,4-b]pyridine-5-carboxamide;
1-ethyl-N-{1-[4-(methylsulfonyl)phenyl]ethyl}-4-(tetrahydro-2H-pyran-4-ylamino)-1H-pyrazolo[3,4-b]pyridine-5-carboxamide;
N-(diphenylmethyl)-1-ethyl-4-(tetrahydro-2H-pyran-4-ylamino)-1H-pyrazolo[3,4-b]pyridine-5-carboxamide;
1-ethyl-N-[1-(3-pyridinyl)ethyl]-4-(tetrahydro-2H-pyran-4-ylamino)-1H-pyrazolo[3,4-b]pyridine-5-carboxamide;
1-ethyl-N-[(1S)-1-phenylpropyl]-4-(tetrahydro-2H-pyran-4-ylamino)-1H-pyrazolo[3,4-b]pyridine-5-carboxamide;
1-ethyl-N-[(1S)-1-phenylethyl]-4-(tetrahydro-2H-pyran-4-ylamino)-1H-pyrazolo[3,4-b]pyridine-5-carboxamide;
1-ethyl-N-[(1R)-1-phenylethyl]-4-(tetrahydro-2H-pyran-4-ylamino)-1H-pyrazolo[3,4-b]pyridine-5-carboxamide;
1-ethyl-N-[1-methyl-1-(4-pyridinyl)ethyl]-4-(tetrahydro-2H-pyran-4-ylamino)-1H-pyrazolo[3,4-b]pyridine-5-carboxamide;
1-ethyl-N-[(1R)-1-phenylethyl]-4-(tetrahydro-2H-pyran-4-ylamino)-1H-pyrazolo[3,4-b]pyridine-5-carboxamide;
N-[1-(4-chlorophenyl)propyl]-1-ethyl-4-(tetrahydro-2H-pyran-4-ylamino)-1H-pyrazolo[3,4-b]pyridine-5-carboxamide;
1-ethyl-N-{1-[4-(ethyloxy)phenyl]ethyl}-4-(tetrahydro-2H-pyran-4-ylamino)-1H-pyrazolo[3,4-b]pyridine-5-carboxamide;
1-ethyl-N-(3-hydroxy-1-phenylpropyl)-4-(tetrahydro-2H-pyran-4-ylamino)-1H-pyrazolo[3,4-b]pyridine-5-carboxamide;
1-ethyl-N-[1-(3-hydroxyphenyl)ethyl]-4-(tetrahydro-2H-pyran-4-ylamino)-1H-pyrazolo[3,4-b]pyridine-5-carboxamide;
N-[2-(dimethylamino)-1-phenylethyl]-1-ethyl-4-(tetrahydro-2H-pyran-4-ylamino)-1H-pyrazolo[3,4-b]pyridine-5-carboxamide;
1-ethyl-N-[1-phenyl-2-(1-pyrrolidinyl)ethyl]-4-(tetrahydro-2H-pyran-4-ylamino)-1H-pyrazolo[3,4-b]pyridine-5-carboxamide;
1-ethyl-N-[1-(hydroxymethyl)-1-phenylpropyl]-4-(tetrahydro-2H-pyran-4-ylamino)-1H-pyrazolo[3,4-b]pyridine-5-carboxamide;
1-ethyl-N-{1-[4-(propyloxy)phenyl]ethyl}-4-(tetrahydro-2H-pyran-4-ylamino)-1H-pyrazolo[3,4-b]pyridine-5-carboxamide;
methyl 3-({[1-ethyl-4-(tetrahydro-2H-pyran-4-ylamino)-1H-pyrazolo[3,4-b]pyridin-5-yl]carbonyl}amino)-3-phenylpropanoate;
1-ethyl-N-[1-(4-fluorophenyl)ethyl]-4-(tetrahydro-2H-pyran-4-ylamino)-1H-pyrazolo[3,4-b]pyridine-5-carboxamide;
N-[1-(4-chlorophenyl)ethyl]-1-ethyl-4-(tetrahydro-2H-pyran-4-ylamino)-1H-pyrazolo[3,4-b]pyridine-5-carboxamide;
ethyl ({[1-ethyl-4-(tetrahydro-2H-pyran-4-ylamino)-1H-pyrazolo[3,4-b]pyridin-5-yl]carbonyl}amino)(phenyl)acetate;
1-ethyl-N-{(1R)-1-[3-(methyloxy)phenyl]ethyl}-4-(tetrahydro-2H-pyran-4-ylamino)-1H-pyrazolo[3,4-b]pyridine-5-carboxamide;
1-ethyl-N-[(1S)-2-(methyloxy)-1-phenylethyl]-4-(tetrahydro-2H-pyran-4-ylamino)-1H-pyrazolo[3,4-b]pyridine-5-carboxamide;
N-[(1R)-2-amino-2-oxo-1-phenylethyl]-1-ethyl-4-(tetrahydro-2H-pyran-4-ylamino)-1H-pyrazolo[3,4-b]pyridine-5-carboxamide;
1-ethyl-N-[(1R)-2-hydroxy-1-phenylethyl]-4-(tetrahydro-2H-pyran-4-ylamino)-1H-pyrazolo[3,4-b]pyridine-5-carboxamide;
1-ethyl-N-[(1R)-1-(4-nitrophenyl)ethyl]-4-(tetrahydro-2H-pyran-4-ylamino)-1H-pyrazolo[3,4-b]pyridine-5-carboxamide;
1-ethyl-N-[(1S)-2-hydroxy-1-phenylethyl]-4-(tetrahydro-2H-pyran-4-ylamino)-1H-pyrazolo[3,4-b]pyridine-5-carboxamide;
1-ethyl-N-[(1R)-2-(methyloxy)-1-phenylethyl]-4-(tetrahydro-2H-pyran-4-ylamino)-1H-pyrazolo[3,4-b]pyridine-5-carboxamide;
1-ethyl-N-(2-hydroxy-1,1-diphenylethyl)-4-(tetrahydro-2H-pyran-4-ylamino)-1H-pyrazolo[3,4-b]pyridine-5-carboxamide;
N-[1-(3-cyanophenyl)ethyl]-1-ethyl-4-(tetrahydro-2H-pyran-4-ylamino)-1H-pyrazolo[3,4-b]pyridine-5-carboxamide;
N-[cyano(phenyl)methyl]-1-ethyl-4-(tetrahydro-2H-pyran-4-ylamino)-1H-pyrazolo[3,4-b]pyridine-5-carboxamide;
N-{cyclopropyl[4-(methyloxy)phenyl]methyl}-1-ethyl-4-(tetrahydro-2H-pyran-4-ylamino)-1H-pyrazolo [3,4-b]pyridine-5-carboxamide;
1-ethyl-N-[1-(1-naphthalenyl)ethyl]-4-(tetrahydro-2H-pyran-4-ylamino)-1H-pyrazolo[3,4-b]pyridine-5-carboxamide;
N-(1,2-diphenylethyl)-1-ethyl-4-(tetrahydro-2H-pyran-4-ylamino)-1H-pyrazolo[3,4-b]pyridine-5-carboxamide;
1-ethyl-N-{1-[4-(methyloxy)phenyl]butyl}-4-(tetrahydro-2H-pyran-4-ylamino)-1H-pyrazolo[3,4-b]pyridine-5-carboxamide;
1-ethyl-N-[(1R)-1-(1-naphthalenyl)ethyl]-4-(tetrahydro-2H-pyran-4-ylamino)-1H-pyrazolo[3,4-b]pyridine-5-carboxamide;
1-ethyl-N-[(1S)-1-(1-naphthalenyl)ethyl]-4-(tetrahydro-2H-pyran-4-ylamino)-1H-pyrazolo[3,4-b]pyridine-5-carboxamide;
N-[1-(aminocarbonyl)-1-phenylpropyl]-1-ethyl-4-(tetrahydro-2H-pyran-4-ylamino)-1H-pyrazolo[3,4-b]pyridine-5-carboxamide;
1-ethyl-N-(1-phenylcyclopentyl)-4-(tetrahydro-2H-pyran-4-ylamino)-1H-pyrazolo[3,4-b]pyridine-5-carboxamide;
1-ethyl-N-(4-phenyltetrahydro-2H-pyran-4-yl)-4-(tetrahydro-2H-pyran-4-ylamino)-1H-pyrazolo[3,4-b]pyridine-5-carboxamide;
1-ethyl-N-(1-phenylcyclopropyl)-4-(tetrahydro-2H-pyran-4-ylamino)-1H-pyrazolo[3,4-b]pyridine-5-carboxamide;
N-{1-[4-(cyclohexyloxy)-3-methylphenyl]ethyl}-1-ethyl-4-(tetrahydro-2H-pyran-4-ylamino)-1H-pyrazolo[3,4-b]pyridine-5-carboxamide;
N-{1-[3-(cyclohexyloxy)-4-(methyloxy)phenyl]ethyl}-1-ethyl-4-(tetrahydro-2H-pyran-4-ylamino)-1H-pyrazolo[3,4-b]pyridine-5-carboxamide;
N-[1-(2,3-dichlorophenyl)ethyl]-1-ethyl-4-(tetrahydro-2H-pyran-4-ylamino)-1H-pyrazolo[3,4-b]pyridine-5-carboxamide;
N-{1-[4-(cyclohexyloxy)-3-hydroxyphenyl]ethyl}-1-ethyl-4-(tetrahydro-2H-pyran-4-ylamino)-1H-pyrazolo[3,4-b]pyridine-5-carboxamide;
N-{1-[4-(cyclopentyloxy)phenyl]ethyl}-1-ethyl-4-(tetrahydro-2H-pyran-4-ylamino)-1H-pyrazolo[3,4-b]pyridine-5-carboxamide;
1-ethyl-N-[1-(4-methylphenyl)ethyl]-4-(tetrahydro-2H-pyran-4-ylamino)-1H-pyrazolo[3,4-b]pyridine-5-carboxamide;
N-{1-[4-(1,1-dimethylethyl)phenyl]cycloheptyl}-1-ethyl-4-(tetrahydro-2H-pyran-4-ylamino)-1H-pyrazolo[3,4-b]pyridine-5-carboxamide;
N-[1-(4-bromophenyl)ethyl]-1-ethyl-4-(tetrahydro-2H-pyran-4-ylamino)-1H-pyrazolo[3,4-b]pyridine-5-carboxamide;
1-ethyl-N-[(1S)-1-(4-iodophenyl)ethyl]-4-(tetrahydro-2H-pyran-4-ylamino)-1H-pyrazolo[3,4-b]pyridine-5-carboxamide;
N-{1-[4-(aminosulfonyl)phenyl]ethyl}-1-ethyl-4-(tetrahydro-2H-pyran-4-ylamino)-1H-pyrazolo[3,4-b]pyridine-5-carboxamide;
1-ethyl-N-(1-methyl-1-phenylpropyl)-4-(tetrahydro-2H-pyran-4-ylamino)-1H-pyrazolo[3,4-b]pyridine-5-carboxamide;
N-[1-(1,3-benzodioxol-5-yl)cyclohexyl]-1-ethyl-4-(tetrahydro-2H-pyran-4-ylamino)-1H-pyrazolo[3,4-b]pyridine-5-carboxamide;
1-ethyl-N-{1-[4-(methyloxy)phenyl]cyclohexyl}-4-(tetrahydro-2H-pyran-4-ylamino)-1H-pyrazolo[3,4-b]pyridine-5-carboxamide;
1-ethyl-N-[1-(4-fluorophenyl)cyclohexyl]-4-(tetrahydro-2H-pyran-4-ylamino)-1H-pyrazolo[3,4-b]pyridine-5-carboxamide;
N-[1-(3-chlorophenyl)cyclopentyl]-1-ethyl-4-(tetrahydro-2H-pyran-4-ylamino)-1H-pyrazolo[3,4-b]pyridine-5-carboxamide;
N-[1-(2-chlorophenyl)cyclopentyl]-1-ethyl-4-(tetrahydro-2H-pyran-4-ylamino)-1H-pyrazolo[3,4-b]pyridine-5-carboxamide;
N-{1-[4-(1,1-dimethylethyl)phenyl]cyclohexyl}-1-ethyl-4-(tetrahydro-2H-pyran-4-ylamino)-1H-pyrazolo[3,4-b]pyridine-5-carboxamide;
1-ethyl-N-{1-[4-(1-methylethyl)phenyl]ethyl}-4-(tetrahydro-2H-pyran-4-ylamino)-1H-pyrazolo[3,4-b]pyridine-5-carboxamide;
N-[1-(3,5-dimethylphenyl)ethyl]-1-ethyl-4-(tetrahydro-2H-pyran-4-ylamino)-1H-pyrazolo[3,4-b]pyridine-5-carboxamide;
1-ethyl-N-[(1S,2R)-2-hydroxy-1-phenylpropyl]-4-(tetrahydro-2H-pyran-4-ylamino)-1H-pyrazolo[3,4-b]pyridine-5-carboxamide;
1-ethyl-N-{(1R)-1-[4-(methyloxy)phenyl]ethyl}-4-(tetrahydro-2H-pyran-4-ylamino)-1H-pyrazolo[3,4-b]pyridine-5-carboxamide;
1-ethyl-N-{(1S)-1-[4-(methyloxy)phenyl]ethyl}-4-(tetrahydro-2H-pyran-4-ylamino)-1H-pyrazolo[3,4-b]pyridine-5-carboxamide;
1-ethyl-N-(1-phenylhexyl)-4-(tetrahydro-2H-pyran-4-ylamino)-1H-pyrazolo[3,4-b]pyridine-5-carboxamide;
1-ethyl-N-(1-phenylpentyl)-4-(tetrahydro-2H-pyran-4-ylamino)-1H-pyrazolo[3,4-b]pyridine-5-carboxamide;
1-ethyl-N-(2-methyl-1-phenylpropyl)-4-(tetrahydro-2H-pyran-4-ylamino)-1H-pyrazolo[3,4-b]pyridine-5-carboxamide;
1-ethyl-N-(1-phenylbutyl)-4-(tetrahydro-2H-pyran-4-ylamino)-1H-pyrazolo[3,4-b]pyridine-5-carboxamide;
1-ethyl-4-(tetrahydro-2H-pyran-4-ylamino)-N-(2,2,2-trifluoro-1-phenylethyl)-1H-pyrazolo[3,4-b]pyridine-5-carboxamide;
N-[cyclopropyl(phenyl)methyl]-1-ethyl-4-(tetrahydro-2H-pyran-4-ylamino)-1H-pyrazolo[3,4-b]pyridine-5-carboxamide;
1-ethyl-N-[1-(4-fluorophenyl)propyl]-4-(tetrahydro-2H-pyran-4-ylamino)-1H-pyrazolo[3,4-b]pyridine-5-carboxamide;
N-[1-(2,3-dichlorophenyl)propyl]-1-ethyl-4-(tetrahydro-2H-pyran-4-ylamino)-1H-pyrazolo[3,4-b]pyridine-5-carboxamide;
1-ethyl-N-(1-phenylethyl)-4-(tetrahydro-2H-pyran-4-ylamino)-1H-pyrazolo[3,4-b]pyridine-5-carboxamide;
N-[1-(4-chlorophenyl)-2-hydroxyethyl]-1-ethyl-4-(tetrahydro-2H-pyran-4-ylamino)-1H-pyrazolo[3,4-b]pyridine-5-carboxamide;
N-[1-(3,4-dichlorophenyl)-2-hydroxyethyl]-1-ethyl-4-(tetrahydro-2H-pyran-4-ylamino)-1H-pyrazolo[3,4-b]pyridine-5-carboxamide;
1-ethyl-N-{1-[3-(methyloxy)phenyl]propyl}-4-(tetrahydro-2H-pyran-4-ylamino)-1H-pyrazolo[3,4-b]pyridine-5-carboxamide;
1-ethyl-N-{1-[4-(methyloxy)phenyl]propyl}-4-(tetrahydro-2H-pyran-4-ylamino)-1H-pyrazolo[3,4-b]pyridine-5-carboxamide;
N-[1-(4-bromophenyl)propyl]-1-ethyl-4-(tetrahydro-2H-pyran-4-ylamino)-1H-pyrazolo[3,4-b]pyridine-5-carboxamide;
1-ethyl-N-{1-[4-(propyloxy)phenyl]propyl}-4-(tetrahydro-2H-pyran-4-ylamino)-1H-pyrazolo[3,4-b]pyridine-5-carboxamide;
N-[1-(3,5-dimethylphenyl)propyl]-1-ethyl-4-(tetrahydro-2H-pyran-4-ylamino)-1H-pyrazolo[3,4-b]pyridine-5-carboxamide;
1-ethyl-N-[1-(4-methylphenyl)propyl]-4-(tetrahydro-2H-pyran-4-ylamino)-1H-pyrazolo[3,4-b]pyridine-5-carboxamide;
1-ethyl-N-{1-[4-(1-methylethyl)phenyl]propyl}-4-(tetrahydro-2H-pyran-4-ylamino)-1H-pyrazolo[3,4-b]pyridine-5-carboxamide;
1-ethyl-N-[1-(2-methylphenyl)ethyl]-4-(tetrahydro-2H-pyran-4-ylamino)-1H-pyrazolo[3,4-b]pyridine-5-carboxamide;
N-(1-{4-[(difluoromethyl)oxy]phenyl}ethyl)-1-ethyl-4-(tetrahydro-2H-pyran-4-ylamino)-1H-pyrazolo[3,4-b]pyridine-5-carboxamide;
1-ethyl-4-(tetrahydro-2H-pyran-4-ylamino)-N-{1-[4-(trifluoromethyl)phenyl]ethyl}-1H-pyrazolo[3,4-b]pyridine-5-carboxamide;
1-ethyl-N-[1-(2-methylphenyl)propyl]-4-(tetrahydro-2H-pyran-4-ylamino)-1H-pyrazolo[3,4-b]pyridine-5-carboxamide;
1-ethyl-N-{1-[4-(ethyloxy)phenyl]propyl}-4-(tetrahydro-2H-pyran-4-ylamino)-1H-pyrazolo[3,4-b]pyridine-5-carboxamide;
N-(1-{4-[(difluoromethyl)oxy]phenyl}propyl)-1-ethyl-4-(tetrahydro-2H-pyran-4-ylamino)-1H-pyrazolo[3,4-b]pyridine-5-carboxamide;
1-ethyl-4-(tetrahydro-2H-pyran-4-ylamino)-N-{1-[4-(trifluoromethyl)phenyl]propyl}-1H-pyrazolo[3,4-b]pyridine-5-carboxamide;
N-[1-(3,4-dimethylphenyl)propyl]-1-ethyl-4-(tetrahydro-2H-pyran-4-ylamino)-1H-pyrazolo[3,4-b]pyridine-5-carboxamide;
N-[1-(2,3-dimethylphenyl)ethyl]-1-ethyl-4-(tetrahydro-2H-pyran-4-ylamino)-1H-pyrazolo[3,4-b]pyridine-5-carboxamide;
N-[1-(2,4-dimethylphenyl)ethyl]-1-ethyl-4-(tetrahydro-2H-pyran-4-ylamino)-1H-pyrazolo[3,4-b]pyridine-5-carboxamide;
N-[1-(4-chloro-2-fluorophenyl)ethyl]-1-ethyl-4-(tetrahydro-2H-pyran-4-ylamino)-1H-pyrazolo[3,4-b]pyridine-5-carboxamide;
N-[1-(3-chloro-4-methylphenyl)ethyl]-1-ethyl-4-(tetrahydro-2H-pyran-4-ylamino)-1H-pyrazolo[3,4-b]pyridine-5-carboxamide;
N-[1-(2,3-dimethylphenyl)propyl]-1-ethyl-4-(tetrahydro-2H-pyran-4-ylamino)-1H-pyrazolo[3,4-b]pyridine-5-carboxamide;
N-[1-(2,4-dimethylphenyl)propyl]-1-ethyl-4-(tetrahydro-2H-pyran-4-ylamino)-1H-pyrazolo[3,4-b]pyridine-5-carboxamide;
N-[1-(4-chloro-2-fluorophenyl)propyl]-1-ethyl-4-(tetrahydro-2H-pyran-4-ylamino)-1H-pyrazolo[3,4-b]pyridine-5-carboxamide;
N-[1-(3-chloro-4-methylphenyl)propyl]-1-ethyl-4-(tetrahydro-2H-pyran-4-ylamino)-1H-pyrazolo[3,4-b]pyridine-5-carboxamide;
1-ethyl-N-[1-(3-hydroxyphenyl)propyl]-4-(tetrahydro-2H-pyran-4-ylamino)-1H-pyrazolo[3,4-b]pyridine-5-carboxamide;
N-[1-(2,3-dihydro-1H-inden-5-yl)ethyl]-1-ethyl-4-(tetrahydro-2H-pyran-4-ylamino)-1H-pyrazolo[3,4-b]pyridine-5-carboxamide;
1-ethyl-N-[1-(5,6,7,8-tetrahydro-2-naphthalenyl)ethyl]-4-(tetrahydro-2H-pyran-4-ylamino)-1H-pyrazolo[3,4-b]pyridine-5-carboxamide;
N-[1-(4-bromophenyl)-2,2,2-trifluoroethyl]-1-ethyl-4-(tetrahydro-2H-pyran-4-ylamino)-1H-pyrazolo[3,4-b]pyridine-5-carboxamide;
1-ethyl-4-(tetrahydro-2H-pyran-4-ylamino)-N-{2,2,2-trifluoro-1-[3-(methyloxy)phenyl]ethyl}-1H-pyrazolo[3,4-b]pyridine-5-carboxamide;
4-(cyclohexylamino)-1-ethyl-N-{1-[4-(methylsulfonyl)phenyl]ethyl}-1H-pyrazolo[3,4-b]pyridine-5-carboxamide;
4-(cyclohexylamino)-1-ethyl-N-[(1R)-1-phenylpropyl]-1H-pyrazolo[3,4-b]pyridine-5-carboxamide;
4-(cyclohexylamino)-N-(diphenylmethyl)-1-ethyl-1H-pyrazolo[3,4-b]pyridine-5-carboxamide;
4-(cyclohexylamino)-1-ethyl-N-[(1R)-1-phenylethyl]-1H-pyrazolo[3,4-b]pyridine-5-carboxamide;
ethyl({[4-(cyclohexylamino)-1-ethyl-1H-pyrazolo[3,4-b]pyridin-5-yl]carbonyl}amino)(phenyl)acetate;
N-[1-(4-chlorophenyl) ethyl]-4-(cyclohexylamino)-1-ethyl-1H-pyrazolo[3,4-b]pyridine-5-carboxamide;
4-(cyclohexylamino)-1-ethyl-N-(1-methyl-1-phenylethyl)-1H-pyrazolo[3,4-b]pyridine-5-carboxamide;
4-(cyclohexylamino)-1-ethyl-N-[1-(4-fluorophenyl)ethyl]-1H-pyrazolo[3,4-b]pyridine-5-carboxamide;
N-[1-(4-chlorophenyl)propyl]-4-(cyclohexylamino)-1-ethyl-1H-pyrazolo[3,4-b]pyridine-5-carboxamide;
4-(cyclohexylamino)-N-(1,2-diphenylethyl)-1-ethyl-1H-pyrazolo[3,4-b]pyridine-5-carboxamide;
4-(cyclohexylamino)-1-ethyl-N-{1-[4-(propyloxy)phenyl]ethyl}-1H-pyrazolo[3,4-b]pyridine-5-carboxamide;
methyl 3-({[4-(cyclohexylamino)-1-ethyl-1H-pyrazolo[3,4-b]pyridin-5-yl]carbonyl}amino)-3-phenylpropanoate;
4-(cyclohexylamino)-1-ethyl-N-[1-(hydroxymethyl)-1-phenylpropyl]-1H-pyrazolo[3,4-b]pyridine-5-carboxamide;
4-(cyclohexylamino)-1-ethyl-N-(3-hydroxy-1-phenylpropyl)-1H-pyrazolo[3,4-b]pyridine-5-carboxamide;
4-(cyclohexylamino)-1-ethyl-N-{1-[4-(ethyloxy)phenyl]ethyl}-1H-pyrazolo[3,4-b]pyridine-5-carboxamide;
4-(cyclohexylamino)-1-ethyl-N-[1-(3-hydroxyphenyl)ethyl]-1H-pyrazolo[3,4-b]pyridine-5-carboxamide;
4-(cyclohexylamino)-1-ethyl-N-[1-phenyl-2-(1-pyrrolidinyl)ethyl]-1H-pyrazolo[3,4-b]pyridine-5-carboxamide;
4-(cyclohexylamino)-N-[2-(dimethylamino)-1-phenylethyl]-1-ethyl-1H-pyrazolo[3,4-b]pyridine-5-carboxamide;
4-(cyclohexylamino)-1-ethyl-N-[(1R)-2-(methyloxy)-1-phenylethyl]-1H-pyrazolo[3,4-b]pyridine-5-carboxamide;
N-[(1R)-2-amino-2-oxo-1-phenylethyl]-4-(cyclohexylamino)-1-ethyl-1H-pyrazolo[3,4-b]pyridine-5-carboxamide;
4-(cyclohexylamino)-1-ethyl-N-[(1R)-2-hydroxy-1-phenylethyl]-1H-pyrazolo[3,4-b]pyridine-5-carboxamide;
4-(cyclohexylamino)-1-ethyl-N-[(1S)-2-hydroxy-1-phenylethyl]-1H-pyrazolo[3,4-b]pyridine-5-carboxamide;
4-(cyclohexylamino)-1-ethyl-N-{(1R)-1-[3-(methyloxy)phenyl]ethyl}-1H-pyrazolo[3,4-b]pyridine-5-carboxamide;
4-(cyclohexylamino)-1-ethyl-N-[(1S)-2-(methyloxy)-1-phenylethyl]-1H-pyrazolo[3,4-b]pyridine-5-carboxamide;
4-(cyclohexylamino)-1-ethyl-N-[(1R)-1-(4-nitrophenyl)ethyl]-1H-pyrazolo[3,4-b]pyridine-5-carboxamide;
4-(cyclohexylamino)-1-ethyl-N-[(1S)-1-(1-naphthalenyl)ethyl]-1H-pyrazolo[3,4-b]pyridine-5-carboxamide;
4-(cyclohexylamino)-1-ethyl-N-[phenyl(4-phenyl-1,3-thiazol-2-yl)methyl]-1H-pyrazolo[3,4-b]pyridine-5-carboxamide;
N-[cyano(phenyl)methyl]-4-(cyclohexylamino)-1-ethyl-1H-pyrazolo[3,4-b]pyridine-5-carboxamide;
4-(cyclohexylamino)-1-ethyl-N-[1-(1-naphthalenyl)ethyl]-1H-pyrazolo[3,4-b]pyridine-5-carboxamide;
4-(cyclohexylamino)-1-ethyl-N-(2-hydroxy-1,1-diphenylethyl)-1H-pyrazolo[3,4-b]pyridine-5-carboxamide;
4-(cyclohexylamino)-1-ethyl-N-{(1R)-1-[4-(methyloxy)phenyl]ethyl}-1H-pyrazolo[3,4-b]pyridine-5-carboxamide;
4-(cyclohexylamino)-1-ethyl-N-[1-(4-fluorophenyl)propyl]-1H-pyrazolo[3,4-b]pyridine-5-carboxamide;
4-(cyclohexylamino)-N-[1-(2,3-dichlorophenyl)propyl]-1-ethyl-1H-pyrazolo[3,4-b]pyridine-5-carboxamide;
4-(cyclohexylamino)-1-ethyl-N-[(1R)-1-(4-methylphenyl)ethyl]-1H-pyrazolo[3,4-b]pyridine-5-carboxamide;
4-(cyclohexylamino)-1-ethyl-N-(1-phenylethyl)-1H-pyrazolo[3,4-b]pyridine-5-carboxamide;
N-[(1R)-1-(4-bromophenyl)ethyl]-4-(cyclohexylamino)-1-ethyl-1H-pyrazolo[3,4-b]pyridine-5-carboxamide;
4-(cyclohexylamino)-N-[1-(2,3-dichlorophenyl)ethyl]-1-ethyl-1H-pyrazolo[3,4-b]pyridine-5-carboxamide;
4-(cyclohexylamino)-1-ethyl-N-{1-[3-(methyloxy)phenyl]propyl}-1H-pyrazolo[3,4-b]pyridine-5-carboxamide;
4-(cyclohexylamino)-1-ethyl-N-{1-[4-(methyloxy)phenyl]propyl}-1H-pyrazolo[3,4-b]pyridine-5-carboxamide;
N-[1-(4-bromophenyl)propyl]-4-(cyclohexylamino)-1-ethyl-1H-pyrazolo[3,4-b]pyridine-5-carboxamide;
4-(cyclohexylamino)-1-ethyl-N-{1-[4-(propyloxy)phenyl]propyl}-1H-pyrazolo[3,4-b]pyridine-5-carboxamide;
4-(cyclohexylamino)-N-[1-(3,5-dimethylphenyl)propyl]-1-ethyl-1H-pyrazolo[3,4-b]pyridine-5-carboxamide;
4-(cyclohexylamino)-1-ethyl-N-[1-(4-methylphenyl)propyl]-1H-pyrazolo[3,4-b]pyridine-5-carboxamide;
4-(cyclohexylamino)-1-ethyl-N-{1-[4-(1-methylethyl)phenyl]propyl}-1H-pyrazolo[3,4-b]pyridine-5-carboxamide;
4-(cyclohexylamino)-1-ethyl-N-[1-(2-methylphenyl)ethyl]-1H-pyrazolo[3,4-b]pyridine-5-carboxamide;
4-(cyclohexylamino)-N-(1-{4-[(difluoromethyl)oxy]phenyl}ethyl)-1-ethyl-1H-pyrazolo[3,4-b]pyridine-5-carboxamide;
4-(cyclohexylamino)-1-ethyl-N-{1-[4-(trifluoromethyl)phenyl]ethyl}-1H-pyrazolo[3,4-b]pyridine-5-carboxamide;
4-(cyclohexylamino)-1-ethyl-N-[1-(2-methylphenyl)propyl]-1H-pyrazolo[3,4-b]pyridine-5-carboxamide;
4-(cyclohexylamino)-1-ethyl-N-{1-[4-(ethyloxy)phenyl]propyl}-1H-pyrazolo[3,4-b]pyridine-5-carboxamide;
4-(cyclohexylamino)-N-(1-{4-[(difluoromethyl)oxy]phenyl}propyl)-1-ethyl-1H-pyrazolo[3,4-b]pyridine-5-carboxamide;
4-(cyclohexylamino)-1-ethyl-N-{1-[4-(trifluoromethyl)phenyl]propyl}-1H-pyrazolo[3,4-b]pyridine-5-carboxamide;
4-(cyclohexylamino)-N-[1-(3,4-dimethylphenyl)propyl]-1-ethyl-1H-pyrazolo[3,4-b]pyridine-5-carboxamide;
4-(cyclohexylamino)-N-[1-(2,3-dimethylphenyl)ethyl]-1-ethyl-1H-pyrazolo[3,4-b]pyridine-5-carboxamide;
4-(cyclohexylamino)-N-[1-(2,4-dimethylphenyl)ethyl]-1-ethyl-1H-pyrazolo[3,4-b]pyridine-5-carboxamide;
N-[1-(4-chloro-2-fluorophenyl)ethyl]-4-(cyclohexylamino)-1-ethyl-1H-pyrazolo[3,4-b]pyridine-5-carboxamide;
N-[1-(3-chloro-4-methylphenyl)ethyl]-4-(cyclohexylamino)-1-ethyl-1H-pyrazolo[3,4-b]pyridine-5-carboxamide;
4-(cyclohexylamino)-N-[1-(2,3-dimethylphenyl)propyl]-1-ethyl-1H-pyrazolo[3,4-b]pyridine-5-carboxamide;
4-(cyclohexylamino)-N-[1-(2,4-dimethylphenyl)propyl]-1-ethyl-1H-pyrazolo[3,4-b]pyridine-5-carboxamide;
N-[1-(4-chloro-2-fluorophenyl)propyl]-4-(cyclohexylamino)-1-ethyl-1H-pyrazolo[3,4-b]pyridine-5-carboxamide;
N-[1-(3-chloro-4-methylphenyl)propyl]-4-(cyclohexylamino)-1-ethyl-1H-pyrazolo[3,4-b]pyridine-5-carboxamide;
4-(cyclohexylamino)-1-ethyl-N-[1-(3-hydroxyphenyl)propyl]-1H-pyrazolo[3,4-b]pyridine-5-carboxamide;
N-[1-(4-chlorophenyl)-2-hydroxyethyl]-4-(cyclohexylamino)-1-ethyl-1H-pyrazolo[3,4-b]pyridine-5-carboxamide;
4-(cyclohexylamino)-N-[1-(2,3-dihydro-1H-inden-5-yl)ethyl]-1-ethyl-1H-pyrazolo[3,4-b]pyridine-5-carboxamide;
4-(cyclohexylamino)-1-ethyl-N-[1-(5,6,7,8-tetrahydro-2-naphthalenyl)ethyl]-1H-pyrazolo[3,4-b]pyridine-5-carboxamide;
4-[(1-acetyl-4-piperidinyl)amino]-1-ethyl-N-[(1S)-1-phenylpropyl]-1H-pyrazolo[3,4-b]pyridine-5-carboxamide;
4-[(1-acetyl-4-piperidinyl)amino]-1-ethyl-N-[(1R)-1-phenylethyl]-1H-pyrazolo[3,4-b]pyridine-5-carboxamide;
4-[(1-acetyl-4-piperidinyl)amino]-N-(diphenylmethyl)-1-ethyl-1H-pyrazolo[3,4-b]pyridine-5-carboxamide;
4-[(1-acetyl-4-piperidinyl)amino]-1-ethyl-N-{1-[4-(methylsulfonyl)phenyl]ethyl}-1H-pyrazolo[3,4-b]pyridine-5-carboxamide;
4-[(1-acetyl-4-piperidinyl)amino]-1-ethyl-N-[(1R)-1-phenylpropyl]-1H-pyrazolo[3,4-b]pyridine-5-carboxamide;
N-[1-(4-chlorophenyl)ethyl]-1-ethyl-4-[(4-oxocyclohexyl)amino]-1H-pyrazolo[3,4-b]pyridine-5-carboxamide;
N-[1-(4-chlorophenyl)propyl]-1-ethyl-4-[(4-oxocyclohexyl)amino]-1H-pyrazolo[3,4-b]pyridine-5-carboxamide;
1-ethyl-N-[(1S)-1-(4-nitrophenyl)ethyl]-4-[(4-oxocyclohexyl)amino]-1H-pyrazolo[3,4-b]pyridine-5-carboxamide;
1-ethyl-N-[(1R)-1-(4-nitrophenyl)ethyl]-4-[(4-oxocyclohexyl)amino]-1H-pyrazolo[3,4-b]pyridine-5-carboxamide;
1-ethyl-N-{1-[4-(ethyloxy)phenyl]ethyl}-4-[(4-oxocyclohexyl)amino]-1H-pyrazolo[3,4-b]pyridine-5-carboxamide;
1-ethyl-4-[(4-oxocyclohexyl)amino]-N-{1-[4-(propyloxy)phenyl]ethyl}-1H-pyrazolo[3,4-b]pyridine-5-carboxamide;
1-ethyl-N-[1-(4-fluorophenyl)ethyl]-4-[(4-oxocyclohexyl)amino]-1H-pyrazolo[3,4-b]pyridine-5-carboxamide;
1-ethyl-N-[(1R)-2-hydroxy-1-phenylethyl]-4-[(4-oxocyclohexyl)amino]-1H-pyrazolo[3,4-b]pyridine-5-carboxamide;
1-ethyl-4-[(4-oxocyclohexyl)amino]-N-(1-phenylpropyl)-1H-pyrazolo[3,4-b]pyridine-5-carboxamide;
(2R)-[({1-ethyl-4-[(4-oxocyclohexyl)amino]-1H-pyrazolo[3,4-b]pyridin-5-yl}carbonyl)amino][3-(methyloxy)phenyl]ethanoic acid;
1-ethyl-N-{1-[4-(1-methylethyl)phenyl]ethyl}-4-[(4-oxocyclohexyl)amino]-1H-pyrazolo[3,4-b]pyridine-5-carboxamide;
1-ethyl-N-[1-(2-methylphenyl)ethyl]-4-[(4-oxocyclohexyl)amino]-1H-pyrazolo[3,4-b]pyridine-5-carboxamide;
N-[1-(3,5-dimethylphenyl)ethyl]-1-ethyl-4-[(4-oxocyclohexyl)amino]-1H-pyrazolo[3,4-b]pyridine-5-carboxamide;
1-ethyl-N-{(1R)-1-[4-(methyloxy)phenyl]ethyl}-4-[(4-oxocyclohexyl)amino]-1H-pyrazolo[3,4-b]pyridine-5-carboxamide;
1-ethyl-N-[1-(4-fluorophenyl)propyl]-4-[(4-oxocyclohexyl)amino]-1H-pyrazolo[3,4-b]pyridine-5-carboxamide;
N-[1-(2,3-dichlorophenyl)propyl]-1-ethyl-4-[(4-oxocyclohexyl)amino]-1H-pyrazolo[3,4-b]pyridine-5-carboxamide;
1-ethyl-N-[(1R)-1-(4-methylphenyl)ethyl]-4-[(4-oxocyclohexyl)amino]-1H-pyrazolo[3,4-b]pyridine-5-carboxamide;
1-ethyl-4-[(4-oxocyclohexyl)amino]-N-(1-phenylethyl)-1H-pyrazolo[3,4-b]pyridine-5-carboxamide;
N-[(1R)-1-(4-bromophenyl)ethyl]-1-ethyl-4-[(4-oxocyclohexyl)amino]-1H-pyrazolo[3,4-b]pyridine-5-carboxamide;
1-ethyl-N-[(1S)-2-hydroxy-1-phenylethyl]-4-[(4-oxocyclohexyl)amino]-1H-pyrazolo[3,4-b]pyridine-5-carboxamide;
N-[1-(4-chlorophenyl)-2-hydroxyethyl]-1-ethyl-4-[(4-oxocyclohexyl)amino]-1H-pyrazolo[3,4-b]pyridine-5-carboxamide;
N-(1-{4-[(difluoromethyl)oxy]phenyl}ethyl)-1-ethyl-4-[(4-oxocyclohexyl)amino]-1H-pyrazolo[3,4-b]pyridine-5-carboxamide;
1-ethyl-4-[(4-oxocyclohexyl)amino]-N-{1-[4-(trifluoromethyl)phenyl]ethyl}-1H-pyrazolo[3,4-b]pyridine-5-carboxamide;
1-ethyl-N-[1-(2-methylphenyl)propyl]-4-[(4-oxocyclohexyl)amino]-1H-pyrazolo[3,4-b]pyridine-5-carboxamide;
1-ethyl-N-{1-[4-(ethyloxy)phenyl]propyl}-4-[(4-oxocyclohexyl)amino]-1H-pyrazolo[3,4-b]pyridine-5-carboxamide;
N-(1-{4-[(difluoromethyl)oxy]phenyl}propyl)-1-ethyl-4-[(4-oxocyclohexyl)amino]-1H-pyrazolo[3,4-b]pyridine-5-carboxamide;
1-ethyl-4-[(4-oxocyclohexyl)amino]-N-{1-[4-(trifluoromethyl)phenyl]propyl}-1H-pyrazolo[3,4-b]pyridine-5-carboxamide;
N-[1-(3,4-dimethylphenyl)propyl]-1-ethyl-4-[(4-oxocyclohexyl)amino]-1H-pyrazolo[3,4-b]pyridine-5-carboxamide;
1-ethyl-4-[(4-oxocyclohexyl)amino]-N-[(1R)-1-phenylpropyl]-1H-pyrazolo[3,4-b]pyridine-5-carboxamide;
1-ethyl-N-{(1R)-1-[3-(methyloxy)phenyl]ethyl}-4-[(4-oxocyclohexyl)amino]-1H-pyrazolo[3,4-b]pyridine-5-carboxamide;
N-[1-(2,3-dimethylphenyl)ethyl]-1-ethyl-4-[(4-oxocyclohexyl)amino]-1H-pyrazolo[3,4-b]pyridine-5-carboxamide;
N-[1-(2,4-dimethylphenyl)ethyl]-1-ethyl-4-[(4-oxocyclohexyl)amino]-1H-pyrazolo[3,4-b]pyridine-5-carboxamide;
N-[1-(4-chloro-2-fluorophenyl)ethyl]-1-ethyl-4-[(4-oxocyclohexyl)amino]-1H-pyrazolo[3,4-b]pyridine-5-carboxamide;
N-[1-(3-chloro-4-methylphenyl)ethyl]-1-ethyl-4-[(4-oxocyclohexyl)amino]-1H-pyrazolo[3,4-b]pyridine-5-carboxamide;
N-[1-(2,3-dimethylphenyl)propyl]-1-ethyl-4-[(4-oxocyclohexyl)amino]-1H-pyrazolo[3,4-b]pyridine-5-carboxamide;
N-[1-(2,4-dimethylphenyl)propyl]-1-ethyl-4-[(4-oxocyclohexyl)amino]-1H-pyrazolo[3,4-b]pyridine-5-carboxamide;
N-[1-(4-chloro-2-fluorophenyl)propyl]-1-ethyl-4-[(4-oxocyclohexyl)amino]-1H-pyrazolo[3,4-b]pyridine-5-carboxamide;
N-[1-(3-chloro-4-methylphenyl)propyl]-1-ethyl-4-[(4-oxocyclohexyl)amino]-1H-pyrazolo[3,4-b]pyridine-5-carboxamide;
1-ethyl-N-[1-(3-hydroxyphenyl)ethyl]-4-[(4-oxocyclohexyl)amino]-1H-pyrazolo[3,4-b]pyridine-5-carboxamide;
1-ethyl-N-[1-(3-hydroxyphenyl)propyl]-4-[(4-oxocyclohexyl)amino]-1H-pyrazolo[3,4-b]pyridine-5-carboxamide;
N-[1-(2,3-dichlorophenyl)ethyl]-1-ethyl-4-[(4-oxocyclohexyl)amino]-1H-pyrazolo[3,4-b]pyridine-5-carboxamide;
1-ethyl-N-{1-[3-(methyloxy)phenyl]propyl}-4-[(4-oxocyclohexyl)amino]-1H-pyrazolo[3,4-b]pyridine-5-carboxamide;
1-ethyl-N-{1-[4-(methyloxy)phenyl]propyl}-4-[(4-oxocyclohexyl)amino]-1H-pyrazolo[3,4-b]pyridine-5-carboxamide;
N-[1-(4-bromophenyl)propyl]-1-ethyl-4-[(4-oxocyclohexyl)amino]-1H-pyrazolo[3,4-b]pyridine-5-carboxamide;
1-ethyl-4-[(4-oxocyclohexyl)amino]-N-{1-[4-(propyloxy)phenyl]propyl}-1H-pyrazolo[3,4-b]pyridine-5-carboxamide;
N-[1-(3,5-dimethylphenyl)propyl]-1-ethyl-4-[(4-oxocyclohexyl)amino]-1H-pyrazolo[3,4-b]pyridine-5-carboxamide;
1-ethyl-N-[1-(4-methylphenyl)propyl]-4-[(4-oxocyclohexyl)amino]-1H-pyrazolo[3,4-b]pyridine-5-carboxamide;
1-ethyl-N-{1-[4-(1-methylethyl)phenyl]propyl}-4-[(4-oxocyclohexyl)amino]-1H-pyrazolo[3,4-b]pyridine-5-carboxamide;
1-ethyl-N-(1-{4-[(1-methylethyl)oxy]phenyl}ethyl)-4-[(4-oxocyclohexyl)amino]-1H-pyrazolo[3,4-b]pyridine-5-carboxamide;
1-ethyl-4-[(4-oxocyclohexyl)amino]-N-[1-(5,6,7,8-tetrahydro-2-naphthalenyl)ethyl]-1H-pyrazolo[3,4-b]pyridine-5-carboxamide;
N-[1-(4-bromophenyl)-2,2,2-trifluoroethyl]-1-ethyl-4-[(4-oxocyclohexyl)amino]-1H-pyrazolo[3,4-b]pyridine-5-carboxamide;
1-ethyl-4-[(4-oxocyclohexyl)amino]-N-{2,2,2-trifluoro-1-[3-(methyloxy)phenyl]ethyl}-1H-pyrazolo[3,4-b]pyridine-5-carboxamide;
1-ethyl-4-{[4-(hydroxyimino)cyclohexyl]amino}-N-[1-(5,6,7,8-tetrahydro-2-naphthalenyl)ethyl]-1H-pyrazolo[3,4-b]pyridine-5-carboxamide;
1-ethyl-4-{[4-(hydroxyimino)cyclohexyl]amino}-N-[(1S)-2-hydroxy-1-phenylethyl]-1H-pyrazolo[3,4-b]pyridine-5-carboxamide;
N-[1-(2,3-dihydro-1H-inden-5-yl)ethyl]-1-ethyl-4-{[4-(hydroxyimino)cyclohexyl]amino }-1H-pyrazolo[3,4-b]pyridine-5-carboxamide;
N-[1-(4-chlorophenyl)-2-hydroxyethyl]-1-ethyl-4-{[4-(hydroxyimino)cyclohexyl]amino}-1H-pyrazolo[3,4-b]pyridine-5-carboxamide;
1-ethyl-N-{1-[4-(ethyloxy)phenyl]ethyl}-4-{[4-(hydroxyimino)cyclohexyl]amino}-1H-pyrazolo[3,4-b]pyridine-5-carboxamide;
1-ethyl-4-{[4-(hydroxyimino)cyclohexyl]amino}-N-{1-[4-(propyloxy)phenyl]ethyl}-1H-pyrazolo[3,4-b]pyridine-5-carboxamide;
1-ethyl-N-[1-(4-fluorophenyl)ethyl]-4-{[4-(hydroxyimino)cyclohexyl]amino}-1H-pyrazolo[3,4-b]pyridine-5-carboxamide;
1-ethyl-4-{[4-(hydroxyimino)cyclohexyl]amino}-N-[(1R)-2-hydroxy-1-phenylethyl]-1H-pyrazolo[3,4-b]pyridine-5-carboxamide;
1-ethyl-4-{[4-(hydroxyimino)cyclohexyl]amino}-N-(1-phenylpropyl)-1H-pyrazolo[3,4-b]pyridine-5-carboxamide;
1-ethyl-4-{[4-(hydroxyimino)cyclohexyl]amino}-N-{1-[4-(1-methylethyl)phenyl]ethyl}-1H-pyrazolo[3,4-b]pyridine-5-carboxamide;
N-[1-(3,5-dimethylphenyl)ethyl]-1-ethyl-4-{[4-(hydroxyimino)cyclohexyl]amino}-1H-pyrazolo[3,4-b]pyridine-5-carboxamide;
1-ethyl-4-{[4-(hydroxyimino)cyclohexyl]amino}-N-{(1R)-1-[4-(methyloxy)phenyl]ethyl}-1H-pyrazolo[3,4-b]pyridine-5-carboxamide;
1-ethyl-N-[1-(4-fluorophenyl)propyl]-4-{[4-(hydroxyimino)cyclohexyl]amino}-1H-pyrazolo[3,4-b]pyridine-5-carboxamide;
N-[1-(2,3-dichlorophenyl)propyl]-1-ethyl-4-{[4-(hydroxyimino)cyclohexyl]amino}-1H-pyrazolo[3,4-b]pyridine-5-carboxamide;
1-ethyl-4-{[4-(hydroxyimino)cyclohexyl]amino}-N-[(1R)-1-(4-methylphenyl)ethyl]-1H-pyrazolo[3,4-b]pyridine-5-carboxamide;
1-ethyl-4-{[4-(hydroxyimino)cyclohexyl]amino}-N-(1-phenylethyl)-1H-pyrazolo[3,4-b]pyridine-5-carboxamide;
N-[(1R)-1-(4-bromophenyl)ethyl]-1-ethyl-4-{[4-(hydroxyimino)cyclohexyl]amino}-1H-pyrazolo[3,4-b]pyridine-5-carboxamide;
N-[1-(2,3-dichlorophenyl)ethyl]-1-ethyl-4-{[4-(hydroxyimino)cyclohexyl]amino}-1H-pyrazolo[3,4-b]pyridine-5-carboxamide;
N-[1-(4-chlorophenyl)propyl] -1-ethyl-4-{[4-(hydroxyimino)cyclohexyl]amino}-1H-pyrazolo[3,4-b]pyridine-5-carboxamide;
N-[1-(4-chlorophenyl)ethyl]-1-ethyl-4-{[4-(hydroxyimino)cyclohexyl]amino}-1H-pyrazolo[3,4-b]pyridine-5-carboxamide;
1-ethyl-4-{[4-(hydroxyimino)cyclohexyl]amino}-N-{1-[3-(methyloxy)phenyl]propyl}-1H-pyrazolo[3,4-b]pyridine-5-carboxamide;
1-ethyl-4-{[4-(hydroxyimino)cyclohexyl]amino}-N-{1-[4-(methyloxy)phenyl]propyl}-1H-pyrazolo[3,4-b]pyridine-5-carboxamide;
N-[1-(4-bromophenyl)propyl]-1-ethyl-4-{[4-(hydroxyimino)cyclohexyl]amino}-1H-pyrazolo[3,4-b]pyridine-5-carboxamide;
1-ethyl-4-{[4-(hydroxyimino)cyclohexyl]amino}-N-{1-[4-(propyloxy)phenyl]propyl}-1H-pyrazolo[3,4-b]pyridine-5-carboxamide;
N-[1-(3,5-dimethylphenyl)propyl]-1-ethyl-4-{[4-(hydroxyimino)cyclohexyl]amino}-1H-pyrazolo[3,4-b]pyridine-5-carboxamide;
1-ethyl-4-{[4-(hydroxyimino)cyclohexyl]amino}-N-[1-(4-methylphenyl)propyl]-1H-pyrazolo[3,4-b]pyridine-5-carboxamide;
1-ethyl-4-{[4-(hydroxyimino)cyclohexyl]amino}-N-{1-[4-(1-methylethyl)phenyl]propyl}-1H-pyrazolo[3,4-b]pyridine-5-carboxamide;
1-ethyl-4-{[4-(hydroxyimino)cyclohexyl]amino}-N-[1-(2-methylphenyl)ethyl]-1H-pyrazolo[3,4-b]pyridine-5-carboxamide;
N-(1-{4-[(difluoromethyl)oxy]phenyl}ethyl)-1-ethyl-4-{[4-(hydroxyimino)cyclohexyl]amino}-1H-pyrazolo[3,4-b]pyridine-5-carboxamide;
1-ethyl-4-{[4-(hydroxyimino)cyclohexyl]amino}-N-{1-[4-(trifluoromethyl)phenyl]ethyl}-1H-pyrazolo[3,4-b]pyridine-5-carboxamide;
1-ethyl-4-{[4-(hydroxyimino)cyclohexyl]amino}-N-[1-(2-methylphenyl)propyl]-1H-pyrazolo[3,4-b]pyridine-5-carboxamide;
1-ethyl-N-{1-[4-(ethyloxy)phenyl]propyl}-4-{[4-(hydroxyimino)cyclohexyl]amino}-1H-pyrazolo[3,4-b]pyridine-5-carboxamide;
N-(1-{4-[(difluoromethyl)oxy]phenyl}propyl)-1-ethyl-4-{[4-(hydroxyimino)cyclohexyl]amino}-1H-pyrazolo[3,4-b]pyridine-5-carboxamide;
1-ethyl-4-{[4-(hydroxyimino)cyclohexyl]amino}-N-{1-[4-(trifluoromethyl)phenyl]propyl}-1H-pyrazolo[3,4-b]pyridine-5-carboxamide;
N-[1-(3,4-dimethylphenyl)propyl]-1-ethyl-4-{[4-(hydroxyimino)cyclohexyl]amino}-1H-pyrazolo[3,4-b]pyridine-5-carboxamide;
1-ethyl-4-{[4-(hydroxyimino)cyclohexyl]amino}-N-[(1R)-1-phenylpropyl]-1H-pyrazolo[3,4-b]pyridine-5-carboxamide;
1-ethyl-4-{[4-(hydroxyimino)cyclohexyl]amino}-N-{(1R)-1-[3-(methyloxy)phenyl]ethyl}-1H-pyrazolo[3,4-b]pyridine-5-carboxamide;
N-[1-(2,3-dimethylphenyl)ethyl]-1-ethyl-4-{[4-(hydroxyimino)cyclohexyl]amino}-1H-pyrazolo[3,4-b]pyridine-5-carboxamide;
N-[1-(2,4-dimethylphenyl)ethyl]-1-ethyl-4-{[4-(hydroxyimino)cyclohexyl]amino}-1H-pyrazolo[3,4-b]pyridine-5-carboxamide;
N-[1-(4-chloro-2-fluorophenyl)ethyl]-1-ethyl-4-{[4-(hydroxyimino)cyclohexyl]amino}-1H-pyrazolo[3,4-b]pyridine-5-carboxamide;
N-[1-(3-chloro-4-methylphenyl)ethyl]-1-ethyl-4-{[4-(hydroxyimino)cyclohexyl]amino}-1H-pyrazolo[3,4-b]pyridine-5-carboxamide;
N-[1-(2,3-dimethylphenyl)propyl]-1-ethyl-4-{[4-(hydroxyimino)cyclohexyl]amino}-1H-pyrazolo[3,4-b]pyridine-5-carboxamide;
N-[1-(2,4-dimethylphenyl)propyl]-1-ethyl-4-{[4-(hydroxyimino)cyclohexyl]amino}-1H-pyrazolo[3,4-b]pyridine-5-carboxamide;
N-[1-(4-chloro-2-fluorophenyl)propyl]-1-ethyl-4-{[4-(hydroxyimino)cyclohexyl]amino}-1H-pyrazolo[3,4-b]pyridine-5-carboxamide;
N-[1-(3-chloro-4-methylphenyl)propyl]-1-ethyl-4-{[4-(hydroxyimino)cyclohexyl]amino}-1H-pyrazolo[3,4-b]pyridine-5-carboxamide;
1-ethyl-4-{[4-(hydroxyimino)cyclohexyl]amino}-N-[1-(3-hydroxyphenyl)ethyl]-1H-pyrazolo[3,4-b]pyridine-5-carboxamide;
1-ethyl-4-{[4-(hydroxyimino)cyclohexyl]amino}-N-[1-(3-hydroxyphenyl)propyl]-1H-pyrazolo[3,4-b]pyridine-5-carboxamide;
N-[1-(2,4-dimethylphenyl)ethyl]-1-ethyl-4-{[4-(hydroxyimino)cyclohexyl]amino}-1H-pyrazolo[3,4-b]pyridine-5-carboxamide;
N-[1-(2,4-dimethylphenyl)ethyl]-1-ethyl-4-{[4-(hydroxyimino)cyclohexyl]amino}-1H-pyrazolo[3,4-b]pyridine-5-carboxamide;
N-[1-(3,5-dimethylphenyl)ethyl]-1-ethyl-4-{[4-(hydroxyimino)cyclohexyl]amino}-1H-pyrazolo[3,4-b]pyridine-5-carboxamide;
N-[1-(3,5-dimethylphenyl)ethyl]-1-ethyl-4-{[4-(hydroxyimino)cyclohexyl]amino}-1H-pyrazolo[3,4-b]pyridine-5-carboxamide;
1-ethyl-4-{[4-(hydroxyimino)cyclohexyl]amino}-N-(1-{4-[(1-methylethyl)oxy]phenyl}ethyl)-1H-pyrazolo[3,4-b]pyridine-5-carboxamide;
1-ethyl-4-{[4-(hydroxyimino)cyclohexyl]amino}-N-(1-{4-[(1-methylethyl)oxy]phenyl}ethyl)-1H-pyrazolo[3,4-b]pyridine-5-carboxamide;
1-ethyl-N-[1-(4-fluorophenyl)ethyl]-4-{[4-(hydroxyimino)cyclohexyl]amino}-1H-pyrazolo[3,4-b]pyridine-5-carboxamide;
1-ethyl-N-[1-(4-fluorophenyl)ethyl]-4-{[4-(hydroxyimino)cyclohexyl]amino}-1H-pyrazolo[3,4-b]pyridine-5-carboxamide;
N-[1-(4-chlorophenyl)propyl]-1-ethyl-4-{[(1S,3R)— and/or (1R,3S)-3-hydroxycyclohexyl]amino}-1H-pyrazolo [3,4-b]pyridine-5-carboxamide;
1-ethyl-4-{[(1S,3R)— and/or (1R,3S)-3-hydroxycyclohexyl]amino}-N-[(1R)-1-(4-methylphenyl)ethyl]-1H-pyrazolo[3,4-b]pyridine-5-carboxamide;
N-[1-(2,4-dimethylphenyl)ethyl]-1-ethyl-4-{[(1S,3R)— and/or (1R,3S)-3-hydroxycyclohexyl]amino}-1H-pyrazolo[3,4-b]pyridine-5-carboxamide; (Isomer 1)
N-[1-(2,4-dimethylphenyl)ethyl]-1-ethyl-4-{[(1S,3R)— and/or (1R,3S)-3-hydroxycyclohexyl]amino}-1H-pyrazolo[3,4-b]pyridine-5-carboxamide; (Isomer 2)
N-[1-(3,4-dimethylphenyl)propyl]-1-ethyl-4-{[(1S,3R)— and/or (1R,3S)-3-hydroxycyclohexyl]amino}-1H-pyrazolo[3,4-b]pyridine-5-carboxamide;
N-[1-(4-chlorophenyl)propyl]-1-ethyl-6-methyl-4-(tetrahydro-2H-pyran-4-ylamino)-1H-pyrazolo[3,4-b]pyridine-5-carboxamide;
N-[1-(4-chlorophenyl)ethyl]-1-ethyl-6-methyl-4-(tetrahydro-2H-pyran-4-ylamino)-1H-pyrazolo[3,4-b]pyridine-5-carboxamide;
N-[1-(4-chlorophenyl)ethyl]-1-ethyl-4-(tetrahydro-2H-pyran-4-ylamino)-1H-pyrazolo[3,4-b]pyridine-5-carboxamide; (Enantiomer 1)
N-[1-(4-chlorophenyl)ethyl]-1-ethyl-4-(tetrahydro-2H-pyran-4-ylamino)-1H-pyrazolo[3,4-b]pyridine-5-carboxamide; (Enantiomer 2)
N-[1-(4-chlorophenyl)propyl]-1-ethyl-4-(tetrahydro-2H-pyran-4-ylamino)-1H-pyrazolo[3,4-b]pyridine-5-carboxamide; (Enantiomer 1)
N-[1-(4-chlorophenyl)propyl]-1-ethyl-4-(tetrahydro-2H-pyran-4-ylamino)-1H-pyrazolo[3,4-b]pyridine-5-carboxamide; (Enantiomer 2)
1-ethyl-N-{1-[4-(ethyloxy)phenyl]ethyl}-4-(tetrahydro-2H-pyran-4-ylamino)-1H-pyrazolo[3,4-b]pyridine-5-carboxamide; (Enantiomer 1)
1-ethyl-N-{1-[4-(ethyloxy)phenyl]ethyl}-4-(tetrahydro-2H-pyran-4-ylamino)-1H-pyrazolo[3,4-b]pyridine-5-carboxamide; (Enantiomer 2)
N-[1-(2,4-dimethylphenyl)ethyl]-1-ethyl-4-[(4-oxocyclohexyl)amino]-1H-pyrazolo[3,4-b]pyridine-5-carboxamide; (Enantiomer 1)
N-[1-(2,4-dimethylphenyl)ethyl]-1-ethyl-4-[(4-oxocyclohexyl)amino]-1H-pyrazolo[3,4-b]pyridine-5-carboxamide; (Enantiomer 2)
N-[1-(3,5-dimethylphenyl)ethyl]-1-ethyl-4-[(4-oxocyclohexyl)amino]-1H-pyrazolo[3,4-b]pyridine-5-carboxamide; (Enantiomer 1)
N-[1-(3,5-dimethylphenyl)ethyl]-1-ethyl-4-[(4-oxocyclohexyl)amino]-1H-pyrazolo[3,4-b]pyridine-5-carboxamide; (Enantiomer 2)
1-ethyl-N-(1-{4-[(1-methylethyl)oxy]phenyl}ethyl)-4-[(4-oxocyclohexyl)amino]-1H-pyrazolo[3,4-b]pyridine-5-carboxamide; (Enantiomer 1)
1-ethyl-N-(1-{4-[(1-methylethyl)oxy]phenyl}ethyl)-4-[(4-oxocyclohexyl)amino]-1H-pyrazolo[3,4-b]pyridine-5-carboxamide; (Enantiomer 2)
1-ethyl-N-[1-(4-fluorophenyl)ethyl]-4-[(4-oxocyclohexyl)amino]-1H-pyrazolo[3,4-b]pyridine-5-carboxamide; (Enantiomer 1)
1-ethyl-N-[1-(4-fluorophenyl)ethyl]-4-[(4-oxocyclohexyl)amino]-1H-pyrazolo[3,4-b]pyridine-5-carboxamide; (Enantiomer 2)
N-[1-(2,4-dimethylphenyl)propyl]-1-ethyl-4-(tetrahydro-2H-pyran-4-ylamino)-1H-pyrazolo[3,4-b]pyridine-5-carboxamide; (Enantiomer 1)
N-[1-(2,4-dimethylphenyl)propyl]-1-ethyl-4-(tetrahydro-2H-pyran-4-ylamino)-1H-pyrazolo[3,4-b]pyridine-5-carboxamide; (Enantiomer 2)
1-ethyl-4-{[(1S,3R)— and/or (1R,3S)-3-hydroxycyclohexyl]amino}-N-[(1-(4-methylphenyl)ethyl]-1H-pyrazolo[3,4-b]pyridine-5-carboxamide; (Diastereoisomer 1)
1-ethyl-4-{[(1S,3R)— and/or (1R,3S)-3-hydroxycyclohexyl]amino}-N-[(1R)-1-(4-methylphenyl)ethyl]-1H-pyrazolo[3,4-b]pyridine-5-carboxamide; (Diastereoisomer 2);
4-{[1-(aminocarbonyl)-4-piperidinyl]amino}-1-ethyl-N-[(1R)-1-(4-methylphenyl)ethyl]-1H-pyrazolo [3,4-b]pyridine-5-carboxamide;
4-{[1-(aminocarbonyl)-4-piperidinyl]amino}-1-ethyl-N-[(1R)-1-phenylethyl]-1H-pyrazolo[3,4-b]pyridine-5-carboxamide;
4-{[1-(aminocarbonyl)-4-piperidinyl]amino}-N-[1-(3-chloro-4-methylphenyl)propyl]-1-ethyl-1H-pyrazolo[3,4-b]pyridine-5-carboxamide;
4-{[1-(aminocarbonyl)-4-piperidinyl]amino}-N-[1-(4-chloro-2-fluorophenyl)propyl]-1-ethyl-1H-pyrazolo [3,4-b]pyridine-5-carboxamide; or
4-{[4-(aminocarbonyl)cyclohexyl]amino}-1-ethyl-N-[(1R)-1-phenylethyl]-1H-pyrazolo [3,4-b]pyridine-5-carboxamide;
or a salt thereof.

121. A compound of formula (I) according to claim 73 which is:
N-[(1S)-1-(2,4-dimethylphenyl)propyl]-1-ethyl-4-(tetrahydro-2H-pyran-4-ylamino)-1H-pyrazolo[3,4-b]pyridine-5-carboxamide;
N-[(1R)-1-(2,5-dimethylphenyl)ethyl]-1-ethyl-4-(tetrahydro-2H-pyran-4-ylamino)-1H-pyrazolo [3,4-b]pyridine-5-carboxamide;
1-ethyl-4-(tetrahydro-2H-pyran-4-ylamino)-N-[(1R)-1-(2,4,6-trimethylphenyl)ethyl]-1 H-pyrazolo [3,4-b]pyridine-5-carboxamide;
1-ethyl-N-[(1R)-1-(2-ethylphenyl)ethyl]-4-(tetrahydro-2H-pyran-4-ylamino)-1H-pyrazolo[3,4-b]pyridine-5-carboxamide;
1-ethyl-N-[(1R)-1-(4-ethylphenyl)ethyl]-4-(tetrahydro-2H-pyran-4-ylamino)-1H-pyrazolo[3,4-b]pyridine-5-carboxamide;
1-ethyl-N-[(1R)-1-(4-ethylphenyl)propyl]-4-(tetrahydro-2H-pyran-4-ylamino)-1H-pyrazolo [3,4-b]pyridine-5-carboxamide;
1-ethyl-N-{(1R)-1-[4-(1-methylethyl)phenyl]propyl}-4-(tetrahydro-2H-pyran-4-ylamino)-1H-pyrazolo [3,4-b]pyridine-5-carboxamide;
N-[(1R)-1-(2,6-dimethylphenyl)propyl]-1-ethyl-4-(tetrahydro-2H-pyran-4-ylamino)-1H-pyrazolo[3,4-b]pyridine-5-carboxamide;
4-{[1-(aminocarbonyl)-4-piperidinyl]amino}-N-[(1R)-1-(2,5-dimethylphenyl)ethyl]-1-ethyl-1H-pyrazolo[3,4-b]pyridine-5-carboxamide;
4-{[1-(aminocarbonyl)-4-piperidinyl]amino}-1-ethyl-N-{(1R)-1-[4-(1-methylethyl)phenyl]propyl}-1H-pyrazolo[3,4-b]pyridine-5-carboxamide;
4-{[1-(aminocarbonyl)-4-piperidinyl]amino}-N-[(1R)-1-(4-chloro-2-fluorophenyl)propyl]-1-ethyl-1H-pyrazolo[3,4-b]pyridine-5-carboxamide;
4-{[1-(aminocarbonyl)-4-piperidinyl]amino}-N-[(1R)-1-(2,6-dimethylphenyl)propyl]-1-ethyl-1H-pyrazolo[3,4-b]pyridine-5-carboxamide;
4-{[4-(aminocarbonyl)cyclohexyl]amino}-N-[1-(4-chlorophenyl)propyl]-1-ethyl-1H-pyrazolo[3,4-b]pyridine-5-carboxamide;
4-{[4-(aminocarbonyl)cyclohexyl]amino}-1-ethyl-N-[(1R)-1-phenylpropyl]-1H-pyrazolo[3,4-b]pyridine-5-carboxamide;
4-{[4-(aminocarbonyl)cyclohexyl]amino}-N-(1-{4-[(difluoromethyl)oxy]phenyl}ethyl)-1-ethyl-1H-pyrazolo[3,4-b]pyridine-5-carboxamide;
4-{[4-(aminocarbonyl)cyclohexyl]amino}-N-[1-(4-chlorophenyl)ethyl]-1-ethyl-1H-pyrazolo[3,4-b]pyridine-5-carboxamide;
4-{[4-(aminocarbonyl)cyclohexyl]amino}-1-ethyl-N-[1-(4-fluorophenyl)propyl]-1 H-pyrazolo[3,4-b]pyridine-5-carboxamide;
4-{[4-(aminocarbonyl)cyclohexyl]amino}-N-[(1R)-1-(4-bromophenyl)ethyl]-1-ethyl-1H-pyrazolo[3,4-b]pyridine-5-carboxamide;
4-{[cis-4-(aminocarbonyl)cyclohexyl]amino}-N-[(1R)-1-(2,4-dimethylphenyl)propyl]-1-ethyl-1H-pyrazolo[3,4-b]pyridine-5-carboxamide;
4-{[cis-4-(aminocarbonyl)cyclohexyl]amino}-1-ethyl-N-[(1R)-1-(4-methylphenyl)ethyl]-1H-pyrazolo[3,4-b]pyridine-5-carboxamide;
4-{[cis-4-(aminocarbonyl)cyclohexyl]amino}-1-ethyl-N-[(1R)-1-phenylethyl]-1H-pyrazolo[3,4-b]pyridine-5-carboxamide;
4-{[cis-4-(aminocarbonyl)cyclohexyl]amino}-N-[(1R)-1-(4-bromophenyl)ethyl]-1-ethyl-1H-pyrazolo[3,4-b]pyridine-5-carboxamide;
4-{[trans-4-(aminocarbonyl)cyclohexyl]amino}-N-[(1R)-1-(2,4-dimethylphenyl)propyl]-1-ethyl-1H-pyrazolo[3,4-b]pyridine-5-carboxamide;
4-{[trans-4-(aminocarbonyl)cyclohexyl]amino}-1-ethyl-N-[(1R)-1-(4-methylphenyl)ethyl]-1H-pyrazolo[3,4-b]pyridine-5-carboxamide;
4-{[trans-4-(aminocarbonyl)cyclohexyl]amino}-1-ethyl-N-[(1R)-1-phenylethyl]-1 H-pyrazolo[3,4-b]pyridine-5-carboxamide;
4-{[trans-4-(aminocarbonyl)cyclohexyl]amino}-N-[(1R)-1-(4-bromophenyl)ethyl]-1-ethyl-1H-pyrazolo[3,4-b]pyridine-5-carboxamide;
4-{[(3S)-1-(aminocarbonyl)pyrrolidin-3-yl]amino}-N-[1-(2,4-dimethylphenyl)propyl]-1-ethyl-1H-pyrazolo[3,4-b]pyridine-5-carboxamide;
4-{[(3S)-1-(aminocarbonyl)pyrrolidin-3-yl]amino}-1-ethyl-N-[(1R)-1-(4-methylphenyl)ethyl]-1H-pyrazolo[3,4-b]pyridine-5-carboxamide;
4-{[(3S)-1-(aminocarbonyl)pyrrolidin-3-yl]amino}-N-[1-(3,4-dimethylphenyl)propyl]-1-ethyl-1H-pyrazolo[3,4-b]pyridine-5-carboxamide;
4-{[(3S)-1-(aminocarbonyl)pyrrolidin-3-yl]amino}-N-[(1R)-1-(4-bromophenyl)ethyl]-1-ethyl-1H-pyrazolo[3,4-b]pyridine-5-carboxamide;
4-{[(3R)-1-(aminocarbonyl)pyrrolidin-3-yl]amino}-N-[1-(2,4-dimethylphenyl)propyl]-1-ethyl-1H-pyrazolo[3,4-b]pyridine-5-carboxamide;
4-{[(3R)-1-(aminocarbonyl)pyrrolidin-3-yl]amino}-1-ethyl-N-[(1R)-1-(4-methylphenyl)ethyl]-1H-pyrazolo[3,4-b]pyridine-5-carboxamide;
4-{[(3R)-1-(aminocarbonyl)pyrrolidin-3-yl]amino}-N-[1-(3,4-dimethylphenyl)propyl]-1-ethyl-1H-pyrazolo[3,4-b]pyridine-5-carboxamide;
4-{[(3R)-1-(aminocarbonyl)pyrrolidin-3-yl]amino}-N-[(1R)-1-(4-bromophenyl)ethyl]-1-ethyl-1H-pyrazolo[3,4-b]pyridine-5-carboxamide;
4-{[cis-3-(aminocarbonyl)cyclobutyl]amino}-1-ethyl-N-[(1R)-1-(4-methylphenyl)ethyl]-1H-pyrazolo[3,4-b]pyridine-5-carboxamide;
4-{[cis-3-(aminocarbonyl)cyclobutyl]amino}-N-[1-(2,4-dimethylphenyl)propyl]-1-ethyl-1H-pyrazolo[3,4-b]pyridine-5-carboxamide;
4-[(trans-4-acetylcyclohexyl)amino]-1-ethyl-N-[(1R)-1-(4-methylphenyl)ethyl]-1H-pyrazolo[3,4-b]pyridine-5-carboxamide;
4-[(4-acetylcyclohexyl)amino]-N-[(1R)-1-(2,4-dimethylphenyl)propyl]-1-ethyl-1H-pyrazolo[3,4-b]pyridine-5-carboxamide;
4-[(cis-4-acetylcyclohexyl)amino]-1-ethyl-N-[(1R)-1-(4-methylphenyl)ethyl]-1H-pyrazolo[3,4-b]pyridine-5-carboxamide;
1-ethyl-4-{[trans-3-hydroxycyclohexyl]amino}-N-[(1R)-1-(4-methylphenyl)ethyl]-1 H-pyrazolo [3,4-b]pyridine-5-carboxamide;
N-[(1S)-1-(2,4-dimethylphenyl)ethyl]-1-ethyl-4-{[trans-3-hydroxycyclohexyl]amino}-1 H-pyrazolo[3,4-b]pyridine-5-carboxamide;
N-[(1 R)-1-(2,4-dimethylphenyl)ethyl]-1-ethyl-4-{[trans-3-hydroxycyclohexyl]amino}-1 H-pyrazolo[3,4-b]pyridine-5-carboxamide;
N-[(1 R)-1-(4-bromophenyl)ethyl]-1-ethyl-4-{[trans-3-hydroxycyclohexyl]amino}-1 H-pyrazolo[3,4-b]pyridine-5-carboxamide;
N-[1-(3,4-dimethylphenyl)propyl]-1-ethyl-4-{[trans-3-hydroxycyclohexyl]amino}-1H-pyrazolo[3,4-b]pyridine-5-carboxamide;
N-[4-(dimethylamino)-1-(3-methylphenyl)-4-oxobutyl]-1-ethyl-4-(tetrahydro-2H-pyran-4-ylamino)-1H-pyrazolo[3,4-b]pyridine-5-carboxamide;
4-{[1-(aminocarbonyl)-4-piperidinyl] amino}-N-[4-(dimethylamino)-1-(3-methylphenyl)-4-oxobutyl]-1-ethyl-1H-pyrazolo[3,4-b]pyridine-5-carboxamide;
1-ethyl-N-[(1R)-1-(4-methylphenyl)ethyl]-4-(4-piperidinylamino)-1 H-pyrazolo[3,4-b]pyridine-5-carboxamide; hydrochloride, or
N-[1-(2,4-dimethylphenyl)propyl]-1-ethyl-4-(4-piperidinylamino)-1 H-pyrazolo[3,4-b]pyridine-5-carboxamide; hydrochloride;
or a salt thereof.

122. A compound of formula (I) according to claim 73 which is:
1-ethyl-N-[(1R)-1-(4-methylphenyl)ethyl]-4-(tetrahydro-2H-pyran-4-ylamino)-1H-pyrazolo[3,4-b]pyridine-5-carboxamide;
N-[(1R)-1-(4-bromophenyl)ethyl]-1-ethyl-4-(tetrahydro-2H-pyran-4-ylamino)-1H-pyrazolo[3,4-b]pyridine-5-carboxamide;
N-[1-(2,4-dimethylphenyl)propyl]-1-ethyl-4-(tetrahydro-2H-pyran-4-ylamino)-1H-pyrazolo[3,4-b]pyridine-5-carboxamide;
1-ethyl-4-{[(1S,3R)— and/or (1R,3S)-3-hydroxycyclohexyl]amino}-N-[(1R)-1-(4-methylphenyl)ethyl]-1H-pyrazolo[3,4-b]pyridine-5-carboxamide, or its hydrochloride salt;
1-ethyl-4-{[(1S,3R)— and/or (1R,3S)-3-hydroxycyclohexyl]amino}-N-[(1R)-1-(4-methylphenyl)ethyl]-1H-pyrazolo[3,4-b]pyridine-5-carboxamide; (diastereoisomer 2);
N-[1-(2,4-dimethylphenyl)propyl]-1-ethyl-4-(tetrahydro-2H-pyran-4-ylamino)-1H-pyrazolo[3,4-b]pyridine-5-carboxamide (Enantiomer 2), or its hydrochloride salt;
4-{[1-(aminocarbonyl)-4-piperidinyl]amino}-N-[(1R)-1-(4-bromophenyl)ethyl]-1-ethyl-1H-pyrazolo[3,4-b]pyridine-5-carboxamide;
4-{[1-(aminocarbonyl)-4-piperidinyl]amino}-N-[1-(2,4-dimethylphenyl)propyl]-1-ethyl-1H-pyrazolo[3,4-b]pyridine-5-carboxamide;
N-[(1R)-1-(2,4-dimethylphenyl)propyl]-1-ethyl-4-(tetrahydro-2H-pyran-4-ylamino)-1H-pyrazolo[3,4-b]pyridine-5-carboxamide;
1-ethyl-N-[(1R)-1-(4-methylphenyl)propyl]-4-(tetrahydro-2H-pyran-4-ylamino)-1H-pyrazolo[3,4-b]pyridine-5-carboxamide;
N-[(1R)-1-(4-chloro-2-fluorophenyl)propyl]-1-ethyl-4-(tetrahydro-2H-pyran-4-ylamino)-1H-pyrazolo[3,4-b]pyridine-5-carboxamide;
N-[(1R)-1-(2,5-dimethylphenyl)propyl]-1-ethyl-4-(tetrahydro-2H-pyran-4-ylamino)-1H-pyrazolo[3,4-b]pyridine-5-carboxamide;
1-ethyl-N-[(1R)-1-(2-ethylphenyl)propyl]-4-(tetrahydro-2H-pyran-4-ylamino)-1 H-pyrazolo[3,4-b]pyridine-5-carboxamide;
1-ethyl-4-(tetrahydro-2H-pyran-4-ylamino)-N-[(1R)-1-(2,4,6-trimethylphenyl)propyl]-1H-pyrazolo[3,4-b]pyridine-5-carboxamide;
4-{[1-(aminocarbonyl)-4-piperidinyl]amino}-1-ethyl-N-[(1R)-1-(4-ethylphenyl)ethyl]-1 H-pyrazolo[3,4-b]pyridine-5-carboxamide;
4-{[1-(aminocarbonyl)-4-piperidinyl]amino}-1-ethyl-N-[(1R)-1-(2-ethylphenyl)ethyl]-1H-pyrazolo[3,4-b]pyridine-5-carboxamide;
4-{[1-(aminocarbonyl)-4-piperidinyl]amino}-1-ethyl-N-[(1R)-1-(2,4,6-trimethylphenyl)ethyl]-1H-pyrazolo[3,4-b]pyridine-5-carboxamide;
4-{[1-(aminocarbonyl)-4-piperidinyl]amino}-N-[(1R)-1-(2,4-dimethylphenyl)propyl]-1-ethyl-1H-pyrazolo[3,4-b]pyridine-5-carboxamide;
4-{[1-(aminocarbonyl)-4-piperidinyl]amino}-N-[1-(4-chlorophenyl)ethyl]-1-ethyl-1H-pyrazolo [3,4-b]pyridine-5-carboxamide;
4-{[1-(aminocarbonyl)-4-piperidinyl]amino}-1-ethyl-N-[(1R)-1-phenylpropyl]-1H-pyrazolo[3,4-b]pyridine-5-carboxamide;
4-{[1-(aminocarbonyl)-4-piperidinyl]amino}-N-[1-(4-chlorophenyl)propyl]-1-ethyl-1H-pyrazolo[3,4-b]pyridine-5-carboxamide;
4-{[1-(aminocarbonyl)-4-piperidinyl]amino}-1-ethyl-N-[1-(4-fluorophenyl)propyl]-1H-pyrazolo[3,4-b]pyridine-5-carboxamide;
4-{[1-(aminocarbonyl)-4-piperidinyl]amino}-1-ethyl-N-[(1R)-1-(4-methylphenyl)propyl]-1H-pyrazolo[3,4-b]pyridine-5-carboxamide;
4-{[1-(aminocarbonyl)-4-piperidinyl]amino}-1-ethyl-N-[(1R)-1-(4-ethylphenyl)propyl]-1H-pyrazolo[3,4-b]pyridine-5-carboxamide;
4-{[1-(aminocarbonyl)-4-piperidinyl]amino}-N-[(1R)-1-(2,5-dimethylphenyl)propyl]-1-ethyl-1H-pyrazolo[3,4-b]pyridine-5-carboxamide;
4-{[1-(aminocarbonyl)-4-piperidinyl]amino}-1-ethyl-N-[(1R)-1-(2-ethylphenyl)propyl]-1H-pyrazolo [3,4-b]pyridine-5-carboxamide;
4-{[1-(aminocarbonyl)-4-piperidinyl]amino}-1-ethyl-N-[(1R)-1-(2,4,6-trimethylphenyl)propyl]-1 H-pyrazolo[3,4-b]pyridine-5-carboxamide;
or a pharmaceutically acceptable salt thereof

123. A compound as claimed in claim 73 which is in a particle-size-reduced form, wherein the particle size of the size-reduced compound or salt is defined by a D50 value of about 0.5 to about 10 microns.

124. A pharmaceutical composition comprising a compound of formula (I) as defined in claim 73 or a pharmaceutically acceptable salt thereof, and one or more pharmaceutically acceptable excipients.

125. A method for the prophylaxis of and/or treating an inflammatory or allergic disease in a human in need thereof, which method comprises administering to said human an effective amount of a compound according to claim 73 or a pharmaceutically acceptable salt or derivative thereof.

126. The method of claim 125 and wherein the disease is chronic obstructive pulmonary disease (COPD), asthma, rheumatoid arthritis, allergic rhinitis or atopic dermatitis.

127. A composition comprising a compound of formula (I) as defined in claim 73 or a pharmaceutically acceptable salt thereof, and one or more of a second pharmaceutically active compound which is a β₂-adrenoreceptor agonist, an anti-histamine, an anti-allergic, an anti-inflammatory agent, or a muscarinic (M) receptor antagonist.

128. A compound of formula (IB) or a salt thereof: 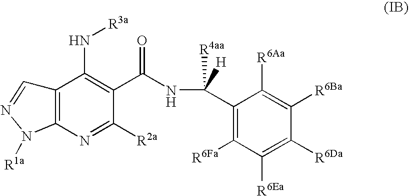
wherein:
R^{1a} is C₂₋₃alkyl, fluoroethyl or —CH₂CH₂OH;
R^{2a} is hydrogen or methyl;
NHR^{3a} is of sub-formula (p14), in which the —NH— connection point of the NHR^{3a} group to the 4-position of the pyrazolopyridine of formula (IB) is underlined: 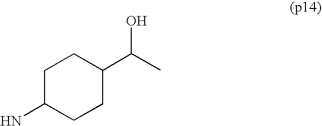
R^{4aa} is methyl, ethyl, fluoromethyl, —CH₂OH, or —CH₂OMe;
R^{6Aa}, R^{6Ba}, R^{6Da}, R^{6Ea} and R^{6Fa}, independently of each other, are hydrogen, fluoro, chloro, bromo, iodo, methyl, ethyl, n-propyl, isopropyl, isobutyl, trifluoromethyl, —CH₂OH, methoxy, ethoxy, n-propoxy, isopropoxy, fluoromethoxy, nitro, OH, C₁₋₃alkylS(O)₂—, C₁₋₂alkylS(O)₂—NH—, —CONH₂, cyano, or C₁₋₂alkylS(O)₂—CH₂—; provided that two or more of R^{6Aa}, R^{6Ba}, R^{6Da}, R^{6Ea} and R^{6Fa} are hydrogen;
and wherein, in Formula (IB), on a molarity basis, more than 50% of the compound present has the stereochemistry shown at the carbon atom bearing the R^{4aa} group.

129. A compound as claimed in claim 128 wherein:
R^{1a} is ethyl;
R^{2a} is H;
R^{4aa} is methyl or ethyl; and
R^{6Aa}, R^{6Ba}, R^{6Da}, R^{6Ea} and R^{6Fa}, independently of each other, are hydrogen, fluoro, chloro, bromo, iodo, methyl, ethyl, n-propyl, isopropyl, trifluoromethyl, —CH₂OH, methoxy, ethoxy, n-propoxy, difluoromethoxy, OH or MeS(O)₂—; provided that three or more of R^{6Aa}, R^{6Ba}, R^{6Da}, R^{6Ea} and R^{6Fa} are hydrogen.

130. A compound as claimed in claim 129 wherein the NHR^{3a} group of sub-formula (p14) is in the cis configuration.

131. A compound as claimed in claim 91 wherein R¹ is ethyl.

132. A compound as claimed in claim 92 wherein R¹ is ethyl.

133. A compound as claimed in claim 132 wherein:
R⁵ is hydrogen; C₁₋₅alkyl; C₁₋₂fluoroalkyl; unsubstituted C₃₋₆cycloalkyl; or —(CH₂)ₙ⁴-unsubstituted-C₃-₆cycloalkyl wherein n⁴ is 1 or 2;
or R⁵ is C₁₋₃alkyl substituted by one R¹¹ wherein R¹¹ is hydroxy; C₁₋₄alkoxy; C₁fluoroalkoxy; —NR¹²R¹³; —NR¹⁵—C(O)R¹⁶; or —NR¹⁵—S(O)₂R¹⁶;
or R⁵ is —(CH₂)ₙ¹¹—C(O)NR¹²R¹³, —(CH₂)ₙ¹¹—C(O)OR¹⁶, —(CH₂)ₙ¹¹—C(O)OH, or —(CH₂)ₙ¹¹—CN; wherein n¹¹ is 0, 1 or 2 and wherein for each R⁵ group n¹¹ is independent of the value of n¹¹ in other R⁵ groups;
or R⁵ is —(CH₂)ₙ¹³-Het, wherein n¹³ is 0 or 1 and Het is 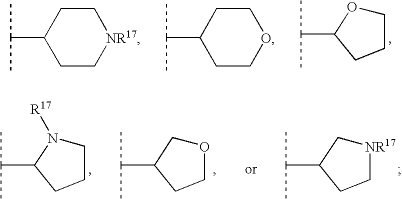
or R⁵ is phenyl or —CH₂-phenyl wherein the phenyl ring is unsubstituted or substituted with one or two substituents independently selected from the group consisting of fluoro, chloro, C₁₋₂alkyl, C₁fluoroalkyl, C₁₋₂alkoxy, and C₁ fluoroalkoxy;
or R⁴ and R⁵ taken together are —(CH₂)₂—O—(CH₂)₂— or —(CH₂)p¹—in which p¹ is 2, 4 or 5.

134. A compound as claimed in claim 118 wherein R¹ is ethyl and NHR³ is sub-formula (c), (c1), (c4), (c5), (h), (i), (), (k), (k2), (m1), (n), (o), (o2), (o3), (p2), (p5), (p6), (p9), (p11) or (q) as defined in claim 22.

135. A compound as claimed in claim 119 wherein R¹ is ethyl and NHR³ is of sub-formula (c), (c1), (c4), (c5), (h), (i), (), (k), (k2), (m1), (n), (o), (o2), (o3), (p2), (p5), (p6), (p9), (p11) or (q).

136. A method of treatment or prophylaxis of an inflammatory or allergic disease, cognitive impairment in Alzheimer's disease, or depression, in a human in need thereof, which method comprises administering to the human a therapeutically effective amount of a compound of formula (I) as defined in claim 133 or a pharmaceutically acceptable salt thereof.
